SEQUENCE LISTING (79 sequences)

SEQ ID NO: 1 - protein, 159 aa, Escherichia coli [sequence omitted]

SEQ ID NO: 2 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 3 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 4 - protein, 327 aa, Homo sapiens
MEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 5 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 6 - protein, 595 aa, Homo sapiens [sequence omitted]

SEQ ID NO: 7 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 8 - DNA, 20 bp, Artificial Sequence
  source: /note=“Description of Artificial Sequence Synthetic oligonucleotide”
agcaacagcgccgctaccag

SEQ ID NO: 9 - DNA, 20 bp, Artificial Sequence
  source: /note=“Description of Artificial Sequence Synthetic oligonucleotide”
caccagagtaacagtctgag

SEQ ID NO: 10 - DNA, 20 bp, Artificial Sequence
  source: /note=“Description of Artificial Sequence Synthetic oligonucleotide”
atcttacaggaactccagga

SEQ ID NO: 11 - DNA, 20 bp, Artificial Sequence
  source: /note=“Description of Artificial Sequence Synthetic oligonucleotide”
gagtccgagcagaagaagaa

SEQ ID NO: 12 - DNA, 20 bp, Artificial Sequence
  source: /note=“Description of Artificial Sequence Synthetic primer”
gaccagggaaaggaagggag

SEQ ID NO: 13 - DNA, 19 bp, Artificial Sequence
  source: /note=“Description of Artificial Sequence Synthetic primer”
gaacgggtgcaatgaggtc

SEQ ID NO: 14 - DNA, 18 bp, Artificial Sequence
  source: /note=“Description of Artificial Sequence Synthetic primer”
ttccctggcaaggtctga

SEQ ID NO: 15 - DNA, 19 bp, Artificial Sequence
  source: /note=“Description of Artificial Sequence Synthetic primer”
atcctcaaggtcacccacc

SEQ ID NO: 16 - DNA, 24 bp, Artificial Sequence
  source: /note=“Description of Artificial Sequence Synthetic primer”
gtctttctgtcttgtatcctttgg

SEQ ID NO: 17 - DNA, 20 bp, Artificial Sequence
  source: /note=“Description of Artificial Sequence Synthetic primer”
aatgttagtgcctttcaccc

SEQ ID NO: 18 - DNA, 28 bp, Artificial Sequence
  source: /note=“Description of Artificial Sequence Synthetic primer”
taaccctatgtagcctcagtcttcccat

SEQ ID NO: 19 - DNA, 30 bp, Artificial Sequence
  source: /note=“Description of Artificial Sequence Synthetic primer”
gcatcaaaacaaaagggagattggagacac

SEQ ID NO: 20 - protein, 9 aa, Artificial Sequence
  source: /note=“Description of Artificial Sequence Synthetic peptide”
GGSGGGSGG

SEQ ID NO: 21 - protein, 5 aa, Artificial Sequence
  source: /note=“Description of Artificial Sequence Synthetic peptide”
GSGSG

SEQ ID NO: 22 - DNA, 12172 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagagtaacagtctgaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 23 - DNA, 12172 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaagcaacagcgccgctaccaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 24 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaagcaacagcgccgctaccaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 25 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaagcaacagcgccgctaccaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 26 - DNA, 12907 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaagcaacagcgccgctaccaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcactggcgctcagccttactgccgaccaaatggtatcagctcttctggacgcagaacccccaattctttattccgagtacgaccccacacgcccgttcagtgaagcttccatgatgggcctccttacgaaccttgccgaccgggaactcgtgcacatgatcaattgggcgaagcgggtgccggggttcgtagatttgacacttcacgaccaagttcatctcttggaatgtgcttggatggagatattgatgatcggactcgtgtggaggtcaatggagcatcctggtaaacttcttttcgcacccaatctgctcttggatagaaatcagggtaagtgcgtcgagggtggcgttgaaatcttcgacatgctccttgcgacatccagccgattccgaatgatgaatcttcaaggagaggaatttgtctgtcttaagagcattatactcctcaatagtggagtttacaccttcttgtcctctacactgaaatcacttgaggaaaaagatcacatacatagggtgttggataaaatcacggatacactcatacatctgatggcaaaagcaggattgaccctgcaacagcagcacgaccgactggcccaactgctgttgatccttagccatatcagacacatgtctaacaaaaggatggaacatttgtacagcatgaaatgtaagaacgtagtgccactgtccgatttgttgctggaaatgctggacgctcatcggctcggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 27 - DNA, 12172 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagaagttcgagggcgacacccgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 28 - DNA, 12172 bp, Artificial Sequence; identical to SEQ ID NO: 22
  source: /note=“Description of Artificial Sequence Synthetic polynucleotide”

SEQ ID NO: 29 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagagtaacagtctgaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 30 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagagtaacagtctgaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 31 - DNA, 12172 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaatcttacaggaactccaggagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 32 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaatcttacaggaactccaggagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 33 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaatcttacaggaactccaggagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 34 - DNA, 12907 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaatcttacaggaactccaggagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcactggcgctcagccttactgccgaccaaatggtatcagctcttctggacgcagaacccccaattctttattccgagtacgaccccacacgcccgttcagtgaagcttccatgatgggcctccttacgaaccttgccgaccgggaactcgtgcacatgatcaattgggcgaagcgggtgccggggttcgtagatttgacacttcacgaccaagttcatctcttggaatgtgcttggatggagatattgatgatcggactcgtgtggaggtcaatggagcatcctggtaaacttcttttcgcacccaatctgctcttggatagaaatcagggtaagtgcgtcgagggtggcgttgaaatcttcgacatgctccttgcgacatccagccgattccgaatgatgaatcttcaaggagaggaatttgtctgtcttaagagcattatactcctcaatagtggagtttacaccttcttgtcctctacactgaaatcacttgaggaaaaagatcacatacatagggtgttggataaaatcacggatacactcatacatctgatggcaaaagcaggattgaccctgcaacagcagcacgaccgactggcccaactgctgttgatccttagccatatcagacacatgtctaacaaaaggatggaacatttgtacagcatgaaatgtaagaacgtagtgccactgtccgatttgttgctggaaatgctggacgctcatcggctcggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 35 - DNA, 12172 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagagtccgagcagaagaagaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 36 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagagtccgagcagaagaagaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 37 - DNA, 12949 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagagtccgagcagaagaagaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 38 - DNA, 12907 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagagtccgagcagaagaagaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcactggcgctcagccttactgccgaccaaatggtatcagctcttctggacgcagaacccccaattctttattccgagtacgaccccacacgcccgttcagtgaagcttccatgatgggcctccttacgaaccttgccgaccgggaactcgtgcacatgatcaattgggcgaagcgggtgccggggttcgtagatttgacacttcacgaccaagttcatctcttggaatgtgcttggatggagatattgatgatcggactcgtgtggaggtcaatggagcatcctggtaaacttcttttcgcacccaatctgctcttggatagaaatcagggtaagtgcgtcgagggtggcgttgaaatcttcgacatgctccttgcgacatccagccgattccgaatgatgaatcttcaaggagaggaatttgtctgtcttaagagcattatactcctcaatagtggagtttacaccttcttgtcctctacactgaaatcacttgaggaaaaagatcacatacatagggtgttggataaaatcacggatacactcatacatctgatggcaaaagcaggattgaccctgcaacagcagcacgaccgactggcccaactgctgttgatccttagccatatcagacacatgtctaacaaaaggatggaacatttgtacagcatgaaatgtaagaacgtagtgccactgtccgatttgttgctggaaatgctggacgctcatcggctcggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 39 - DNA, 12541 bp, Artificial Sequence
  source: /note=“Description of Artificial Sequence Synthetic polynucleotide”
ttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagaagttcgagggcgacacccgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgccc

SEQ ID NO: 40 - DNA, 13318 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagaagttcgagggcgacacccgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 41 - DNA, 12541 bp, Artificial Sequence
  source: /note=“Description of Artificial Sequence Synthetic polynucleotide”
ctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagagtccgagcagaagaagaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggttccggcgctactaacttcagcctgctgaagcaggctggggacgtggaggagaaccctggacctaggacgcgtttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaatcgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttc

SEQ ID NO: 42 - DNA, 9166 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcaacgcgtagatctctagctaatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccactagctactaccagatagcttggtactagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggaggtagtggtggaggcagtggtggttcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatcctgaatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacttcgaagccaccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaattcgaagcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 43 - DNA, 9212 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcaacgcgtagatctctagctaatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagataggcgtgtacggtgggaggcctatataagcagagctcgtttagtgaaccgtcagatcgcctggactagctactaccagatagcttggtactagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggaggtagtggtggaggcagtggtggttcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatcctgaatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacttcgaagccaccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaattcgaagcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 44 - DNA, 9169 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcaacgcgtagatctctagctaatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccactactaccagatagcttggtaccgagctctgatccactagtgccaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccggttcaggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatcctaaatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacttcgaagccaccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaattcgaagcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 45 - DNA, 10006 bp, Artificial Sequence
  source: /note=“Description of Artificial Sequence Synthetic polynucleotide”
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggaggtagtggtggaggcagtggtggttcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatccggagctactaacttcagcctgctgaagcaggctggagacgtggaggagaaccctggaccttctgagctgattaaggagaatatgcacatgaagctgtacatggaaggaactgtggacaatcatcactttaagtgcacatcggagggagaaggcaagccctacgaaggcacccagaccatgaggatcaaggtggttgagggcggaccgctgcccttcgccttcgatatcctggcgacttcattcctctacggaagcaaaacctttattaaccacactcagggtataccagacttctttaagcaatccttccctgagggttttacatgggagagagtcactacatatgaagatgggggcgtgctaaccgctactcaggacacctctttacaagatggatgtctcatctacaacgtaaaaattaggggggtgaacttcacatccaacggccctgtgatgcagaagaaaacattggggtgggaagcctttacggagacgctgtatccagctgatggcggactggaaggccggaatgatatggcccttaagttagttggtgggtcacatttgatagcaaacatcaagaccacatatcgtagtaagaaacccgctaaaaacctcaagatgcctggtgtctactatgttgactatagactggaacgaatcaaagaggcaaataatgagacctacgtcgagcagcatgaagtagcagtggcccgctactgcgacctcccaagcaaactggggcacaaacttaattgaatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 46 - DNA, 9964 bp, Artificial Sequence
  source: /note=“Description of Artificial Sequence Synthetic polynucleotide”
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggaggtagtggtggaggcagtggtggttcactggcgctcagccttactgccgaccaaatggtatcagctcttctggacgcagaacccccaattctttattccgagtacgaccccacacgcccgttcagtgaagcttccatgatgggcctccttacgaaccttgccgaccgggaactcgtgcacatgatcaattgggcgaagcgggtgccggggttcgtagatttgacacttcacgaccaagttcatctcttggaatgtgcttggatggagatattgatgatcggactcgtgtggaggtcaatggagcatcctggtaaacttcttttcgcacccaatctgctcttggatagaaatcagggtaagtgcgtcgagggtggcgttgaaatcttcgacatgctccttgcgacatccagccgattccgaatgatgaatcttcaaggagaggaatttgtctgtcttaagagcattatactcctcaatagtggagtttacaccttcttgtcctctacactgaaatcacttgaggaaaaagatcacatacatagggtgttggataaaatcacggatacactcatacatctgatggcaaaagcaggattgaccctgcaacagcagcacgaccgactggcccaactgctgttgatccttagccatatcagacacatgtctaacaaaaggatggaacatttgtacagcatgaaatgtaagaacgtagtgccactgtccgatttgttgctggaaatgctggacgctcatcggctcggatccggagctactaacttcagcctgctgaagcaggctggagacgtggaggagaaccctggaccttctgagctgattaaggagaatatgcacatgaagctgtacatggaaggaactgtggacaatcatcactttaagtgcacatcggagggagaaggcaagccctacgaaggcacccagaccatgaggatcaaggtggttgagggcggaccgctgcccttcgccttcgatatcctggcgacttcattcctctacggaagcaaaacctttattaaccacactcagggtataccagacttctttaagcaatccttccctgagggttttacatgggagagagtcactacatatgaagatgggggcgtgctaaccgctactcaggacacctctttacaagatggatgtctcatctacaacgtaaaaattaggggggtgaacttcacatccaacggccctgtgatgcagaagaaaacattggggtgggaagcctttacggagacgctgtatccagctgatggcggactggaaggccggaatgatatggcccttaagttagttggtgggtcacatttgatagcaaacatcaagaccacatatcgtagtaagaaacccgctaaaaacctcaagatgcctggtgtctactatgttgactatagactggaacgaatcaaagaggcaaataatgagacctacgtcgagcagcatgaagtagcagtggcccgctactgcgacctcccaagcaaactggggcacaaacttaattgaatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 47 - DNA, 12473 bp, Artificial Sequence
  source: /note=“Description of Artificial Sequence Synthetic polynucleotide”
atgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcaattaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagagtaacagtctgaggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtcgcgtagatctctagctaatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccactagtctactaccagatagcttggtaccgagctctgatccagccaccatgggatccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagtaaatcgggtagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacttcgaagccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaattcgaagcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctta

SEQ ID NO: 48 - DNA, 241 bp, Artificial Sequence
  source: /note=“Description of Artificial Sequence Synthetic polynucleotide”
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 49 - DNA, 212 bp, Artificial Sequence
  source: /note=“Description of Artificial Sequence Synthetic polynucleotide”
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 50 - DNA, 48 bp, Artificial Sequence
  source: /note=“Description of Artificial Sequence Synthetic oligonucleotide”
aagcgacctgccgccacaaagaaggctggacaggctaagaagaagaaa

SEQ ID NO: 51 - DNA, 24 bp, Artificial Sequence
  source: /note=“Description of Artificial Sequence Synthetic oligonucleotide”
gattacaaagacgatgacgataag

SEQ ID NO: 52 - DNA, 4101 bp, Artificial Sequence
  source: /note=“Description of Artificial Sequence Synthetic polynucleotide”
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 53 - DNA, 57 bp, Artificial Sequence
  source: /note=“Description of Artificial Sequence Synthetic oligonucleotide”
gctactaacttcagcctgctgaagcaggctggggacgtggaggagaaccctggacct

SEQ ID NO: 54 - DNA, 708 bp, Artificial Sequence
  source: /note=“Description of Artificial Sequence Synthetic polynucleotide”
ttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaa

SEQ ID NO: 55 - DNA, 145 bp, Artificial Sequence
  source: /note=“Description of Artificial Sequence Synthetic polynucleotide”
taatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagc

SEQ ID NO: 56 - DNA, 387 bp, Artificial Sequence
  source: /note=“Description of Artificial Sequence Synthetic polynucleotide”
gcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaac

SEQ ID NO: 57 - DNA, 25 bp, Artificial Sequence
  source: /note=“Description of Artificial Sequence Synthetic oligonucleotide”
tctagagggtatataatgggggcca

SEQ ID NO: 58 - DNA, 65 bp, Artificial Sequence
  source: /note=“Description of Artificial Sequence Synthetic oligonucleotide”
taggcgtgtacggtgggaggcctatataagcagagctcgtttagtgaaccgtcagatcgcctgga

SEQ ID NO: 59 - DNA, 1184 bp, Artificial Sequence
  source: /note=“Description of Artificial Sequence Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 60 - DNA, 306 bp, Artificial Sequence
  source: /note=“Description of Artificial Sequence Synthetic polynucleotide”
ctgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 61 - DNA, 696 bp, Artificial Sequence
  source: /note=“Description of Artificial Sequence Synthetic polynucleotide”
tctgagctgattaaggagaatatgcacatgaagctgtacatggaaggaactgtggacaatcatcactttaagtgcacatcggagggagaaggcaagccctacgaaggcacccagaccatgaggatcaaggtggttgagggcggaccgctgcccttcgccttcgatatcctggcgacttcattcctctacggaagcaaaacctttattaaccacactcagggtataccagacttctttaagcaatccttccctgagggttttacatgggagagagtcactacatatgaagatgggggcgtgctaaccgctactcaggacacctctttacaagatggatgtctcatctacaacgtaaaaattaggggggtgaacttcacatccaacggccctgtgatgcagaagaaaacattggggtgggaagcctttacggagacgctgtatccagctgatggcggactggaaggccggaatgatatggcccttaagttagttggtgggtcacatttgatagcaaacatcaagaccacatatcgtagtaagaaacccgctaaaaacctcaagatgcctggtgtctactatgttgactatagactggaacgaatcaaagaggcaaataatgagacctacgtcgagcagcatgaagtagcagtggcccgctactgcgacctcccaagcaaactggggcacaaacttaat

SEQ ID NO: 62 - DNA, 591 bp, Artificial Sequence
  source: /note=“Description of Artificial Sequence Synthetic polynucleotide”
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 63 - DNA, 309 bp, Artificial Sequence
  source: /note=“Description of Artificial Sequence Synthetic polynucleotide”
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggct

SEQ ID NO: 64 - DNA, 483 bp, Artificial Sequence
  source: /note=“Description of Artificial Sequence Synthetic polynucleotide”
aacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctg

SEQ ID NO: 65 - DNA, 777 bp, Artificial Sequence
  source: /note=“Description of Artificial Sequence Synthetic polynucleotide”
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 66 - DNA, 777 bp, Artificial Sequence
  source: /note=“Description of Artificial Sequence Synthetic polynucleotide”
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 67 - DNA, 777 bp, Artificial Sequence
  source: /note=“Description of Artificial Sequence Synthetic polynucleotide”
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 68 - DNA, 735 bp, Artificial Sequence
  source: /note=“Description of Artificial Sequence Synthetic polynucleotide”
tcactggcgctcagccttactgccgaccaaatggtatcagctcttctggacgcagaacccccaattctttattccgagtacgaccccacacgcccgttcagtgaagcttccatgatgggcctccttacgaaccttgccgaccgggaactcgtgcacatgatcaattgggcgaagcgggtgccggggttcgtagatttgacacttcacgaccaagttcatctcttggaatgtgcttggatggagatattgatgatcggactcgtgtggaggtcaatggagcatcctggtaaacttcttttcgcacccaatctgctcttggatagaaatcagggtaagtgcgtcgagggtggcgttgaaatcttcgacatgctccttgcgacatccagccgattccgaatgatgaatcttcaaggagaggaatttgtctgtcttaagagcattatactcctcaatagtggagtttacaccttcttgtcctctacactgaaatcacttgaggaaaaagatcacatacatagggtgttggataaaatcacggatacactcatacatctgatggcaaaagcaggattgaccctgcaacagcagcacgaccgactggcccaactgctgttgatccttagccatatcagacacatgtctaacaaaaggatggaacatttgtacagcatgaaatgtaagaacgtagtgccactgtccgatttgttgctggaaatgctggacgctcatcggctc

SEQ ID NO: 69 - protein, 16 aa, Artificial Sequence
  source: /note=“Description of Artificial Sequence Synthetic peptide”
KRPAATKKAGQAKKKK

SEQ ID NO: 70 - protein, 8 aa, Artificial Sequence
  source: /note=“Description of Artificial Sequence Synthetic peptide”
DYKDDDDK

SEQ ID NO: 71 - protein, 1367 aa, Artificial Sequence
  source: /note=“Description of Artificial Sequence Synthetic polypeptide”
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 72 - protein, 19 aa, Artificial Sequence
  source: /note=“Description of Artificial Sequence Synthetic peptide”
ATNFSLLKQAGDVEENPGP

SEQ ID NO: 73 - protein, 235 aa, Artificial Sequence
  source: /note=“Description of Artificial Sequence Synthetic polypeptide”
LSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 74 - protein, 129 aa, Artificial Sequence
  source: /note=“Description of Artificial Sequence Synthetic polypeptide”
APKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRIN

SEQ ID NO: 75 - protein, 102 aa, Artificial Sequence
  source: /note=“Description of Artificial Sequence Synthetic polypeptide”
LGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 76 - protein, 232 aa, Artificial Sequence
  source: /note=“Description of Artificial Sequence Synthetic polypeptide”
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 77 - protein, 161 aa, Artificial Sequence
  source: /note=“Description of Artificial Sequence Synthetic polypeptide”
NPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELQVSGANPMSSNKSISVYRDKLVKCGGISLLVQNTSWMLLLLLSLSLLQALDFISL

SEQ ID NO: 78 - protein, 259 aa, Artificial Sequence
  source: /note=“Description of Artificial Sequence Synthetic polypeptide”
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 79 - protein, 245 aa, Artificial Sequence
  source: /note=“Description of Artificial Sequence Synthetic polypeptide”
SLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHDRLAQLLLILSHIRHMSNKRMEHLYSMKCKNVVPLSDLLLEMLDAHRL

(19) United States
(12) Patent Application Publication
(10) Pub. No.: US 2022/0170011 A1
(43) Pub. Date: Jun. 2, 2022

(54) COMPOSITIONS AND METHODS FOR TUNABLE REGULATION OF CAS NUCLEASES

(71) Applicant: OBSIDIAN THERAPEUTICS, INC., Cambridge, MA (US)

(72) Inventors: Mara Christine INNISS, Beverly, MA (US); Grace Y. OLINGER, Cambridge, MA (US); Samantha FLEURY, Houston, TX (US); Jennifer Leah GORI, Jamaica Plain, MA (US)

(73) Assignee: OBSIDIAN THERAPEUTICS, INC., Cambridge, MA (US)

(21) Appl. No.: 17/548,076

(22) Filed: Dec. 10, 2021

Related U.S. Application Data
(63) Continuation of application No. PCT/US2021/038558, filed on Jun. 23, 2021.
(60) Provisional application No. 63/042,551, filed on Jun. 22, 2020.

(51) Int. Cl. C12N 15/11; C12N 9/22; C12N 15/90; C12N 15/86; A61K 35/22; A61K 31/7105

(52) U.S. Cl. CPC C12N 15/11; C12N 9/22; C12N 15/907; C12N 15/86; A61K 35/22; C07K 2319/09; C12N 2310/20; C12N 2740/15043; C12N 2800/80; C12N 2510/00; A61K 31/7105

(57) ABSTRACT

The present disclosure provides compositions and methods related to regulatable Cas systems. Such systems provide for ligand-dependent, modular and tunable Cas protein expression and activity.

### CROSS REFERENCE TO RELATED APPLICATIONS

This application claims benefit of priority to U.S. Provisional Application No. 63/042,551, filed Jun. 22, 2020. The entire contents of the aforementioned application are incorporated herein by reference in their entireties.

### REFERENCE TO THE SEQUENCE LISTING

The instant application contains a Sequence Listing which has been submitted electronically in ASCII format and is hereby incorporated by reference in its entirety. Said ASCII copy, created on Jun. 21, 2021, is named 268052_494055_SL.txt and is 485,500 bytes in size.

### FIELD

The present disclosure relates to systems, compositions and methods for tunable regulation of Cas nucleases. Provided in the present disclosure are systems and components thereof for direct ligand-dependent regulation of Cas protein expression and activity and ligand-dependent transcriptional regulation of Cas protein expression and activity. Also provided herein are polynucleotides, polypeptides, vectors, cells, compositions and methods for use in regulation of Cas nucleases.

### BACKGROUND

The prokaryotic clustered regularly interspaced short palindromic repeats (CRISPR)-Cas adaptive immune system has been adopted and repurposed for use in a broad range of applications as a powerful DNA targeting platform. This platform enables specific, RNA-guided manipulation of genomic sequences, offering the means and tools for design of new technologies in genome editing, regulation of gene expression, epigenetic modulation, genome imaging, and other forms of genome engineering. Importantly, gene editing and regulation of gene expression with CRISPR-Cas technology promises to deliver new treatments or even cures for previously intractable conditions. However, despite the versatility and transformative potential of the CRISPR-Cas platform, there remain concerns about safety and effectiveness that limit its implementation in medicine. Such concerns include, among other things, limited tools and methods that are available for more precise control of CRISPR-Cas technology and its applications. Thus, there is a need to develop new tools and approaches for regulating CRISPR-Cas systems for safe and effective use in therapeutic settings.

### SUMMARY

The present disclosure provides systems, compositions and methods for regulating CRISPR-Cas technology.

Systems of the disclosure include regulation of Cas through the use of drug responsive domains (DRDs). Systems include direct Cas-DRD regulation systems and Cas-transcription factor systems.

A direct Cas-DRD regulation system comprises one or more polynucleotides that comprise (1) a nucleic acid sequence that encodes a Cas protein; (2) a first promoter that drives expression of the Cas protein; (3) a nucleic acid sequence that encodes a drug responsive domain (DRD), wherein the Cas protein is operably linked to the DRD; (4) a guide RNA sequence; and (5) a promoter that mediates transcription of the guide RNA. A Cas-transcription factor system comprises one or more polynucleotides that comprise (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; (3) a nucleic acid sequence that encodes a Cas protein and is operably linked to an inducible first promoter comprising the specific polynucleotide binding site; (4) a guide RNA sequence; and (5) a second promoter that mediates transcription of the guide RNA.

Compositions provided by the present disclosure include nucleic acid molecules, vectors, polypeptides, cells and tissues comprising direct Cas-DRD regulation systems and Cas-transcription factor systems. Polypeptide compositions of the disclosure include polypeptides comprising protein domains displaying small molecule-dependent stability. Such protein domains are called drug responsive domains (DRDs). In the absence of a binding ligand, a DRD is destabilized and causes degradation of the polypeptide or protein fused to the DRD, while in the presence of its binding ligand, the fused DRD and polypeptide or protein are stabilized. The stability of the fused DRD and polypeptide or protein is dependent upon the dose of the binding ligand. Thus, the dose of the ligand may be used to modulate the expression or activity of the polypeptide or protein. Additionally, compositions of the disclosure include the binding ligands to which the DRDs are responsive. Cell compositions of the disclosure include modified cells comprising direct Cas-DRD regulation systems and Cas-transcription factor systems.

Methods related to direct Cas-DRD regulation systems and Cas-transcription factor systems that are provided by the present disclosure include methods of producing modified cells, methods of tunable regulation of Cas expression and/or activity, and methods of treating or preventing disease.

In a first aspect, the present disclosure provides a modified cell comprising one or more polynucleotides, said one or more polynucleotides comprising: i) a first nucleic acid sequence that encodes a Cas protein; ii) a first promoter operably linked to the first nucleic acid sequence; iii) a second nucleic acid sequence that encodes a drug responsive domain (DRD); iv) a third nucleic acid sequence that encodes a first guide RNA; and v) a second promoter operably linked to the third nucleic acid sequence; wherein the Cas protein is operably linked to the DRD; and wherein the DRD is derived from a parent protein selected from human carbonic anhydrase 2 (CA2), human DHFR (hDHFR), human estrogen receptor (ER), and human PDE5 (hPDE5) as described herein.

In a second aspect, the present disclosure provides a modified cell comprising: a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and a second polynucleotide comprising a fourth nucleic acid sequence that encodes a Cas protein, said fourth nucleic acid sequence being operably linked to an exogenous inducible first promoter comprising the specific polynucleotide binding site; a fifth nucleic acid sequence that encodes a first guide RNA, said fifth nucleic acid sequence being operably linked to an exogenous second promoter that mediates transcription of the first guide RNA; wherein the transcription factor activation domain and the transcription factor DNA binding domain interact to form a transcription factor that is able to activate transcription upon binding to the specific polynucleotide binding site.

In a third aspect, the present disclosure provides a modified cell comprising: a first polynucleotide comprising a first nucleic acid sequence encoding a transcription factor that is able to bind to a specific polynucleotide binding site and activate transcription, and a second nucleic acid sequence encoding a drug responsive domain (DRD); wherein the transcription factor is operably linked to the DRD; and a second polynucleotide comprising a third nucleic acid sequence encoding a Cas protein, said third nucleic acid sequence being operably linked to an exogenous inducible first promoter comprising the specific polynucleotide binding site; a fourth nucleic acid sequence that encodes a first guide RNA, said fourth nucleic acid sequence being operably linked to an exogenous second promoter that mediates transcription of the first guide RNA.

In a fourth aspect, the present disclosure provides a modified cell comprising one or more polynucleotides, said one or more polynucleotides comprising: i) a first nucleic acid sequence that encodes a transcription factor activation domain; ii) a second nucleic acid sequence that encodes a transcription factor DNA binding domain that binds to a specific polynucleotide binding site; iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; iv) a fourth nucleic acid sequence that encodes a Cas protein, said fourth nucleic acid sequence being operably linked to an exogenous inducible first promoter comprising the specific polynucleotide binding site; v) a fifth nucleic acid sequence that encodes a first guide RNA, said fifth nucleic acid sequence being operably linked to an exogenous second promoter that mediates transcription of the first guide RNA.

In a fifth aspect, the present disclosure provides a nucleic acid molecule comprising: i) a first nucleic acid sequence that encodes a Cas protein; ii) a first promoter that mediates transcription of the nucleic acid sequence encoding the Cas protein; iii) a second nucleic acid sequence that encodes a drug responsive domain (DRD); iv) a third nucleic acid sequence that encodes a guide RNA; and v) a second promoter that mediates transcription of the guide RNA; wherein the Cas protein is operably linked to the DRD; and wherein the DRD is derived from a parent protein selected from human carbonic anhydrase 2 (CA2), human DHFR (hDHFR), human estrogen receptor (ER), and human PDE5 (hPDE5).

In a sixth aspect, the present disclosure provides a nucleic acid molecule comprising: i) a first nucleic acid sequence that encodes a Cas protein, said first nucleic acid sequence being operably linked to an exogenous inducible first promoter comprising a specific polynucleotide binding site for a transcription factor; ii) a second nucleic acid sequence that encodes a first guide RNA, said second nucleic acid sequence being operably linked to an exogenous second promoter that mediates transcription of the first guide RNA.

In a seventh aspect, the present disclosure provides a nucleic acid molecule comprising: i) a first nucleic acid sequence that encodes a transcription factor activation domain; ii) a second nucleic acid sequence that encodes a transcription factor DNA binding domain that binds to a specific polynucleotide binding site; iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; iv) a fourth nucleic acid sequence that encodes a Cas protein, said fourth nucleic acid sequence being operably linked to an exogenous inducible first promoter comprising the specific polynucleotide binding site; and v) a fifth nucleic acid sequence that encodes a first guide RNA, said fifth nucleic acid sequence being operably linked to an exogenous second promoter that mediates transcription of the first guide RNA.

In an eighth aspect, the present disclosure provides a method of producing a modified cell, said method comprising introducing into a cell a nucleic acid molecule comprising: i) a first nucleic acid sequence that encodes a Cas protein; ii) a first promoter that mediates transcription of the nucleic acid sequence encoding the Cas protein; iii) a second nucleic acid sequence that encodes a drug responsive domain (DRD); iv) a third nucleic acid sequence that encodes a guide RNA; and v) a second promoter that mediates transcription of the guide RNA; wherein the Cas protein is operably linked to the DRD; and wherein the DRD is derived from a parent protein selected from human carbonic anhydrase 2 (CA2), human DHFR (hDHFR), human estrogen receptor (ER), and human PDE5 (hPDE5).

In a ninth aspect, the present disclosure provides a method of producing a modified cell, said method comprising introducing into a cell a first nucleic acid molecule and a second nucleic acid molecule, wherein the first nucleic acid molecule comprises: i) a first nucleic acid sequence that encodes a transcription factor activation domain; ii) a second nucleic acid sequence that encodes a transcription factor DNA binding domain that binds to a specific polynucleotide binding site; iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and wherein the second nucleic acid molecule comprises: i) a fourth nucleic acid sequence that encodes a Cas protein, said fourth nucleic acid sequence being operably linked to an exogenous inducible first promoter comprising the specific polynucleotide binding site; and ii) a fifth nucleic acid sequence that encodes a guide RNA, said fifth nucleic acid sequence being operably linked to an exogenous second promoter that mediates transcription of the guide RNA.

In a tenth aspect, the present disclosure provides a method of producing a modified cell, said method comprising introducing into a cell a nucleic acid molecule comprising: i) a first nucleic acid sequence that encodes a transcription factor activation domain; ii) a second nucleic acid sequence that encodes a transcription factor DNA binding domain that binds to a specific polynucleotide binding site; iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; iv) a fourth nucleic acid sequence that encodes a Cas protein, said fourth nucleic acid sequence being operably linked to an exogenous inducible first promoter comprising the specific polynucleotide binding site; v) a fifth nucleic acid sequence that encodes a guide RNA, said fifth nucleic acid sequence being operably linked to an exogenous second promoter that mediates transcription of the guide RNA.

In an eleventh aspect, the present disclosure provides a method of producing a modified cell, said method comprising introducing into a cell a first nucleic acid molecule and a second nucleic acid molecule, wherein the first nucleic acid molecule comprises: i) a first nucleic acid sequence that encodes a Cas protein; ii) a first promoter that mediates transcription of the nucleic acid sequence encoding the Cas protein; and iii) a second nucleic acid sequence that encodes a drug responsive domain (DRD); and wherein the second nucleic acid molecule comprises: i) a first nucleic acid sequence that encodes a first guide RNA operably linked to a first promoter that mediates transcription of the first guide RNA; and ii) a second nucleic acid sequence that encodes a second guide RNA operably linked to a second promoter that mediates transcription of the second guide RNA; wherein the Cas protein is operably linked to the DRD; and wherein the DRD is derived from a parent protein selected from human carbonic anhydrase 2 (CA2), human DHFR (hDHFR), human estrogen receptor (ER), and human PDE5 (hPDE5); and wherein the first nucleic acid molecule is introduced into the cell on a first plasmid or viral vector and the second nucleic acid molecule is introduced into the cell on a second plasmid or viral vector.

### BRIEF DESCRIPTION OF THE DRAWING

FIG. 1A-FIG. 1B illustrate direct and indirect regulation of Cas. FIG. 1A is a schematic diagram showing direct regulation of Cas. A vector delivers to a cell polynucleotides encoding a Cas protein operably linked to a DRD as well as an sgRNA that directs the Cas to a target locus in the cellular DNA. Addition of a ligand (for example, a drug) that binds to and stabilizes the DRD stabilizes the Cas protein, enabling recruitment of the Cas protein to the target locus. FIG. 1B is a schematic diagram showing DRD-mediated transcriptional regulation of Cas. One or more vectors deliver to a cell polynucleotides encoding a transcription factor operably linked to a DRD; an inducible promoter comprising the specific binding site to which the transcription factor binds that mediates the transcription of a nucleic acid sequence encoding a Cas protein; and an sgRNA directing the Cas to a target locus in the cellular DNA. Addition of the DRD's ligand stabilizes the transcription factor, which activates transcription and subsequent translation of the Cas protein. In turn, the Cas protein is recruited to the target locus via the sgRNA. Components of the DRD-mediated transcriptional regulation of Cas system may be delivered with one vector (top panel) or two vectors (bottom panel). In both FIG. 1A and FIG. 1B, the Cas nuclease is represented by Cas9.

FIG. 2A-FIG. 2B illustrate representative vectors comprising constructs designed to directly regulate Cas. FIG. 2A is a schematic of a vector comprising a construct including a nucleic acid sequence encoding a Cas protein operably linked to a DRD at the C-terminus. FIG. 2B is a schematic of a transfer vector comprising a construct including a nucleic acid sequence encoding a Cas protein operably linked to a DRD at the N-terminus. In both FIG. 2A and FIG. 2B, the transcription of Cas is mediated by Promoter 1. A second promoter (Promoter 2) mediates transcription of an sgRNA that directs the Cas to a target locus. Representative DRDs may be selected from carbonic anhydrase 2 (CA2) DRDs, human dihydrofolate reductase (hDHFR) DRDs, estrogen receptor (ER) DRDs, or phosphodiesterase 5 (PDE5) DRDs. A nuclear localization sequence (NLS) directs transport of the Cas to the nucleus.

FIG. 3A-FIG. 3B illustrate constructs designed for direct regulation of Cas9 expression and activity. FIG. 3A is a schematic of a construct for direct regulation of SpCas9 in which the DRD may be a CA2 DRD or an ER DRD. FIG. 3B is a schematic of a construct for direct regulation of SpCas9 and expression of mCherry, which permits fluorescent detection of the regulated construct. The P2A sequence enables expression of mCherry independent of DRD-regulated SpCas9 expression. In both FIG. 3A and FIG. 3B, the construct comprises a U6 promoter, an sgRNA, an EFS promoter, and SpCas9. CA2 DRD and ER DRD are shown as examples of DRDs that can be used to regulate expression and activity of the Cas in each construct shown.

FIG. 4 illustrates representative construct components that can be combined to generate constructs designed for direct regulation of Cas expression and activity. The construct components (from left to right) are as follows: a Pol II promoter operably linked to sequence encoding a Cas protein (e.g., the promoter may be selected from a CK8e promoter, an EFS promoter or a PGK promoter); a Cas (e.g., selected from SaCas9, Cas12a, and SpCas9); a DRD (e.g., selected from an DHFR DRD, CA2 DRD, ER DRD and PDE5 DRD), a Pol III promoter operably linked to a gRNA sequence (e.g., selected from H1, U6, and 7SK); and a gRNA corresponding to the Cas in the same construct. The approximate size in kilobases is shown next to each component.

FIG. 5A-FIG. 5B illustrate constructs designed for transcriptional regulation of Cas9 expression and activity. FIG. 5A is a schematic of constructs for transcriptional regulation of SpCas9. FIG. 5B is a schematic of constructs for transcriptional regulation of SpCas9 and expression of fluorescent proteins that enables identification of cells comprising these constructs. A nucleic acid encoding mCherry driven by the SV40 promoter is shown as part of the construct comprising the Cas nucleic acid sequence. A blue fluorescent protein (BFP) tag is encoded by nucleic acids of the construct comprising a transcription factor. A P2A sequence enables expression of BFP independent of the DRD-regulated SpCas9 expression. In both FIG. 5A and FIG. 5B, the transcription of the transcription factor is driven by an EF1a promoter while a U6 promoter drives transcription of the sgRNA. CA2 DRD and ER DRD are shown as examples of DRDs that can be used for the design of a transcriptionally regulated Cas system.

FIG. 6 shows a schematic of constructs designed for transcriptional regulation of Cas9 expression and activity. The top construct, labeled as “synthetic transcription factor” comprises an EFS promoter, a nucleic acid sequence encoding a transcription factor and a nucleic acid sequence encoding a DRD that is operably linked to the transcription factor. The bottom construct, labeled as “gene editing machinery” comprises the transcription factor binding site, a nucleic acid sequence encoding a Cas protein, wherein the transcription factor binding site mediates transcription of a nucleic acid sequence encoding the Cas protein, an H1 promoter and a gRNA sequence, wherein the H1 promoter mediates transcription of the gRNA sequence.

FIG. 7 shows a vector sequence comprising construct OT-Cas9-001 (SEQ ID NO: 22).

FIG. 8 shows a vector sequence comprising construct OT-Cas9-002 (SEQ ID NO: 23).

FIG. 9 shows a vector sequence comprising construct OT-Cas9-003 (SEQ ID NO: 24).

FIG. 10 shows a vector sequence comprising construct OT-Cas9-004 (SEQ ID NO: 25).

FIG. 11 shows a vector sequence comprising construct OT-Cas9-005 (SEQ ID NO: 26).

FIG. 12 shows a vector sequence comprising construct OT-Cas9-006 (SEQ ID NO: 27).

FIG. 13 shows a vector sequence comprising construct OT-Cas9-007 (SEQ ID NO: 28).

FIG. 14 shows a vector sequence comprising construct OT-Cas9-008 (SEQ ID NO: 29).

FIG. 15 shows a vector sequence comprising construct OT-Cas9-009 (SEQ ID NO: 30).

FIG. 16 shows a vector sequence comprising construct OT-Cas9-010 (SEQ ID NO: 31).

FIG. 17 shows a vector sequence comprising construct OT-Cas9-011 (SEQ ID NO: 32).

FIG. 18 shows a vector sequence comprising construct OT-Cas9-012 (SEQ ID NO: 33).

FIG. 19 shows a vector sequence comprising construct OT-Cas9-013 (SEQ ID NO: 34).

FIG. 20 shows a vector sequence comprising construct OT-Cas9-014 (SEQ ID NO: 35).

FIG. 21 shows a vector sequence comprising construct OT-Cas9-015 (SEQ ID NO: 36).

FIG. 22 shows a vector sequence comprising construct OT-Cas9-016 (SEQ ID NO: 37).

FIG. 23 shows a vector sequence comprising construct OT-Cas9-017 (SEQ ID NO: 38).

FIG. 24A-FIG. 24C show ligand-dependent Cas expression and activity with a direct Cas-DRD regulation system. FIG. 24A is a schematic of constructs comprising regulated (Cas9-024) or constitutive (Cas9-021 and Cas9-025) SpCas9. Constructs Cas9-021, Cas9-024 and Cas9-025 comprise: a U6 promoter operably linked to an sgRNA sequence, an EFS promoter operably linked to a nucleic acid sequence encoding a SpCas9 protein, a porcine teschovirus-1 2A (P2A sequence), and a nucleic acid sequence encoding mCherry red fluorescent protein. Construct Cas9-024 also comprises a nucleic acid sequence that encodes a CA2 DRD operably linked to the spCas9 protein. The sgRNA of constructs Cas9-021 and Cas9-024 target EGFP. The sgRNA of construct Cas9-025 targets EMX1. FIG. 24B is a graph showing ACZ-dependent regulation of Cas9 protein levels for construct Cas9-024 and no regulation for the constitutive constructs Cas9-021 and Cas9-025. FIG. 24C is a graph showing ACZ regulated Cas9 activity levels assessed by EGFP expression measured by flow cytometry. Cas9 activity was regulated by ACZ with construct Cas9-024, but not with construct Cas9-021 or Cas9-025. For both FIG. 24B and FIG. 24C, EGFP reporter cells were transiently transfected with the indicated constructs, as described in Example 5. For both FIG. 24B and FIG. 24C, each bar is the mean of 3 replicates and the error bar represents the standard error of the mean (SEM).

FIG. 25 is a dose response curve showing ligand-dependent Cas expression with a direct Cas-DRD regulation system. Each point is the mean of 3 replicates and the error bars are the standard deviation. Cells transfected with the CA2 DRD regulated construct (OT-Cas9-012) show ACZ dose-dependent regulation of Cas9 expression, whereas cells transfected with the constitutive construct (OT-Cas9-006) do not show regulation of Cas9 expression.

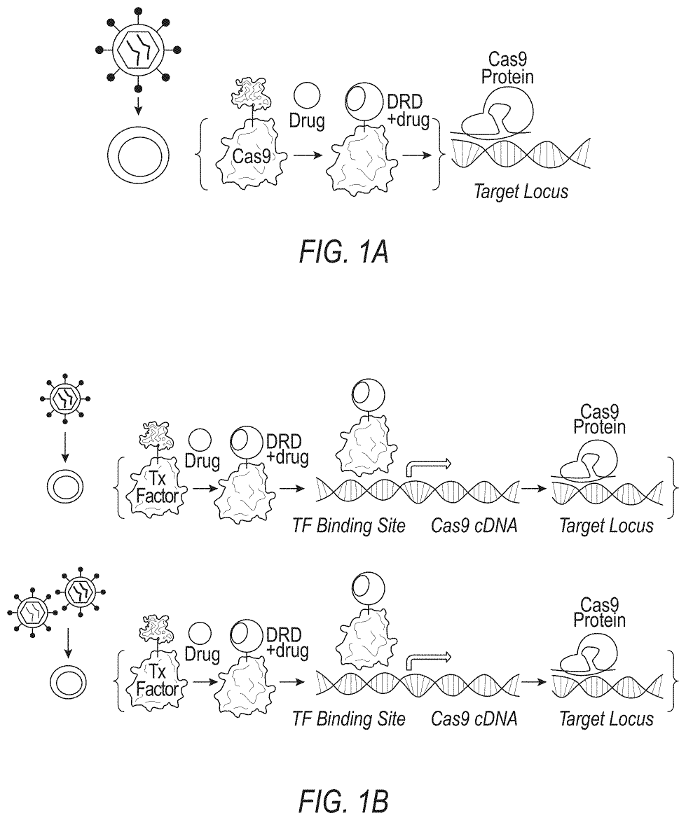

### DETAILED DESCRIPTION

### CRISPR-Cas Systems

CRISPR-Cas systems provide acquired immunity to bacteria and archaea against invasive genetic elements such as viruses, phages and plasmids (Horvath and Barrangou, Science, 2010, 327: 167-170; Bhaya et al., Annu. Rev. Genet., 2011, 45: 273-297; and Brrangou R, RNA, 2013, 4: 267-278). These prokaryotic adaptive immune systems are encoded by CRISPR loci and CRISPR-associated (cas) genes. CRISPR loci include short (about 24-48 nucleotide) DNA sequences of direct repeats separated by similarly sized, unique sequences called spacers (Grissa et al. BMC Bioinformatics 8, 172 (2007)). These sequences are generally adjacent to a set of CRISPR-associated (Cas) protein-coding genes that are required for CRISPR maintenance and function (Barrangou et al., Science 315, 1709 (2007), Brouns et al., Science 321, 960 (2008), Haft et al. PLoS Comput Biol 1, e60 (2005)). In recognition of the characteristic features of this family of repetitive DNA sequences, the acronym “CRISPR” (which stands for clustered regularly interspaced short palindrome repeats) has been adopted by the scientific community.

CRISPR-Cas systems provide acquired immunity to prokaryotes by conferring mechanisms to store nucleic acid fragments from past infections and detect and destroy nucleic acid molecules of similar foreign origin during a subsequent exposure. Upon an initial exposure to a foreign agent, the host prokaryote integrates short fragments of the invading foreign DNA into the CRISPR repeat-spacer array in its chromosome as new spacers. Transcription and processing of the CRISPR array results in short mature CRISPR RNAs (crRNAs) that hybridize to a complementary foreign target sequence (also called “protospacer” sequence), thereby enabling sequence-specific destruction of invading genetic elements by Cas nucleases upon a second infection. In addition to the crRNA-mediated targeting of foreign sequences, most CRISPR-Cas systems involve recognition of a short conserved sequence motif (approximately 2-5 bp) located in close proximity to the crRNA-targeted sequence on the invading DNA, referred to as a protospacer adjacent motif (PAM). The PAM motif can vary between different CRISPR-Cas systems and is considered to be important for the discrimination between self- and non-self sequences.

According to current classification, there are two classes of CRISPR-Cas systems. Class 1 systems use a complex of multiple Cas proteins for crRNA binding and target sequence degradation, whereas Class 2 systems use a single Cas protein for these functions. Class 1 and Class 2 systems are divided into 6 system types (I-VI), which are further divided into 19 subtypes. Of these systems, one of the best studied is the Class 2 Type II CRISPR-Cas system which employs the Cas9 endonuclease.

In the Type II CRISPR-Cas system, a crRNA pairs with an additional noncoding RNA, called the trans-activating crRNA (tracrRNA), and the resulting dual-RNA hybrid structure directs the Cas9 endonuclease to cleave a double stranded DNA (dsDNA) substrate containing a complementary 20-nucleotide target sequence. Target search, recognition and cleavage in the Type II CRISPR-Cas system requires complementary base pairing between the crRNA spacer and the target DNA protospacer, as well as the presence of a PAM sequence adjacent to the target site.

The sequence-specific nucleic acid recognition, Cas recruitment, and nucleic acid cleavage achievable by CRISPR-Cas systems makes them an attractive platform for genome engineering technologies in eukaryotic cells and organisms. Thus, these systems and their components have been repurposed to develop programmable nucleic acid targeting and editing tools.

CRISPR-Cas systems are particularly useful in gene and cell therapy because the Cas endonuclease, which forms a complex with the guide RNA, localizes to a specific target sequence of DNA in the genome following simple guide RNA:genomic DNA base pairing rules. The enzyme then cleaves the DNA at the targeted location, and one or more nucleotides may be inserted or deleted, or an existing DNA segment may be replaced with a different one.

One modification that has simplified the native CRISPR-Cas9 system for use in genome engineering technologies is the design of synthetic single-guide RNA (sgRNA). A sgRNA combines the crRNA and tracrRNA into a single RNA transcript, producing a chimeric structure that mimics the native prokaryotic dual tracrRNA-crRNA structure, while retaining fully functional Cas9-mediated sequence-specific DNA cleavage.

Other modifications to native CRISPR-Cas systems include modifications to Cas proteins. For example, native Cas9 comprises two nuclease domains: an HNH-like nuclease domain that cleaves the DNA strand complementary to the guide RNA sequence (target strand), and a RuvC-like nuclease domain that cleaves the DNA strand opposite the complementary strand (nontarget strand). By mutating either the HNH or RuvC nuclease domains, the resulting Cas9 can function as a nickase. By mutating both nuclease domains (resulting in the so-called “dead Cas9” or dCas9), the resulting dCas9 retains its RNA-guided DNA targeting ability but loses its endonuclease activity. Appending a Cas9 or a modified version of Cas9 to other proteins or protein domains can create fusion proteins with new functionalities. For example, a dCas9 can be fused with a gene activation domain or a gene repression domain to mediate gene activation or repression, respectively.

### Challenges for Therapeutic Applications of CRISPR-Cas Systems

CRISPR-Cas systems have been modified and developed for use in a variety of genome engineering technologies, including genetic editing as well as for modulation of gene expression. These engineered CRISPR-Cas systems have been shown to work in both prokaryotic as well as eukaryotic cells. However, controlling the effects and activity of CRISPR-Cas systems and ensuring the safety and effectiveness of these systems for therapeutic applications has been challenging.

Some of the challenges limiting the use of CRISPR-Cas systems are a consequence of constitutive endonuclease activity when Cas endonucleases are co-expressed with their sgRNAs. Constitutive expression of Cas nucleases can result in elevated off-target activity, increased number of off-target genomic alterations, triggering of DNA damage response, and cytotoxicity. Pre-existing and induced adaptive immunity to CRISPR has also been documented, indicating that there is an immunogenicity risk associated with constitutive expression of Cas nucleases. Such immunity against Cas nucleases could limit the durability of gene and cell therapies that employ CRISPR technology. Controlling the timing, level, and exposure of gene editing could reduce immunogenicity and increase the durability, safety, and tolerability of such therapeutic approaches.

### Regulation of CRISPR-Cas Systems

There have been a number of suggested approaches for regulating CRISPR-Cas systems. These approaches each come with advantages and disadvantages that must be considered with respect to their intended use. Several approaches involve inhibition of Cas protein and some of these are specifically discussed below.

One approach to regulate CRISPR systems involves protein inhibitors of CRISPR-Cas systems called anti-CRISPR (Acr) proteins. Naturally encoded by mobile genetic elements such as plasmids and phages, Acr proteins inhibit prokaryotic CRISPR-Cas immune function by a variety of mechanisms. Some Acr proteins directly interact with a Cas protein to inhibit target DNA binding, DNA cleavage, crRNA loading or effector-complex formation. Acr proteins targeting Type II CRISPR-Cas systems directly interact with Cas proteins, including Cas9, and inhibit binding of the Cas proteins to DNA or allow DNA binding but block target cleavage. The ability of Acr proteins to directly interfere with CRISPR-Cas functions is a feature that has made them attractive for the development of tools to post-translationally regulate CRISPR-Cas systems.

To achieve Cas inhibition, nucleic acids encoding Acr proteins can be delivered to cells on vectors according to known molecular biology techniques. Although suitable for certain applications, methods using Acr proteins to regulate CRISPR-Cas systems have some disadvantages. One disadvantage is that this approach may require more than one vector to deliver both the CRISPR-Cas components and the Acr protein to a cell of interest. This is because the size of genetic elements encoding Acr proteins may require an additional vector, separate vector for delivery. Another disadvantage is that typical Acr proteins (without additional engineering) do not enable control of both timing and level of Cas protein activation/deactivation and typically have slow reversibility kinetics. Varying the degree of CRISPR-Cas inhibition requires titration with Acr proteins of varying potency and/or increasing the amount of Cas protein or decreasing Acr expression, all of which is slower than other approaches for regulating CRISPR-Cas systems. It can also be difficult to achieve a basal off state with minimal Cas activity and typical Acr-based control systems are not easily redosable. Potential immunogenicity to Acr proteins is another drawback. It is worth noting that Acr methods do not eliminate Cas expression; rather, the existing Cas proteins remain in the cell and are bound by the Acr proteins. Other considerations of this approach include potential toxicity, Acr protein stability, optimal expression levels, and potential for off-target interactions.

Another approach to regulate CRISPR-Cas systems involves CRISPR-Cas-mediated self-cleavage to limit the duration of Cas expression. As an example, such an approach may involve expression of a self-targeting sgRNA (e.g., directed to the Cas nuclease-encoding nucleic acid sequence) as well as a second sgRNA targeting a genomic locus of interest. A consequence of this design is self-limiting expression of the Cas nuclease, which reduces the amount and duration of intracellular nuclease expression. While this approach may work for certain applications that require transient expression of Cas nuclease, there are some drawbacks. For instance, such an approach does not allow for more flexible control of timing and level of activation/deactivation of the Cas protein. Also, this approach is not considered to be redosable, in that it does not provide a way to readily reactivate the Cas nuclease if there is insufficient editing after the initial dose.

Another approach to regulate CRISPR-Cas systems involves ligand-mediated regulation of CRISPR-Cas components using drug responsive domain (DRD) technology. The present disclosure describes two different systems that employ DRDs to directly or indirectly regulate Cas protein expression and activity. These approaches offer several advantageous properties, some of which are lacking in other approaches, including the approaches described above. Some of the advantages of DRD-mediated regulation of Cas include (1) the potential for a basal off state with minimal to no “leakiness” of residual Cas activity; (2) the potential for an activated state that reaches wild-type functionality; (3) accessibility of the full system to a target tissue of interest, including muscle tissue; (4) potential for single vector delivery of all system components; (5) ability to control timing and level of activated and deactivated states; and (5) ability to redose the system by addition of a DRD-specific ligand.

### Direct Regulation of Cas Proteins by Drug Responsive Domains (DRDs)

In some aspects of the present disclosure, a Cas protein is directly regulated by a DRD in a direct Cas-DRD regulation system. A direct Cas-DRD regulation system comprises one or more polynucleotides that comprise (1) a nucleic acid sequence that encodes a Cas protein; (2) a first promoter that mediates transcription of the nucleic acid sequence encoding the Cas protein; (3) a nucleic acid sequence that encodes a drug responsive domain (DRD), wherein the Cas protein is operably linked to the DRD; (4) a nucleic acid sequence that encodes a guide RNA; and (5) a second promoter that mediates transcription of the guide RNA.

The one or more polynucleotides of a direct Cas-DRD regulation system may also be referred to herein as one or more nucleic acid constructs. The polynucleotides or nucleic acid constructs may comprise different arrangements of nucleic acid sequences, and/or may be uniquely combined as part of a direct Cas-DRD regulation system, so long as the resulting polynucleotides or nucleic acid constructs comprise (1) a nucleic acid sequence that encodes a Cas protein; (2) a first promoter that mediates transcription of the nucleic acid sequence encoding the Cas protein; (3) a nucleic acid sequence that encodes a drug responsive domain (DRD), wherein the Cas protein is operably linked to the DRD; (4) a nucleic acid sequence that encodes a guide RNA; and (5) a second promoter that mediates transcription of the guide RNA.

In various embodiments of the direct Cas-DRD regulation system described herein, the nucleic acid sequence that encodes a Cas protein is operably linked to the first promoter and/or the nucleic acid sequence that encodes a guide RNA is operably linked to the second promoter. In various embodiments, the first promoter is a Pol II promoter and the second promoter is a Pol III promoter.

In some embodiments, a direct Cas-DRD regulation system comprises one or more additional nucleic acid sequences that encode a different guide RNA; therefore, in such a system, there are at least two different guide RNA sequences. In some embodiments, the nucleic acid sequences encoding the different guide RNAs are operably linked to the same Pol III promoter. In some embodiments, the nucleic acid sequences encoding the different guide RNAs are operably linked to separate promoters. In some embodiments, the nucleic acid sequences encoding the different guide RNAs are operably linked to different promoters.

In some embodiments, a direct Cas-DRD regulation system comprises additional nucleic acid sequences including, but not limited to, regulatory elements, polyadenylation sequences, and sequences encoding linkers, protein tags, and cleavage sites.

In some embodiments, the nucleic acid sequence encoding the DRD is adjacent to the nucleic acid sequence encoding the Cas protein. In some embodiments, the nucleic acid sequence encoding the DRD is positioned 5′ to the nucleic acid sequence encoding the Cas protein. In some embodiments, the nucleic acid sequence encoding the DRD is positioned 3′ to the nucleic acid sequence encoding the Cas protein.

In several embodiments of the present disclosure, a direct Cas-DRD regulation system is comprised of a single construct. The single construct comprises all of the components of the direct Cas-DRD regulation system. In some embodiments, a single-construct direct Cas-DRD regulation system can be incorporated into a single nucleic acid molecule or vector, such as a plasmid or viral vector. In some embodiments, a single construct direct Cas-DRD regulation system may be introduced into a cell on a single nucleic acid molecule or vector, such as a plasmid or viral vector.

In some embodiments, a direct Cas-DRD regulation system is present in a cell or a population of cells. In some embodiments, one or more polynucleotides of a direct Cas-DRD regulation system are introduced into a cell or population of cells. In some embodiments, a direct Cas-DRD regulation system is introduced into a cell or population of cells via one vector or two vectors, wherein the vector is a viral vector.

The present disclosure also provides components of a direct Cas-DRD regulation system, including polynucleotides that comprise (1) a nucleic acid sequence that encodes a Cas protein; (2) a first promoter that mediates transcription of the nucleic acid sequence encoding the Cas protein; (3) a nucleic acid sequence that encodes a drug responsive domain (DRD), wherein the Cas protein is operably linked to the DRD; (4) a nucleic acid sequence that encodes a guide RNA; and (5) a second promoter that mediates transcription of the guide RNA. RNA and proteins that are encoded by these polynucleotides and/or encoded by these nucleic acid sequences are also considered to be components of a direct Cas-DRD regulation system.

In some embodiments, components of a direct Cas-DRD regulation system include complexes formed by the RNA and/or proteins encoded by the polynucleotides and/or encoded by the nucleic acid sequences of a direct Cas-DRD regulation system. For example, a Cas protein complexed with a guide RNA molecule (i.e., a “Cas molecule/gRNA molecule complex”) is a component of a direct Cas-DRD regulation system.

In some embodiments, components of a direct Cas-DRD regulation system include fusion proteins or engineered proteins encoded by the polynucleotides and/or encoded by the nucleic acid sequences of a direct Cas-DRD regulation system. For example, a Cas protein operably linked to a DRD is a component of a direct Cas-DRD regulation system. In some embodiments, a Cas protein operably linked to a DRD is referred to as a Cas-DRD fusion protein (e.g., Cas9-DRD fusion protein).

In some embodiments, a vector comprises one or more components of a direct Cas-DRD regulation system.

### Transcriptional Regulation of Cas

In some aspects of the present disclosure, the Cas protein is regulated transcriptionally by a transcription factor that is regulated by a DRD. This method of regulation is referred to herein as indirect Cas regulation and the components that together result in such indirect Cas regulation are referred to herein as a Cas-transcription factor system.

According to the present disclosure, a Cas-transcription factor system comprises one or more polynucleotides that comprise (1) one or more nucleic acid sequences that encode a transcription factor able to bind to a specific polynucleotide binding site and activate transcription; (2) a nucleic acid sequence that encodes a drug responsive domain (DRD), wherein the transcription factor is operably linked to the DRD; (3) a nucleic acid sequence that encodes a Cas protein and is operably linked to an inducible first promoter comprising the specific polynucleotide binding site; (4) a nucleic acid sequence that encodes a guide RNA; and (5) a second promoter that mediates transcription of the guide RNA. The nucleic acid sequence that encodes the transcription factor comprises a third promoter that mediates transcription of the transcription factor. The third promoter may be a constitutive promoter or an inducible promoter.

The one or more polynucleotides of a Cas-transcription factor system may also be referred to herein as one or more nucleic acid constructs. The polynucleotides or nucleic acid constructs may comprise different arrangements of nucleic acid sequences, and/or may be uniquely combined as part of a Cas-transcription factor system, so long as the resulting polynucleotides or nucleic acid constructs comprises (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; (3) a nucleic acid sequence that encodes a Cas protein and is operably linked to an inducible first promoter comprising the specific polynucleotide binding site; (4) a nucleic acid sequence that encodes a guide RNA; and (5) a second promoter that mediates transcription of the guide RNA.

In various embodiments of the Cas-transcription factor system described herein, the nucleic acid sequence that encodes a Cas protein is operably linked to the first promoter, wherein the first promoter is a Pol II promoter, and the nucleic acid sequence that encodes a guide RNA is operably linked to the second promoter, wherein the second promoter is a Pol III promoter.

In some embodiments, a Cas-transcription factor system comprises multiple constructs. In some embodiments, a Cas-transcription factor system comprises a transcription factor construct comprising one or more nucleic acid sequences encoding the transcription factor operably linked to a DRD and a payload construct comprising a nucleic acid sequence encoding the Cas protein.

In some embodiments, the transcription factor construct comprises a nucleic acid sequence that encodes a transcription factor and a nucleic acid sequence that encodes a DRD, wherein the transcription factor is operably linked to the DRD. The nucleic acid sequence that encodes the transcription factor is operably linked to a promoter that mediates transcription of the transcription factor. In some embodiments, the transcription factor construct comprises a nucleic acid sequence that encodes a transcription factor activation domain, a nucleic acid sequence that encodes a transcription factor DNA binding domain, and a nucleic acid sequence that encodes a DRD, wherein either or both of the activation domain and the DNA binding domain are operably linked to the DRD. In some embodiments, the promoter in a transcription factor construct is EF1a. In some embodiments, the promoter in a transcription factor construct is an inducible promoter comprising the specific polynucleotide binding site to which the transcription factor is able to bind and activate transcription (referred to herein as a “self-inducing transcription factor”). A self-inducing transcription factor employed in a Cas-transcription factor system of the present disclosure is an example of a double-off transcription system for Cas regulation. As used herein, the phrase “double-off transcription system” refers to a system of the present disclosure that comprises two modes of regulation. In the case of a double-off transcription system for Cas regulation comprising a self-inducing transcription factor, one mode of regulation comprises the DRD-regulated transcription factor and another mode of regulation comprises the self-inducing transcriptional regulation of the transcription factor.

In some embodiments, a payload construct comprises nucleic acid sequences encoding: a specific polynucleotide binding site comprising at least one nucleic acid site with a specific sequence recognized and bound by the transcription factor DNA binding domain, a nucleic acid sequence encoding a Cas protein, wherein the specific polynucleotide binding site enables transcription of the nucleic acid sequence encoding the Cas protein when the transcription factor-DRD binds to it; a guide RNA sequence, and a promoter that mediates transcription of the guide RNA.

In some embodiments, a Cas-transcription factor system comprises one or more additional nucleic acid sequences that encode a different guide RNA; therefore, in such a system, there are at least two different guide RNA sequences. In some embodiments, the nucleic acid sequences encoding the different guide RNAs are operably linked to the same Pol III promoter. In some embodiments, the nucleic acid sequences encoding the different guide RNAs are operably linked to separate promoters. In some embodiments, the nucleic acid sequences encoding the different guide RNAs are operably linked to different promoters.

In some embodiments, a Cas-transcription factor system comprises additional nucleic acid sequences including, but not limited to, regulatory elements, polyadenylation sequences, and nucleic acid sequences encoding linkers, protein tags, and cleavage sites.

Examples of constructs that may be used in Cas-transcription factor systems are described in Table 6.

In some embodiments of the present disclosure, a Cas-transcription factor system comprises two constructs. Together, the two constructs comprise all of the components of the Cas-transcription factor system. In some embodiments of the present disclosure, a Cas-transcription factor system comprising the transcription factor construct and the payload construct is incorporated into a single nucleic acid molecule, such as a plasmid or viral vector. A Cas-transcription factor system comprising a single nucleic acid molecule or polynucleotide may be referred to herein as a single vector Cas-transcription factor system. In some embodiments, a single nucleic acid molecule Cas-transcription factor system may be supplied for the methods of the present disclosure on the same plasmid or viral vector. In some embodiments, a single construct Cas-transcription factor system may be introduced into a cell on a single nucleic acid molecule, such as a single plasmid or single viral vector.

In some embodiments of the present disclosure, a Cas-transcription factor system comprises two constructs. Together, the two constructs comprise all of the components of the Cas-transcription factor system. In some embodiments, the two constructs are each incorporated into two separate nucleic acid molecules. In some embodiments, a two-construct Cas-transcription factor system may be supplied for the methods of the present disclosure in separate plasmids or separate viral vectors. In some embodiments, a first polynucleotide comprises nucleic acid sequences encoding the transcription factor operably linked to the DRD, and a second polynucleotide comprises nucleic acid sequences encoding a Cas protein operably linked to a transcription factor polynucleotide binding site. In some embodiments, the transcription factor construct comprises the guide RNA and its promoter. In some embodiments, the Cas protein construct comprises the guide RNA and its promoter. In some embodiments, the two constructs may be introduced into a cell on two nucleic acid molecules, such as two plasmids or two viral vectors, wherein one of the two molecules comprises a first construct and the second of the two molecules comprises a second construct.

In some embodiments, the inducible first promoter of a Cas-transcription factor system is an exogenous inducible promoter. An exogenous inducible promoter as used herein is a promoter that is not normally present in a cell but can be introduced into a cell by one or more genetic, biochemical or other methods.

According to the present disclosure, a Cas-transcription factor system encodes a transcription factor that can drive expression of a Cas protein. In some embodiments, the transcription factor is encoded by a first nucleic acid sequence that encodes a transcription factor activation domain and a second nucleic acid sequence that encodes a transcription factor DNA binding domain that binds to a specific polynucleotide binding site. The transcription factor activation domain and the transcription factor DNA binding domain interact to form a transcription factor that activates transcription of the nucleic acid sequence encoding the Cas protein upon binding to the specific polynucleotide binding site. In some embodiments, the transcription factor DNA binding domain and the transcription factor activation domain are expressed as a transcription factor fusion protein.

In some embodiments, the nucleic acid sequence encoding the DRD is adjacent to a nucleic acid sequence encoding at least one of the transcription factor domains. In some embodiments, the nucleic acid sequence encoding the DRD is positioned between a nucleic acid sequence encoding the transcription factor DNA binding domain and the transcription factor activation domain.

The transcription factor activation domain, the transcription factor DNA binding domain, and/or the combination of the transcription factor activation domain and the transcription factor DNA binding domain may be operably linked to the DRD (any of which is a DRD-TF).

In some embodiments, the transcription factor DNA binding domain is operably linked to the DRD. In some embodiments, the transcription factor activation domain is operably linked to the DRD. In some embodiments, both the transcription factor DNA binding domain and the transcription factor activation domain are operably linked to the DRD.

In some embodiments, upon stabilization of the operably linked DRD through binding of an exogenous stabilizing ligand, the stabilized DRD-TF is able to transcribe the nucleic acid sequence encoding the Cas protein of the Cas-transcription factor system. In the absence of the exogenous stabilizing ligand, the DRD-TF is degraded and unable to activate transcription. Thus, both the amount and the timing of Cas protein expression can be controlled by the exogenous stabilizing ligand.

In some embodiments, the specific polynucleotide binding site comprises at least one nucleic acid site with a specific sequence that is recognized and bound by the transcription factor DNA binding domain. In some embodiments, the specific polynucleotide binding site comprises two or more tandem nucleic acid sites, each with a specific sequence that is recognized and bound by the transcription factor DNA binding domain. In some embodiments, said tandem nucleic acid sites comprise identical nucleic acid sequences.

As described herein, a transcription factor or part thereof, is operably linked to a DRD in a Cas-transcription factor system of the present disclosure. The presence, absence or an amount of a ligand that binds to or interacts with the DRD, can, upon such binding or interaction modulate the stability of the transcription factor and consequently the function of the transcription factor. Thus, a Cas-transcription factor system can exhibit ligand-dependent activity of the transcription factor and consequently ligand-dependent activity of the Cas protein.

In various embodiments, the Cas-transcription factor system provides for the tunable, ligand-dependent transcription of a Cas protein. In various embodiments, the nucleic acid sequence encoding the Cas protein is operably linked to an exogenous inducible promoter comprising a specific polynucleotide binding site, that is, a defined DNA polynucleotide sequence, that specifically binds to the transcription factor DNA binding domain. The transcription factor binding domain, in combination with the transcription factor DNA activation domain, is then able to regulate transcription of the Cas transgene.

In some embodiments, the Cas protein of a Cas-transcription factor system is operably linked to a DRD. The DRD that is operably linked to the Cas protein can be the same as or different from the DRD that is operably linked to the transcription factor. In the absence of any DRD ligand, both the transcription factor and the Cas protein are destabilized. In the presence of the DRD ligand or ligands, the transcription factor and Cas protein are stabilized. Such a system comprising a DRD operably linked to a transcription factor and a DRD operably linked to a Cas protein that is transcriptionally regulated by the transcription factor is an example of a double-off transcription system for Cas regulation. This double-off transcription system comprises a first mode of regulation comprising the DRD-regulated transcription factor and a second mode of regulation comprising the DRD-regulated Cas protein.

In some embodiments, one or more components of a direct Cas-DRD regulation system is combined with one or more components of a Cas-transcription factor system. Such a combined system may be a double-off transcription system. As a non-limiting example, the combined system is a combination of one or more polynucleotides that comprise (1) one or more nucleic acid sequences that encode a transcription factor that is able to bind to a specific polynucleotide binding site and activate transcription; (2) a nucleic acid sequence that encodes a first drug responsive domain (first DRD), wherein the transcription factor is operably linked to the first DRD; (3) a nucleic acid sequence that encodes a Cas protein, wherein the nucleic acid sequence encoding the Cas protein is operably linked to an inducible first promoter comprising the specific polynucleotide binding site and wherein the Cas protein is operably linked to a second DRD; (4) a nucleic acid sequence that encodes a guide RNA; and (5) a second promoter that mediates transcription of the guide RNA. The first and second DRD can be the same or different. In some embodiments, the first and second DRD are responsive to the same stimulating agent. In some embodiments, the first and second DRD are responsive to different stimulating agents.

In some embodiments, a Cas-transcription factor system is present in a cell or a population of cells or an organism. In some embodiments, one or more polynucleotides of a Cas-transcription factor system are introduced into a cell, a population of cells or an organism. When a cell, population of cells or organism comprising a Cas-transcription factor system is exposed to an exogenous stabilizing ligand, the DRD-TF is stabilized. The stabilized DRD-TF is then able to bind to the specific polynucleotide binding site to which the DRD-TF binds, and thus regulate transcription of the polynucleotide encoding the Cas protein. In some embodiments, the binding of the stabilized DRD-TF activates transcription of the polynucleotide encoding the Cas protein, which results in protein expression in the cell or organism. In the absence of the exogenous stabilizing ligand, the DRD-TF is degraded and unable to activate transcription. Thus, both the amount and the timing of Cas protein expression can be controlled by administering the exogenous stabilizing ligand to the cell or organism.

The present disclosure also provides components of a Cas-transcription factor system, including polynucleotides that comprise (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; (3) a nucleic acid sequence that encodes a Cas protein and is operably linked to an inducible first promoter comprising the specific polynucleotide binding site; (4) a nucleic acid sequence that encodes a guide RNA; and (5) a second promoter that mediates transcription of the guide RNA. RNA and proteins that are encoded by these polynucleotides and/or nucleic acid sequences are also considered to be components of a Cas-transcription factor system.

In some embodiments, components of a Cas-transcription factor system include complexes formed by the RNA and/or proteins encoded by the polynucleotides and/or encoded by the nucleic acid sequences of a Cas-transcription factor system. For example, a Cas protein complexed with a guide RNA molecule (i.e., a “Cas molecule/gRNA molecule complex”) is a component of a Cas-transcription factor system.

In some embodiments, components of a Cas-transcription factor system include fusion proteins or engineered proteins encoded by the polynucleotides and/or encoded by the nucleic acid sequences of a Cas-transcription factor system. For example, a transcription factor operably linked to a DRD is a component of a Cas-transcription factor system. In some embodiments, a transcription factor operably linked to a DRD is referred to as a DRD-transcription factor fusion protein.

In some embodiments, a vector comprises one or more components of a Cas-transcription factor system.

### Transcription Factors of Cas-Transcription Factor Systems

In various embodiments, a transcription factor for use in the Cas-transcription factor systems, compositions and methods described herein includes a transcription factor DNA binding domain and a transcription factor activation domain. In some embodiments, the combination of the transcription factor DNA binding domain and a transcription factor activation domain results in a functional transcription factor. In various embodiments, the transcription factor binding domain and/or the transcription factor activation domain may interact with other transcription regulatory elements.

In various embodiments of the present disclosure, suitable transcription factors useful in a Cas-transcription factor system can include any known transcription factor for which the transcription factor-binding site is known. Some examples of such transcription factors include (but are not limited to) the STAT family (STATs 1, 2, 3, 4, 5a, 5b, and 6), c-Fos, FosB, Fra-1, Fra-2, c-Jun, JunB and JunD, fos/jun, NF kappa B, HIV-TAT, E2F family, T-Box Gene Family, Helix-Loop-Helix Transcription Factors, Zinc Finger Transcription Factors (e.g., Oct4 and Zif268), synthetic transcription factors, including those derived from zinc finger proteins and transcription-activator like effectors (TALEs) (e.g., ZFHD1), and transcription factors from the following families: bHLH, bZIP, Forkhead, Nuclear receptor, HMG/Sox, Ets, T-box, AT hook, Homeodomain+POU, Myb/SANT, THAP finger, CENPB, E2F, BED ZF, GATA, Rel, CxxC, IRF, SAND, SMAD, HSF, MBD, RFX, CUT+Homeodomain, DM, STAT, ARID/BRIGHT, Grainyhead, MADS box, AP-2, CSD, and Homeodomain+PAX.

In some embodiments, the encoded transcription factor DNA binding domain in a transcription factor construct is from a synthetic transcription factor, such as artificial zinc finger DNA-binding domain or a TALE transcription factor. In some embodiments, the encoded transcription factor DNA binding domain is ZFHD1. In some embodiments, the encoded transcription factor activation domain in a transcription factor construct is p65.

In some embodiments, a payload construct may comprise a specific polynucleotide binding site comprising at least one nucleic acid site with a specific sequence recognized and bound by the transcription factor DNA binding domain. An exemplary binding site comprises eight (8) nucleic acid sites that are recognized by a ZFHD1 DNA binding domain.

In various embodiments, the transcription factor DNA binding domain and the transcription factor activation domain are operably linked or may be separated by one or more intervening sequences, for example, a linker or a cleavage site.

### Cas Proteins of Direct Cas-DRD Regulation Systems and Cas-Transcription Factor Systems

The Cas protein of a direct Cas-DRD regulation system or a Cas-transcription factor system is able to localize to the nucleus of a cell. In several embodiments of the present disclosure, a nuclear localization signal (NLS) operably linked to the Cas protein enables transport of the Cas nuclease to the cell nucleus.

In some embodiments, the Cas protein of a Cas-DRD regulation system or a Cas-transcription factor system may be selected from a Cas9 or a Cas12a. In some embodiments, the Cas protein is a Cas9 protein or is encoded by a sequence derived from a Cas9 protein sequence. In some embodiments, the Cas protein is a Cas9 protein that is encoded by a polynucleotide or nucleic acid sequence that encodes a prokaryotic Cas9 protein or functional variant thereof. In some embodiments, the Cas protein is a Cas12a protein or is encoded by a sequence derived from a Cas12a protein sequence. In some embodiments, the Cas protein is a Cas12a protein that is encoded by a polynucleotide or nucleic acid sequence that encodes a prokaryotic Cas12a protein or functional variant thereof.

In some embodiments, the Cas protein of a Cas-DRD regulation system or a Cas-transcription factor system is derived from a Cas protein of a Type II CRISPR system. In some embodiments, the Cas protein is derived from a Cas9 protein. The Cas9 protein may be selected from Streptococcus pyogenes Cas 9 (SpCas9), Staphylococcus aureus (SaCas9), and Neisseria meningitidis Cas9 (NmeCas9).

The Cas protein may be derived from a number of species, including Cas molecules derived from S. pyogenes, S. aureus, N. meningitidis, S. thermophiles, Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteo bacterium, Gluconacetobacter diazotrophicus, Haemophilus parainjluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some embodiments, the Cas protein is a naturally-occurring Cas protein. In some embodiments, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein of a Cas-DRD regulation system or a Cas-transcription factor system is a CasD or is derived from a CasD protein (Pausch, P et al., Science, 2020, 369, 6501: 333-337). In some embodiments, the Cas protein of a Cas-DRD regulation system or a Cas-transcription factor system is a CasX or is derived from a CasX protein (Liu, J. et al., Nature, 2019, 566: 218-223).

In some embodiments, the Cas protein of a Cas-DRD regulation system or a Cas-transcription factor system has the same amino acid sequence as a parent Cas protein, such as a parent Cas9 or a parent Cas12a. In some embodiments, a Cas protein of the present disclosure is mutated relative to a parent Cas protein. In some embodiments, a Cas protein of the present disclosure is truncated at the N- or C-terminus relative to a parent Cas protein. In some embodiments, the amino acid sequences of the Cas proteins encompassed in the present disclosure have at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence of a parent Cas protein from which it is derived.

In some embodiments, the Cas protein of a Cas-DRD regulation system or a Cas-transcription factor system that is derived from a parent Cas protein retains the functions of the parent Cas protein. In some embodiments, the Cas proteins encompassed in the present disclosure retain RNA-guided DNA binding functionality. In some embodiments, the Cas proteins encompassed in the present disclosure retain endonuclease functionality.

In some embodiments, the Cas proteins encompassed in the present disclosure comprise one or more mutations in their nuclease domains. In some embodiments, a Cas protein of the present disclosure comprises a mutation in the HNH domain. In some embodiments, a Cas protein of the present disclosure comprises a mutation in the RuvC domain. In some embodiments, a Cas protein of the present disclosure comprises mutations in both the HNH domain and the RuvC domain.

In some embodiments, the Cas proteins encompassed in the present disclosure are capable of nucleic acid binding. In some embodiments, the Cas proteins encompassed in the present disclosure are capable of cleaving a phosphodiester bond in a polynucleotide chain. In some embodiments, the Cas proteins encompassed in the present disclosure are capable of both nucleic acid binding and cleaving a phosphodiester bond in a polynucleotide chain.

### Drug Responsive Domains (DRDs)

Drug responsive domains (DRDs) are protein domains that are unstable and degraded in the absence of a stabilizing DRD-binding ligand, but whose stability is rescued by binding to a corresponding DRD-binding ligand. The term drug responsive domain (DRD) is interchangeable with the term destabilizing domain (DD). Drug responsive domains (DRDs) can be appended to a polypeptide or protein and can render the attached polypeptide or protein unstable in the absence of a DRD-binding ligand. DRDs convey their destabilizing property to the attached polypeptide or protein via protein degradation. Without wishing to be bound by any theory, in the absence of a DRD-binding ligand, the appended polypeptide or protein is rapidly degraded by the ubiquitin-proteasome system of a cell. A ligand that binds to or interacts with a DRD can, upon such binding or interaction, modulate the stability of the appended polypeptide or protein. When a ligand binds its intended DRD, the instability is reversed and function of the appended polypeptide or protein can be restored. The conditional nature of DRD stability allows a rapid and non-perturbing switch from stable protein to unstable substrate for degradation. Moreover, its dependency on the concentration of its ligand further provides tunable control of degradation rates.

In some embodiments, DRDs of the present disclosure may be derived from known polypeptides that are capable of post-translational regulation of proteins. In some embodiments, DRDs of the present disclosure may be developed or derived from known proteins. Regions or portions or domains of wild type proteins may be utilized as DRDs in whole or in part. They may be combined or rearranged to create new peptides, proteins, regions or domains of which any may be used as DRDs or the starting point for the design of further DRDs.

In some embodiments, a DRD may be derived from a parent protein or from a mutant protein having one, two, three, or more amino acid mutations compared to the parent protein sequence. In some embodiments, the parent protein may be selected from, but is not limited to, FKBP; human protein FKBP; human DHFR (hDHFR); E. coli DHFR (ecDHFR); PDE5 (phosphodiesterase 5); CA2 (Carbonic anhydrase II); and ER (estrogen receptor). Examples of proteins that may be used to develop DRDs and their ligands are listed in Table 1.

TABLE 1
Proteins and their binding ligands

| | | Protein | |
|---|---|---|---|
| | | SEQ ID | Exemplary |
| Protein | Parent Protein Sequence | NO: | Ligands |
| E. coli | MISLIAALAVDRVIGMENAMPWNLP | 1 | Methotrexate |
| Dihydrofolate | ADLAWFKRNTLNKPVIMGRHTWESI | | (MTX) |
| reductase | GRPLPGRKNIILSSQPGTDDRVTWV | | Trimethoprim |
| (ecDHFR) | KSVDEAIAACGDVPEIMVIGGGRVY | | (TMP) |
| (Uniprot ID: | EQFLPKAQKLYLTHIDAEVEGDTHF | | |
| P0ABQ4) | PDYEPDDWESVFSEFHDADAQNSHS | | |
| | YCFEILERR | | |
| Human | MVGSLNCIVAVSQNMGIGKNGDLPW | 2 | Methotrexate |
| Dihydrofolate | PPLRNEFRYFQRMTTTSSVEGKQNL | | (MTX) |
| reductase | VIMGKKTWFSIPEKNRPLKGRINLV | | Trimethoprim |
| (hDHFR) | LSRELKEPPQGAHFLSRSLDDALKL | | (TMP) |
| (Uniprot ID: | TEQPELANKVDMVWIVGGSSVYKEA | | |
| P00374) | MNHPGHLKLFVTRIMQDFESDTFFP | | |
| | EIDLEKYKLLPEYPGVLSDVQEEKG | | |
| | IKYKFEVYEKND | | |
| Human FKBP | GVQVETISPGDGRTFPKRGQTCVVH | 3 | Shield-1 |
| (FK506 | YTGMLEDGKKFDSSRDRNKPFKFML | | |
| binding | GKQEVIRGWEEGVAQMSVGQRAKLT | | |
| protein) | ISPDYAYGATGHPGIIPPHATLVFD | | |
| (Uniprot | VELLKLE | | |
| ID: P62942) | | | |
| Phosphodiesterase | MEETRELQSLAAAVVPSAQTLKITD | 4 | Sildenafil; |
| 5 (PDE5), | FSFSDFELSDLETALCTIRMFTDLN | | Vardenafil; |
| ligand binding | LVQNFQMKHEVLCRWILSVKKNYRK | | Tadalafil |
| domain (Uniprot | NVAYHNWRHAFNTAQCMFAALKAGK | | |
| ID: Uniprot ID | IQNKLTDLEILALLIAALSHDLDHR | | |
| O76074) | GVNNSYIQRSEHPLAQLYCHSIMEH | | |
| | HHFDQCLMILNSPGNQILSGLSIEE | | |
| | YKTTLKIIKQAILATDLALYIKRRG | | |
| | EFFELIRKNQFNLEDPHQKELFLAM | | |
| | LMTACDLSAITKPWPIQQRIAELVA | | |
| | TEFFDQGDRERKELNIEPTDLMNRE | | |
| | KKNKIPSMQVGFIDAICLQLYEALT | | |
| | HVSEDCFPLLDGCRKNRQKWQALAE | | |
| | QQ | | |
| Phosphodiesterase | MERAGPSFGQ QRQQQQPQQQ KQQQR | 7 | Sildenafil; |
| 5 (PDE5), full- | DQDSV EAWLDDHWDF TFSYFVRKAT | | Vardenafil; |
| length (Uniprot | REMVNAWFAERVHTIPV CKE GIRGH | | Tadalafil |
| ID: Uniprot ID | TESCS CPLQQSPRAD NSAPGTPTRK | | |
| O76074) | ISASEFDRPL RPIVVKDSEGTVSFLS | | |
| | DSE KKEQMPLTPPR FDHDEGDQCS | | |
| | RLLELVKDIS SHLDVTALCH KIFLH | | |
| | IHGL ISADRYSLFLV CEDSSNDKFL | | |
| | ISRLFD VAEGSTLEEVSNNC IRLEW | | |
| | NKGIV GHVAALGEPLNIKDAYEDPR | | |
| | FNAEVDQITGYKTQSILCMP IKNHRE | | |
| | EVVG VAQAI NKKSG NGGTFTEKDE | | |
| | KDFAAYLAFC GIVLHNAQLY ETSLL | | |
| | ENKRN QVLLDLAS LIFEEQQSLEVI | | |
| | LKKIAATIISFM QVQK CTIFIVDED | | |
| | CSDSF SSVFHMECEE LEKSSDTLTR | | |
| | EHDANKINYM YAQYVKN TMEPLNIP | | |
| | DVSKD KRFPWTTENT GNVNQQCIRS | | |
| | LLCTPIKNGK KNKVIGVCQL VNKME | | |
| | ENTGKVKPFNRND EQ FLEAFVIFCG | | |
| | LGIQNTQMYE AVERAMAKQM VTLEV | | |
| | LSYHA SAAEEETRELQSLAAAV VPS | | |
| | AQTLKITDFS FSDFELSDLE TALCT | | |
| | IRMFT DLNLVQNFQM KHEVLCRWIL | | |
| | SVKKNYR KNVAYHNWRHAFN TAQCM | | |
| | FAALK AGKIQNKLTD LEILALLIAA | | |
| | LSHDLDHRG VNNSYIQRSEH PLAQL | | |
| | YCHSI MEHHHFDQCLMILNSPGNQI | | |
| | LSGLSIEEYK TTLKIIKQAILATDL | | |
| | ALYIK RRGEFFELIR KNQFNLEDP | | |
| | H QKELFLAMLM TACDLSAITKPWP | | |
| | IQQRIAELVATEFFDQG DRERKELN | | |
| | IE PTDLMNREKK NKIPSMQVGF I | | |
| | DAICLQLYE ALTHVSED CFPLLDG | | |
| | C RK NRQKWQALAEQQ EKMLINGE | | |
| | SG QAKRN | | |
| Carbonic | MSHHWGYGKHNGPEHWHKDFPIAKGER | 5 | Celecoxib |
| anhydrase II | QSPVDIDTHTAKYDPSLKPLSVSYDQA | | Acetazolamide |
| (CA2) (Uniprot | TSLRILNNGHAFNVEFDDSQDKAVLKG | | |
| ID: P00918) | GPLDGTYRLIQFHFHWGSLDGQGSEHT | | |
| | VDKKKYAAELHLVHWNTKYGDFGKAVQ | | |
| | QPDGLAVLGIFLKVGSAKPGLQKVVDV | | |
| | LDSIKTKGKSADFTNFDPRGLLPESLD | | |
| | YWTYPGSLTTPPLLECVTWIVLKEPIS | | |
| | VSSEQVLKFRKLNFNGEGEPEELMVDN | | |
| | WRPAQPLKNRQIKASFK | | |
| (Human estrogen | MTMTLHTKASGMALLHQIQGNELEPLNR | 6 | Bazedoxifene |
| receptor (ER) | PQLKIPLERPLG EVYLDSSKPA VYNY | | Raloxifene |
| Uniprot ID: | PEGAAYEFNAAAAANA QVYGQTGLPYGP | | |
| P03372.2) | GSEAAAFG SNGLGGFPPLNSVSPSPLML | | |
| | LHPPPQLSPFLQPHGQQVPY YLENEPSG | | |
| | YTVREAGPPAFY RPNSDNRRQGGRERLA | | |
| | STND KGSMAMESAKETRYCAVCNDYASG | | |
| | YHYGVWSCEGCKAFFK RSIQGHNDYMCP | | |
| | ATNQCTID KNRRKSCQACRLRKCYEVGM | | |
| | MKGGIRKDRRGGRMLKHKRQRDDGEGRGE | | |
| | VGSAGDMRAAN LWPSPLMIKRSKKNSLA | | |
| | LSL TADQMVSALLDAEPPILYSE YDPT | | |
| | RPFSEASMMGLLTNLA DRELVHMINWAK | | |
| | RVPGFVDLTLHDQVHLLE CAWLEILMIG | | |
| | LVWRSMEHPG KLLFAPNLLL DRNQGKC | | |
| | VEGMVEIFDMLLATSSRFRMMNLQGEEFV | | |
| | CLKSIILLNSGVYT FLSSTLKSLEEKDH | | |
| | IHRVLDKITDTLIHLM AKAGLTLQQQHQ | | |
| | RLAQLLLI LSHIRHMSN KGMEHLYSMK | | |
| | C KNVVPLYDLLLEMLDAHRLHAPTSRGG | | |
| | ASV EETDQSHLATAGSTSSHSLQ KYYI | | |
| | TGEAEG FPATV | | |

In some embodiments, the sequence of a protein used to develop DRDs may comprise all, part of, or a region thereof of a protein sequence in Table 1. In some embodiments, proteins that may be used to develop DRDs include isoforms of proteins listed in Table 1.

The amino acid sequences of the DRDs encompassed in the present disclosure have at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence of a parent protein from which it is derived, wherein the parent protein comprises a domain that binds a ligand.

Examples of DRDs of the present disclosure include those derived from: human carbonic anhydrase 2 (CA2), human DHFR, ecDHFR, human estrogen receptor (ER), FKBP, human protein FKBP, and human PDE5. Suitable DRDs, which may be referred to as destabilizing domains or ligand binding domains, are also known in the art. See, e.g., WO2018/161000; WO2018/231759; WO2019/241315; U.S. Pat. Nos. 8,173,792; 8,530,636; WO2018/237323; WO2017/181119; US2017/0114346; US2019/0300864; WO2017/156238; Miyazaki et al., J Am Chem Soc, 134:3942 (2012); Banaszynski et al. (2006) Cell 126:995-1004; Stankunas, K. et al. (2003) Mol. Cell 12:1615-1624; Banaszynski et al. (2008) Nat. Med. 14:1123-1127; Iwamoto et al. (2010) Chem. Biol. 17:981-988; Armstrong et al. (2007) Nat. Methods 4:1007-1009; Madeira da Silva et al. (2009) Proc. Natl. Acad. Sci. USA 106:7583-7588; Pruett-Miller et al. (2009) PLoS Genet. 5:e1000376; and Feng et al. (2015) Elife 4:e10606.

hPDE5 DRDs

In some embodiments, a DRD of the present disclosure is derived from hPDE5. In some embodiments, a DRD of the present disclosure is derived from hPDE5 isoform 2. In some embodiments, a DRD of the present disclosure is derived from hPDE5 isoform 3. In some embodiments, a DRD of the present disclosure is derived from hPDE5 isoform X1.

In some embodiments, a DRD of the present disclosure is derived from a cGMP-specific 3′,5′-cyclic phosphodiesterase (hPDE5) comprising the amino acid sequence of SEQ ID NO: 7.

In some embodiments, a DRD of the present disclosure may include the whole hPDE5 (SEQ ID NO: 7). In some embodiments, DRDs derived from hPDE5 may comprise the catalytic domain of hPDE5 (e.g., 535-860 of SEQ ID NO: 7). In some embodiments, hPDE5 DRDs of the present disclosure may include a methionine at the N terminal of the catalytic domain of hPDE5, i.e. amino acids 535-860 of hPDE5 wild-type (WT).

In some embodiments, a DRD of the present disclosure comprises, in whole or in part, a cGMP-specific 3′,5′-cyclic phosphodiesterase (hPDE5; SEQ ID NO: 7), and further comprises a mutation in the amino acid at position 732 (R732) of SEQ ID NO: 7. In some embodiments, the mutation in the amino acid at position 732 (R732) is selected from the group consisting of R732L, R732A, R732G, R732V, R732I, R732P, R732F, R732W, R732Y, R732H, R732S, R732T, R732D, R732E, R732Q, R732N, R732M, R732C, and R732K.

In some embodiments, a hPDE5 DRD of the present disclosure may further comprise one or more mutations independently selected from the group consisting of H653A, F736A, D764A, D764N, Y612F, Y612W, Y612A, W853F, I821A, Y829A, F787A, D656L, Y728L, M625I, E535D, E536G, Q541R, K555R, F559L, F561L, F564L, F564S, K591E, N587S, K604E, K608E, N609H, K630R, K633E, N636S, N661S, Y676D, Y676N, C677R, H678R, D687A, T712S, D724N, D724G, L738H, N742S, A762S, D764G, D764V, S766F, K795E, L797F, I799T, T802P, S815C, M816A, I824T, C839S, K852E, S560G, V585A, I599V, I648V, S663P, L675P, T711A, F744L, L746S, F755L, L804P, M816T, and F840S.

In some embodiments, a DRD of the present disclosure comprises, in whole or in part, a cGMP-specific 3′,5′-cyclic phosphodiesterase (hPDE5; SEQ ID NO: 7), and further comprises a mutation in the amino acid at position 732 (R732) of SEQ ID NO: 7. In some such embodiments, the DRD further comprises (i) a mutation in the amino acid at position 764 (D764) of SEQ ID NO: 7, wherein the mutation at D764 is selected from D764N and D764A; (ii) a mutation in the amino acid at position 612 (Y612) of SEQ ID NO: 7, wherein the mutation at Y612 is selected from the group consisting of Y612A, Y612F, and Y612W; (iii) an F736A mutation in the amino acid at position 736 (F736) of SEQ ID NO: 7; or (iv) an H653A mutation in the amino acid at position 653 (H653) of SEQ ID NO: 7.

In some embodiments, a DRD of the present disclosure comprises, in whole or in part, a cGMP-specific 3′,5′-cyclic phosphodiesterase (hPDE5; SEQ ID NO: 7), and further comprises a mutation in the amino acid at a position relative to SEQ ID NO: 7, said mutation selected from the group consisting of: W853F, I821A, Y829A, F787A, F736A, D656L, Y728L, M625I, and H653A.

In some embodiments, a hPDE5 DRD of the present disclosure may comprise one or more mutations independently selected from the group consisting of T537A, E539G, V548E, D558G, F559S, E565G, C574N, R577Q, R577W, N583S, Q586R, Q589L, K591R, K591R, L595P, C596R, W615R, F619S, Q623R, K633I, Q635R, N636S, T639S, D640N, E642G, I643T, L646S, A649V, A650T, S652G, H653A, D654G, V660A, V660A, L672P, A673T, C677Y, M681T, E682G, H685R, F686S, Q688R, M691T, S695G, G697D, S702I, I706T, E707K, Y709H, Y709C, I715V, I720V, A722V, D724G, Y728C, K730E, R732L, L738I, I739M, K741N, K741R, F744L, D748N, K752E, K752E, K752E, E753K, L756V, M758T, M760T, A762V, C763R, D764N, D764N, I774V, L781F, L781P, E785K, R794G, M805T, R807G, K812R, I813T, I813T, M816R, Q817R, V818A, F820S, I821V, C825R, Y829C, E830K, L832P, S836L, C846Y, C846S, L856P, L856P, A857T, or E858G.

In some embodiments, a hPDE5 DRD of the present disclosure may comprise two mutations independently selected from E536K, I739W; H678F, S702F; E669G, I700T; G632S, I648T; T639S, M816R; Q586R, D724G; E539G, L738I; L672P, S836L; M691T, D764N; I720V, F820S; E682G, D748N; S652G, Q688R; Y728C, Q817R; H653, R732L; L595P, K741R; R732D, F736S; R732E, F736D; R732V, F736G; R732W, F736G; R732W, F736V; R732L, F736W; R732P, F736Q; R732A, F736A; R732S, F736G; R732T, F736P; R732M, F736H; R732Y, F736M; R732P, F736D; R732P, F736G; R732W, F736L; R732L, F736S; R732D, F736T; R732L, F736V; R732G, F736V; and R732W, F736A.

In some embodiments, a hPDE5 DRD of the present disclosure may comprise two mutations independently selected from Q623R, D654G, K741N; A673T, L756V, C846Y; E642G, G697D, I813T; C677Y, H685R, A722V; Q635R, E753K, I813T; Y709H, K812R, L832P; N583S, K752E, C846S; K591R, I643T, L856P; F619S, V818A, Y829C; and F559S, Y709C, M760T. In some embodiments, a DRD of the present disclosure may comprise two mutations independently selected from S695G, E707K, I739M, C763R; A649V, A650T, K730E, E830K; and R577W, W615R, M805T, I821V.

In some embodiments, a hPDE5 DRD of the present disclosure may comprise multiple mutations independently selected from V660A, L781F, R794G, C825R, E858G; T537A, D558G, I706T, F744L, D764N; R577Q, C596R, V660A, I715V, E785K, L856P; and V548E, Q589L, K633I, M681T, S702I, K752E, L781P, A857T.

hDHFR DRDs

In some embodiments, a DRD of the present disclosure is derived from a human dihydrofolate reductase (hDHFR) protein such as, but not limited to, human dihydrofolate reductase 1 (hDHFR1), human dihydrofolate reductase 2 (hDHFR2), or a fragment or variant thereof.

In some embodiments, the DRD may be derived from a hDHFR protein and include at least one mutation. In some embodiments, the DRD may be derived from a hDHFR protein and include more than one mutation. In some embodiments, the DRD may be derived from a hDHFR protein and include two, three, four or five mutations.

In some embodiments, a DRD of the present disclosure may include the whole hDHFR (SEQ ID NO: 2). In some embodiments, DRDs derived from hDHFR may comprise amino acids 2-187 of the parent hDHFR sequence (e.g., amino acids 2-187 of SEQ ID NO: 2). This is referred to herein as an hDHFR M1del mutation.

In some embodiments, a DRD of the present disclosure comprises a region of or the whole hDHFR (SEQ ID NO: 2), and further comprises a mutation relative to SEQ ID NO: 2 selected from I17V, F59S, N65D, K81R, Y122I, N127Y, M140I, K185E, N186D, and M140I.

In some embodiments, a DRD of the present disclosure comprises a region of or the whole hDHFR (SEQ ID NO: 2), and further comprises two or more mutations relative to SEQ ID NO: 2.

In some embodiments, a hDHFR DRD of the present disclosure comprises two or more mutations selected from (A10V, H88Y); (C7R/Y163C); (I17V, Y122I); (Q36H, Y122I); (Q36K, Y122I); (Q36R, Y122I); (Q36S, Y122I); (Q36T, Y122I); (N65H, Y122I); (N65L, Y122I); (N65R, Y122I); (N65W, Y122I); (Q103E, Y122I); (Q103S, Y122I); (N108D; Y122I); (V121A, Y122I); (Y122I, K174N); (Y122I, E162G); (A125F, Y122I); (N127Y, Y122I); (H131R/E144G); (E162G/1176F); (K55R, N65K, Y122I); (Q36E, Q103H, Y122I); (Q36F, N65F, Y122I); and (V110A/V136M/K177R).

In some embodiments, a hDHFR DRD of the present disclosure comprises two or more mutations selected from (I17V, Y122I); (G21T, Y122N); (Q36H, Y122I); (Q36K, Y122I); (Q36R, Y122I); (Q36S, Y122I); (Q36T, Y122I); (N65H, Y122I); (N65L, Y122I); (N65R, Y122I); (N65W, Y122I); (L74N, Y122I); (Q103E, Y122I); (Q103S, Y122I); (N108D; Y122I); (V121A, Y122I); (Y122I, K174N); (Y122I, E162G); (A125F, Y122I); (N127Y, Y122I); (K55R, N65K, Y122I); (Q36E, Q103H, Y122I); and (Q36F, N65F, Y122I).

In some embodiments, a DRD of the present disclosure comprises, in whole or in part, a human dihydrofolate reductase (hDHFR; SEQ ID NO: 2), and further comprises a Y122I mutation in the amino acid at position 122 (Y122) of SEQ ID NO: 2. In some such embodiments, the DRD further comprises: (i) a Q36K mutation in the amino acid at position 36 (Q36) of SEQ ID NO: 2; (ii) an A125F mutation in the amino acid at position 125 (A125) of SEQ ID NO: 2; or (iii) a N65F mutation in the amino acid at position 65 (N65) of SEQ ID NO: 2 and a substitution of F or K at the amino acid position 36 (Q36) of SEQ ID NO: 2.

In some embodiments, a hDHFR DRD of the present disclosure may comprise one or more mutations independently selected from the group consisting of M1del, V2A, C7R, I8V, V9A, A10T, A10V, Q13R, N14S, G16S, I17N, I17V, K19E, N20D, G21T, G21E, D22S, L23S, P24S, L28P, N30D, N30H, N30S, E31G, E31D, F32M, R33G, R33S, F35L, Q36R, Q36S, Q36K, Q36F, R37G, M38V, M38T, T40A, V44A, K47R, N49S, N49D, M53T, G54R, K56E, K56R, T57A, F59S, I61T, K64R, N65A, N65S, N65D, N65F, L68S, K69E, K69R, R71G, I72T, I72A, I72V, N73G, L74N, V75F, R78G, L80P, K81R, E82G, H88Y, F89L, R92G, S93G, S93R, L94A, D96G, A97T, L98S, K99G, K99R, L100P, E102G, Q103R, P104S, E105G, A107T, A107V, N108D, K109E, K109R, V110A, D111N, M112T, M112V, V113A, W114R, I115V, I115L, V116I, G117D, V121A, Y122C, Y122D, Y122I, K123R, K123E, A125F, M126I, N127R, N127S, N127Y, H128R, H128Y, H131R, L132P, K133E, L134P, F135P, F135L, F135S, F135V, V136M, T137R, R138G, R138I, I139T, I139V, M140I, M140V, Q141R, D142G, F143S, F143L, E144G, D146G, T147A, F148S, F148L, F149L, P150L, E151G, I152V, D153A, D153G, E155G, K156R, Y157R, Y157C, K158E, K158R, L159P, L160P, E162G, Y163C, V166A, S168C, D169G, V170A, Q171R, E172G, E173G, E173A, K174R, I176A, I176F, I176T, K177E, K177R, Y178C, Y178H, F180L, E181G, V182A, Y183C, Y183H, E184R, E184G, K185R, K185del, K185E, N186S, N186D, D187G, and D187N.

In some embodiments, a DRD of the present disclosure comprises hDHFR (C7R, Y163C); hDHFR (E162G, I176F); hDHFR (G21T, Y122I); hDHFR (H131R, E144G); hDHFR (I17V, Y122I; hDHFR (L74N, Y122I; hDHFR (L94A, T147A); hDHFR (M53T, R138I); hDHFR (N127Y, Y122I); hDHFR (Q36K, Y122I); hDHFR (T137R, F143L); hDHFR (T57A, I72A); hDHFR (V121A, Y122I); hDHFR (V75F, Y122I); hDHFR (Y122I, A125F); hDHFR (Y122I, M140I); hDHFR (Y178H, E181G); hDHFR (Y183H, K185E); hDHFR (Amino acid 2-187 of WT) (G21T, Y122I); hDHFR (Amino acid 2-187 of WT) (I17V, Y122I); hDHFR (Amino acid 2-187 of WT) (L74N, Y122I); hDHFR (Amino acid 2-187 of WT) (L94A, T147A); hDHFR (Amino acid 2-187 of WT) (M53T, R138I); hDHFR (Amino acid 2-187 of WT) (N127Y, Y122I); hDHFR (Amino acid 2-187 of WT) (Q36K, Y122I); hDHFR (Amino acid 2-187 of WT) (V121A, Y122I); hDHFR (Amino acid 2-187 of WT) (V75F, Y122I); hDHFR (Amino acid 2-187 of WT) (Y122I, A125F); hDHFR (Amino acid 2-187 of WT) (Y122I, M140I); hDHFR (E31D, F32M, VI 161); hDHFR (G21E, I72V, I176T); hDHFR (I8V, K133E, Y163C); hDHFR (K19E, F89L, E181G); hDHFR (L23S, V121A, Y157C); hDHFR (N49D, F59S, D153G); hDHFR (Q36F, N65F, Y122I); hDHFR (Q36F, Y122I, A125F); hDHFR (V110A, V136M, K177R); hDHFR (V9A, S93R, P150L); hDHFR (Y122I, H131R, E144G); hDHFR (G54R, I115L, M140V, S168C); hDHFR (Amino acid 2-187 of WT) (E31D, F32M, VI 161); hDHFR (Amino acid 2-187 of WT) (Q36F, N65F, Y122I); hDHFR (Amino acid 2-187 of WT) (Q36F, Y122I, A125F); hDHFR (Amino acid 2-187 of WT) (Y122I, H131R, E144G); hDHFR (V2A, R33G, Q36R, L100P, K185R); hDHFR(D22S, F32M, R33S, Q36S, N65S); hDHFR (Amino acid 2-187 of WT) (D22S, F32M, R33S, Q36S, N65S); hDHFR (I17N, L98S, K99R, M112T, E151G, E162G, E172G); hDHFR (G16S, I17V, F89L, D96G, K123E, M140V, D146G, K156R); hDHFR (K81R, K99R, L100P, E102G, N108D, K123R, H128R, D142G, F180L, K185E); hDHFR (R138G, D142G, F143S, K156R, K158E, E162G, V166A, K177E, Y178C, K185E, N186S); hDHFR (N14S, P24S, F35L, M53T, K56E, R92G, S93G, N127S, H128Y, F135L, F143S, L159P, L160P, E173A, F180L); hDHFR (F35L, R37G, N65A, L68S, K69E, R71G, L80P, K99G, G117D, L132P, I139V, M140I, D142G, D146G, E173G, D187G); hDHFR (L28P, N30H, M38V, V44A, L68S, N73G, R78G, A97T, K99R, A107T, K109R, D111N, L134P, F135V, T147A, I152V, K158R, E172G, V182A, E184R); hDHFR (V2A, I17V, N30D, E31G, Q36R, F59S, K69E, I72T, H88Y, F89L, N108D, K109E, V110A, I115V, Y122D, L132P, F135S, M140V, E144G, T147A, Y157C, V170A, K174R, N186S); hDHFR (L100P, E102G, Q103R, P104S, E105G, N108D, V113A, W114R, Y122C, M126I, N127R, H128Y, L132P, F135P, I139T, F148S, F149L, I152V, D153A, D169G, V170A, I176A, K177R, V182A, K185R, N186S); and hDHFR (A10T, Q13R, N14S, N20D, P24S, N30S, M38T, T40A, K47R, N49S, K56R, 161T, K64R, K69R, 172A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N).

ecDHFR DRDs

In some embodiments, a DRD of the present disclosure is derived from E. coli dihydrofolate reductase (ecDHFR). In some embodiments, the DRD may be derived from an ecDHFR protein and include at least one mutation. In some embodiments, the DRD may be derived from an ecDHFR protein and include more than one mutation. In some embodiments, the DRD may be derived from an ecDHFR protein and include two, three, four or five mutations. In some embodiments, the DRD may be derived from an ecDHFR protein and comprise at least one mutation selected from Y100I, F103L, and G121V. In some embodiments, the DRD may be derived from an ecDHFR protein and comprise at least two mutations selected from R12Y, Y100I; R12H, E129K; H12Y, Y100I; H12L, Y100I; R98H, F103S; M42T, H114R; N18T, A19V; and I61F, T68S.

### FKBP DRDs

In some embodiments, a DRD of the present disclosure is derived from a FK506 binding protein (FKBP) protein or a fragment or variant thereof. In some embodiments, the DRD may be derived from a FKBP protein and include at least one mutation. In some embodiments, the DRD may be derived from a FKBP protein and include more than one mutation. In some embodiments, the DRD may be derived from an FKBP protein and include two, three, four or five mutations.

In some embodiments, a DRD of the present disclosure is derived from, in whole or in part, a human FKBP protein (SEQ ID NO: 3) and comprises at least one mutation selected from F36V, F15S, V24A, H25R, E60G, L106P, D100G, M66T, R71G, D100N, E102G, and K105I. In some embodiments, a DRD of the present disclosure comprises more than one mutation selected from F36P, L106P; and E31G, F36V, R71G, K105E.

### ER DRDs

In some embodiments, a DRD of the present disclosure is derived from an Estrogen Receptor (ER) protein or a fragment or variant thereof. In some embodiments, the DRD may be derived from an ER protein and include at least one mutation. In some embodiments, the DRD may be derived from an ER protein and include more than one mutation. In some embodiments, the DRD may be derived from an ER protein and include two, three, four or five mutations.

In some embodiments, a DRD of the present disclosure comprises the ligand binding domain of ER (amino acids 305 to 509 of SEQ ID NO: 6). In some embodiments, a DRD may include at least one mutation relative to the ligand binding domain of ER, wherein the mutation occurs at position 413 (N413) and/or at position 502 (Q502). In some embodiments, the mutation is at position N413 and is N413D, N413T, N413H, N413A, N413Q, N413V, N413C, N413K, N413M, N413R, N413S, N413W, N413I, N413E, N413L, N413P, N413F, N413Y or N413G. In some embodiments, the mutation is at position Q502 and is Q502H, Q502D, Q502E, Q502V, Q502A, Q502T, Q502N, Q502K, Q502S, Q502L, Q502Y, Q502W, Q502F, Q502I, Q502G, Q502P, Q502M, or Q502C. In some embodiments, the DRD comprises mutations at position N413 and at position Q502, wherein the mutation at position M413 is selected from N413D, N413T, N413H, N413A, N413Q, N413V, N413C, N413K, N413M, N413R, N413S, N413W, N413I, N413E, N413L, N413P, N413F, N413Y or N413G and the mutation at position Q502 is selected from Q502H, Q502D, Q502E, Q502V, Q502A, Q502T, Q502N, Q502K, Q502S, Q502L, Q502Y, Q502W, Q502F, Q502I, Q502G, Q502P, Q502M, or Q502C.

In some embodiments, the at least one mutation is N413D. In some embodiments, the at least one mutation is N413T. In some embodiments, the at least one mutation is Q502H. In some embodiments, the DRD comprises at least two mutations and is N413T, Q502H or N413D, Q502H.

In some embodiments, an ER DRD may further comprise one or more mutations independently selected from L384M, M421G, G521R or Y537S.

In some embodiments, a DRD of the present disclosure comprises the following: ER (aa 305-549 of WT, L384M, N413F, M421G, G521R, Y537S), ER (aa 305-549 of WT, L384M, N413L, M421G, G521R, Y537S), ER (aa 305-549 of WT, L384M, N413Y, M421G, G521R, Y537S), ER (aa 305-549 of WT, L384M, N413H, M421G, G521R, Y537S), ER (aa 305-549 of WT, L384M, N413Q, M421G, G521R, Y537S), ER (aa 305-549 of WT, L384M, N413I, M421G, G521R, Y537S), ER (aa 305-549 of WT, L384M, N413M, M421G, G521R, Y537S), ER (aa 305-549 of WT, L384M, N413K, M421G, G521R, Y537S), ER (aa 305-549 of WT, L384M, N413V, M421G, G521R, Y537S), ER (aa 305-549 of WT, L384M, N413S, M421G, G521R, Y537S), ER (aa 305-549 of WT, L384M, N413C, M421G, G521R, Y537S), ER (aa 305-549 of WT, L384M, N413W, M421G, G521R, Y537S), ER (aa 305-549 of WT, L384M, N413P, M421G, G521R, Y537S), ER (aa 305-549 of WT, L384M, N413R, M421G, G521R, Y537S), ER (aa 305-549 of WT, L384M, N413T, M421G, G521R, Y537S), ER (aa 305-549 of WT, L384M, N413A, M421G, G521R, Y537S), ER (aa 305-549 of WT, L384M, N413E, M421G, G521R, Y537S), ER (aa 305-549 of WT, L384M, N413G, M421G, G521R, Y537S), ER (aa 305-549 of WT, L384M, M421G, Q502F, G521R, Y537S), ER (aa 305-549 of WT, L384M, M421G, Q502L, G521R, Y537S), ER (aa 305-549 of WT, L384M, M421G, Q502Y, G521R, Y537S), ER (aa 305-549 of WT, L384M, M421G, Q502H, G521R, Y537S), ER (aa 305-549 of WT, L384M, M421G, Q502I, G521R, Y537S), ER (aa 305-549 of WT, L384M, M421G, Q502M, G521R, Y537S), ER (aa 305-549 of WT, L384M, M421G, Q502N, G521R, Y537S), ER (aa 305-549 of WT, L384M, M421G, Q502K, G521R, Y537S), ER (aa 305-549 of WT, L384M, M421G, Q502V, G521R, Y537S), ER (aa 305-549 of WT, L384M, M421G, Q502S, G521R, Y537S), ER (aa 305-549 of WT, L384M, M421G, Q502C, G521R, Y537S), ER (aa 305-549 of WT, L384M, M421G, Q502W, G521R, Y537S), ER (aa 305-549 of WT, L384M, M421G, Q502P, G521R, Y537S), ER (aa 305-549 of WT, L384M, M421G, Q502T, G521R, Y537S), ER (aa 305-549 of WT, L384M, M421G, Q502A, G521R, Y537S), ER (aa 305-549 of WT, L384M, M421G, Q502D, G521R, Y537S), ER (aa 305-549 of WT, L384M, M421G, Q502E, G521R, Y537S), and ER (aa 305-549 of WT, L384M, M421G, Q502G, G521R, Y537S).

### CA2 DRDs

In some embodiments, a DRD of the present disclosure may be derived from human carbonic anhydrase 2 (hCA2), which is a member of the carbonic anhydrases, a superfamily of metalloenzymes. In some embodiments, the DRD may be derived from a hCA2 protein and include at least one mutation. In some embodiments, the DRD may be derived from a hCA2 protein and include more than one mutation. In some embodiments, the DRD may be derived from an hCA2 protein and include two, three, four or five mutations.

In some embodiments, a DRD of the present disclosure may be derived from amino acids 1-260 of CA2 (SEQ ID NO: 5). In some embodiments, DRDs are derived from CA2 comprising amino acids 2-260 of the parent CA2 sequence (e.g., amino acids 2-260 of SEQ ID NO: 5). This is referred to herein as a CA2 M1del mutation. In one embodiment, DRDs derived from CA2 may comprise amino acids 2-237 of the parent CA2 sequence (e.g., amino acids 2-237 of SEQ ID NO: 5).

In some embodiments, a DRD of the present disclosure comprises a region of or the whole human carbonic anhydrase 2 (CA2; SEQ ID NO: 5), and further comprises a mutation relative to SEQ ID NO: 5 selected from E106D, G63D, H122Y, I59N, L156H, L183S, L197P, S56F, S56N, W208S, Y193I, and Y51T.

In some embodiments, a DRD of the present disclosure comprises a region of or the whole human carbonic anhydrase 2 (CA2; SEQ ID NO: 5), and further comprises a mutation relative to SEQ ID NO: 5 selected from A115L, A116Q, A116V, A133L, A133T, A141P, A152D, A152L, A152R, A173C, A173G, A173L, A173T, A23P, A247L, A247S, A257L, A257S, A38P, A38V, A54Q, A54V, A54X, A65L, A65N, A65V, A77I, A77P, A77Q, C205M, C205R, C205V, C205W, C205Y, D101G, D101M, D110I, D129I, D138G, D138M, D138N, D161*, D161M, D161V, D164G, D164I, D174*, D174T, D179E, D179I, D179R, D189G, D189I, D19T, D19V, D242G, D242T, D32T, D34T, D41T, D52I, D52L, D71F, D71G, D71K, D71M, D71S, D71Y, D72I, D72S, D72T, D72X, D75T, D75V, D85M, E106D, E106G, E106S, E117*, E117N, E14N, E186*, E186N, E204A, E204D, E204G, E204N, E213*, E213G, E213N, E220K, E220R, E220S, E233D, E233G, E233R, E235*, E235G, E235N, E237K, E237R, E238*, E238N, E238R, E26S, E69D, E69K, E69S, F130L, F146V, F175I, F175L, F175S, F178L, F178S, F20L, F20S, F225I, F225L, F225S, F225Y, F230I, F230L, F230S, F259L, F259S, F66S, F70I, F70L, F95Y, G102D, G104R, G104V, G128R, G12D, G12E, G131E, G131R, G131W, G139D, G144D, G144V, G150A, G150S, G150W, G155A, G155C, G155D, G155S, G170A, G170D, G182A, G182W, G195A, G195R, G232R, G232W, G234L, G234V, G25E, G63D, G63V, G81E, G81V, G82D, G86A, G86D, G98V, H107I, H107Q, H119T, H119Y, H122T, H122Y, H15L, H15T, H15Y, H17D, H17I, H36I, H36Q, H64M, H94T, H96T, I145F, I145M, I166H, I166L, I209D, I209L, I215H, I215S, I22L, I255N, I255S, I33S, I59F, I59N, I59S, I91F, K111E, K111N, K112R, K113I, K113N, K126N, K132E, K132R, K148E, K148R, K153*, K153N, K158E, K158N, K167*, K169N, K169R, K171Q, K171R, K18R, K212N, K212Q, K212R, K212W, K224E, K224N, K227*, K227N, K24R, K251E, K251R, K256Q, K260F, K260L, K260Q, K39S, K45N, K45S, K80M, K80R, L118F, L120W, L140V, L140W, L143*, L147*, L147F, L156F, L156H, L156P, L156Q, L163A, L163W, L183P, L183S, L184F, L184P, L188P, L188W, L197*, L197M, L197P, L197R, L197T, L202F, L202H, L202I, L202P, L202R, L202S, L203P, L203S, L203W, L211*, L211A, L211S, L223*, L223I, L223V, L228F, L228H, L228T, L239*, L239F, L239T, L250*, L250P, L250T, L44*, L44M, L47C, L47V, L57*, L57X, L60S, L79F, L79S, L84W, L90*, L90V, M240D, M240L, M240R, M240W, N11D, N11K, N124T, N177*, N177T, N229*, N229T, N231D, N231F, N231K, N231L, N231M, N231Q, N231T, N243Q, N243T, N252E, N252T, N61R, N61T, N61Y, N62K, N62M, N67D, N67T, P137L, P13A, P13H, P13L, P13S, P154L, P154R, P154T, P180L, P180S, P185L, P185S, P185V, P194Q, P200A, P200L, P200S, P200T, P201A, P201L, P201R, P201S, P214T, P236L, P236T, P246L, P246Q, P249A, P249F, P249H, P249I, P249X, P30L, P30S, P42L, P83A, Q103K, Q135S, Q136N, Q157R, Q157S, Q221A, Q221R, Q248F, Q248L, Q248S, Q254A, Q254K, Q28S, Q53H, Q53K, Q53N, Q74R, Q92H, Q92S, R181H, R181S, R181V, R226H, R226P, R226V, R245A, R253G, R253Q, R27A, R58G, R89D, R89F, R89I, R89X, R89Y, S105L, S105Q, S151A, S151I, S151Q, S165F, S165P, S172E, S172V, S187I, S187P, S196H, S196L, S216A, S216Q, S218A, S218Q, S219A, S219Q, S258F, S258P, S29C, S29P, S43P, S43T, S48L, S50P, S56F, S56N, S56P, S56X, S73L, S73N, S73X, S99H, T108L, T125I, T125P, T168K, T168N, T168Q, T176H, T176L, T192D, T192F, T192I, T192N, T192P, T192X, T198D, T198I, T198P, T199A, T199H, T199P, T207D, T207I, T207P, T207S, T35I, T35L, T37Q, T55L, T87L, V109M, V109W, V121F, V134C, V134F, V142F, V149G, V149L, V159L, V159S, V160C, V160L, V162A, V162C, V206*, V206C, V206M, V210C, V217L, V217R, V217S, V222A, V222C, V222G, V241G, V241W, V241X, V31L, V49F, V68L, V68W, V78C, W123G, W123R, W16G, W191*, W191G, W191L, W208G, W208L, W208S, W244*, W244G, W244L, W97C, W97G, Y114H, Y114M, Y127M, Y190*, Y190L, Y190T, Y193C, Y193F, Y193I, Y193L, Y193T, Y193V, Y193X, Y40M, Y51F, Y51M, Y51T, Y51X, Y88T, K9N, and S29A. As used herein “*” indicates the translation of the stop codon and X indicates any amino acid.

In some embodiments, a DRD of the present disclosure comprises a region of or the whole human carbonic anhydrase 2 (CA2; SEQ ID NO: 5), and further comprises two or more mutations relative to SEQ ID NO: 5.

In some embodiments, a DRD of the present disclosure comprises CA2 (aa 2-260 of WT, R27L, H122Y), CA2 (aa 2-260 of WT, T87I, H122Y), CA2 (aa 2-260 of WT, H122Y, N252D), CA2 (aa 2-260 of WT, D72F, V241F), CA2 (aa 2-260 of WT, V241F, P249L), CA2 (aa 2-260 of WT, D72F, P249L), CA2 (aa 2-260 of WT, D71L, L250R), CA2 (aa 2-260 of WT, D72F, P249F), CA2 (aa 2-260 of WT, T55K, G63N, Q248N), CA2 (aa 2-260 of WT, L156H, A257del, S258del, F259del, K260del), CA2 (aa 2-260 of WT, L156H, S2del, H3del, H4del, W5del), CA2 (aa 2-260 of WT, W4Y, L156H), CA2 (aa 2-260 of WT, L156H, G234del, E235del, P236del), CA2 (aa 2-260 of WT, L156H, F225L), CA2 (aa 2-260 of WT, D70N, D74N, D100N, L156H), (CA2 (aa 2-260 of WT, I59N, G102R), CA2 (aa 2-260 of WT, G63D, E69V, N231I), CA2 (aa 2-260 of WT, R27L, T87I, H122Y, N252D), CA2 (aa 2-260 of WT, D72F, V241F, P249L), CA2 (aa 2-260 of WT, D71L, T87N, L250R), CA2 (aa 2-260 of WT, L156H, S172C, F178Y, E186D), CA2 (aa 2-260 of WT, A77I, P249F), CA2 (aa 2-260 of WT, E106D, C205S), CA2 (aa 2-260 of WT, C205S, W208S), CA2 (aa 2-260 of WT, S73N, R89Y), CA2 (aa 2-260 of WT, D71K, T192F), CA2 (aa 2-260 of WT, S73N, R89F), CA2 (aa 2-260 of WT, G63D, M240L), CA2 (aa 2-260 of WT, V134F, L228F), or CA2 (aa 2-260 of WT, S56F, D71S).

In some embodiments, a DRD of the present disclosure comprises CA2 (aa 2-260 of WT, R27L, H122Y), CA2 (aa 2-260 of WT, T87I, H122Y), CA2 (aa 2-260 of WT, H122Y, N252D), CA2 (aa 2-260 of WT, D72F, V241F), CA2 (aa 2-260 of WT, V241F, P249L), CA2 (aa 2-260 of WT, D72F, P249L), CA2 (aa 2-260 of WT, D71L, L250R), CA2 (aa 2-260 of WT, D72F, P249F), CA2 (aa 2-260 of WT, T55K, G63N, Q248N), CA2 (aa 2-260 of WT, L156H, A257del, S258del, F259del, K260del), CA2 (aa 2-260 of WT, L156H, S2del, H3del, H4del, W5del), CA2 (aa 2-260 of WT, W4Y, L156H), CA2 (aa 2-260 of WT, L156H, G234del, E235del, P236del), CA2 (aa 2-260 of WT, L156H, F225L), CA2 (aa 2-260 of WT, D70N, D74N, D100N, L156H), (CA2 (aa 2-260 of WT, I59N, G102R), CA2 (aa 2-260 of WT, G63D, E69V, N231I), CA2 (aa 2-260 of WT, R27L, T87I, H122Y, N252D), CA2 (aa 2-260 of WT, D72F, V241F, P249L), CA2 (aa 2-260 of WT, D71L, T87N, L250R), CA2 (aa 2-260 of WT, L156H, S172C, F178Y, E186D), CA2 (aa 2-260 of WT, D71F, N231F), CA2 (aa 2-260 of WT, A77I, P249F), CA2 (aa 2-260 of WT, D71K, P249H), CA2 (aa 2-260 of WT, D72F, P249H), CA2 (aa 2-260 of WT, Q53N, N61Y), CA2 (aa 2-260 of WT, E106D, C205S), CA2 (aa 2-260 of WT, C205S, W208S), CA2 (aa 2-260 of WT, S73N, R89Y), CA2 (aa 2-260 of WT, D71K, T192F), CA2 (aa 2-260 of WT, Y193L, K260L), CA2 (aa 2-260 of WT, D71F, V241F, P249L), CA2 (aa 2-260 of WT, L147F, Q248F), CA2 (aa 2-260 of WT, D52I, S258P), CA2 (aa 2-260 of WT, D72S, T192N), CA2 (aa 2-260 of WT, D179E, T192I), CA2 (aa 2-260 of WT, S56N, Q103K), CA2 (aa 2-260 of WT, D71Y, Q248L), CA2 (aa 2-260 of WT, S73N, R89F), CA2 (aa 2-260 of WT, D71K, N231L, E235G, L239F), CA2 (aa 2-260 of WT, D72F, P249I), CA2 (aa 2-260 of WT, D72X, V241X, P249X), CA2 (aa 2-260 of WT, A54X, S56X, L57X, T192X), CA2 (aa 2-260 of WT, Y193V, K260F), CA2 (aa 2-260 of WT, G63D, M240L), CA2 (aa 2-260 of WT, V134F, L228F), CA2 (aa 2-260 of WT, D71G, N231K), CA2 (aa 2-260 of WT, S56F, D71S), CA2 (aa 2-260 of WT, D52L, G128R, Q248F), CA2 (aa 2-260 of WT, S73X, R89X), CA2 (aa 2-260 of WT, Y51X, D72X, V241X, P249X), CA2 (aa 2-260 of WT, D72I, W97C), CA2 (aa 2-260 of WT, D71K, T192F, N231F), CA2 (aa 2-260 of WT, H36Q, S43T, Y51F, N67D, G131W, R226H), CA2 (aa 2-260 of WT, F70I, F146V), CA2 (aa 2-260 of WT, K45N, V68L, H119Y, K169R, D179E), CA2 (aa 2-260 of WT, H15L, A54V, K111E, E220K, F225I), CA2 (aa 2-260 of WT, P13S, P83A, D101G, K111N, F230I), CA2 (aa 2-260 of WT, G63D, W123R, E220K), CA2 (aa 2-260 of WT, N11D, E69K, G86D, V109M, K113I, T125I, D138G, G155S), CA2 (aa 2-260 of WT, I59N, G102R, A173T), CA2 (aa 2-260 of WT, L79F, P180S), CA2 (aa 2-260 of WT, A77P, G102R, D138N), CA2 (aa 2-260 of WT, F20L, K45N, G63D, E69V, N231I), CA2 (aa 2-260 of WT, T199N, L202P, L228F), CA2 (aa 2-260 of WT, K9N, H122Y, T168K), CA2 (aa 2-260 of WT, Q53H, L90V, Q92H, G131E), CA2 (aa 2-260 of WT, L44M, L47V, N62K, E69D), CA2 (aa 2-260 of WT, D75V, K169N, F259L), CA2 (aa 2-260 of WT, T207S, V222A, N231D), CA2 (aa 2-260 of WT, I59F, V206M, G232R), CA2 (aa 2-260 of WT, P13A, A133T), CA2 (aa 2-260 of WT, I59N, R89I), CA2 (aa 2-260 of WT, A65N, G86D, G131R, G155D, K158N, V162A, G170D, P236L), CA2 (aa 2-260 of WT, G12R, H15Y, D19V), CA2 (aa 2-260 of WT, A65V, F95Y, E106G, H107Q, I145M, F175I), CA2 (aa 2-260 of WT, G63D, E69V, N231I), CA2 (aa 2-260 of WT, S29A, C205S) and/or CA2 (aa 2-260 of WT, S29C, C205S).

In some embodiments, a DRD of the present disclosure comprises, in whole or in part, a human carbonic anhydrase 2 (CA2; SEQ ID NO: 5), and further comprises a H122Y mutation in the amino acid at position 122 (H122) of SEQ ID NO: 5. In some such embodiments, the DRD further comprises: (i) a R27L mutation in the amino acid at position 27 (R27) of SEQ ID NO: 5; (ii) a T87I mutation in the amino acid at position 87 (T87) of SEQ ID NO: 5; (iii) a N252D mutation in the amino acid at position 252 (N252) of SEQ ID NO: 5; or a combination of (i), (ii) and/or (iii).

In some embodiments, a DRD of the present disclosure comprises, in whole or in part, a human carbonic anhydrase 2 (CA2; SEQ ID NO: 5), and further comprises an E106D mutation in the amino acid at position 106 (E106) of SEQ ID NO: 5. In some such embodiments, the DRD further comprises a C205S mutation in the amino acid at position 205 (C205) of SEQ ID NO: 5.

In some embodiments, a DRD of the present disclosure comprises, in whole or in part, a human carbonic anhydrase 2 (CA2; SEQ ID NO: 5), and further comprises a W208S mutation in the amino acid at position 208 (W208) of SEQ ID NO: 5. In some such embodiments, the DRD further comprises a C205S mutation in the amino acid at position 205 (C205) of SEQ ID NO: 5.

In some embodiments, a DRD of the present disclosure comprises, in whole or in part, a human carbonic anhydrase 2 (CA2; SEQ ID NO: 5), and further comprises a I59N mutation in the amino acid at position 59 (I59) of SEQ ID NO: 5. In some such embodiments, the DRD further comprises a G102R mutation in the amino acid at position 102 (G102) of SEQ ID NO: 5.

In some embodiments, a DRD of the present disclosure comprises, in whole or in part, a human carbonic anhydrase 2 (CA2; SEQ ID NO: 5), and further comprises a L156H mutation in the amino acid at position 156 (L156) of SEQ ID NO: 5. In some such embodiments, the DRD further comprises (i) a W4Y mutation in the amino acid at position 4 (W4) of SEQ ID NO: 5; (ii) a F225L mutation in the amino acid at position 225 (F225) of SEQ ID NO: 5; (iii) a deletion of amino acids at positions 257-260 of SEQ ID NO: 5; (iv) a deletion of amino acids at positions 1-5 of SEQ ID NO: 5; or (v) a deletion of amino acids G234, E235 and P236 of SEQ ID NO: 5.

In some embodiments, a DRD of the present disclosure comprises, in whole or in part, a human carbonic anhydrase 2 (CA2; SEQ ID NO: 5), and further comprises four mutations relative to SEQ ID NO: 5, said mutations corresponding to: (i) L156H, S172C, F178Y, and E186D; or (ii) D70N, D74N, D100N, and L156H.

In some embodiments, a DRD of the present disclosure comprises, in whole or in part, a human carbonic anhydrase 2 (CA2; SEQ ID NO: 5), and further comprises a first mutation and a second mutation relative to SEQ ID NO: 5, wherein: (i) the first mutation is a S73N mutation in the amino acid at position 73 (S73) of SEQ ID NO: 5; and (ii) the second mutation is a substitution of F or Y at the amino acid position 89 (R89) of SEQ ID NO: 5.

In some embodiments, a DRD of the present disclosure comprises, in whole or in part, a human carbonic anhydrase 2 (CA2; SEQ ID NO: 5), and further comprises a substitution of N or F at the amino acid position 56 (S56) of SEQ ID NO: 5. In some such embodiments, the DRD comprises two substitutions relative to SEQ ID NO: 5 that correspond to S56F and D71S.

In some embodiments, a DRD of the present disclosure comprises, in whole or in part, a human carbonic anhydrase 2 (CA2; SEQ ID NO: 5), and further comprises one or more substitutions relative to SEQ ID NO: 5, wherein at least one substitution is a substitution of D or N at the amino acid position 63 (G63) of SEQ ID NO: 5, and wherein the one or more substitutions correspond to: (i) G63D; (ii) G63D and M240L; (iii) G63D, E69V and N231I; or (iv) T55K, G63N and Q248N.

In some embodiments, a DRD of the present disclosure comprises, in whole or in part, a human carbonic anhydrase 2 (CA2; SEQ ID NO: 5), and further comprises two or more substitutions relative to SEQ ID NO: 5, wherein one of the two or more substitutions is a substitution of L or K at the amino acid position 71 (D71) of SEQ ID NO: 5, and wherein said two or more substitutions correspond to: (i) D71L and T87N; (ii) D71L and L250R; (iii) D71L, T87N and L250R; or (iv) D71K and T192F.

In some embodiments, a DRD of the present disclosure comprises, in whole or in part, a human carbonic anhydrase 2 (CA2; SEQ ID NO: 5), and further comprises two or more substitutions relative to SEQ ID NO: 5, wherein at least one of the two or more substitutions is: (i) a substitution of F at the amino acid position 241 (V241) of SEQ ID NO: 5; or (ii) a substitution of F or L at the amino acid position 249 (P249) of SEQ ID NO: 5; and wherein the two or more substitutions correspond to: (i) D72F and V241F; (ii) D72F and P249L; (iii) D72F and P249F; (iv) D72F, V241F and P249L; (v) A77I and P249F; or (vi) V241F and P249L.

In some embodiments, a DRD of the present disclosure comprises, in whole or in part, a human carbonic anhydrase 2 (CA2; SEQ ID NO: 5), and further comprises one or more substitutions relative to SEQ ID NO: 5, selected from Y51T, L183S, Y193I, L197P and the combination of V134F and L228F.

### Stimuli of Direct Cas-DRD Regulation Systems and Cas-Transcription Factor Systems

A direct Cas-DRD regulation system of the present disclosure and a Cas-transcription factor system of the present disclosure can be responsive to a stimulus, also referred to herein as a stimulating agent.

In some embodiments, a stimulus is a ligand. In some embodiments, a stimulus is an exogenous ligand. Ligands may be nucleic acid-based, protein-based, lipid-based, organic, inorganic or any combination of the foregoing. In some embodiments, ligands may be synthetic molecules. In some embodiments, ligands may be small molecule compounds. In some embodiments, ligands may be small molecule therapeutic drugs previously approved by a regulatory agency, such as the U.S. Food and Drug Administration (FDA).

As described in the present disclosure, a direct Cas-DRD regulation system and a Cas-transcription factor system can exhibit ligand-dependent activity. In the direct Cas-DRD regulation system, a ligand can bind to a DRD and stabilize a Cas protein that is operably linked to the DRD. In a Cas-transcription factor system, a ligand can bind to a DRD and stabilize a transcription factor or a domain of a transcription factor that is operably linked to the DRD. Ligands that are known to bind candidate DRDs can be tested for their effect on the activity of each system.

In some embodiments, a ligand is cell permeable. In some embodiments, a ligand may be designed to be lipophilic to improve cell permeability.

In some embodiments, a ligand is a small molecule. A small molecule ligand may be clinically approved to be safe and have appropriate pharmaceutical kinetics and distribution.

In some embodiments, the ligand may be complexed or bound to one or more other molecules such as, but not limited to, another ligand, a protein, peptide, nucleic acid, lipid, lipid derivative, sterol, steroid, metabolite, metabolite derivative or small molecule. In some embodiments, the ligand stimulus is complexed or bound to one or more different kinds and/or numbers of other molecules. In some embodiments, the ligand stimulus is a multimer of the same kind of ligand. In some embodiments, the ligand stimulus multimer comprises 2, 3, 4, 5, 6, or more monomers.

### CA2 Ligands

In some embodiments, a ligand of the present disclosure binds to carbonic anhydrases. In some embodiments, the ligand binds to and inhibits carbonic anhydrase function and is herein referred to as a carbonic anhydrase inhibitor.

In some embodiments, the ligand is a small molecule that binds to carbonic anhydrase 2. In one embodiment, the small molecule is a CA2 inhibitor. Examples of CA2 inhibitors include but are not limited to Celecoxib (also referred to as Celebrex), Valdecoxib, Rofecoxib, Acetazolamide, Methazolamide, Dorzolamide, Brinzolamide, Diclofenamide, Ethoxzolamide, Zonisamide, Dansylamide, and Dichlorphenamide.

In some embodiments, the ligands may comprise portions of small molecules known to mediate binding to CA2. Ligands may also be modified to reduce off-target binding to carbonic anhydrases other than CA2 and increase specific binding to CA2.

In some embodiments, the stimulus may be a ligand that binds to more than one carbonic anhydrase. In one embodiment, the stimulus is a pan carbonic anhydrase inhibitor that may bind to two or more carbonic anhydrases.

### DHFR Ligands

In some embodiments, a ligand of the present disclosure binds to dihydrofolate reductase. In some embodiments, the ligand binds to and inhibits dihydrofolate reductase function and is herein referred to as a dihydrofolate inhibitor.

In some embodiments, the ligand may be a selective inhibitor of human DHFR. Ligands of the disclosure may also be selective inhibitors of dihydrofolate reductases of bacteria and parasitic organisms such as Pneumocystis spp., Toxoplasma spp., Trypanosoma spp., Mycobacterium spp., and Streptococcus spp. Ligands specific to other DHFR may be modified to improve binding to human dihydrofolate reductase.

Examples of dihydrofolate reductase inhibitors include, but are not limited to, Trimethoprim (TMP), Methotrexate (MTX), Pralatrexate, Piritrexim, Pyrimethamine, Talotrexin, Chloroguanide, Pentamidine, Trimetrexate, aminopterin, C1 898 trihydrochloride, Pemetrexed Disodium, Raltitrexed, Sulfaguanidine, Folotyn, Iclaprim and Diaveridine.

In some embodiments, ligands of the present disclosure may include dihydrofolic acid or any of its derivatives that may bind to human DHFR. In some embodiments, ligands of the present disclosure may be 2,4, diaminohetrocyclic compounds. In some embodiments, the 4-oxo group in dihydrofolate may be modified to generate DHFR inhibitors. In one example, the 4-oxo group may be replaced by 4-amino group. Various diamino heterocycles, including pteridines, quinazolines, pyridopyrimidines, pyrimidines, and triazines, may also be used as scaffolds to develop DHFR inhibitors.

In some embodiments, ligands include TMP-derived ligands containing portions of the ligand known to mediate binding to DHFR. Ligands may also be modified to reduce off-target binding to other folate metabolism enzymes and increase specific binding to DHFR.

### ER Ligands

In some embodiments, a ligand of the present disclosure binds to ER. Ligands may be agonists or antagonists. In some embodiments, the ligand binds to and inhibits ER function and is herein referred to as an ER inhibitor. In some embodiments, the ligand may be a selective inhibitor of human ER. Ligands of the disclosure may also be selective inhibitors of ER of other species. Ligands specific to other ER may be modified to improve binding to human ER.

Ligands may be ER agonists such as but not limited to endogenous estrogen 17b-estradiol (E2) and the synthetic nonsteroidal estrogen diethylstilbestrol (DES). In some embodiments, the ligands may be ER antagonists, such as ICI-164,384, RU486, tamoxifen, 4-hydroxytamoxifen (4-OHT), fulvestrant, oremifene, lasofoxifene, clomifene, femarelle and ormeloxifene and raloxifene (RAL).

In some embodiments, the stimulus of the current disclosure may be ER antagonists such as, but not limited to, Bazedoxifene and/or Raloxifene.

In some embodiments, ligands include Bazedoxifene-derived ligands containing portions of the ligand known to mediate binding to ER. Ligands may also be modified to reduce off-target binding to other folate metabolism enzymes and increase specific binding to ER derived DRDs.

### Phosphodiesterase Ligands

In some embodiments, ligands of the present disclosure bind to phosphodiesterases. In some embodiments, the ligands bind to and inhibit phosphodiesterase function and are herein referred to as phosphodiesterase inhibitors.

In some embodiments, the ligand is a small molecule that binds to phosphodiesterase 5. In one embodiment, the small molecule is a hPDE5 inhibitor. Examples of hPDE5 inhibitors include, but are not limited to, Sildenafil, Vardenafil, Tadalafil, Avanafil, Lodenafil, Mirodenafil, Udenafil, Benzamidenafil, Dasantafil, Beminafil, SLx-2101, LAS 34179, UK-343,664, UK-357903, UK-371800, and BMS-341400.

In some embodiments, ligands include sildenafil-derived ligands containing portions of the ligand known to mediate binding to hPDE5. Ligands may also be modified to reduce off-target binding to phosphodiesterases and increase specific binding to hPDE5.

In some embodiments, the stimulus may be a ligand that binds to more than one phosphodiesterase. In one embodiment, the stimulus is a pan-phosphodiesterase inhibitor that may bind to two or more hPDEs such as Aminophyline, Paraxanthine, Pentoxifylline, Theobromine, Dipyridamole, Theophyline, Zaprinast, Icariin, CDP-840, Etazolate and Glaucine.

In some embodiments, the ligand is a hPDE1 inhibitor. In some embodiments, the ligand is a hPDE2 inhibitor. In some embodiments, the ligand is a hPDE3 inhibitor. In some embodiments, the ligand is a hPDE4 inhibitor. In some embodiments, the ligand is a hPDE6 inhibitor. In some embodiments, the ligand is a hPDE7 inhibitor. In some embodiments, the ligand is a hPDE8 inhibitor. In some embodiments, the ligand is a hPDE9 inhibitor. In some embodiments, the ligand is a hPDE10 inhibitor.

### FKBP Ligands

In some embodiments, ligands of the present disclosure bind to FKBP, including human FKBP. In some embodiments, the ligand is SLF or Shield-1.

### Pharmaceutical Compositions

The present teachings further comprise pharmaceutical compositions comprising one or more of the direct Cas-DRD regulation systems, Cas-transcription factor systems, nucleic acids, polynucleotides, modified cells or payloads of the present disclosure, and optionally at least one pharmaceutically acceptable excipient or inert ingredient.

As used herein the term “pharmaceutical composition” refers to a preparation of one or more of the systems, nucleic acids, polynucleotides, payloads or components described herein, or pharmaceutically acceptable salts thereof, optionally with other chemical components such as physiologically suitable carriers and excipients.

The term “excipient” or “inactive ingredient” refers to an inert or inactive substance added to a pharmaceutical composition to further facilitate administration of a compound.

In some embodiments, compositions are administered to humans, human patients or subjects. For the purposes of the present disclosure, the phrase “active ingredient” generally refers to any one or more components of the direct Cas-DRD regulation system or Cas-transcription factor system to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Subjects to which administration of the pharmaceutical compositions is contemplated include, but are not limited to, non-human mammals, including agricultural animals such as cattle, horses, chickens and pigs, domestic animals such as cats, dogs, or research animals such as mice, rats, rabbits, dogs and non-human primates.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient or inert ingredient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Inactive Ingredients

In some embodiments, pharmaceutical or other formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

### Dosing, Delivery and Administration

Polynucleotides and compositions of the disclosure may be delivered to a cell or a subject through one or more routes and modalities. Polynucleotides may be delivered to a cell or subject using a viral vector system, which include DNA and RNA viruses and have either episomal or integrated genomes after delivery to the cell. Viruses, which are useful as vectors include, but are not limited to an adenovirus, adeno-associated virus (AAV), alphavirus, flavivirus, herpes virus, measles virus, rhabdovirus, retrovirus, lentivirus, Newcastle disease virus (NDV), poxvirus, and picornavirus vectors. In some embodiments, the virus is selected from a lentivirus vector, a gamma retrovirus vector, adeno-associated virus (AAV) vector, adenovirus vector, and a herpes virus vector (e.g., HSV).

Non-viral vector delivery systems include, but are not limited to, DNA plasmids, DNA minicircles, cosmids, naked nucleic acid molecules, which may be modified to prevent degradation, and nucleic acid complexed with a delivery vehicle such as a liposome or poloxamer.

Non-viral delivery of nucleic acids include, without limitation, the use of electroporation, lipofection, microinjection, biolistics, sonoporation, cell deformation, virosomes, liposomes, immunoliposomes, agent-enhanced uptake of nucleic acids, artificial virions, polycation- or lipid-nucleic acid conjugates; nucleic acids may comprise naked DNA, modified DNA, naked RNA or capped RNA or modified RNA.

In some embodiments, viral vectors containing one or more polynucleotides as described herein are used to deliver them to a cell and/or a subject.

### Delivery

The polynucleotides, viral vectors, non-viral delivery systems and pharmaceutical compositions thereof may be delivered to cells, tissues, organs and/or organisms by methods and routes of administration known in the art. In some embodiments, the polynucleotides, viral vectors, non-viral delivery systems and pharmaceutical compositions thereof are delivered free from agents or modifications which promote transfection or permeability. In some embodiments, delivery may include formulation in a simple buffer such as saline or PBS.

In some embodiments, the polynucleotides, viral vectors, non-viral delivery systems and pharmaceutical compositions thereof may be formulated to include, without limitation, cell penetration agents, pharmaceutically acceptable carriers, delivery agents, bioerodible or biocompatible polymers, solvents, and/or sustained-release delivery depots. Formulations of the present disclosure may be delivered to cells using routes of administration known in the art and described herein.

The polynucleotides, viral vectors, non-viral delivery systems and pharmaceutical compositions thereof may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

The polynucleotides, viral vectors, non-viral delivery systems and pharmaceutical compositions thereof may be formulated in any manner suitable for delivery. The formulation may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

In one embodiment, a polynucleotide or vector formulation may be a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA.

For polynucleotides of the disclosure, the formulation may be selected from any of those taught, for example, in International Application PCT/US2012/069610.

In another aspect of the disclosure, polynucleotides encoding compositions of the disclosure, direct Cas-DRD regulation systems, Cas-transcription factor systems, or components thereof, and vectors comprising said polynucleotides may be introduced into cells such as, without limitation, immune effector cells, skeletal muscle cells, neuronal cells or hepatocytes.

In one aspect of the disclosure, polynucleotides encoding compositions of the disclosure, direct Cas-DRD regulation systems, Cas-transcription factor systems, or components thereof, may be packaged into plasmids, viral vectors or integrated into viral genomes allowing transient or stable expression of the polynucleotides. Preferable viral vectors are retroviral vectors including lentiviral vectors and gamma retroviral vectors. In some embodiments, lentiviral vectors may be preferred as they are capable of infecting both dividing and non-dividing cells.

Vectors may also be transferred to cells by non-viral methods, including by physical methods such as needles, electroporation, sonoporation, hydroporation; chemical carriers such as inorganic particles (e.g. calcium phosphate, silica, gold) and/or chemical methods. In some embodiments, synthetic or natural biodegradable agents may be used for delivery such as cationic lipids, lipid-nano emulsions, nanoparticles, peptide-based vectors, or polymer-based vectors. In some embodiments, vectors may be transferred to cells by temporary membrane disruption, for example, by high speed cell deformation.

In some embodiments, vectors of the present disclosure possess an origin of replication (ori) which permits amplification of the vector, for example in bacteria. Additionally, or alternatively, the vector includes selectable markers such as antibiotic resistance genes, genes for colored markers and suicide genes.

In some embodiments, the recombinant expression vector may comprise regulatory sequences, such as transcription and translation initiation and termination codons, which are specific to the type of host cell into which the vector is to be introduced.

### Lentiviral Vehicles/Particles

In some embodiments, lentiviral vectors may be used for gene delivery.

Lentiviral particles may be generated by co-expressing the virus packaging elements and the vector genome itself in a producer cell such as human HEK293T cells. These elements are usually provided in three or four separate plasmids. The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, of the direct Cas-DRD regulation systems, Cas-transcription factor systems, or components thereof of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther., 2005, 11: 452-459), FreeStyle™ 293 Expression System (ThermoFisher, Waltham, Mass.), and other HEK293T-based producer cell lines (e.g., Stewart et al., Hum Gene Ther._2011, 22(3):357-369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelope proteins from other viruses, such as the G protein of vesicular stomatitis virus (VSV-G) or baculoviral gp64 envelope proteins. In some aspects, the envelope proteins may be RD 114, RD 115 or derived from gibbon ape leukemia virus (GaLV) or a baboon retroviral envelope glycoprotein (BaEV).

Other elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof.

Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionII.

### Adeno-Associated Viral Particles

Delivery of polynucleotides of any of the direct Cas-DRD regulation systems, Cas-transcription factor systems, or components thereof of the present disclosure may be achieved using recombinant adeno-associated viral (rAAV) vectors. Such vectors or viral particles may be designed to utilize any of the known serotype capsids or combinations of serotype capsids.

AAV vectors include not only single stranded vectors but self-complementary AAV vectors (scAAVs). scAAV vectors contain DNA which anneals together to form double stranded vector genomes. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The direct Cas-DRD regulation systems, Cas-transcription factor systems, or components thereof of the present disclosure may be encoded in one or more viral genomes to be packaged in the AAV capsids taught herein.

Such vector or viral genomes may also include, in addition to at least one or two ITRs (inverted terminal repeats), certain regulatory elements necessary for expression from the vector or viral genome. Such regulatory elements are well known in the art and include for example promoters, introns, spacers, stuffer sequences, and the like.

The direct Cas-DRD regulation systems, Cas-transcription factor systems, or components thereof of the disclosure may be administered in one or more or separate AAV particles.

### Retroviral Vehicles/Particles (γ-Retroviral Vectors)

In some embodiments, retroviral vehicles/particles may be used to deliver the direct Cas-DRD regulation systems, Cas-transcription factor systems, or components thereof of the present disclosure. Retroviral vectors (RVs) allow the permanent integration of a transgene in target cells. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions of the present disclosure that is to be packaged in newly formed viral particles.

In some embodiments, the recombinant gamma-retroviral vectors are pseudotyped with envelope proteins from other viruses. Envelope glycoproteins are incorporated in the outer lipid layer of the viral particles which can increase/alter the cell tropism. In some aspects, the envelope proteins may be RD 114, RD 115 or derived from gibbon ape leukemia virus (GaLV) or a baboon retroviral envelope glycoprotein (BaEV).

In some embodiments, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promotor of choice, and/or an enhancer element. The choice of the internal promotors may be made according to specific requirements of gene expression needed for a particular purpose of the disclosure.

In some embodiments, polynucleotides of direct Cas-DRD regulation systems, Cas-transcription factor systems, or components thereof of the disclosure are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

In some embodiments, the direct Cas-DRD regulation systems, Cas-transcription factor systems, or components thereof of the disclosure may be administered in one or more AAV particles. In some embodiments, more than one direct Cas-DRD regulation system, Cas-transcription factor system, or components thereof of the disclosure may be encoded in a viral genome.

### Oncolytic Viral Vector

In some embodiments, polynucleotides of present disclosure may be packaged into oncolytic viruses. As used herein, the term “oncolytic virus” refers to a virus that preferentially infects and kills cancer cells such as vaccine viruses. An oncolytic virus can occur naturally or can be a genetically modified virus such as oncolytic adenovirus, and oncolytic herpes virus. In some embodiments, oncolytic vaccine viruses may include viral particles of a thymidine kinase (TK)-deficient, granulocyte macrophage (GM)-colony stimulating factor (CSF)-expressing, replication-competent vaccinia virus vector sufficient to induce oncolysis of cells in the tumor; See e.g., U.S. Pat. No. 9,226,977.

Messenger RNA (mRNA)

In some embodiments, the direct Cas-DRD regulation systems, Cas-transcription factor systems, or components thereof of the disclosure may be designed as messenger RNAs (mRNAs). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Such mRNA molecules may have the structural components or features of any of those taught in International Application number PCT/US2013/030062.

### Dosing

The present disclosure provides methods comprising administering any one or more components or compositions of a direct Cas-DRD regulation system and/or a Cas-transcription factor system to a subject in need thereof. These may be administered to a subject using any amount and any route of administration effective for preventing or treating or imaging a disease, disorder, and/or condition. The exact amount required will vary from subject to subject, depending on the species, age, and general condition of the subject, the severity of the disease, the particular composition, its mode of administration, its mode of activity, and the like.

Compositions in accordance with the present disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In one embodiment, a dose of genetically modified cells is delivered to a subject intramuscularly, subcutaneously, intravenously, stereo-tactically. In preferred embodiments, genetically modified cells are intravenously administered to a subject in need of gene editing.

In particular embodiments, patients receive a dose of genetically modified cells, of about 1×10⁵ cells/kg to at least 1×10⁸ cells/kg. In some embodiments, patients receive a dose of genetically modified cells of about 1×10⁵ cells/kg, about 5×10⁵ cells/kg, about 1×10⁶ cells/kg, about 5×10⁶ cells/kg about 1×10⁷ cells/kg, about 5×10⁷ cells/kg, about 1×10⁸ cells/kg, or more in one single intravenous dose.

In various embodiments, the methods of the invention provide more robust and safe gene therapy than existing methods and comprise administering a population or dose of cells comprising about 5% genetically modified cells, about 10% genetically modified cells, about 25% genetically modified cells, about 50% genetically modified cells, about 75% genetically modified cells, or about 90% genetically modified cells, or greater genetically modified cells to a subject.

### Ligand Dosing

Also provided herein are methods of administering ligands or DRD ligands in accordance with the disclosure to a subject in need thereof. Non-limiting examples of ligands for DRDs are provided in Table 1. The ligand may be administered to a subject or to cells, using any amount and any route of administration effective for tuning the system, DRD, or Cas proteins of the disclosure. The exact amount required will vary from subject to subject, depending on the species, age, and general condition of the subject, the severity of the disease, the particular composition, its mode of administration, its mode of activity, and the like. The subject may be a human, a mammal, or an animal. Ligand compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment.

The present disclosure provides methods for delivering to a cell or tissue any of the ligands described herein, comprising contacting the cell or tissue with said ligand and can be accomplished in vitro, ex vivo, or in vivo. In certain embodiments, the ligand is administered to a cell or tissue in vivo. In certain embodiments, the ligands in accordance with the present disclosure may be administered to cells at dosage levels sufficient to stabilize a Cas-DRD fusion protein or the DRD-TF.

The desired dosage of the ligands of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event. The desired dosage of the ligand of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

### Administration

DNA encoding Cas proteins (e.g., Cas9 proteins) operably linked directly or indirectly with a DRD of the present disclosure and/or gRNA molecules, can be administered to subjects or delivered into cells by methods known in the art or described herein. For example, Cas-encoding and/or gRNA-encoding nucleic acids can be delivered, e.g., by vectors (e.g., viral or non-viral vectors), non-vector based methods (e.g., using naked DNA or DNA complexes), or a combination thereof. Systemic modes of administration include oral and parenteral routes. Parenteral routes include, by way of example, intravenous, intrarterial, intraosseous, intramuscular, intradermal, subcutaneous, epidural, transdermal, oral, enteral, intranasal and intraperitoneal routes. Components administered systemically may be modified or formulated to target the components to the eye.

Local modes of administration include, by way of example, intrathecal, intracerebroventricular, intraparenchymal (e.g., localized intraparenchymal delivery to the striatum (e.g., into the caudate or into the putamen)), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum or substantia nigra intraocular, intraorbital, subconjuctival, intravitreal, subretinal or transscleral routes. In an embodiment, significantly smaller amounts of the components (compared with systemic approaches) may exert an effect when administered locally (for example, intraparenchymal or intravitreal) compared to when administered systemically (for example, intravenously). Local modes of administration can reduce or eliminate the incidence of potentially toxic side effects that may occur when therapeutically effective amounts of a genetic construct are administered systemically.

In some embodiments, compositions of the present disclosure may be administered to cells ex vivo and subsequently administered to the subject. In further embodiments, the cell is selected from a B cell, a T cell, a natural killer cell (NK cell), or a tumor infiltrating lymphocyte (TIL). Immune cells can be isolated and expanded ex vivo using a variety of methods known in the art. For example, methods of isolating cytotoxic T cells are described in U.S. Pat. Nos. 6,805,861 and 6,531,451. Isolation of NK cells is described in U.S. Pat. No. 7,435,596.

In some embodiments, depending upon the nature of the cells, the cells may be introduced into a host organism, e.g., a mammal, in a wide variety of ways including by injection, transfusion, infusion, or implantation. In some embodiments, the cells of the disclosure may be introduced at a specified site in the body, such as at the site of a tumor. The number of cells that are employed will depend upon a number of circumstances, the purpose for the introduction, the lifetime of the cells, the protocol to be used, for example, the number of administrations, the ability of the cells to multiply, or the like. The cells may be in a physiologically-acceptable medium.

In some embodiments, the cells of the disclosure may be administrated in multiple doses to subjects having a disease or condition. The administrations generally effect an improvement in one or more symptoms of a clinical condition and/or treat or prevent a clinical condition or symptom thereof.

In some embodiments, compositions of the present disclosure may be administered in vivo. In some embodiments, polynucleotides of the present disclosure may be delivered in vivo to the subject via gene therapy.

In some embodiments, the guide RNA of the present disclosure may be delivered directly to a cell as a native species by methods known to those of skill in the art, including injection or lipofection, or as transcribed from its cognate DNA, with the cognate DNA introduced into cells through electroporation, lipofection, microinjection, biolistics, sonoporation, high-velocity cell deformation, virosomes, liposomes, immunoliposomes, agent-enhanced uptake of nucleic acids, transient and stable transfection and viral transduction.

### Routes of Delivery

The pharmaceutical compositions, direct Cas-DRD regulation systems, Cas-transcription factor systems, nucleic acids, polynucleotides, payloads, vectors and cells of the present disclosure may be administered by any route to achieve a therapeutically effective outcome.

### Parenteral and Injectable Administration

In some embodiments, pharmaceutical compositions, direct Cas-DRD regulation systems, Cas-transcription factor systems, nucleic acids, polynucleotides, payloads, vectors and cells of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Applications and Uses

Gene and Cell Therapies with Regulated Cas

While there are several uses for the compositions and methods of the present disclosure that do not involve a medical treatment, for example, to generate cell lines and reagents for scientific research, many uses contemplated herein involve the administration of the compositions of the present disclosure to generate in vivo gene therapy or modified cells for adoptive cell therapy.

The present disclosure provides methods of correcting, regulating, altering and deleting the target genes and their corresponding functional proteins described herein using components of a direct Cas-DRD regulation system and/or a Cas-transcription factor system. It is to be understood that one of skill in the art will be able to design suitable guide RNAs for recognition of and hybridization with a target nucleic acid including a target gene as described herein.

In certain embodiments, correcting comprises changing a mutant gene that encodes a truncated protein or no protein at all, such that full-length functional or partially full-length functional protein expression is obtained. Correcting a mutant gene can comprise replacing the region of the gene that has the mutation or replacing the entire mutant gene with a copy of the gene that does not have the mutation using a repair mechanism such as homology-directed repair (HDR). Correcting a mutant gene can also comprise repairing a frameshift mutation that causes a premature stop codon, an aberrant splice acceptor site or an aberrant splice donor site, by generating a double stranded break in the gene that is then repaired using non-homologous end joining (NHEJ). NHEJ can add or delete at least one base pair during repair, which may restore the proper reading frame and eliminate the premature stop codon. Correcting a mutant gene can also comprise disrupting an aberrant splice acceptor site or splice donor sequence. Correcting can also comprise deleting a non-essential gene segment by the simultaneous action of two nucleases on the same DNA strand in order to restore the proper reading frame by removing the DNA between the two nuclease target sites and repairing the DNA break by NHEJ.

In certain embodiments, “Homology-directed repair” or “HDR” refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with components of a direct Cas-DRD regulation system and/or a Cas-transcription factor system, then the cellular machinery will repair the break by homologous recombination, which is enhanced several orders of magnitude in the presence of DNA cleavage. When the homologous DNA piece is absent, nonhomologous end joining may take place instead.

In various embodiments, one or more vectors comprising components of a direct Cas-DRD regulation system and/or a Cas-transcription factor system provides curative, preventative, or ameliorative benefits to a subject diagnosed with or that is suspected of having a monogenic, or polygenic disease, disorder, or condition or a disease, disorder, or condition amenable to genome editing. In some embodiments, viral constructs or vectors of the present disclosure can infect the target cells or tissues in vivo, ex vivo, or in vitro. In some ex vivo and in vitro embodiments, the infected cells can then be administered to a subject in need of therapy. In various embodiments, vectors, viral particles, and genetically modified cells of the invention are used to treat, prevent, and/or ameliorate a monogenic or polygenic disease, disorder, or condition, or a disease, disorder, or condition amenable to genome editing in a subject.

Cas molecules and gRNA molecules, e.g., a Cas9 molecule/gRNA molecule complex, can be used to manipulate a cell (e.g., an animal cell or a plant cell), e.g., to deliver a payload, or edit a target nucleic acid, in a wide variety of cells. Typically a Cas protein directly regulated by a DRD as in a direct Cas-DRD regulation system and/or a Cas protein regulated by a transcription factor as in a Cas-transcription factor system forms a Cas molecule/gRNA molecule complex that is used to edit or alter the structure of a target nucleic acid. Delivery or editing can be performed in vitro, ex vivo, or in vivo.

In some embodiments, a cell is manipulated by editing (e.g., introducing a mutation or correcting) one or more target genes, e.g., as described herein. In some embodiments, the expression of one or more target genes (e.g., one or more target genes described herein) is modulated, e.g., in vivo. In some embodiments, the expression of one or more target genes (e.g., one or more target genes described herein) is modulated, e.g., ex vivo.

In some embodiments, the cells are manipulated (e.g., converted or differentiated) from one cell type to another. In some embodiments, a pancreatic cell is manipulated into a beta islet cell. In some embodiments, a fibroblast is manipulated into an iPS cell. In some embodiments, a preadipocyte is manipulated into a brown fat cell. Other exemplary cells include, e.g., muscle cells, neural cells, leukocytes, and lymphocytes.

In some embodiments, the cell is a diseased or mutant-bearing cell. Such cells can be manipulated to treat the disease, e.g., to correct a mutation, or to alter the phenotype of the cell, e.g., to inhibit the growth of a cancer cell, to insert or delete a nucleotide, or nucleotide sequence, cut a portion of an exon, intron, or an entire gene or open reading frame, and optionally, insert a corrected portion of a gene. For example, a cell is associated with one or more diseases or conditions describe herein. In some embodiments, the cell is a cancer stem cell.

In some embodiments, the manipulated cell is a normal cell.

In some embodiments, the manipulated cell is a stem cell or progenitor cell (e.g., iPS, embryonic, hematopoietic, adipose, germline, lung, or neural stem or progenitor cells).

In some embodiments, the manipulated cells are suitable for producing a recombinant biological product. For example, the cells can be CHO cells or fibroblasts. In an embodiment, a manipulated cell is a cell that has been engineered to express a protein.

In some embodiments, the cell being manipulated is selected from fibroblasts, monocytic precursors, B cells, exocrine cells, pancreatic progenitors, endocrine progenitors, hepatoblasts, myoblasts, or preadipocytes. In some embodiments, the cell is manipulated (e.g., converted or differentiated) into muscle cells, erythroid-megakaryocytic cells, eosinophils, iPS cells, macrophages, T cells, islet beta-cells, neurons, cardiomyocytes, blood cells, endocrine progenitors, exocrine progenitors, ductal cells, acinar cells, alpha cells, beta cells, delta cells, pancreatic polypeptide cells (PP cells), hepatocytes, cholangiocytes, or brown adipocytes.

In some embodiments, the cell is a muscle cell, erythroid-megakaryocytic cell, eosinophil, iPS cell, macrophage, T cell, islet beta-cell, neuron, cardiomyocyte, blood cell, endocrine progenitor, exocrine progenitor, ductal cell, acinar cell, alpha cell, beta cell, delta cell, pancreatic polypeptide cell (PP cell), hepatocyte, cholangiocyte, or white or brown adipocyte.

The Cas molecule/gRNA molecule complex of a direct Cas-DRD regulation system and/or a Cas-transcription factor system described herein can be delivered to a target cell. In an embodiment, the target cell is a normal cell.

In an embodiment, the target cell is a stem cell or progenitor cell (e.g., iPS, embryonic, hematopoietic, adipose, germline, lung, or neural stem or progenitor cell).

In an embodiment, the target cell is a CHO cell.

In an embodiment, the target cell is a fibroblast, monocytic precursor, B cell, exocrine cell, pancreatic progenitor, endocrine progenitor, hepatoblast, myoblast, or preadipocyte.

In an embodiment, the target cell is a muscle cell, erythroid-megakaryocytic cell, eosinophil, iPS cell, macrophage, T cell, islet beta-cell, neuron (e.g., a neuron in the brain, e.g., a neuron in the striatum (e.g., a medium spiny neuron), cerebral cortex, precentral gyms, hippocampus (e.g., a neuron in the dentate gyrus or the CA3 region of the hippocampus), temporal cortex, amygdala, frontal cortex, thalamus, cerebellum, medulla, putamen, hypothalamus, tectum, tegmentum or substantia nigra), cardiomyocyte, blood cell, endocrine progenitor, exocrine progenitor, ductal cell, acinar cell, alpha cell, beta cell, delta cell, PP cell, hepatocyte, cholangiocyte, or brown adipocyte.

In an embodiment, the target cell is manipulated ex vivo by editing (e.g., introducing a mutation or correcting) one or more target genes and/or modulating the expression of one or more target genes, and administered to the subject.

In various embodiments, viral vectors are administered by direct injection to a cell, tissue, or organ of a subject in need of gene therapy, in vivo. In various other embodiments, cells are infected and optionally expanded in vitro or ex vivo with vectors contemplated herein. The infected cells are then administered to a subject in need of therapy. The cells may be allogeneic, or autologous.

A “subject,” as used herein, includes any animal that exhibits a symptom of a disease, disorder, or condition that can be treated with the direct Cas-DRD regulation systems and components thereof, Cas-transcription factor systems and components thereof, vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (lower or higher amounts than a “normal” or “healthy” subject) of one or more physiological activities that can be modulated by genome editing.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In various embodiments, a subject in need of a cell-based therapy is administered a population of cells comprising an effective amount of genetically modified cells contemplated herein.

As used herein, the term “amount” refers to “an amount effective” or “an effective amount” of a virus or genetically modified therapeutic cell to achieve a beneficial or desired prophylactic or therapeutic result, including clinical results.

A “prophylactically effective amount” refers to an amount of a virus or genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or modified therapeutic cell may vary according to factors such as the disease state, age, sex, and weight of the individual, and the ability of the virus or therapeutic cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient).

In one embodiment, the present invention includes a method of providing a genetically modified cell to a subject that comprises administering, e.g., parenterally, one or more cells transduced with a vector contemplated herein.

In various embodiments, one or more vectors comprising components of a direct Cas-DRD regulation system and/or a Cas-transcription factor system contemplated herein can be used to knockout or disrupt a gene or genetic regulatory sequence, correct a sequence in the genome, or insert genetic material into the genome. Such vectors comprise one or more nucleic acid sequences that encode guide RNA(s) that function to target the Cas nuclease (e.g., Cas9 nuclease) to one or more target sites to facilitate altering the genome of a target cell, tissue or organ.

Illustrative examples of target nucleic acids comprising target sites include sequences associated with a signaling biochemical pathway, e.g., a signaling biochemical pathway-associated gene or polynucleotide. Further illustrative examples of target nucleic acids include a disease-associated gene or polynucleotide. A “disease-associated” gene or polynucleotide refers to any gene or polynucleotide which is yielding transcription or translation products at an abnormal level or in an abnormal form in cells derived from disease-affected tissues compared with tissues or cells of a non-disease control. It may be a gene that becomes expressed at an abnormally high level; it may be a gene that becomes expressed at an abnormally low level, or it may be a rearrangement of two or more genes that provide a knock-in or knock-out function that did not previously exist in the cell, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

In a particular embodiment, editing of the genome in a cell comprises insertion of a direct Cas-DRD regulation system or Cas-transcription factor system. The regulated Cas nuclease (e.g., Cas9 nuclease) of the inserted system can be activated or repressed in the presence or absence of an exogenous ligand or small molecule, referred to herein as a stimulus molecule or stimulating agent.

In various embodiments, one or more crRNAs or sgRNAs contemplated herein, can be designed to target a polynucleotide sequence involved in the pathogenesis of a monogenetic disease, or a polygenic disease, to modify a disease-causing gene.

In some embodiments, compositions and methods of the disclosure may be used to modify genes in immune cells, for example, in T cells, NK cells, in Tumor Infiltrating Lymphocytes, used for T cell therapy; to modify nociceptive genes; to modify genes in viral genomes; to modify genes involved in neurodegenerative diseases, for example, Duchenne Muscular Dystrophy, (DMD); to modify genes involved in kidney disease; to modify genes involved in hemoglobinopathies, to modify genes involved in trinucleotide repeat diseases; to modify genes involved in inflammatory disease; to modify genes involved in cancer; to modify genes involved in cardiovascular disease; to modify genes involved in liver disease; to modify genes involved in retinal diseases; and to modify polynucleotide sequences that contribute to aberrant splicing.

In a particular embodiment, vectors contemplated herein can be used to knockout or disrupt a gene or genetic regulatory sequence, correct a sequence in the genome, or insert genetic material into the genome.

As used herein, the term “monogenic disease” refers to a disease in which modification of a single gene is associated with a disorder, disease, or condition in a subject. Though relatively rare, monogenic diseases affect millions of people worldwide. Scientists currently estimate that over 10,000 human diseases are known to be monogenic. Pure genetic diseases are caused by a single error in a single gene in the human DNA. The nature of disease depends on the functions performed by the modified gene. The single-gene or monogenic diseases can be classified into three main categories: Dominant, Recessive, and X-linked. Exemplary diseases that can be treated using the direct Cas-DRD regulation system or Cas-transcription factor system of the present disclosure can include recessive diseases that occur due to damages in both copies or alleles. Dominant diseases are monogenic disorders that involve damage to only one gene copy. X-linked diseases are monogenic disorders that are linked to defective genes on the X chromosome which is the sex chromosome. The X-linked alleles can also be dominant or recessive.

Further illustrative examples of conditions treatable with the direct Cas-DRD regulation systems and/or Cas-transcription factor systems and components thereof contemplated herein include: metabolic diseases, neurological diseases, neuromuscular diseases, cardiovascular diseases, hyper-proliferative diseases, hematological diseases, immunological diseases, autoimmune diseases, inflammatory diseases, lysosome storage diseases, congenital and genetic diseases, inherited diseases, for example, Duchenne muscular dystrophy.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. A healthcare practitioner skilled in the art may monitor efficacy of treatment or prevention by measuring any one of such parameters, or any combination of parameters. In connection with the administration of compositions of the present disclosure, “effective against” for example a cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given composition or formulation of the present disclosure can also be judged using an experimental animal model for the given disease as known in the art. When using an experimental animal model, efficacy of treatment is evidenced when a statistically significant change is observed.

### Modifying Expression of Dystrophin

DMD is caused by mutations in the dystrophin gene. With a genomic region of over 2.2 megabases in length, dystrophin is the second largest human gene. The dystrophin gene contains 79 exons that are processed into an 11,000 base pair mRNA that is translated into a functional 427 kDa protein. Provided herein are in vivo, ex vivo and direct cellular treatment methods for gene editing of diseased muscle and cardiac myocyte cells to create permanent changes to the genome that can restore the dystrophin reading frame and restore dystrophin protein activity in these cells. Such methods use endonucleases, such as CRISPR/Cas9 nucleases, to permanently delete (excise), insert, or replace (delete and insert) exons (i.e., mutations in the coding and/or splicing sequences) in the genomic locus of the dystrophin gene. In some embodiments, an endonuclease such as Cas9 is operably linked to a DRD, such as a CA2 or ER DRD, which permits regulated expression of the endonuclease. The endonuclease may be turned on or off, its expression level may be regulated, and the timing of its expression may be controlled. In some embodiments, a regulated endonuclease such as Cas9 may be turned off once gene editing is deemed complete. By removing the mutations present in the exon or intron, the present invention mimics the product produced by exon skipping, and/or restores the reading frame with as few as a single treatment (rather than deliver exon skipping oligos for the lifetime of the patient). The specific mutation can be targeted using at least one short guide RNAs that hybridize upstream, downstream or in regions containing sequences containing the one or more mutations.

In certain embodiments, a presently disclosed genetic construct (e.g., a vector) encodes at least one inducible Cas (e.g., Cas9) fusion protein, or an inducible transcription factor that selectively transcribes a Cas (e.g., Cas9) nuclease of the present disclosure and is coupled with one or more gRNA molecules that target a dystrophin gene, for example, a human dystrophin gene which are disclosed in PCT/US16/025738, the contents of which are incorporated by reference in its entirety. In various embodiments, an exemplary inducible Cas9 gene editing vector restores dystrophin protein expression in cells from DMD patients. Exons 50 and 51 are frequently adjacent to frame-disrupting deletions in DMD. Elimination of exon 51 from the dystrophin transcript by exon skipping can be used to treat approximately 15% of all DMD patients. This class of dystrophin mutations is ideally suited for permanent correction by NHEJ-based genome editing and HDR. The genetic constructs (e.g., vectors) described herein may be used for targeted modification of exon 51 in the human dystrophin gene. An exemplified inducible Cas9 genetic construct (e.g., a vector) is transfected into human DMD cells and mediates efficient gene modification and conversion to the correct reading frame. Protein restoration is concomitant with frame restoration and detected in a bulk population of cells treated with components of the direct Cas-DRD regulation system and/or the Cas-transcription factor system of the present disclosure. The treated cells are administered a stimulus molecule that stabilizes the DRD linked to the Cas (e.g., Cas9) nuclease, or the transcription factor that specifically acts on the transcription of Cas (e.g., Cas9) nucleases described herein. The activity of the Cas (e.g., Cas9) nuclease on editing the dystrophin gene can be modulated as needed by increasing or decreasing the amount of stimulus molecule that is administered. The Cas (e.g., Cas9) nuclease activity may be turned off by withdrawal of the stimulus molecule after gene editing is deemed complete.

### Modifying Expression of CD47 as a Treatment for Myeloid Malignancies

CD47 (also known as integrin associated protein) is a transmembrane protein that mainly functions as an anti-phagocytic or “do not eat me” signal, enabling CD47-expressing cells to evade phagocytic elimination by macrophages and other phagocytes. Tumor cells express high levels of CD47 that binds to signal-regulatory protein alpha (SIRPα), an inhibitory receptor on macrophages, allowing tumor cells to evade phagocytosis. Recent studies have shown that blocking CD47 with a monoclonal antibody with an IgG4 constant region (IgG4-Fc (fragment crystallizable region)) or a fusion protein consisting of the soluble ectodomain of SIRPα or a derivative thereof (e.g., CV1) and IgG4-Fc (SIRPα-Fc) has potent antitumor activity in preclinical animal models.

The role of CD47 in cancer-mediated evasion of phagocytosis was first described in acute myeloid leukemia (AML). In initial studies, CD47 was found to be overexpressed in both mouse and human AML compared to normal cell counterparts and its upregulation was directly tied to disease pathogenesis via macrophage evasion. AML is organized as a cellular hierarchy initiated and maintained by a subset of self-renewing leukemia stem cells (LSC). These LSC have been hypothesized to be a disease-initiating cell population and thus eradication of disease-initiating clones is presumably required for cure. LSC phenotype and function have been well-characterized. Clinically, LSC gene signatures have been shown to predict prognosis in AML patients, with LSC gene enrichment as an independent poor prognostic factor.

Identification and therapeutic targeting of markers of LSC is an attractive therapeutic strategy to selectively eliminate the disease-initiating cell population thus leading to potential cure. In AML patients, CD47 was identified as an LSC marker. CD47 cell surface protein expression was shown to be increased on CD34+CD38−CD90−Lin− leukemia stem cells (LSCs) compared to normal CD34+CD38−CD90+Lin− hematopoietic stem cell (HSC) counterparts. Pre-clinical data also demonstrate that CD47 is an LSC marker in AML. Thus, anti-CD47 therapies using tunable and regulatable Cas (e.g., Cas9) gene editing constructs in accordance with the present disclosure that delete expression of CD47 and lead to the eradication of LSCs may lead to long term remission.

In various embodiments, a genetic construct (e.g., a vector) which is designed to abrogate the expression of CD47 in a cancer cell or a LSC, encodes at least one gRNA molecule that targets a CD47 gene (e.g., human CD47 gene). The at least one gRNA molecules can recognize and bind a target region of DNA which encodes the CD47 molecule or a region thereof. The target region(s) can be chosen immediately upstream of possible out-of-frame stop codons such that insertions or deletions during the gene editing process disrupts the reading frame of the CD47 gene by insertion or deletion of nucleotides (INDELS), for example by NHEJ-mediated INDELS, thereby provoking a frame-shift deletion or missense mutation of the CD47 gene. The DRD-inducible constructs comprising a Cas9-DRD fusion protein or a Cas9 transcriptionally regulated by a DRD-transcription factor fusion protein of the present disclosure are engineered to contain at least one pair of offset guide RNAs designed to hybridize with target sites in the CD47 genomic locus, such that the Cas9 endonuclease activity at the region of DNA which encodes CD47 results in a break in the CD47 genomic locus, which when repaired by a cellular DNA repair process results in a modification to the genomic locus, preferably an INDEL.

In certain embodiments, a presently disclosed genetic construct (e.g., a vector) encodes at least one Cas9-DRD fusion protein, or Cas9 transcriptionally regulated by a DRD-transcription factor fusion protein of the present disclosure that is coupled with one or more gRNA molecules that target a CD47 gene, for example, a human CD47 gene expressed by cancer cells. In these embodiments, the CD47-targeting genetic constructs of the present disclosure are delivered to a tumor directly with a virus that is known to efficiently target and infect cancer cells, and turned “on” by the administration of a stimulus molecule.

CD47 is ubiquitously expressed on normal cells, which can present a major concern for potential toxicity with CD47 targeting agents. The ability to regulate expression of the anti-CD47 Cas (e.g., Cas9) gene editing, including the ability to turn off such gene editing, provides a scalable and drug-like control to gene editing. This control provides reduced risk of immunogenicity of Cas nucleases, limits off-target editing, for example, CD47 elimination in RBCs and other normal cells, and increases the duration of treatment.

In some embodiments, the methods of treatment contemplated herein can include one or more combination therapies with the tunable Cas (e.g., Cas9 or Cas12) editing genetic constructs described herein, in combination with one or more, effector molecules, such as, but not limited to, macrophage checkpoint inhibitors, T-cell PD1 and PD-L1 immune checkpoint inhibitors and other known treatments such as Rituximab, can also improve tumor CD47 specificity and limit off-target activity, when each of the combination elements are dosed suboptimally, but which when combined work synergistically.

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference and understanding, and the inclusion of such definitions herein should not necessarily be construed to mean a substantial difference over what is generally understood in the art. Commonly understood definitions of molecular biology terms and/or methods and/or protocols can be found in Rieger et al., Glossary of Genetics: Classical and Molecular, 5th edition, Springer-Verlag: New York, 1991; Lewin, Genes V, Oxford University Press: New York, 1994; Sambrook et al., Molecular Cloning, A Laboratory Manual (3d ed. 2001) and Ausubel et al., Current Protocols in Molecular Biology (1994), Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929. Articles such as “a,” “an,” and “the” may mean one or more than one unless indicated to the contrary or otherwise evident from the context. As appropriate, procedures involving the use of commercially available kits and/or reagents are generally carried out in accordance with manufacturer's guidance and/or protocols and/or parameters unless otherwise noted. When referring to illustrative constructs of the disclosure, such as constructs designed according to the direct Cas-DRD regulation systems or the Cas-transcription factor systems, the present disclosure may interchangeably identify these constructs with or without the term “OT-” at the beginning of the construct name. For example, the names “Cas9-024” and “OT-Cas9-024” refer to the same construct.

Adoptive cell therapy (ACT): The terms “adoptive cell therapy” or “adoptive cell transfer”, as used herein, refer to a cell therapy involving the transfer of cells into a patient, wherein cells may have originated from the patient, or from another individual, and are modified or engineered (altered) before being transferred back into the patient.

Agent: As used herein, the term “agent” refers to a biological, pharmaceutical, or chemical compound or composition. Non-limiting examples include simple or complex organic or inorganic molecule, a peptide, a protein, an oligonucleotide, an antibody, an antibody derivative, antibody fragment, a receptor, and soluble factor.

Agonist: The term “agonist” as used herein, refers to a compound that binds to and activates a receptor, either directly or indirectly by, for example, (a) forming a complex with another molecule that directly binds to and activates the receptor, or (b) otherwise resulting in the modification of another compound so that the other compound directly binds to and activates the receptor. An agonist may be referred to as an agonist of a particular receptor or family of receptors, e.g., agonist of a co-stimulatory receptor.

Antagonist: The term “antagonist” as used herein refers to any agent that inhibits or reduces the biological activity of the receptor or target(s) to which it binds.

Binding: As used herein, the term “binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

Cleavage: As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides (e.g., Cas9-DRD) are used for targeted double-stranded DNA cleavage.

Construct: The term “construct” and “nucleic acid construct” are used interchangeably and refer to a polynucleotide or a portion of a polynucleotide, typically comprising one or more nucleic acid sequences encoding one or more transcriptional products and/or proteins. A polynucleotide can comprise one or more constructs. A construct may be a recombinant nucleic acid molecule or a part thereof, such as a recombinant nucleic acid molecule selected from a plasmid, cosmid, virus, autonomously replicating nucleic acid molecule, phage, or linear or circular single-stranded or double-stranded DNA or RNA nucleic acid molecule, derived from any source, capable of genomic integration or autonomous replication. Constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the construct.

Delivery: The term “delivery” as used herein refers to the act or manner of delivering a compound, substance, entity, moiety, cargo or payload. A “delivery agent” refers to any agent which facilitates, at least in part, the delivery of one or more substances (including, but not limited to a compound and/or composition of the present disclosure) to a cell, subject or other biological system.

Derived from: As used herein, the phrase “derived from” refers to a polypeptide or polynucleotide that originates from the stated parent molecule or region or domain thereof or the stated parent sequence (e.g., nucleic acid sequence or amino acid sequence) and retains similarity to one or more structural and/or functional characteristics of the parent molecule or region or domain thereof or parent sequence. In some embodiments, a polypeptide or polynucleotide is derived from either (i) a full-length wild-type parent molecule or sequence; or (ii) a region or domain of a full-length wild-type parent molecule or sequence and retains the structural and/or functional characteristics of either (i) the full-length wild-type parent molecule or sequence; or (ii) the region or domain thereof, respectively. Structural characteristics include an amino acid sequence, a nucleic acid sequence, or a protein structure (e.g., such as a secondary protein structure, a tertiary protein structure, and/or quaternary protein structure). Functional characteristics include biological activity such as catalytic activity, binding ability, and/or subcellular localization. As a non-limiting example, a polypeptide or polynucleotide retains similarity to a parent molecule or sequence if it has at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to a parent nucleic acid sequence or amino acid sequence, over the entire length of the parent molecule or sequence. As another non-limiting example, a polypeptide retains similarity to a parent molecule or sequence if it comprises a region of amino acids that shares 100% identity to a parent amino acid sequence and said region ranges from 10-1,000 amino acids in length (e.g., greater than 20, 30, 40, 45, 50, 55, 60, 70, 80, 90, 100, 120, 140, 160, 180, 200, 250, 300, 350, 400, 450, 500, 600, 700, 800, and 900 amino acids or at least 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, and 1,000 amino acids). As another non-limiting example, a polypeptide retains similarity to a parent molecule or amino acid sequence if it comprises one, two, three, four, or five amino acid mutations as compared to the parent amino acid sequence. In some embodiments, a polypeptide or polynucleotide is considered to retain similarity to a parent molecule or region or domain thereof or a parent sequence if it has substantially the same biological activity as compared to the parent molecule or region or domain thereof or the parent sequence. In some embodiments, a polypeptide or polynucleotide is considered to retain similarity to a parent molecule or region or domain thereof or a parent sequence if there is overlap of at least one biological activity as compared to the parent molecule or region or domain thereof or parent sequence. In some embodiments, a polypeptide or polynucleotide is considered to retain similarity to a parent molecule or region or domain thereof or a parent sequence if it has improvement or optimization of one or more biological activities as compared to the parent molecule or region or domain thereof or parent sequence. For example, a DRD may be derived from a domain or region of a naturally occurring protein and is modified in any of the ways taught herein to optimize DRD function. As another example, a Cas protein of a Cas-DRD regulation system or a Cas-transcription factor system of the present disclosure may be derived from a naturally occurring parent Cas protein and retains RNA-guided DNA binding functionality and/or endonuclease functionality of the parent Cas protein even though the Cas protein may not have 100 percent sequence identity to the parent Cas protein. In some embodiments, biological activity may be optimized for a specified purpose, such as by retaining or enhancing certain activity while reducing or eliminating another activity as compared to a parent molecule.

Destabilized: As used herein, the term “destabilize,” “destabilizing region” or “destabilizing domain” refers to a region or molecule that is less stable than a starting, reference, wild-type or native form of the same region or molecule.

Engineered: As used herein, embodiments of the disclosure are “engineered” when they are designed to have a feature or property, whether structural or chemical, that varies from a starting point, wild type or native molecule.

Exogenous: An “exogenous” molecule is a molecule that is not normally present in a cell but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids.

An exogenous molecule can be the same type of molecule as an endogenous molecule. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, lipofection, microinjection, biolistics, sonoporation, high velocity cell deformation, virosomes, liposomes, immunoliposomes, agent-enhanced uptake of nucleic acids, direct injection, cell fusion, particle bombardment, calcium phosphate co-precipitation, DEAE-dextran-mediated transfer and viral vector-mediated transfer. An exogeneous molecule can also be the same type of molecule as an endogenous molecule but derived from a different species. For example, a human nucleic acid sequence may be introduced into a cell line originally derived from a mouse or hamster.

The term “exogenous” can also be used to refer to a part of a molecule, which part is exogenous with respect to a cell. For example, an exogenous promoter is a promoter that is not normally present in a cell but can be introduced into a cell by one or more genetic, biochemical or other methods.

By contrast, an “endogenous” molecule is one that is normally present in a particular cell at a particular developmental stage under particular environmental conditions. For example, an endogenous nucleic acid can comprise a chromosome, the genome of a mitochondrion, or other organelle, or a naturally occurring episomal nucleic acid. Additional endogenous molecules can include proteins, for example, transcription factors and enzymes.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Fragment: The term “fragment,” as applied to polynucleotide sequences, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent.

Functional Fragment: A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

Functional: As used herein, a “functional” biological molecule is a biological entity with a structure and in a form in which it exhibits a property and/or activity by which it is characterized.

Fusion: A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins, for example, a fusion between a DNA-binding domain (e.g., ZFP, TALE and/or meganuclease DNA-binding domains) and a nuclease (cleavage) domain (e.g., endonuclease, meganuclease, etc.) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

Gene: A “gene” refers to a polynucleotide comprising nucleotides that encode a functional molecule including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence.

The transcribed polynucleotide can have a sequence encoding a polypeptide, such as a functional protein, which can be translated into the encoded polypeptide when placed under the control of an appropriate regulatory region. A gene may comprise several operably linked fragments, such as a promoter, a 5′ leader sequence, a coding sequence and a 3′ nontranslated sequence, such as a polyadenylation site, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

Gene expression: “Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

Gene delivery: “Gene delivery” or “gene transfer” refers to methods for introduction of recombinant or foreign DNA into host cells. The transferred DNA can remain non-integrated or preferably integrates into the genome of the host cell. Gene delivery can take place for example by transduction, using viral vectors, or by transformation of cells, using known methods, including, without limitation, electroporation, cell bombardment, lipofection, microinjection, biolistics, sonoporation, cell deformation, liposomes, immunoliposomes or agent-enhanced uptake of nucleic acids

Genome: The term “genome” includes chromosomal as well as mitochondrial, chloroplast and viral DNA or RNA.

Genome engineering: The term “genome engineering” as used herein refers to the process of making specific modifications or alterations in the genome of an organism. According to the present disclosure, genome engineering may be used in reference to an entire organism or to a cell or a population of cells.

Guide RNA: The term “guide RNA” or “gRNA” as used in the present disclosure refers to the RNA or sequence encoding the RNA that functions to confer target sequence specificity to a CRISPR-Cas system. Guide RNAs are typically understood to be non-coding short RNA sequences that bind to a complementary target DNA sequence and guide a Cas protein to a specific location on the DNA. It is known in the art that different Cas proteins have different requirements for guide RNAs. Synthetic guide RNA can be designed to mimic the structures and functions of RNA molecules that enable sequence-specific destruction of invading genetic elements in prokaryotic adaptive immunity. In a prokaryotic Type II CRISPR-Cas system, a two-RNA structure formed from a mature crRNA and a tracrRNA (i.e., “a dual tracrRNA:crRNA”) directs Cas9 endonuclease to cleave target DNA. In one type of synthetic system mimicking the prokaryotic system, a synthetic tracrRNA and a synthetic crRNA are designed to direct Cas endonuclease activity to a DNA target of interest. In another type of synthetic system, a synthetic single guide RNA (sgRNA) is engineered as a single RNA chimera (mimicking both the crRNA and the tracrRNA combined) to also direct sequence-specific Cas endonuclease activity. The terms “guide RNA” and “gRNA” may be used in the present disclosure to refer to a designed sgRNA.

Immune cells: The term “an immune cell”, as used herein, refers to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

Modified: As used herein, the term “modified” refers to a changed state or structure of a molecule or entity as compared with a parent or reference molecule or entity. Molecules may be modified in many ways including chemically, structurally, and functionally. For example, a targeted genetic alteration is a type of modification.

Modulation of gene expression: “Modulation of gene expression” refers to a change in the activity of a gene. Modulation of expression includes, but is not limited to, gene activation and gene repression. Genome editing (e.g., cleavage, alteration, inactivation, random mutation) can be used to modulate expression. “Modulating gene expression” includes increasing or decreasing transcription of a gene.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and/or deletion of one or more amino acids (in the case of proteins and/or peptides) and/or nucleotides (in the case of nucleic acids and or polynucleic acids e.g., polynucleotides). According to the present disclosure, mutations such as the addition, substitution and/or deletion of one or more amino acids may be represented by reference to an amino acid position in a reference polypeptide. For example, an amino acid substitution may be referred to in the present disclosure by reference to the amino acid at a position in a reference polypeptide followed by the substituted amino acid (e.g., “L156H” refers to a substitution of histidine for leucine at the position 156 of a reference polypeptide). In some embodiments, wherein mutations comprise the addition and/or substitution of amino acids and/or nucleotides, such additions and/or substitutions may comprise 1 or more amino acid and/or nucleotide residues and may include modified amino acids and/or nucleotides. The resulting construct, molecule or sequence of a mutation, change or alteration may be referred to herein as a mutant.

Nucleic acid: “Nucleic acid,” “nucleic acid molecule,” “oligonucleotide,” “nucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). DNA includes, but is not limited to, cDNA, genomic DNA, plasmid DNA, synthetic DNA, and semi-synthetic DNA.

Operably linked: As used herein, the phrase “operably linked” refers to a functional connection between two or more molecules, constructs, transcripts, entities, moieties or the like. “Operably-linked” or “functionally linked” as it refers to nucleic acid sequences and polynucleotides refers to the association of nucleic acid sequences so that the function of one is affected by the other, while the nucleic acid sequences need not necessarily be adjacent or contiguous to each other, but may have intervening sequences between them. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. A transcriptional regulatory sequence is generally operably linked in cis with a coding sequence but need not be directly adjacent to it. For example, an enhancer is a transcriptional regulatory sequence that is operably linked to a coding sequence, even though it is not contiguous with the coding sequence. A promoter is operably linked to a gene of interest if the promoter regulates or mediates transcription of the gene of interest in a cell.

Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In an association between two or more polypeptides or domains thereof to create a fusion polypeptide, the term “operably linked” means that the state or function of one polypeptide in the fusion protein is affected by the other polypeptide in the fusion protein. For example, with respect to a fusion protein comprising a DRD and a transcription factor or a domain thereof, the DRD and the transcription factor are operably linked if stabilization of the DRD with a ligand results in stabilization of the transcription factor, while destabilization of the DRD in the absence of a ligand results in destabilization of the transcription factor. With respect to a fusion polypeptide in which a DNA-binding domain is fused to an activation domain, the DNA-binding domain and the activation domain are operably linked if, in the fusion polypeptide, the DNA-binding domain portion is able to bind to its specific binding site, and thus enable the activation domain to upregulate gene expression.

Plasmid: The term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

Polypeptide: The terms “polypeptide(s),” “peptide” and “protein(s)” are used interchangeably to refer to a polymer of amino acid residues. The term also applies to amino acid polymers in which one or more amino acids are chemical analogues or modified derivatives of corresponding naturally occurring amino acids.

Promoter: “Promoter” and “promoter sequence” are used interchangeably and refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or may comprise synthetic DNA segments. It is understood by those skilled in the art that different promoters may direct the expression of a gene in different tissues or cell types, or at different stages of development, or in response to different environmental or physiological conditions. A promoter comprising a synthetic DNA segment responsive to a synthetic transcription factor may direct expression of a gene when the synthetic transcription factor is expressed, binds to and activates the promoter. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity. The promoter sequence is typically bounded at its 3′ terminus by the transcription initiation site and extends upstream (5′ direction) to include the minimum number of bases or elements necessary to initiate transcription at levels detectable above background. Within the promoter sequence is found a transcription initiation site, as well as protein binding domains (consensus sequences) responsible for the binding of RNA polymerase.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or an oncopromoter.

Payload: the term “payload” as used herein, refers to any protein or compound whose function is to be altered. In the context of the present disclosure, the payload is a Cas protein or a transcription factor or portion thereof

Pharmaceutically acceptable excipients: the term “pharmaceutically acceptable excipient,” as used herein, refers to any ingredient other than active agents (e.g., as described herein) present in pharmaceutical compositions and having the properties of being substantially nontoxic and non-inflammatory in subjects. It is understood by those of skill in the art that a particular pharmaceutically acceptable excipient may not be suitable for all active agents or modes of administration. For example, some pharmaceutically acceptable excipients may be suitable for a small molecule therapeutic drug but not suitable for a viral vector. Similarly, some pharmaceutically acceptable excipients may be suitable for oral or parenteral administration but not suitable for intravenous administration. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compositions and compounds described herein are forms of the disclosed compositions and compounds wherein the acid or base moiety is in its salt form (e.g., as generated by reacting a free base group with a suitable organic acid). It is understood by those of skill in the art that a particular pharmaceutically acceptable salt may not be suitable for all modes of administration. Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Recombinant: The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell or organism, the term refers to a cell or organism into which a heterologous nucleic acid molecule has been introduced. A recombinant cell may replicate a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

Sequence: The term “sequence” refers to an amino acid or nucleic acid sequence of any length greater than one. As used herein, an amino acid sequence is linear and comprised of amino acids. As used herein, the nucleic acid sequence can be DNA or RNA or a modified form thereof; the nucleic acid sequence can be linear or circular, and can be either single-stranded or double stranded.

Selectable marker: The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

Stabilize: As used herein, the term “stabilize”, “stabilized,” “stabilized region” means to make a polypeptide or region thereof become or remain stable. In some embodiments, stability is measured relative to an absolute value. For example, the stability of a polypeptide comprising a DRD bound to its ligand may be compared to the stability of the wild type polypeptide. In some embodiments, stability is measured relative to a different status or state of the same polypeptide. For example, the stability of a polypeptide comprising a DRD bound to its ligand may be compared to the stability of the polypeptide comprising a DRD in the absence of its ligand.

Subject: The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any patient or subject (e.g. mammalian) to which the systems, nucleic acids, polynucleotides, payloads, components, vectors, or cells of the disclosure can be administered.

Therapeutically effective amount: As used herein, the term “therapeutically effective amount” means an amount of an agent to be delivered (e.g., nucleic acid, construct, protein, composition, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Transcription Factor: A transcription factor is a protein that binds to DNA, typically to a sequence-specific site on the DNA (a transcription factor polynucleotide binding site) located in or near a promoter, which facilitates the binding of transcription machinery to the promoter, thus regulating gene expression by promoting or suppressing transcription. Such entities are also known as transcription regulator proteins. In some embodiments, transcription factors are proteins that recognize and bind to specific short DNA sequences and thereby causally affect gene expression.

Transcription factors typically consist of DNA-binding domains and effector or activation domains that mediate interactions with other proteins necessary for transcription, including with other transcription factors. Transcription factors execute many functions, including gene activation. They are transcribed in the nucleus, translated in the cytoplasm, and find their target sites in the genomic DNA on reentry into the nucleus, mediated by nuclear localization sites included in all transcription factor protein sequences. Transcription factors include basic domains which cause them to be concentrated nonspecifically in the vicinity of the DNA, facilitating the diffusion-limited discovery of their target sites.

The DNA sequence that a transcriptional factor DNA binding domain binds to is called a transcription factor binding site or response element, or as used herein interchangeably, a specific polynucleotide binding site; these binding sites are found in or near the promoter of the regulated DNA sequence. A promoter comprising a specific polynucleotide binding site may be an exogenous promoter. In some embodiments, a promoter may be an exogenous inducible promoter.

Transcription factor binding site: A “transcription factor binding site” as used herein refers to a region of a nucleic acid molecule or polynucleotide to which a transcription factor or transcription factor DNA binding domain binds. Binding of a transcription factor to a transcription factor binding site enables the regulation of gene expression by the transcription factor.

Treatment or treating: As used herein, the terms “treat” in all its verb forms, means to relieve, alleviate, prevent, and/or manage at least one symptom of a disease or a disorder in a subject. The term “treat” also denotes delaying the onset of a disease (i.e., the period prior to clinical manifestation of a disease), decreasing symptoms resulting from a disease, delaying the progression or prolonging survival for individuals with a disease, and/or reducing the risk of developing or worsening of a disease. The term “treatment” means the act of “treating” as defined above.

Target site: The terms “target site,” “target nucleic acid site,” “target sequence,” and “target locus” are used interchangeably and refer to a nucleic acid sequence that defines a portion of a nucleic acid to which a binding molecule will bind, provided sufficient conditions for binding exist. An “intended” target site is one that the binding molecule is designed and/or selected to bind to. In various embodiments of the present disclosure, a target site is recognized and bound by a DNA-binding molecule or domain, for example a crRNA, guide RNA, transcription factor binding domain, or fusion protein. In some embodiments, a target site is recognized and bound by one or more complexes comprising such molecules or domains, including for example, a Cas molecule/gRNA molecule complex. A “target nucleic acid” or “target gene” is a nucleic acid or gene, respectively, that comprises a target site.

Transcription: “Transcription” refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilizing the transcript.

Transcription regulatory element: A transcription regulatory element or sequence include, but is not limited to, a promoter sequence (e.g., the TATA box), an enhancer element, a signal sequence, or an array of transcription factor binding sites. It controls or regulates transcription of a gene operably linked to it.

Transgene: “Transgene” refers to a polynucleotide segment containing a gene sequence that has been introduced into a host cell. The transgene may comprise sequences that are native to the cell, sequences that do not occur naturally in the cell, or combinations thereof. A transgene may contain sequences coding for one or more proteins that may be operably linked to appropriate regulatory sequences for expression of the coding sequences in the cell. A transgene may also be introduced into a population of cells or to an organism, for example into the genome of an organism.

Variant: A “variant” of a molecule is meant to refer to a molecule substantially similar in structure and/or biological activity to either the entire molecule, or to a fragment thereof. Thus, two molecules are considered variants as that term is used herein even if the composition or secondary, tertiary, or quaternary structure of one of the molecules is not identical to that found in the other, or if the sequence of amino acid residues is not identical.

Vector: A “vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. Vectors used in gene and cell therapy include those derived from, without limitation, adenovirus, adeno-associated virus (AAV), alphavirus, flavivirus, herpes virus, measles virus, rhabdovirus, retrovirus, lentivirus, Newcastle disease virus (NDV), poxvirus and picornavirus. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating nucleotide sequences (linkers) into the DNA termini. Such vectors may be engineered to contain selectable marker genes that provide for the selection of cells. Such markers allow identification and/or selection of host cells that incorporate and express the proteins encoded by the marker. Common vectors include plasmids, viral genomes, and (primarily in yeast and bacteria) “artificial chromosomes.” “Expression vectors” are vectors that are designed to enable the expression of an inserted nucleic acid sequence. Expression vectors may comprise elements that provide for or facilitate transcription of nucleic acids that are cloned into the vectors. Such elements can include, e.g., promoters and/or enhancers operably coupled to a nucleic acid of interest.

Wild-type: “Wild-type” refers to a nucleic acid sequence, nucleic acid molecule, amino acid sequence, polypeptide or organism found in nature without any known mutation. The term may also be used to describe the properties of a wild-type nucleic acid sequence, nucleic acid molecule, amino acid sequence, polypeptide or organism.

### EQUIVALENTS AND SCOPE

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects. While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure. The present disclosure is further illustrated by the following nonlimiting examples.

### EXAMPLES

### Example 1: Construct Design for Direct Regulation of Cas

The present example illustrates construct engineering for constructs designed to directly regulate Cas. These constructs can be designed as components of a direct Cas-DRD regulation system described by the present disclosure.

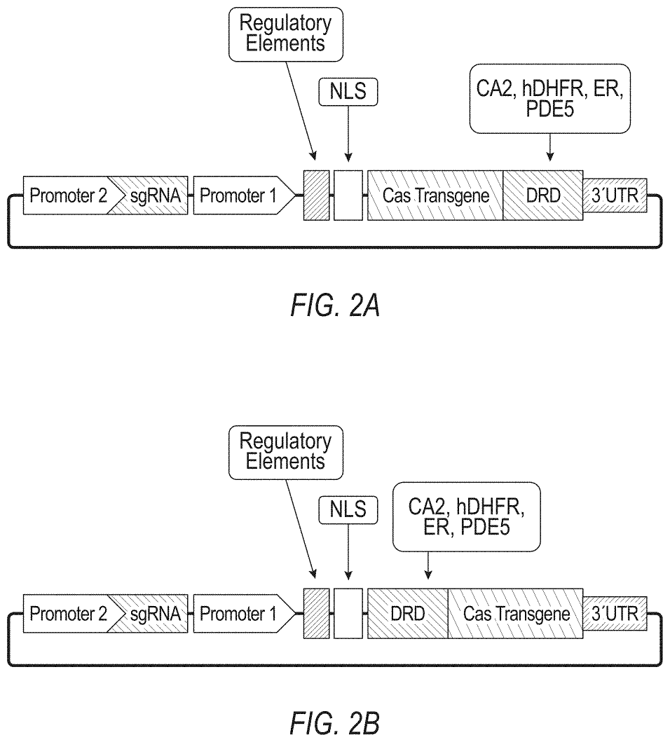

A construct designed to directly regulate Cas comprises nucleic acid sequences encoding a Cas nuclease and a DRD, as well as a first promoter mediating transcription of the Cas nuclease and a second promoter mediating transcription of a guide RNA corresponding to the Cas nuclease. Another feature in the design of such constructs is a sequence that enables a mechanism for transport of the Cas nuclease to the cell nucleus, such as a nuclear localization signal (NLS). A schematic of a construct designed to directly regulate Cas is shown in FIG. 2A-FIG. 2B. In some embodiments, a Cas protein may be operably linked to a DRD at its C-terminus. In some embodiments, a Cas protein may be operably linked to a DRD at its N-terminus. In some embodiments, a Cas protein may be operably linked to a DRD at both its N- and C-termini.

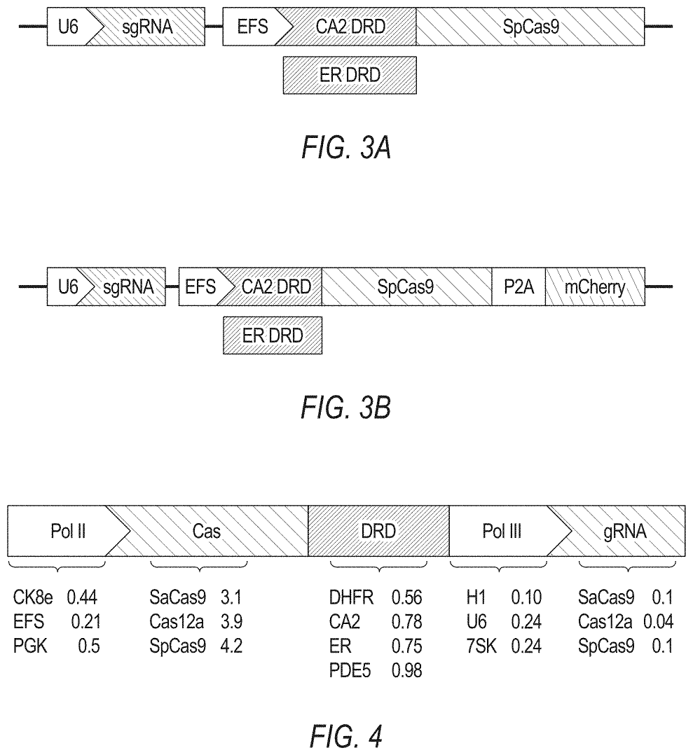

DRDs that can be used for constructs designed to directly regulate Cas may be selected from a CA2 DRD, an ER DRD, a hDHFR DRD, and a hPDE5 DRD. Transcription of the guide RNA is mediated by a Pol III promoter, such as a U6 promoter. The Cas is transcribed from a Pol II promoter, such as EFS. Exemplary constructs engineered according to the design for direct regulation of Cas are shown with specified elements of the present disclosure in FIG. 3A-FIG. 3B, FIG. 4 and Table 2.

TABLE 2
Example constructs for direct regulation of Cas9

| Name | Description |
|---|---|
| OT-Cas9-001 | pELDS-U6prom-DMDe51 sgRNA-EFSprom-Cas9-NLS-FLAG-P2A-mCherry |
| OT-Cas9-002 | pELDS-U6prom-CD47 sgRNA-EFSprom-Cas9-NLS-FLAG-P2A-mCherry |
| OT-Cas9-003 | pELDS-U6prom-CD47 sgRNA-EFSprom-CA2(wt)-Cas9-NLS-FLAG-P2A-mCherry |
| OT-Cas9-004 | pELDS-U6prom-CD47 sgRNA-EFSprom-CA2(L156H)-Cas9-NLS-FLAG-P2A-mCherry |
| OT-Cas9-005 | pELDS-U6prom-CD47 sgRNA-EFSprom-ER(Q502D)-Cas9-NLS-FLAG-P2A-mCherry |
| OT-Cas9-006 | pELDS-U6prom-EGFP sgRNA-EFSprom-Cas9-NLS-FLAG-P2A-mCherry |
| OT-Cas9-007 | pELDS-U6prom-DMDe51 sgRNA-EFSprom-CA2(wt)-Cas9-NLS-FLAG-P2A-mCherry |
| OT-Cas9-008 | pELDS-U6prom-DMDe51 sgRNA-EFSprom-CA2(L156H)-Cas9-NLS-FLAG-P2A-mCherry |
| OT-Cas9-009 | pELDS-U6prom-DMDe51 sgRNA-EFSprom-ER(Q502D)-Cas9-NLS-FLAG-P2A-mCherry |
| OT-Cas9-010 | pELDS-U6prom-DMDe45 sgRNA-EFSprom-Cas9-NLS-FLAG-P2A-mCherry |
| OT-Cas9-011 | pELDS-U6prom-DMDe45 sgRNA-EFSprom-CA2(wt)-Cas9-NLS-FLAG-P2A-mCherry |
| OT-Cas9-012 | pELDS-U6prom-DMDe45 sgRNA-EFSprom-CA2(L156H)-Cas9-NLS-FLAG-P2A-mCherry |
| OT-Cas9-013 | pELDS-U6prom-DMDe45 sgRNA-EFSprom-ER(Q502D)-Cas9-NLS-FLAG-P2A-mCherry |
| OT-Cas9-014 | pELDS-U6prom-EMX1 sgRNA-EFSprom-Cas9-NLS-FLAG-P2A-mCherry |
| OT-Cas9-015 | pELDS-U6prom-EMX1 sgRNA-EFSprom-CA2(wt)-Cas9-NLS-FLAG-P2A-mCherry |
| OT-Cas9-016 | pELDS-U6prom-EMX1 sgRNA-EFSprom-CA2(L156H)-Cas9-NLS-FLAG-P2A-mCherry |
| OT-Cas9-017 | pELDS-U6prom-EMX1 sgRNA-EFSprom-ER(Q502D)-Cas9-NLS-FLAG-P2A-mCherry |
| OT-Cas9-021 | pHybrid-U6prom-EGFP sgRNA-EFSprom-Cas9-NLS-FLAG-P2A-mCherry |
| OT-Cas9-024 | pHybrid-U6prom-EGFP sgRNA-EFSprom-CA2(L156H)-Cas9-NLS-FLAG-P2A-mCherry |
| OT-Cas9-025 | pHybrid-U6prom-EMX1 sgRNA-EFSprom-Cas9-NLS-FLAG-P2A-mCherry |

Illustrative components of constructs engineered according to the design for direct regulation of Cas, such as the constructs in Table 2, are provided in Table 3. An asterisk (“*”) in Table 3 indicates the translation of the stop codon.

TABLE 3
Components of illustrative constructs for direct Cas-DRD regulation
systems and Cas-transcription factor systems

| Component | Description | Nucleic Acid Sequence | Amino Acid Sequence |
|---|---|---|---|
| U6prom | the U6 promoter, | gagggcctatttcccatgattcctt | not applicable |
| | which drives | catatttgcatatacgatacaaggc | |
| | expression of the | tgttagagagataattagaattaat | |
| | sgRNA operably | ttgactgtaaacacaaagatattag | |
| | linked to the U6 | tacaaaatacgtgacgtagaaagta | |
| | promoter in the | ataatttcttgggtagtttgcagtt | |
| | construct | ttaaaattatgttttaaaatggact | |
| | | atcatatgcttaccgtaacttgaaa | |
| | | gtatttcgatttcttggctttatat | |
| | | atcttgtggaaaggac | |
| | | (SEQ ID NO: 48) | |
| EFSprom | EFS promoter; a | gggcagagcgcacatcgcccacagt | not applicable |
| | Pol 11 promoter | ccccgagaagttggggggaggggtc | |
| | operably | ggcaattgatccggtgcctagagaa | |
| | linked to | ggtggcgcggggtaaactgggaaag | |
| | sequence | tgatgtcgtgtactggctccgcctt | |
| | encoding a Cas | tttcccgagggtgggggagaaccgt | |
| | protein | atataagtgcagtagtcgccgtgaa | |
| | | cgttctttttcgcaacgggtttgcc | |
| | | gccagaacacag | |
| | | (SEQ ID NO: 49) | |
| NLS | Nucleoplasmin | aagcgacctgccgccacaaagaagg | KRPAATKKA |
| | nuclear | ctggacaggctaagaagaagaaa | GQAKKKK |
| | localization | (SEQ ID NO: 50) | (SEQ ID |
| | signal | | NO: 69) |
| FLAG | FLAG epitope tag | Gattacaaagacgatgacgataag | DYKDDDDK |
| | | (SEQ ID NO: 51) | (SEQ ID |
| | | | NO: 70) |
| Cas9 | SpCas9 nuclease | gacaagaagtacagcatcggcctgg | DKKYSIGLDIG |
| | | acatcggcaccaactctgtgggctg | TNSVGWAVIT |
| | | ggccgtgatcaccgacgagtacaag | DEYKVPSKKF |
| | | gtgcccagcaagaaattcaaggtgc | KVLGNTDRHS |
| | | tgggcaacaccgaccggcacagcat | IKKNLIGALL |
| | | caagaagaacctgatcggagccctg | FDSGETAEAT |
| | | ctgttcgacagcggcgaaacagccg | RLKRTARRRY |
| | | aggccacccggctgaagagaaccgc | TRRKNRICYL |
| | | cagaagaagatacaccagacggaag | QEIFSNEMAK |
| | | aaccggatctgctatctgcaagaga | VDDSFFHRLE |
| | | tcttcagcaacgagatggccaaggt | ESFLVEEDKK |
| | | ggacgacagcttcttccacagactg | HERHPIFGNI |
| | | gaagagtccttcctggtggaagagg | VDEVAYIIEK |
| | | ataagaagcacgagcggcaccccat | YPTIYHLRKK |
| | | cttcggcaacatcgtggacgaggtg | LVDSTDKADL |
| | | gcctaccacgagaagtaccccacca | RLIYLALAHM |
| | | tctaccacctgagaaagaaactggt | IKFRGHFLIE |
| | | ggacagcaccgacaaggccgacctg | GDLNPDNSDV |
| | | cggctgatctatctggccctggccc | DKLFIQLVQT |
| | | acatgatcaagttccggggccactt | YNQLFEENPI |
| | | cctgatcgagggcgacctgaacccc | NASGVDAKAI |
| | | gacaacagcgacgtggacaagctgt | LSARLSKSRR |
| | | tcatccagctggtgcagacctacaa | LENLIAQLPG |
| | | ccagctgttcgaggaaaaccccatc | EKKNGLFGNL |
| | | aacgccagcggcgtggacgccaagg | IALSLGLTPN |
| | | ccatcctgtctgccagactgagcaa | FKSNFDLAED |
| | | gagcagacggctggaaaatctgatc | AKLQLSKDTY |
| | | gcccagctgcccggcgagaagaaga | DDDLDNLLAQ |
| | | atggcctgttcggaaacctgattgc | IGDQYADLFL |
| | | cctgagcctgggcctgacccccaac | AAKNLSDAIL |
| | | ttcaagagcaacttcgacctggccg | LSDILRVNTE |
| | | aggatgccaaactgcagctgagcaa | ITKAPLSASM |
| | | ggacacctacgacgacgacctggac | IKRYDEHHQD |
| | | aacctgctggcccagatcggcgacc | LTLLKALVRQ |
| | | agtacgccgacctgtttctggccgc | QLPEKYKEIF |
| | | caagaacctgtccgacgccatcctg | FDQSKNGYAG |
| | | ctgagcgacatcctgagagtgaaca | YIDGGASQEE |
| | | ccgagatcaccaaggcccccctgag | FYKFIKPILE |
| | | cgcctctatgatcaagagatacgac | KMDGTEELLV |
| | | gagcaccaccaggacctgaccctgc | KLNREDLLRK |
| | | tgaaagctctcgtgcggcagcagct | QRTFDNGSIP |
| | | gcctgagaagtacaaagagattttc | HQIHLGELHA |
| | | ttcgaccagagcaagaacggctacg | ILRRQEDFYP |
| | | ccggctacattgacggcggagccag | FLKDNREKIE |
| | | ccaggaagagttctacaagttcatc | KILTFRIPYY |
| | | aagcccatcctggaaaagatggacgg | VGPLARGNSR |
| | | caccgaggaactgctcgtgaagctg | FAWMTRKSEE |
| | | aacagagaggacctgctgcggaagc | TITPWNFEEV |
| | | agcggaccttcgacaacggcagcat | VDKGASAQSF |
| | | cccccaccagatccacctgggagag | IERMTNFDKN |
| | | ctgcacgccattctgcggcggcagg | LPNEKVLPKH |
| | | aagatttttacccattcctgaagga | SLLYEYFTVY |
| | | caaccgggaaaagatcgagaagatc | NELTKVKYVT |
| | | ctgaccttccgcatcccctactacg | EGMRKPAFLS |
| | | tgggccctctggccaggggaaacag | GEQKKAIVDL |
| | | cagattcgcctggatgaccagaaag | LFKTNRKVTV |
| | | agcgaggaaaccatcaccccctgga | KQLKEDYFKK |
| | | acttcgaggaagtggtggacaaggg | IECFDSVEIS |
| | | cgcttccgcccagagcttcatcgag | GVEDRFNASL |
| | | cggatgaccaacttcgataagaacc | GTYHDLLKII |
| | | tgcccaacgagaaggtgctgcccaa | KDKDFLDNEE |
| | | gcacagcctgctgtacgagtacttc | NEDILEDIVL |
| | | accgtgtataacgagctgaccaaag | TLTLFEDREM |
| | | tgaaatacgtgaccgagggaatgag | IEERLKTYAH |
| | | aaagcccgccttcctgagcggcgag | LFDDKVMKQL |
| | | cagaaaaaggccatcgtggacctgc | KRRRYTGWGR |
| | | tgttcaagaccaaccggaaagtgac | LSRKLINGIR |
| | | cgtgaagcagctgaaagaggactac | DKQSGKTILD |
| | | ttcaagaaaatcgagtgcttcgact | FLKSDGFANR |
| | | ccgtggaaatctccggcgtggaaga | NFMQLIHDDS |
| | | tcggttcaacgcctccctgggcaca | LTFKEDIQKA |
| | | taccacgatctgctgaaaattatca | QVSGQGDSLH |
| | | aggacaaggacttcctggacaatga | EHIANLAGSP |
| | | ggaaaacgaggacattctggaagat | AIKKGILQTV |
| | | atcgtgctgaccctgacactgtttg | KVVDELVKVM |
| | | aggacagagagatgatcgaggaacg | GRHKPENIVI |
| | | gctgaaaacctatgcccacctgttc | EMARENQTTQ |
| | | gacgacaaagtgatgaagcagctga | KGQKNSRERM |
| | | agcggcggagatacaccggctgggg | KRIEEGIKEL |
| | | caggctgagccggaagctgatcaac | GSQILKEEHP |
| | | ggcatccgggacaagcagtccggca | VENTQLQNEK |
| | | agacaatcctggatttcctgaagtc | LYLYYLQNGR |
| | | cgacggcttcgccaacagaaacttc | DMYVDQELDI |
| | | atgcagctgatccacgacgacagcc | NRLSDYDVDH |
| | | tgacctttaaagaggacatccagaa | IVPQSFLKDD |
| | | agcccaggtgtccggccagggcgat | SIDNKVLTRS |
| | | agcctgcacgagcacattgccaatc | DKNRGKSDNV |
| | | tggccggcagccccgccattaagaa | PSEEVVKKMK |
| | | gggcatcctgcagacagtgaaggtg | NYWRQLLNAK |
| | | gtggacgagctcgtgaaagtgatgg | LITQRKFDNL |
| | | gccggcacaagcccgagaacatcgt | TKAERGGLSE |
| | | gatcgaaatggccagagagaaccag | LDKAGFIKRQ |
| | | accacccagaagggacagaagaaca | LVETRQITKH |
| | | gccgcgagagaatgaagcggatcga | VAQILDSRMN |
| | | agagggcatcaaagagctgggcagc | TKYDENDKLI |
| | | cagatcctgaaagaacaccccgtgg | REVKVITLKS |
| | | aaaacacccagctgcagaacgagaa | KLVSDFRKDF |
| | | gctgtacctgtactacctgcagaat | QFYKVREINN |
| | | gggcgggatatgtacgtggaccagg | YHHAHDAYLN |
| | | aactggacatcaaccggctgtccga | AVVGTALIKK |
| | | ctacgatgtggaccatatcgtgcct | YPKLESEFVY |
| | | cagagctttctgaaggacgactcca | GDYKVYDVRK |
| | | tcgacaacaaggtgctgaccagaag | MIAKSEQEIG |
| | | cgacaagaaccggggcaagagcgac | KATAKYFFYS |
| | | aacgtgccctccgaagaggtcgtga | NIMNFFKTEI |
| | | agaagatgaagaactactggcggca | TLANGEIRKR |
| | | gctgctgaacgccaagctgattacc | PLIETNGETG |
| | | cagagaaagttcgacaatctgacca | EIVWDKGRDF |
| | | aggccgagagaggcggcctgagcga | ATVRKVLSMP |
| | | actggataaggccggcttcatcaag | QVNIVKKTEV |
| | | agacagctggtggaaacccggcaga | QTGGFSKESI |
| | | tcacaaagcacgtggcacagatcct | LPKRNSDKLI |
| | | ggactcccggatgaacactaagtac | ARKKDWDPKK |
| | | gacgagaatgacaagctgatccggg | YGGFDSPTVA |
| | | aagtgaaagtgatcaccctgaagtc | YSVLVVAKVE |
| | | caagctggtgtccgatttccggaag | KGKSKKLKSV |
| | | gatttccagttttacaaagtgcgcg | KELLGITIME |
| | | agatcaacaactaccaccacgccca | RSSFEKNPID |
| | | cgacgcctacctgaacgccgtcgtg | FLEAKGYKEV |
| | | ggaaccgccctgatcaaaaagtacc | KKDLIIKLPK |
| | | ctaagctggaaagcgagttcgtgta | YSLFELENGR |
| | | cggcgactacaaggtgtacgacgtg | KRMLASAGEL |
| | | cggaagatgatcgccaagagcgagc | QKGNELALPS |
| | | aggaaatcggcaaggctaccgccaa | KYVNFLYLAS |
| | | gtacttcttctacagcaacatcatg | HYEKLKGSPE |
| | | aactttttcaagaccgagattaccc | DNEQKQLFVE |
| | | tggccaacggcgagatccggaagcg | QHKHYLDEII |
| | | gcctctgatcgagacaaacggcgaa | EQISEFSKRV |
| | | accggggagatcgtgtgggataagg | ILADANLDKV |
| | | gccgggattttgccaccgtgcggaa | LSAYNKHRDK |
| | | agtgctgagcatgccccaagtgaat | PIREQAENII |
| | | atcgtgaaaaagaccgaggtgcaga | HLFTLTNLGA |
| | | caggcggcttcagcaaagagtctat | PAAFKYFDTT |
| | | cctgcccaagaggaacagcgataag | IDRKRYTSTK |
| | | ctgatcgccagaaagaaggactggg | EVLDATLIHQ |
| | | accctaagaagtacggcggcttcga | SITGLYETRI |
| | | cagccccaccgtggcctattctgtg | DLSQLGGD |
| | | ctggtggtggccaaagtggaaaagg | (SEQ ID |
| | | gcaagtccaagaaactgaagagtgt | NO: 71) |
| | | gaaagagctgctggggatcaccatc | |
| | | atggaaagaagcagcttcgagaaga | |
| | | atcccatcgactttctggaagccaa | |
| | | gggctacaaagaagtgaaaaaggac | |
| | | ctgatcatcaagctgcctaagtact | |
| | | ccctgttcgagctggaaaacggccg | |
| | | gaagagaatgctggcctctgccggc | |
| | | gaactgcagaagggaaacgaactgg | |
| | | ccctgccctccaaatatgtgaactt | |
| | | cctgtacctggccagccactatgag | |
| | | aagctgaagggctcccccgaggata | |
| | | atgagcagaaacagctgtttgtgga | |
| | | acagcacaagcactacctggacgag | |
| | | atcatcgagcagatcagcgagttct | |
| | | ccaagagagtgatcctggccgacgc | |
| | | taatctggacaaagtgctgtccgcc | |
| | | tacaacaagcaccgggataagccca | |
| | | tcagagagcaggccgagaatatcat | |
| | | ccacctgtttaccctgaccaatctg | |
| | | ggagcccctgccgccttcaagtact | |
| | | ttgacaccaccatcgaccggaagag | |
| | | gtacaccagcaccaaagaggtgctg | |
| | | gacgccaccctgatccaccagagca | |
| | | tcaccggcctgtacgagacacggat | |
| | | cgacctgtctcagctgggaggcgac | |
| | | (SEQ ID NO: 52) | |
| P2A | porcine | gctactaacttcagcctgct | ATNFSLLKQAG |
| | teschovirus-1 | gaagcaggctggggacgtgg | DVEENPGP |
| | 2A | aggagaaccctggacct | (SEQ |
| | | (SEQ ID NO: 53) | ID NO: 72) |
| mCherry | mCherry red | ttgagcaagggcgaggaggac | LSKGEEDNMA |
| | fluorescent | aacatggccatcatcaagga | IIKEFMRFKV |
| | protein | gttcatgcgcttcaaggtgc | HMEGSVNGHE |
| | | acatggagggctccgtgaac | FEIEGEGEGR |
| | | ggccacgagttcgagatcga | PYEGTQTAKL |
| | | gggcgagggcgagggccgcc | KVTKGGPLPF |
| | | cctacgagggcacccagacc | AWDILSPQFM |
| | | gccaagctgaaggtgaccaa | YGSKAYVKHP |
| | | gggcggccccctgcccttcg | ADIPDYLKLS |
| | | cctgggacatcctgtcccct | FPEGFKWERV |
| | | cagttcatgtacggctccaa | MNFEDGGVVT |
| | | ggcctacgtgaagcaccccg | VTQDSSLQDG |
| | | ccgacatccccgactacttg | EFIYKVKLRG |
| | | aagctgtccttccccgaggg | TNFPSDGPVM |
| | | cttcaagtgggagcgcgtga | QKKTMGWEAS |
| | | tgaacttcgaggacggcggc | SERMYPEDGA |
| | | gtggtgaccgtgacccagga | LKGEIKQRLK |
| | | ctcctccctgcaggacggcg | LKDGGHYDAE |
| | | agttcatctacaaggtgaag | VKTTYKAKKP |
| | | ctgcgcggcaccaacttccc | VQLPGAYNVN |
| | | ctccgacggccccgtaatgc | IKLDITSHNE |
| | | agaagaagaccatgggctgg | DYTIVEQYER |
| | | gaggcctcctccgagcggat | AEGRHSTGGM |
| | | gtaccccgaggacggcgccc | DELYK* |
| | | tgaagggcgagatcaagcag | (SEQ ID |
| | | aggctgaagctgaaggacgg | NO: 73) |
| | | cggccactacgacgccgagg | |
| | | tcaagaccacctacaaggcc | |
| | | aagaagcccgtgcagctgcc | |
| | | cggcgcctacaacgtcaaca | |
| | | tcaagctggacatcacctcc | |
| | | cacaacgaggactacaccat | |
| | | cgtggaacagtacgagcgcg | |
| | | ccgagggccgccactccacc | |
| | | ggcggcatggacgagctgta | |
| | | caagtaa | |
| | | (SEQ ID NO: 54) | |
| CA2 | CA2DRD | variant 1: | SHHWGYGKHN |
| (L15611) | comprising a | TCCCATCACTGGGGGTACGG | GPEHWHKDFP |
| | L15611 | CAAACACAACGGACCTGAGC | IAKGERQSPV |
| | substitution | ACTGGCATAAGGACTTCCCC | DIDTHTAKYD |
| | relative to | ATTGCCAAGGGAGAGCGCCA | PSLKPLSVSY |
| | wild- | GTCCCCTGTTGACATCGACA | DQATSLRILN |
| | type CA2 (SEQ | CTCATACAGCCAAGTATGAC | NGUAFNVEFD |
| | ID NO: 5) | CCTTCCCTGAAGCCCCTGTC | DSQDKAVLKG |
| | | TGTTTCCTATGATCAAGCAA | GPLDGTYRLI |
| | | CTTCCCTGAGAATCCTCAAC | QFHFHWGSLD |
| | | AATGGTCATGCTTTCAACGT | GQGSEHTVDK |
| | | GGAGTTTGATGACTCTCAGG | KKYAAELHLV |
| | | ACAAAGCAGTGCTCAAGGGA | HWNTKYGDFG |
| | | GGACCCCTGGATGGCACTTA | KAVQQPDGLA |
| | | CAGATTGATTCAGTTTCACT | VLGIFLKVGS |
| | | TTCACTGGGGTTCACTTGAT | AKPGHQKVVD |
| | | GGACAAGGTTCAGAGCATAC | VLDSIKTKGK |
| | | TGTGGATAAAAAGAAATATG | SADFTNFDPR |
| | | CTGCAGAACTTCACTTGGTT | GLLPESLDYW |
| | | CACTGGAACACCAAATATGG | TYPGSLTTPP |
| | | GGATTTTGGGAAAGCTGTGC | LLECVTWIVL |
| | | AGCAACCTGATGGACTGGCC | KEPISVSSEQ |
| | | GTTCTAGGTATTTTTTTGAA | VLKFRKLNFN |
| | | GGTTGGCAGCGCTAAACCGG | GEGEPEELMV |
| | | GCCATCAGAAAGTTGTTGAT | DNWRPAQPLK |
| | | GTGCTGGATTCCATTAAAAC | NRQIKASFK |
| | | AAAGGGCAAGAGTGCTGACT | (SEQ ID |
| | | TCACTAACTTCGATCCTCGT | NO: 78) |
| | | GGCCTCCTTCCTGAATCCCT | |
| | | GGATTACTGGACCTACCCAG | |
| | | GCTCACTGACCACCCCTCCT | |
| | | CTTCTGGAATGTGTGACCTG | |
| | | GATTGTGCTCAAGGAACCCA | |
| | | TCAGCGTCAGCAGCGAGCAG | |
| | | GTGTTGAAATTCCGTAAACT | |
| | | TAACTTCAATGGGGAGGGTG | |
| | | AACCCGAAGAACTGATGGTG | |
| | | GACAACTGGCGCCCAGCTCA | |
| | | GCCACTGAAGAACAGGCAAA | |
| | | TCAAAGCTTCCTTCAAA | |
| | | (SEQ ID NO: 65) | |
| | | variant 2: | |
| | | TCCCATCACTGGGGGTACGG | |
| | | CAAACACAACGGACCTGAGC | |
| | | ACTGGCATAAGGACTTCCCC | |
| | | ATTGCCAAGGGAGAGCGCCA | |
| | | GTCCCCTGTTGACATCGACA | |
| | | CTCATACAGCCAAGTATGAC | |
| | | CCTTCCCTGAAGCCCCTGTC | |
| | | TGTTTCCTATGATCAAGCAA | |
| | | CTTCCCTGAGGATCCTCAAC | |
| | | AATGGTCATGCTTTCAACGT | |
| | | GGAGTTTGATGACTCTCAGG | |
| | | ACAAAGCAGTGCTCAAGGGA | |
| | | GGACCCCTGGATGGCACTTA | |
| | | CAGAITGATTCAGTTTCACT | |
| | | TTCACTGGGGTTCACTTGAT | |
| | | GGACAAGGTTCAGAGCATAC | |
| | | TGTGGATAAAAAGAAATATG | |
| | | CTGCAGAACTTC | |
| | | ACTTGGTTCACTGGAACACC | |
| | | AAATATGGGGATTTTGGGAA | |
| | | AGCTGTGCAGCAACCTGATG | |
| | | GACTGGCCGTTCTAGGTATT | |
| | | TTTTTGAAGGTTGGCAGCGC | |
| | | TAAACCGGGCCATCAGAAAG | |
| | | TTGTTGATGTGCTGGATTCC | |
| | | ATTAAAACAAAGGGCAAGAG | |
| | | TGCTGACTTCACTAACTTCG | |
| | | ATCCTCGTGGCCTCCTTCCT | |
| | | GAATCCCTGGATTACTGGAC | |
| | | CTACCCAGGCTCACTGACCA | |
| | | CCCCTCCTCTTCTGGAATGT | |
| | | GTGACCTGGATTGTGCTCAA | |
| | | GGAACCCATCAGCGTCAGCA | |
| | | GCGAGCAGGTGTTGAAATTC | |
| | | CGTAAACTTAACTTCAATG | |
| | | GGGAGGGTGAACCC | |
| | | GAAGAACTGATGGTGGACAA | |
| | | CTGGCGCCCAGCTCAGCCAC | |
| | | TGAAGAACAGGCAAATCAAA | |
| | | GCTTCCTTCAAA | |
| | | (SEQ ID NO: 67) | |
| | | variant 5: | |
| | | TCCCATCACTGGGGGTACGG | |
| | | CAAACACAACGGACCTGAGC | |
| | | ACTGGCATAAGGACTTCCCC | |
| | | ATTGCCAAGGGAGAGCGCCA | |
| | | GTCCCCTGTTGACATCGACA | |
| | | CTCATACAGCCAAGTATGAC | |
| | | CCTTCCCTGAAGCCCCTGTC | |
| | | TGTTTCCTATGATCAAGCAA | |
| | | CTTCCCTGAGGATTCTCAAC | |
| | | AATGGTCATGCTTTCAACGT | |
| | | GGAGTTTGATGACTCTCAGG | |
| | | ACAAAGCAGTGCTCAAGGGA | |
| | | GGACCCCTGGATGGCACTTA | |
| | | CAGATTGATTCAGTTTCACT | |
| | | TTCACTGGGGTTCACTTGAT | |
| | | GGACAAGGTTCAGAGCATAC | |
| | | TGTGGATAAAAAGAAATATG | |
| | | CTGCAGAACTTCACTTGGTT | |
| | | CACTGGAACACCAAATATGG | |
| | | GGATTTTGGGAAAGCTGTGC | |
| | | AGCAACCTGATGGACTGGCC | |
| | | GTTCTAGGTATTTTTTTGAA | |
| | | GGTTGGCAGCGCTAAACCGG | |
| | | GCCATCAGAAAGTTGTTGAT | |
| | | GTGCTGGATTCCATTAAAAC | |
| | | AAAGGGCAAGAGTGCTGACT | |
| | | TCACTAACTTCGATCCTCGT | |
| | | GGCCTCCTTCCTGAATCCCT | |
| | | GGATTACTGGACCTACCCAG | |
| | | GCTCACTGACCACCCCTCCT | |
| | | CTTCTGGAATGTGTGACCTG | |
| | | GATTGTGCTCAAGGAACCCA | |
| | | TCAGCGTCAGCAGCGAGCAG | |
| | | GTGTTGAAATTCCGTAAACT | |
| | | TAACTTCAATGGGGAGGGTG | |
| | | AACCCGAAGAACTGATGGTG | |
| | | GACAACTGGCGCCCAGCTCA | |
| | | GCCACTGAAGAACAGGCAAA | |
| | | TCAAAGCTTCCTTCAAA | |
| | | (SEQ ID NO: 66) | |
| ER | ER DRD | TCACTGGCGCTCAGCCTTAC | SLALSLTADQM |
| (Q502D) | comprising a | TGCCGACCAAATGGTATCAG | VSALLDAEPP |
| | Q502D | CTCTTCTGGACGCAGAACCC | ILYSEYDPTR |
| | substitution | CCAATTCTTTATTCCGAGTA | PFSEASMMGL |
| | relative to | CGACCCCACACGCCCGTTCA | LTNLADRELV |
| | wild-type | GTGAAGCTTCCATGATGGGC | HMINWAKRVP |
| | ER (SEQ ID | CTCCTTACGAACCTTGCCGA | GFVDLTLHDQ |
| | NO: 6) | CCGGGAACTCGTGCACATGA | VHLLECAWME |
| | | TCAATTGGGCGAAGCGGGTG | ILMIGLVWRS |
| | | CCGGGGTTCGTAGATTTGAC | MEHPGKLLFA |
| | | ACTTCACGACCAAGTTCATC | PNLLLDRNQG |
| | | TCTTGGAATGTGCTTGGATG | KCVEGGVEIF |
| | | GAGATATTGATGATCGGACT | DMLLATSSRF |
| | | CGTGTGGAGGTCAATGGAGC | RMMNLQGEEF |
| | | ATCCTGGTAAACTTCTTTTC | VCLKSIILLN |
| | | GCACCCAATCTGCTCTTGGA | SGVYTFLSST |
| | | TAGAAATCAGGGTAAGTGCG | LKSLEEKDHI |
| | | TCGAGGGTGGCGTTGAAATC | HRVLDKITDT |
| | | TTCGACATGCTCCTTGCGAC | LIHLMAKAGL |
| | | ATCCAGCCGATTCCGAATGA | TLQQQHDRLA |
| | | TGAATCTTCAAGGAGAGGAA | QLLLILSHIR |
| | | TTTGTCTGTCTTAAGAGCAT | HMSNKRMEHL |
| | | TATACTCCTCAATAGTGGAG | YSMKCKNVVP |
| | | TTTACACCTTCTTGTCCTCT | LSDLLLEMLD |
| | | ACACTGAAATCACTTGAGGA | AHRL (SEQ |
| | | AAAAGATCACATACATAGGG | ID NO: 79) |
| | | TGTTGGATAAAATCACGGAT | |
| | | ACACTCATACATCTGATGGC | |
| | | AAAAGCAGGATTGACCCTGC | |
| | | AACAGCAGCACgacCGACTG | |
| | | GCCCAACTGCTGTTGATCCT | |
| | | TAGCCATATCAGACACATGT | |
| | | CTAACAAAAGGATGGAACAT | |
| | | TTGTACAGCATGAAATGTAA | |
| | | GAACGTAGTGCCACTGTCCG | |
| | | ATTTGTTGCTGGAAATGCTG | |
| | | GACGCTCATCGGCTC (SEQ | |
| | | ID NO: 68) | |

Table 2 includes a construct comprising a CA2(L156H) DRD (construct OT-Cas9-004) and a corresponding control construct comprising a nucleic acid sequence encoding CA2 wild-type (WT) polypeptide (construct OT-Cas9-003). Table 2 also includes a construct comprising an ER(Q502D) DRD (OT-Cas9-005). These three constructs (OT-Cas9-003, OT-Cas9-004 and OT-Cas9-005) are designed to direct the encoded Cas9 nuclease to a target locus on the CD47 gene. A constitutive Cas9 control construct directing Cas9 nuclease to the CD47 gene is also shown in Table 2 (construct OT-Cas9-002). Constructs OT-Cas9-001 and OT-Cas9-006 direct Cas9 to target loci on the DMD and EGFP gene, respectively, and do not comprise DRDs.

Table 2 includes a construct comprising a CA2(L156H) DRD (construct OT-Cas9-008), a corresponding control construct comprising a nucleic acid sequence encoding CA2 wild-type (WT) polypeptide (construct OT-Cas9-007), and a construct comprising an ER(Q502D) DRD (OT-Cas9-009), all of which are designed to direct the encoded Cas9 nuclease to a target locus on exon 51 of the DMD gene. Table 2 includes a construct comprising a CA2(L156H) DRD (construct OT-Cas9-012), a corresponding control construct comprising a nucleic acid sequence encoding CA2 wild-type (WT) polypeptide (construct OT-Cas9-011), and a construct comprising an ER(Q502D) DRD (OT-Cas9-013), all of which are designed to direct the encoded Cas9 nuclease to a target locus on exon 45 of the DMD gene. A constitutive Cas9 control construct directing Cas9 nuclease to exon 45 of the DMD gene is also shown in Table 2 (construct OT-Cas9-010).

Table 2 includes a construct comprising a CA2(L156H) DRD (construct OT-Cas9-016), a corresponding control construct comprising a nucleic acid sequence encoding CA2 wild-type (WT) polypeptide (construct OT-Cas9-015), and a construct comprising an ER(Q502D) DRD (OT-Cas9-017), all of which are designed to direct the encoded Cas9 nuclease to a target locus on the EMX1 gene. A constitutive Cas9 control construct directing Cas9 nuclease to the EMX1 gene is also shown in Table 2 (construct OT-Cas9-014).

The constructs shown in Table 2 and schematically illustrated in FIG. 3A-FIG. 3B and FIG. 4 can be made according to standard molecular biology techniques.

### Example 2: Testing Ligand-Dependent Cas Expression and Activity for Systems Designed to Directly Regulate Cas

The present example demonstrates methods of detecting and analyzing Cas protein level and gene editing activity for constructs designed to directly regulate Cas. For illustrative purposes, the present example describes methodologies using Cas9 protein and an mCherry protein tag, such as the constructs shown in Table 2. These methods are also applicable to other constructs that are designed to directly regulate Cas in accordance with the present disclosure, such as constructs that are components of direct Cas-DRD regulation systems.

Cas expression and activity is analyzed in cells transiently transfected with constructs designed to directly regulate Cas or transduced with lentivirus made from these constructs. As a non-limiting example, the U20S cell line or the HEK293 cell line may be used for these methods. Untransduced (parental) U20S cells or HEK293 cells may be used as control cell lines.

Construct-expressing cells may be selected for analyses but do not necessarily require selection prior to analysis. For example, cells expressing the constructs described in Table 2 may be selected by sorting for mCherry positive cells. Cells are treated with vehicle control (e.g., DMSO) or drug (e.g., ACZ for constructs comprising a CA2 DRD or bazedoxifene for constructs comprising an ER DRD). For dose response studies, multiple doses are tested (e.g., a 10-point dose response assay including 100 μM ACZ or 1 μM bazedoxifene as top concentrations). Cells are treated for 24, 48, and/or 72 hours. Cas9 protein levels can be assessed by immunoassay. Cas9 mRNA levels can be measured by RT-PCR. To detect and analyze Cas9 activity, genomic DNA is isolated and genome editing is measured. Methods of measuring genome editing include the T7E1 assay (Alt-R Genome Editing Detection Kit from IDT), the TIDE assay (Brinkman et al., Nucleic Acids Res. 2014 Dec. 16; 42(22): e168; Brinkman et al., Methods in Molecular Biology, volume 1961; CRISPR Gene Editing pp. 22-44) and the ICE assay (https://ice.synthego.com/#/; Hsiau et al., bioRxive Aug. 10, 2019, https://doi.org/10.1101/251082). Illustrative sgRNA sequences for target locus sites in CD47, DMD exon 51, DMD exon 44 and EMX1 are shown in Table 4. Illustrative primer sets for assays to detect and analyze genome editing at these loci are shown in Table 5.

TABLE 4
sgRNA sequences

| Target | Name | Sequence |
|---|---|---|
| CD47 | CD47-sgRNA-1 | AGCAACAGCGCCGCTACCAG |
| | | (SEQ ID NO: 8) |
| DMD exon 51 | DMD-e51-sgRNA-1 | CACCAGAGTAACAGTCTGAG |
| | | (SEQ ID NO: 9) |
| DMD exon 44 | DMD-e44-sgRNA-1 | ATCTTACAGGAACTCCAGGA |
| | | (SEQ ID NO: 10) |
| EMX1 | EMX-sgRNA-1 | GAGTCCGAGCAGAAGAAGAA |
| | | (SEQ ID NO: 11) |

TABLE 5
Primer sets

| Name | Sequence |
|---|---|
| CD47-seq-F | GACCAGGGAAAGGAAGGGAG |
| | (SEQ ID NO: 12) |
| CD47-seq-R | GAACGGGTGCAATGAGGTC |
| | (SEQ ID NO: 13) |
| DMD-F | TTCCCTGGCAAGGTCTGA |
| | (SEQ ID NO: 14) |
| DMD-R | ATCCTCAAGGTCACCCACC |
| | (SEQ ID NO: 15) |
| DMD-T7E1-F | GTCTTTCTGTCTTGTATCCTTTGG |
| | (SEQ ID NO: 16) |
| DMD-T7E1-R | AATGTTAGTGCCTTTCACCC |
| | (SEQ ID NO: 17) |
| EMX-T7E1-F | TAACCCTATGTAGCCTCAGTCTTCCCAT |
| | (SEQ ID NO: 18) |
| EMX-T7E1-R | GCATCAAAACAAAAGGGAGATTGGAGACAC |
| | (SEQ ID NO: 19) |

Additionally, in the case of EGFP targeting guide RNAs, Cas9 activity can be assessed by measurement of EGFP expression by flow cytometry.

Cells comprising constructs having a DRD operably linked to Cas9 are expected to show ligand-dependent Cas9 protein levels. These constructs are also expected to show ligand-dependent genome editing.

### Example 3: Construct Design for Transcriptional Regulation of Cas

The present example illustrates construct engineering for constructs designed to transcriptionally regulate Cas. The combination of constructs designed to transcriptionally regulate Cas is referred to by the present disclosure as a Cas-transcription factor system.

Constructs designed to transcriptionally regulate Cas comprise (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; (3) a nucleic acid sequence that encodes a Cas protein and is operably linked to an inducible first promoter comprising the specific polynucleotide binding site; (4) a nucleic acid sequence that encodes a guide RNA; and (5) a second promoter that mediates transcription of the guide RNA. The one or more nucleic acid sequences that encode a transcription factor comprise one or more promoters that mediate transcription of the transcription factor components. The promoter(s) that mediate transcription of the transcription factor components may be selected from a constitutive promoter, such as EFla, or an inducible promoter, such as a promoter comprising the specific polynucleotide binding site (for a self-inducing transcription factor). Another feature in the design of such constructs are sequences that enable transport of the transcription factor and the Cas nuclease to the cell nucleus. In some embodiments, the Cas protein is operably linked to a DRD.

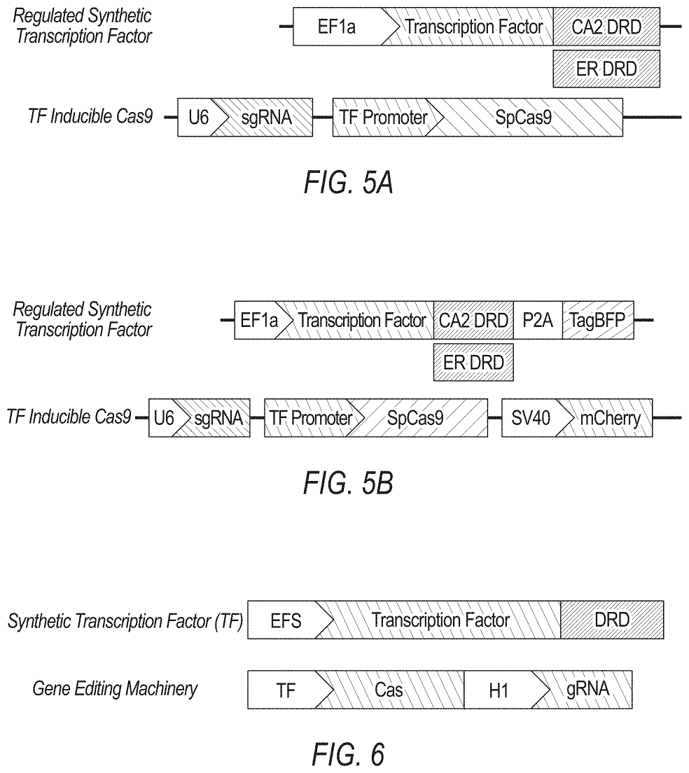

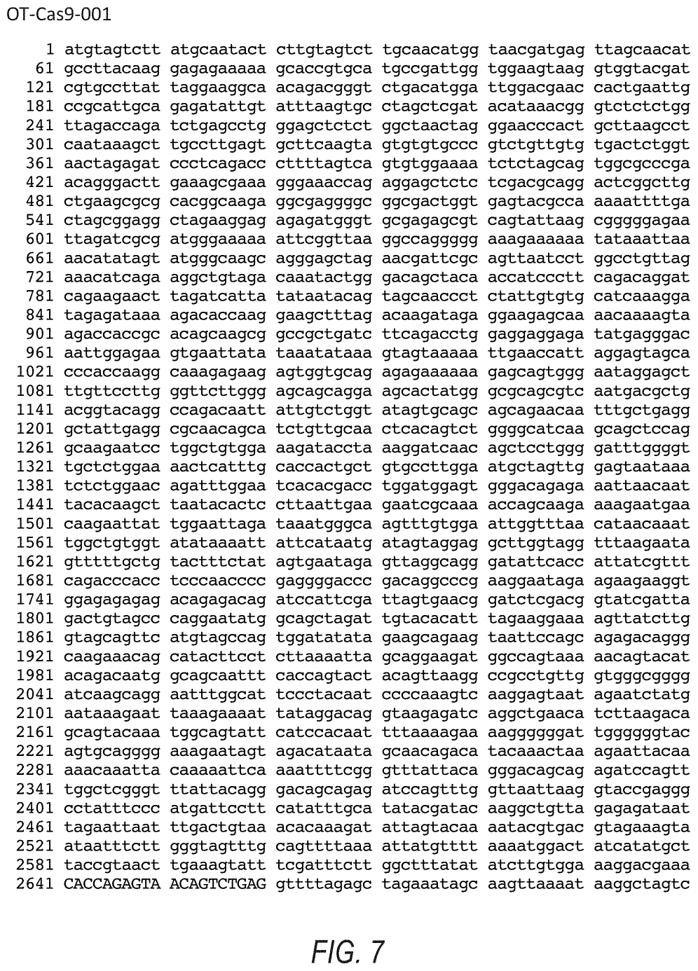

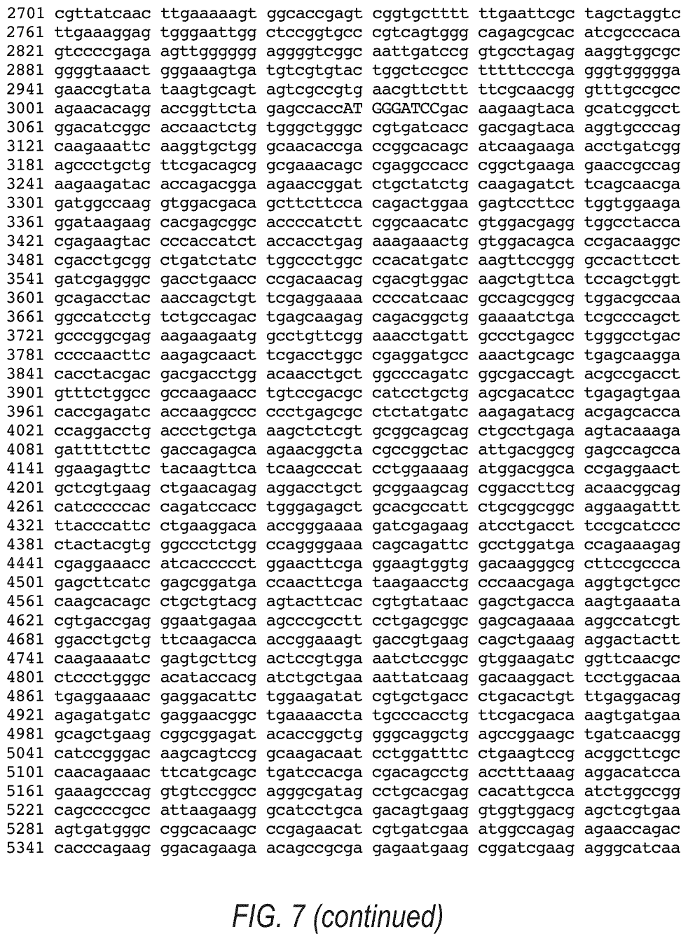

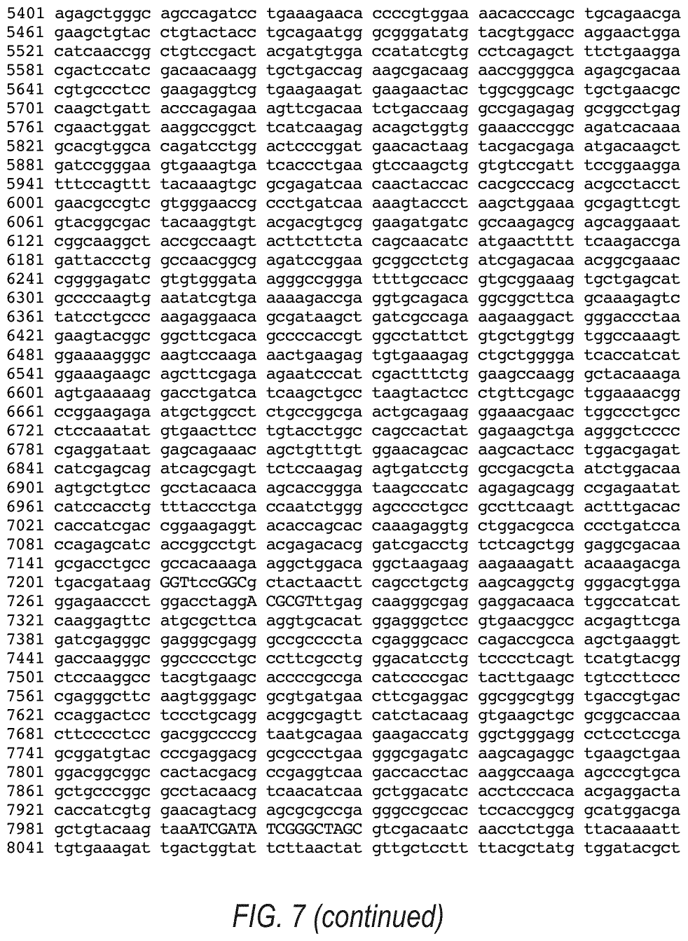

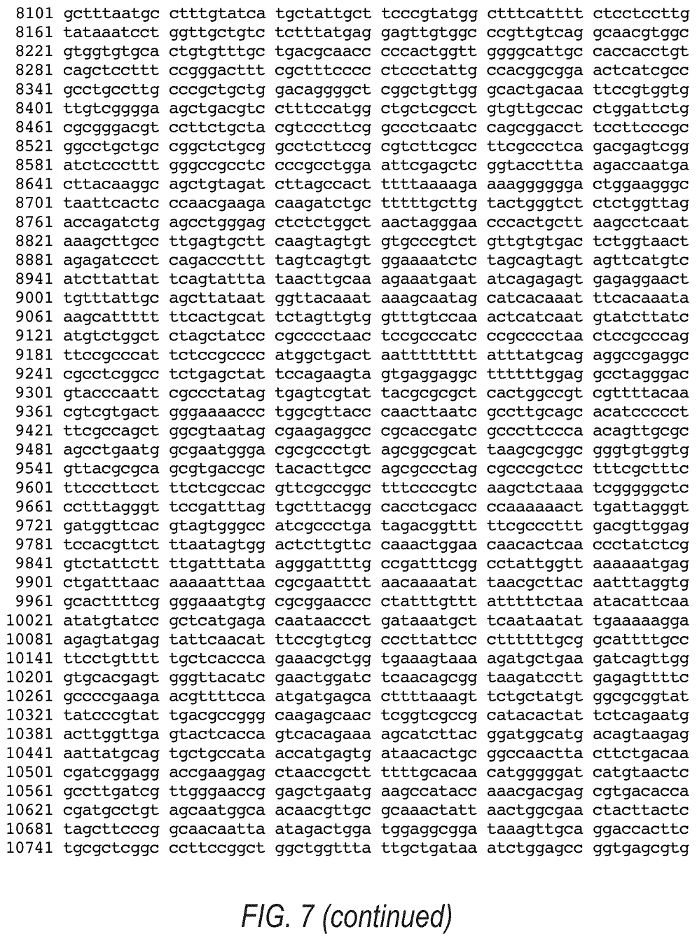

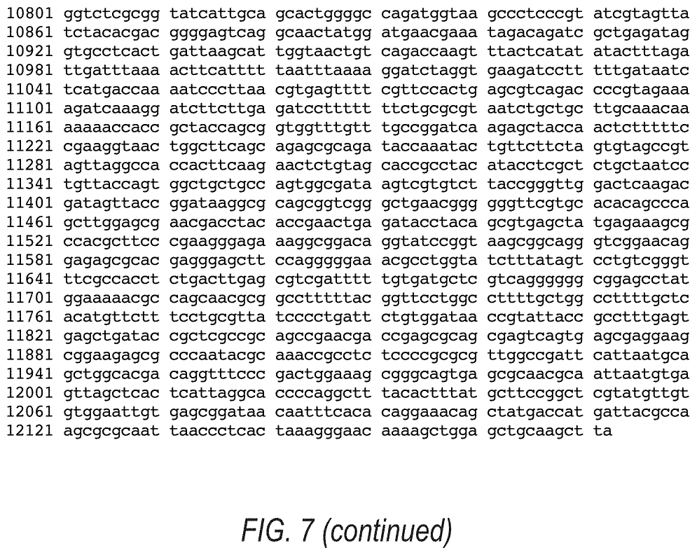

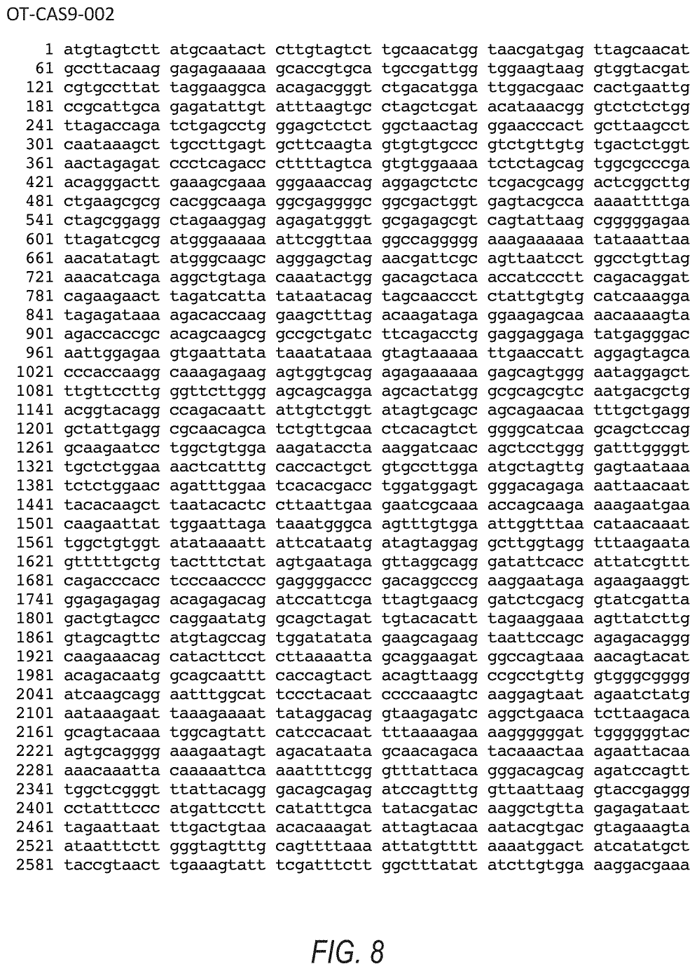

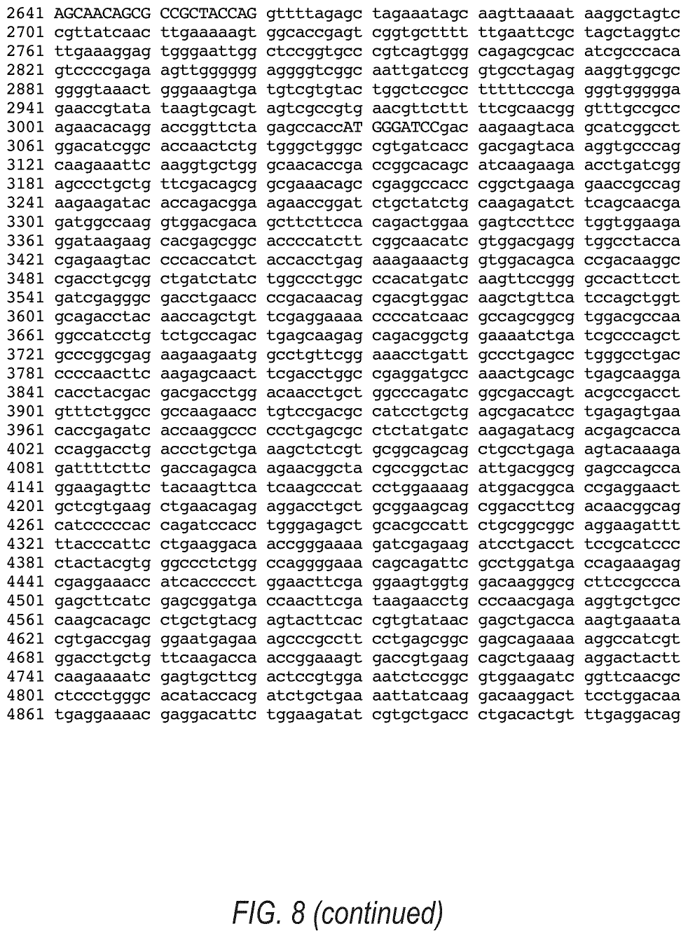

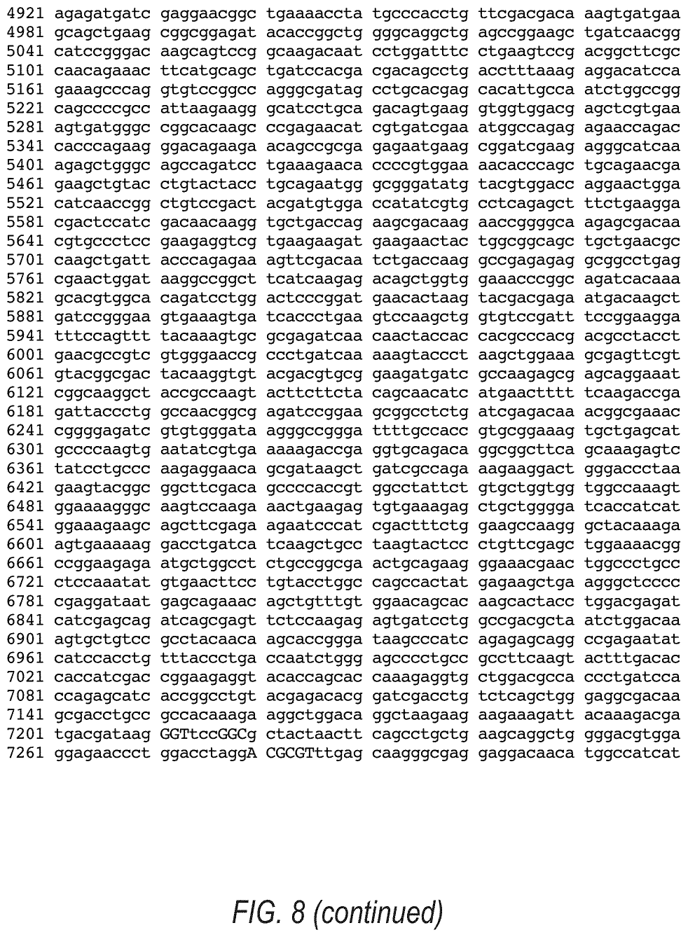

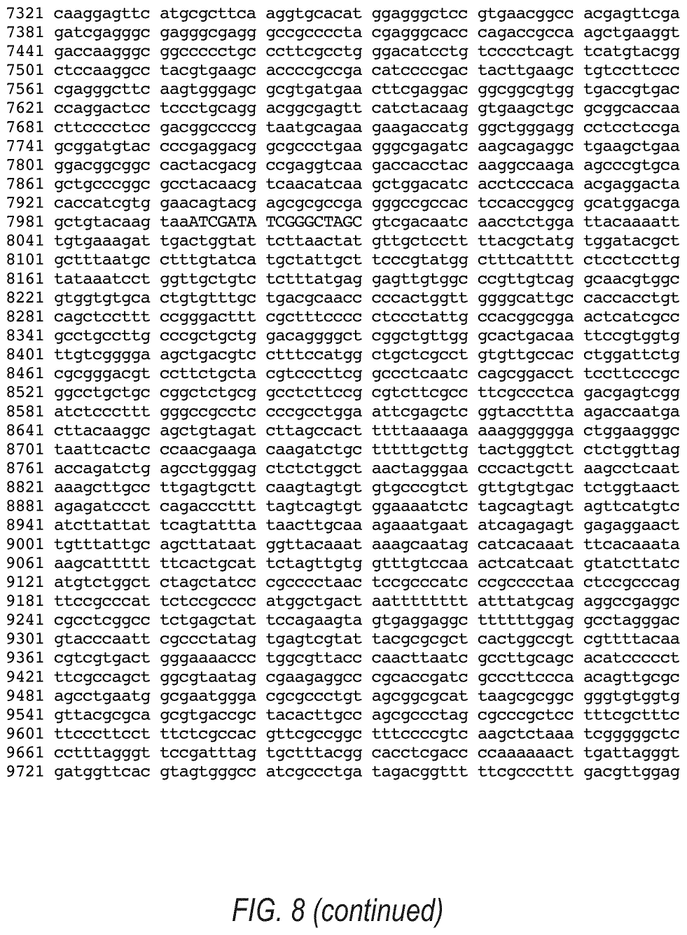

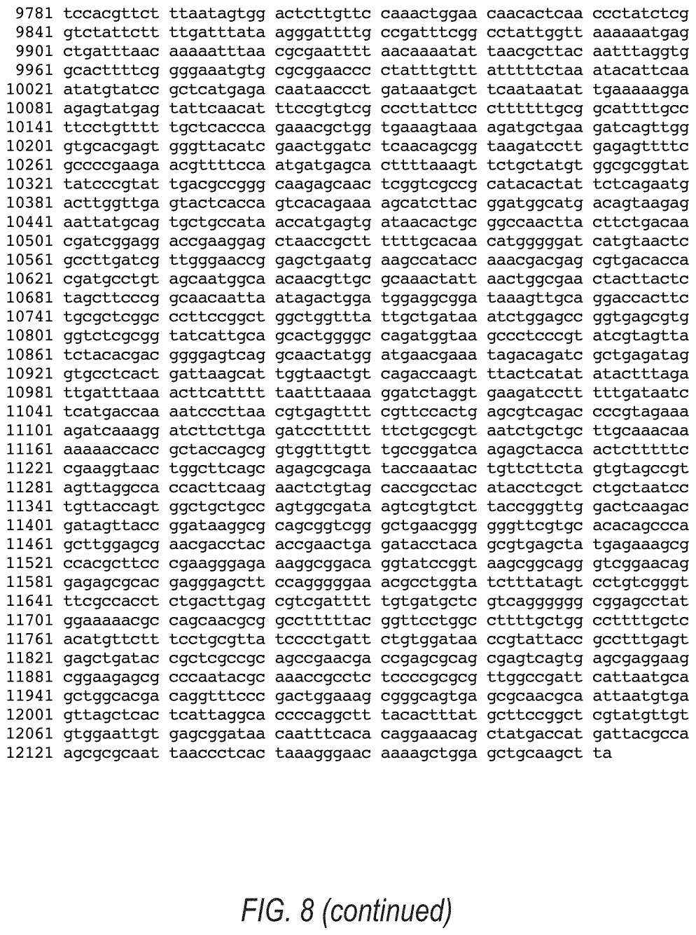

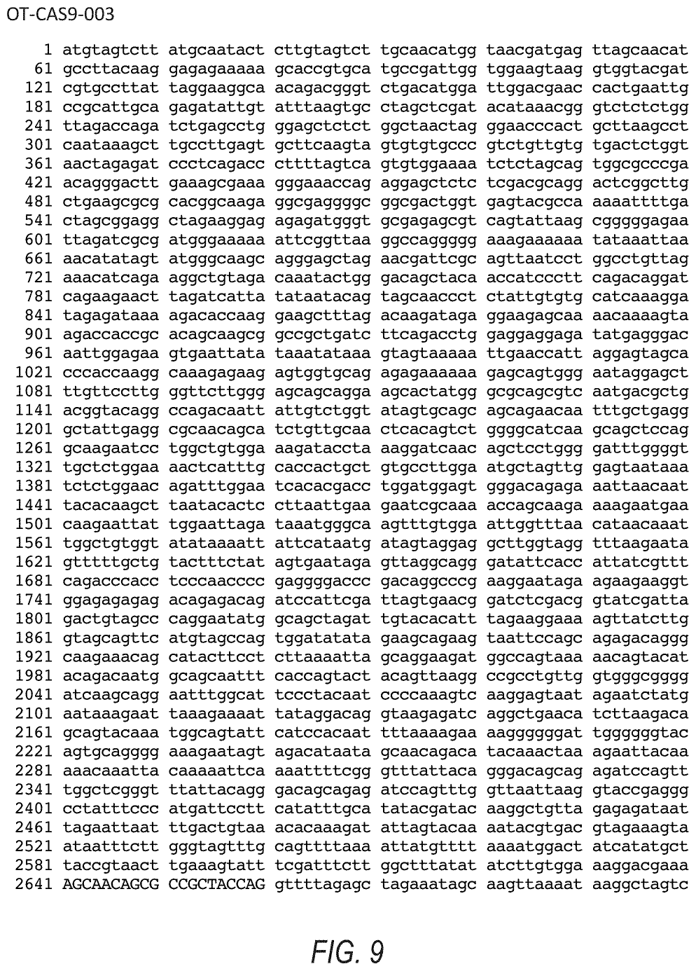

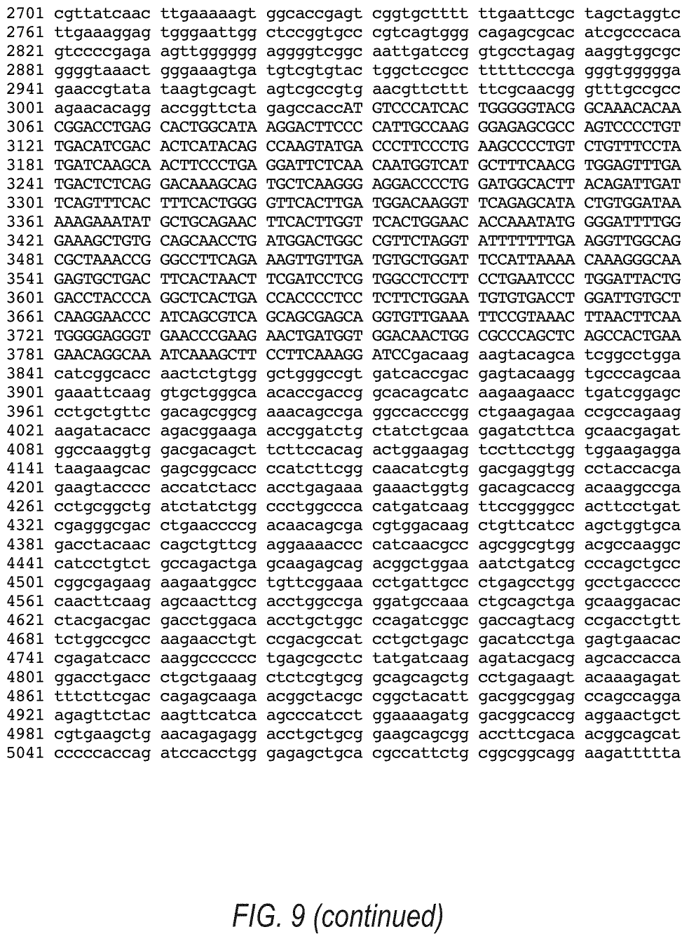

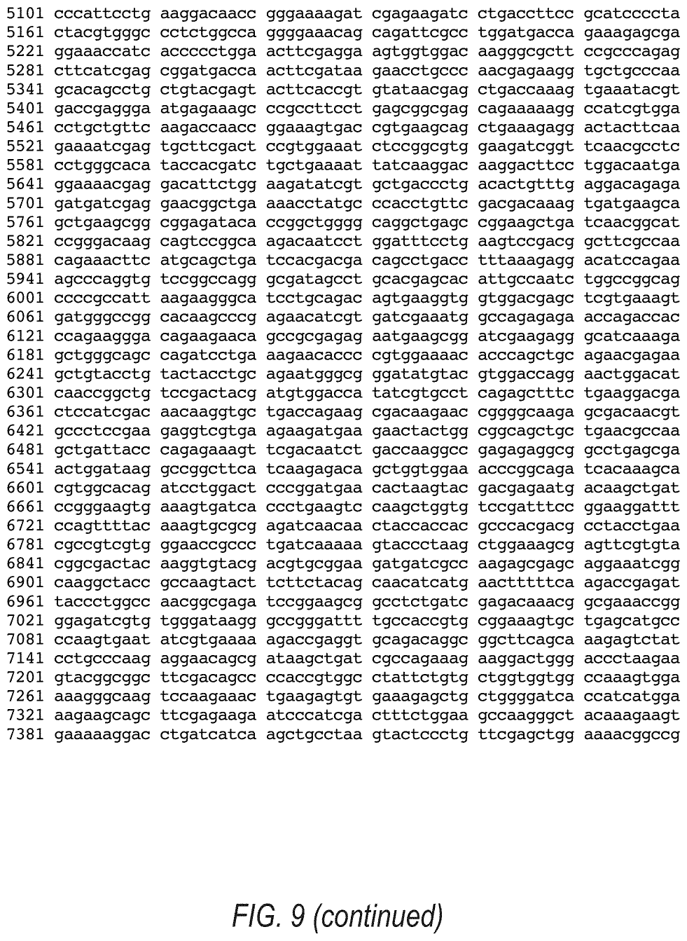

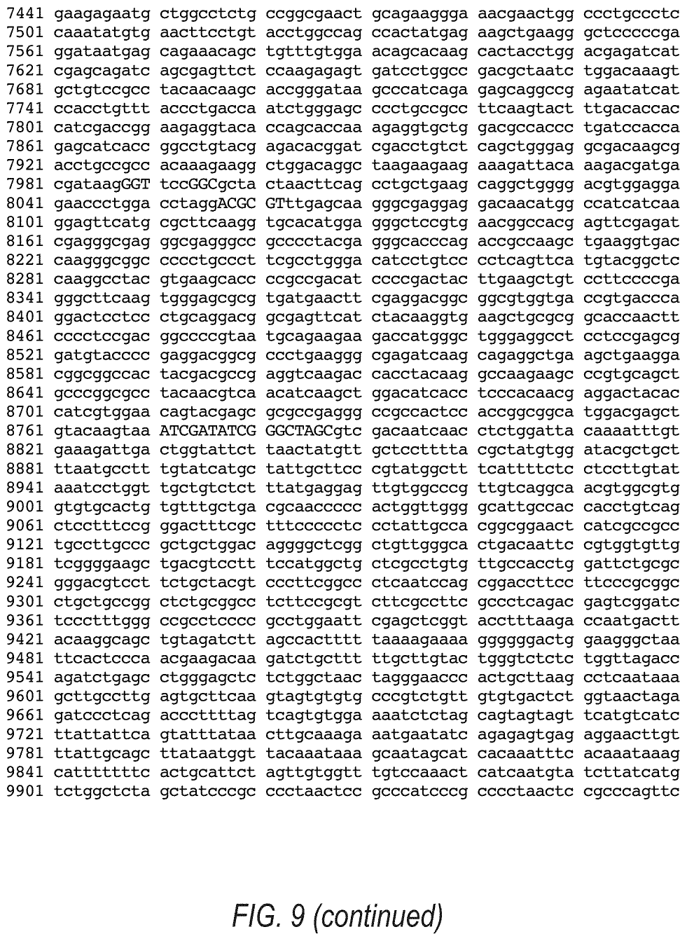

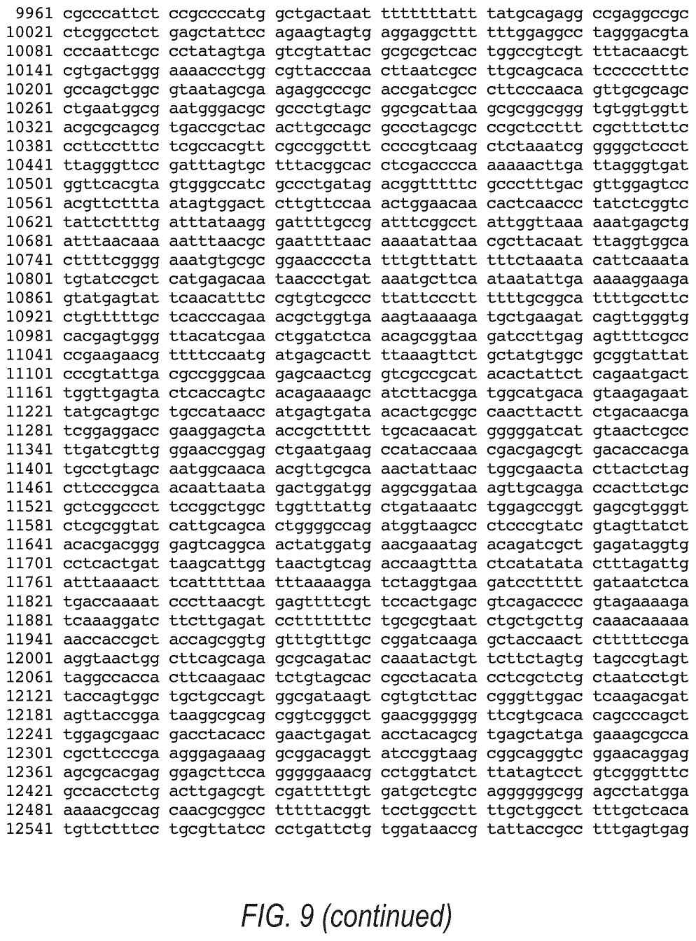

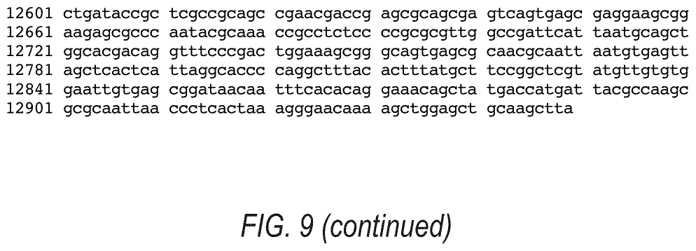

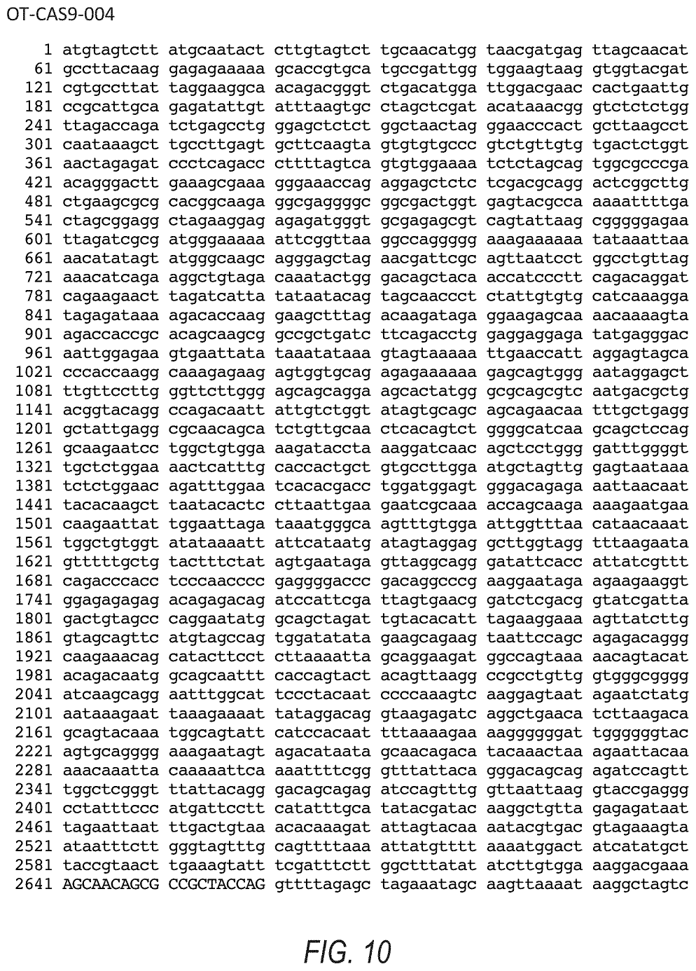

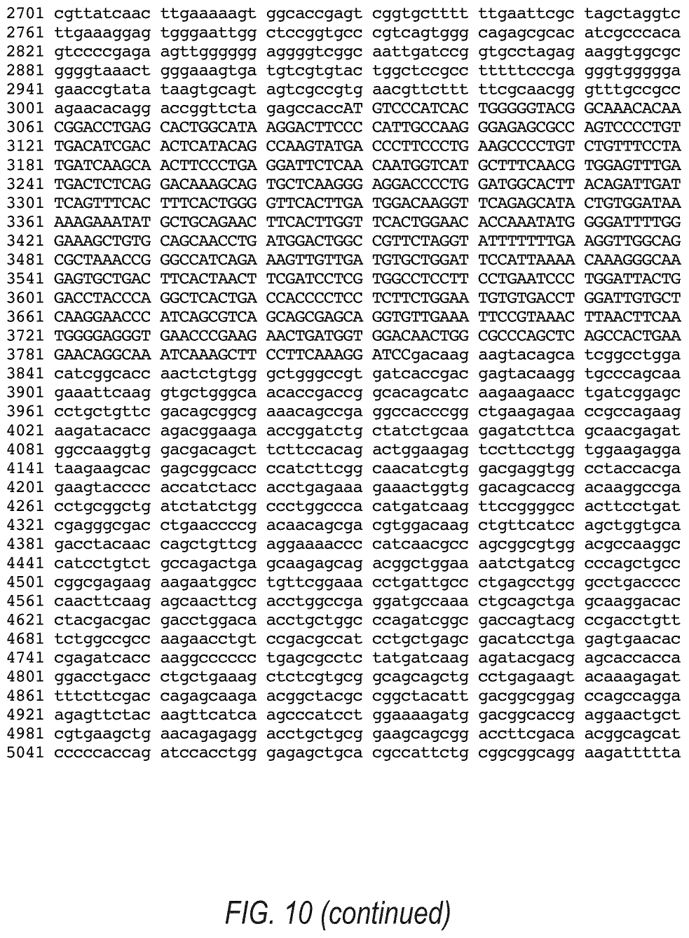

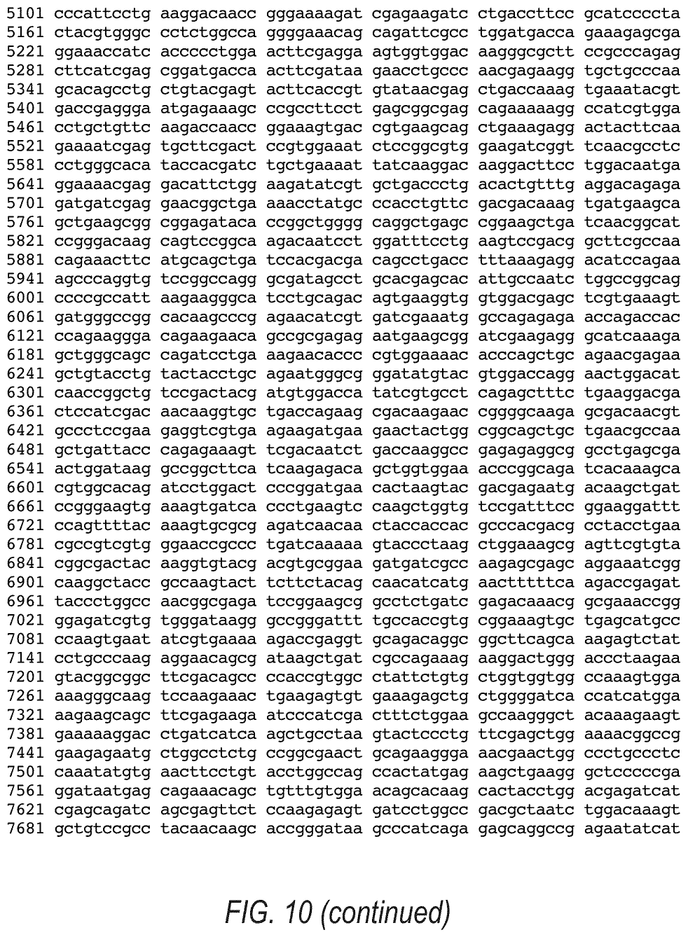

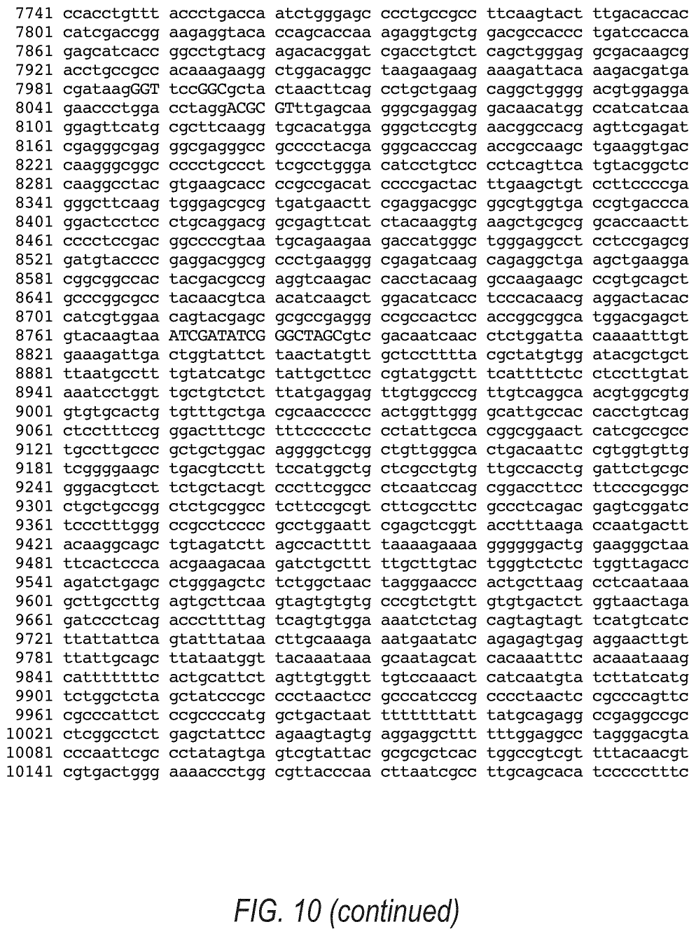

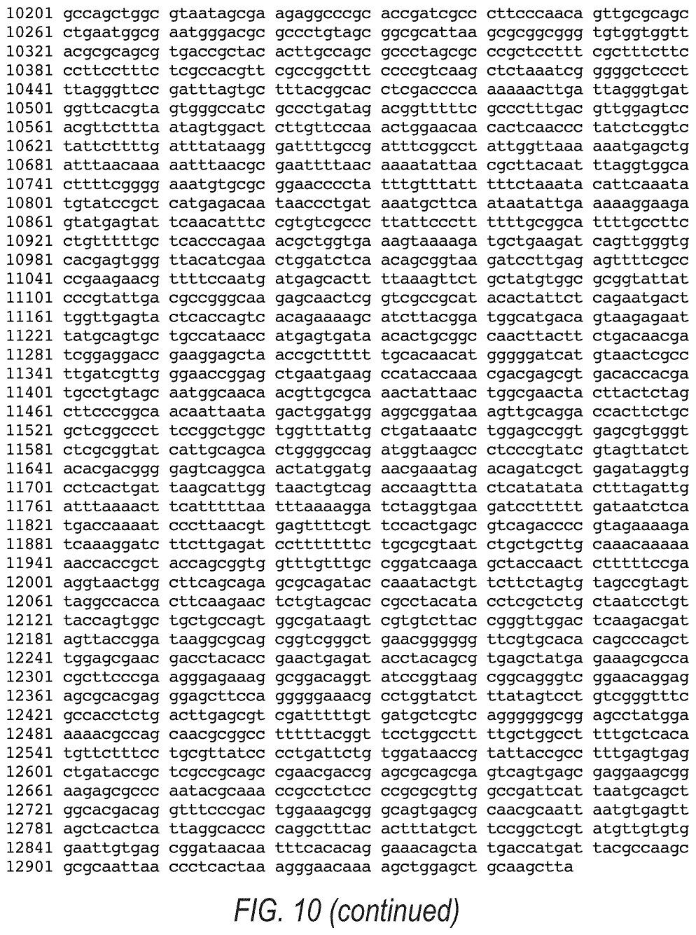

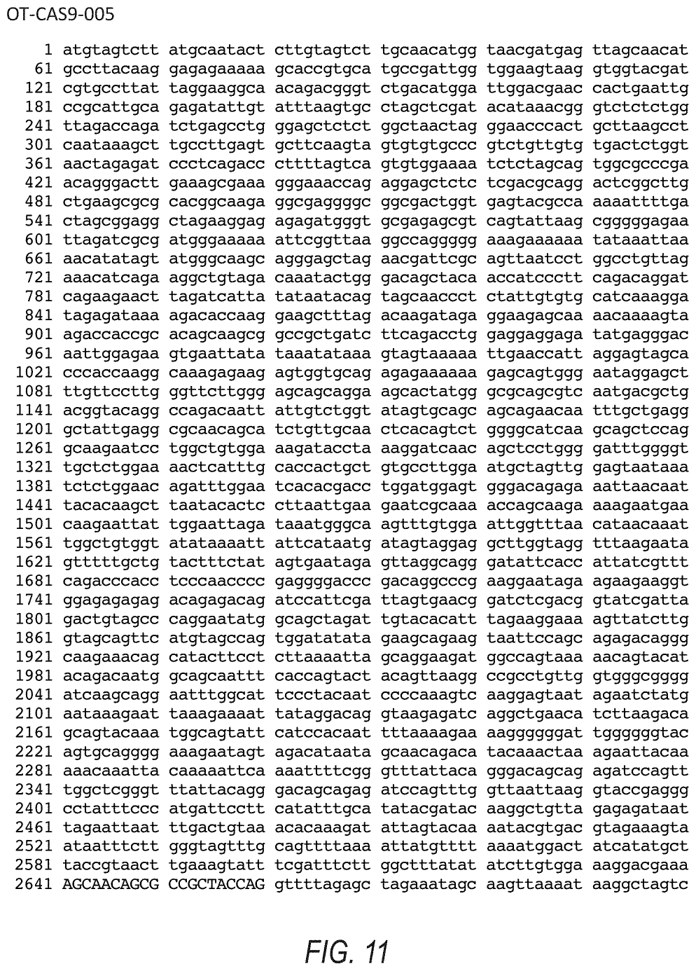

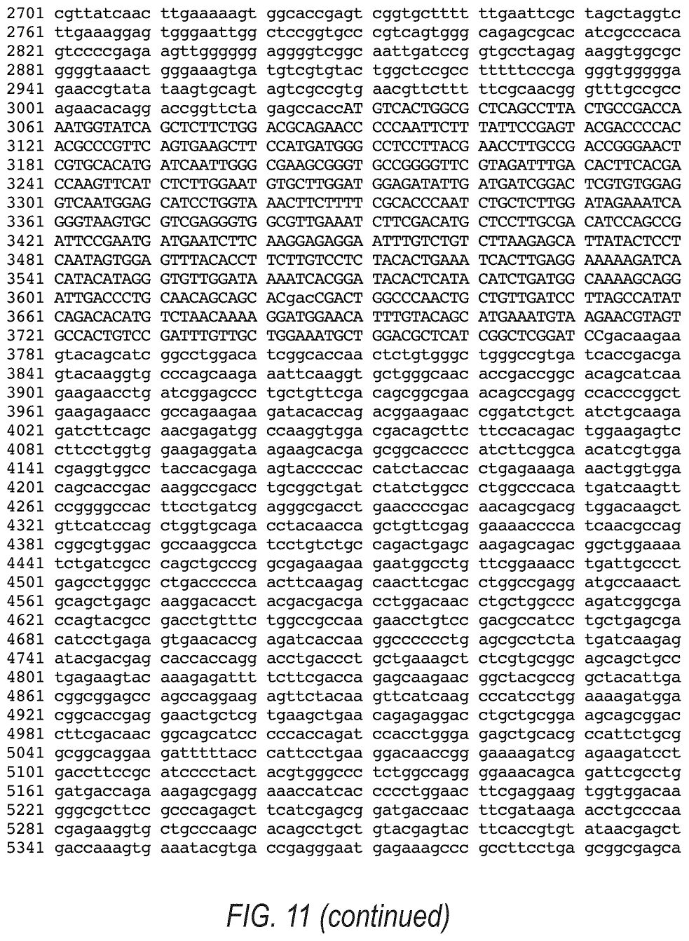

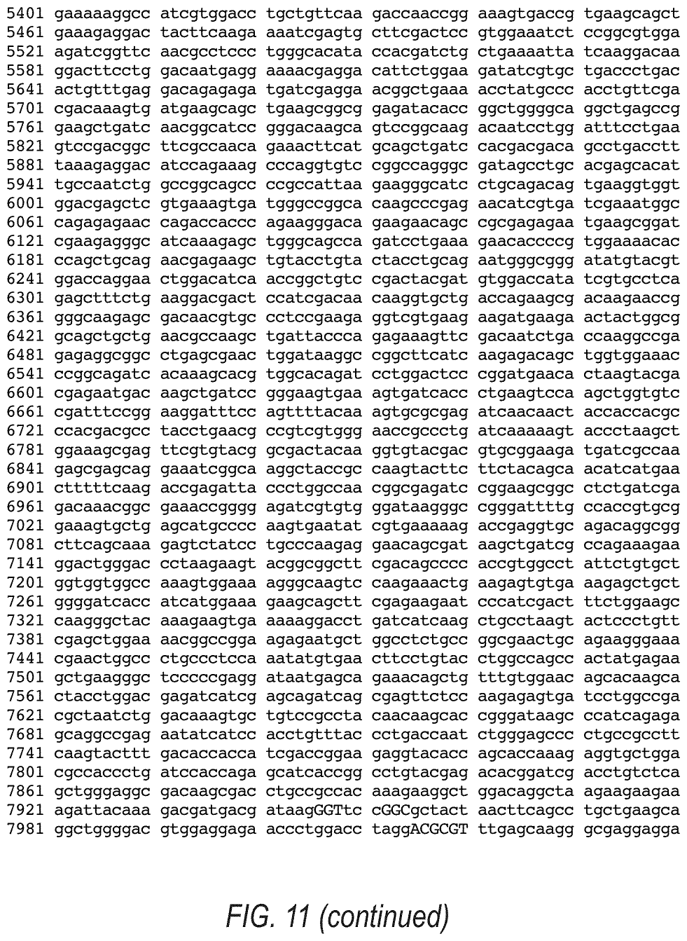

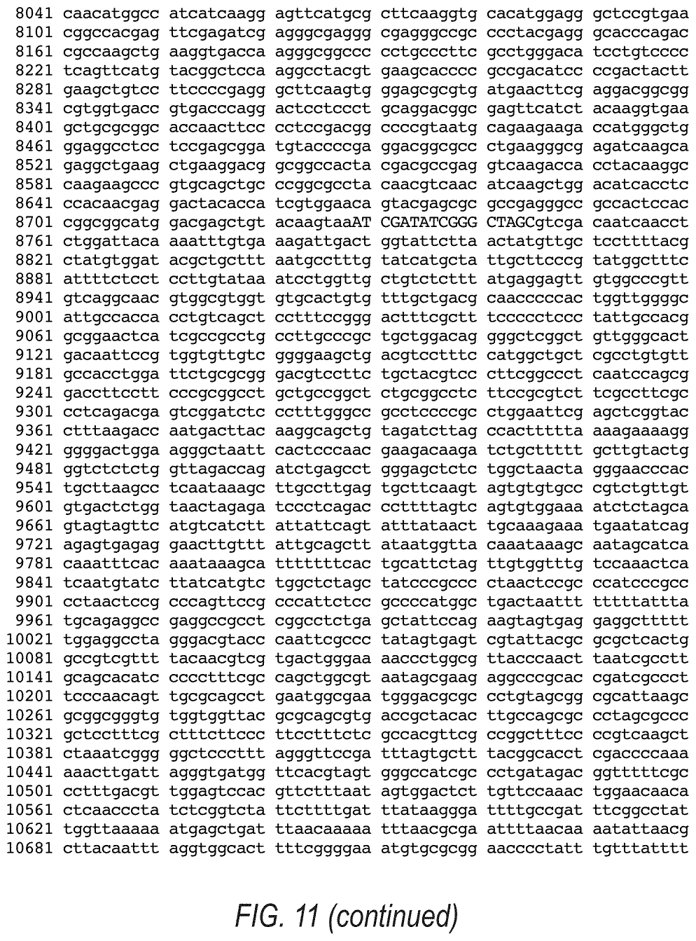

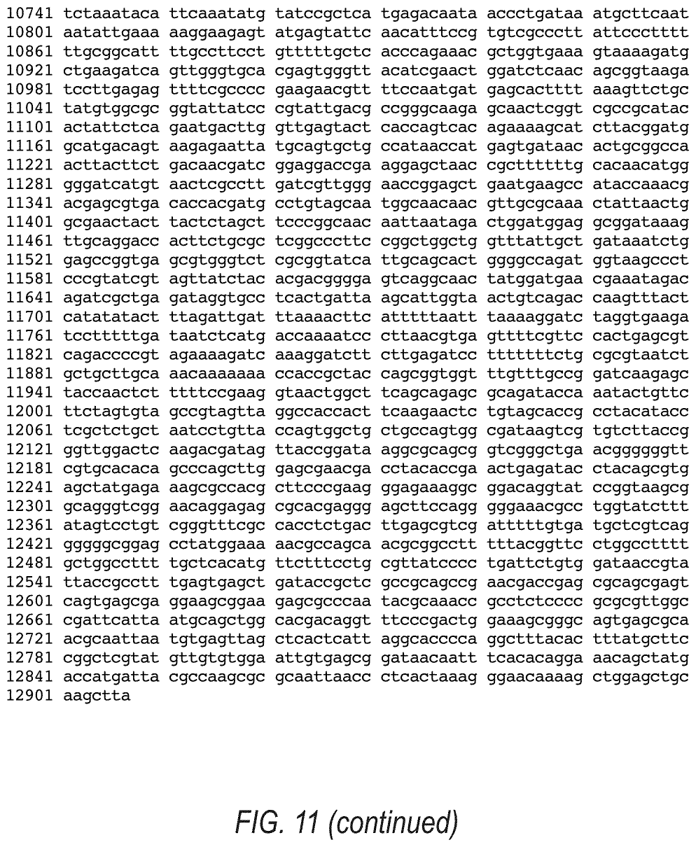

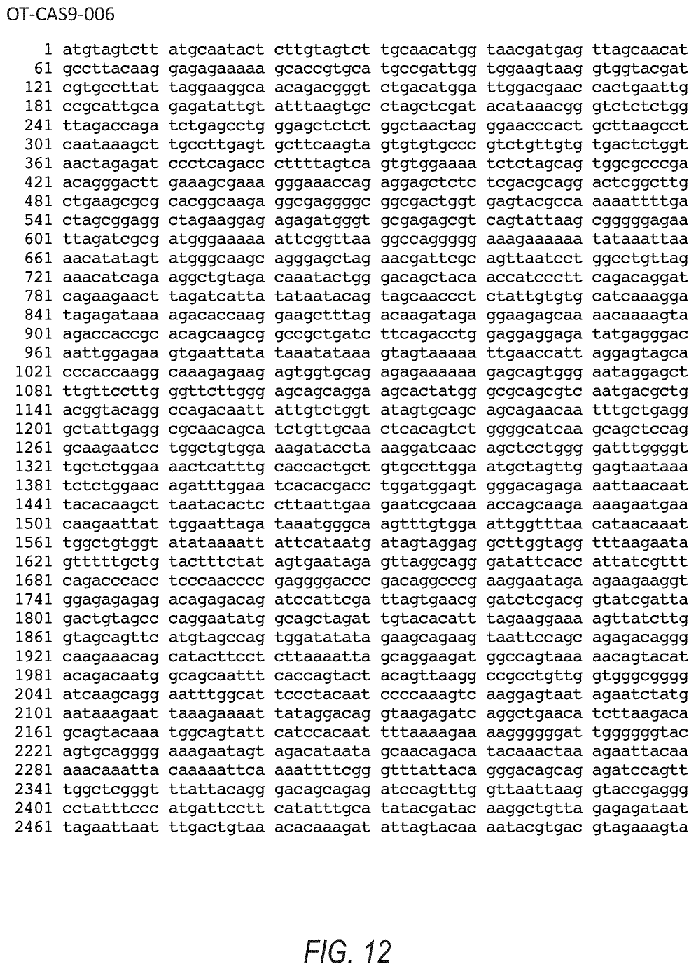

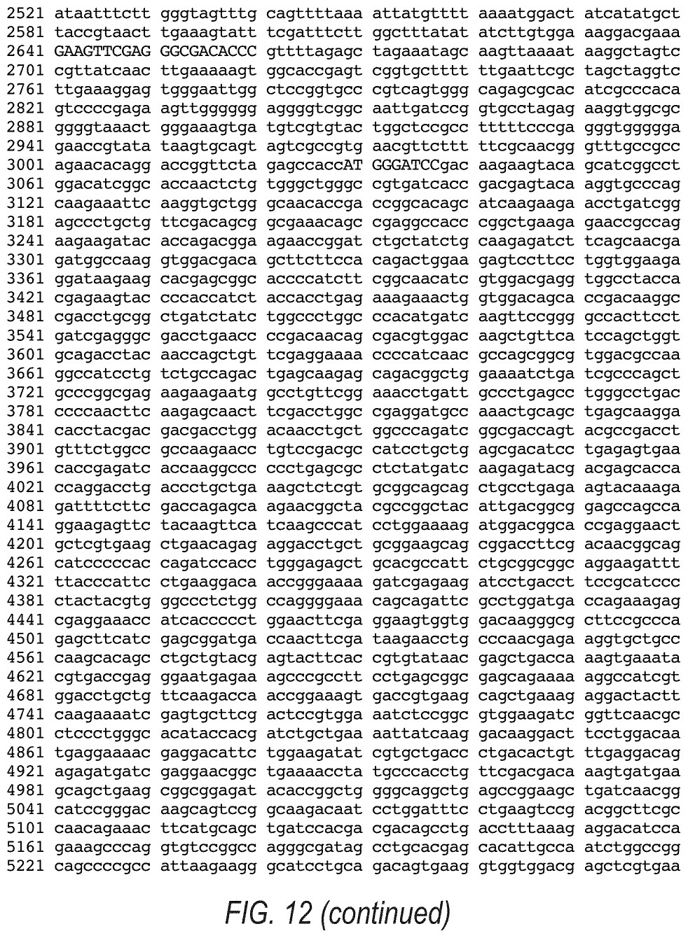

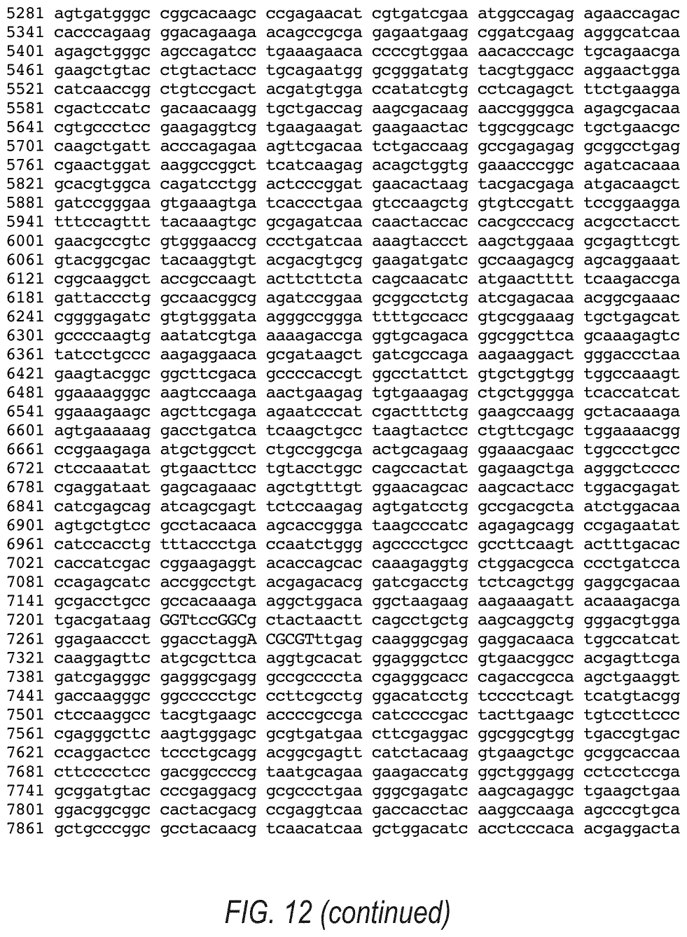

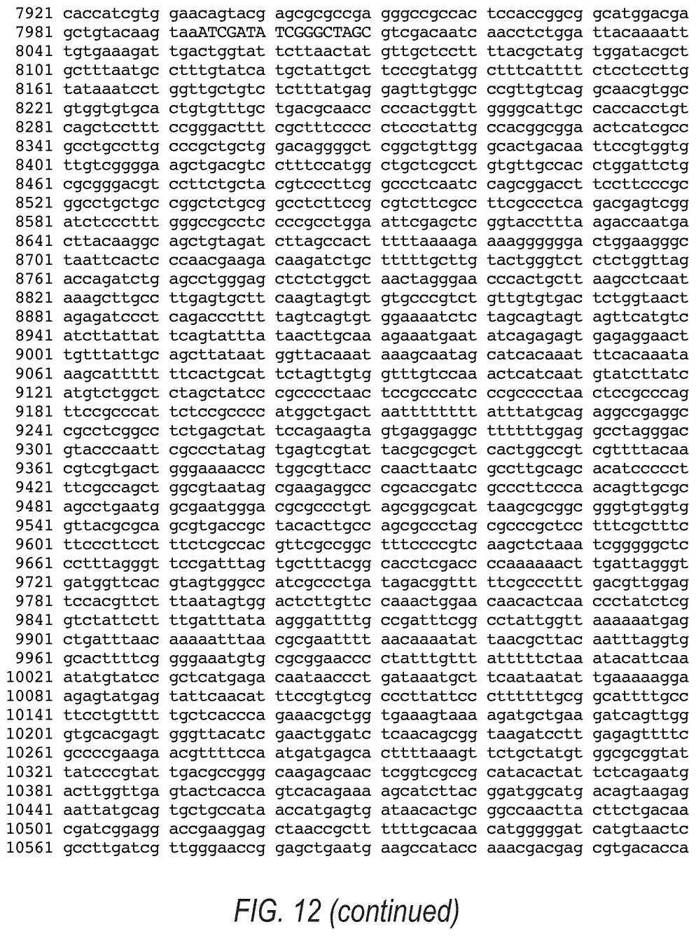

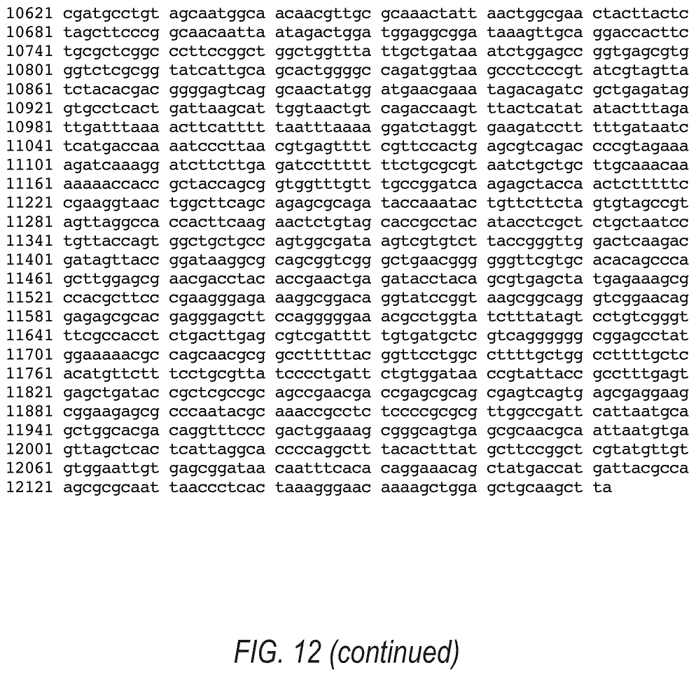

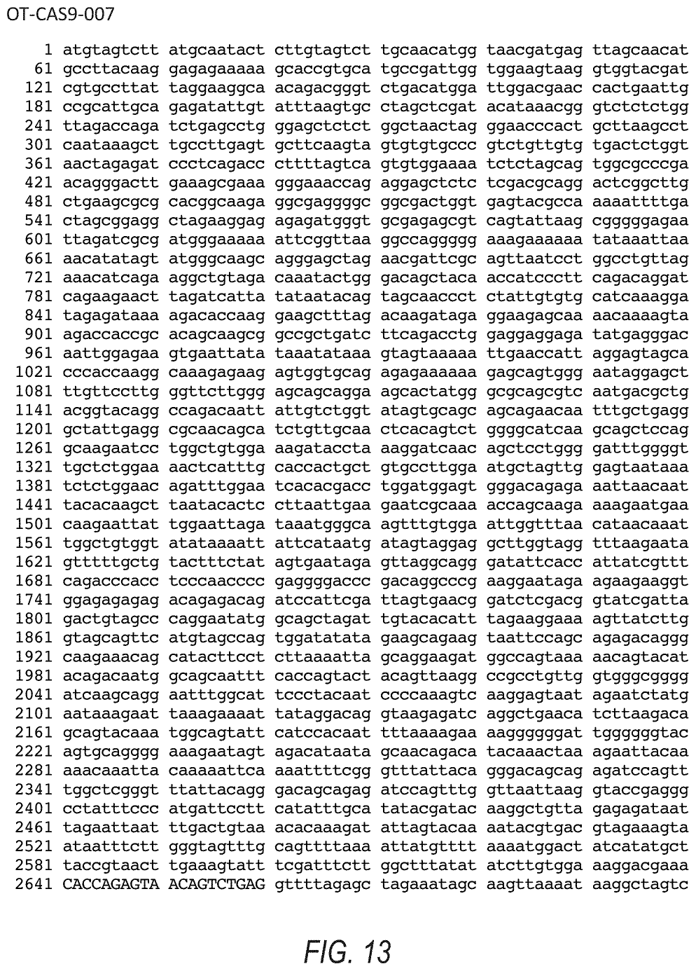

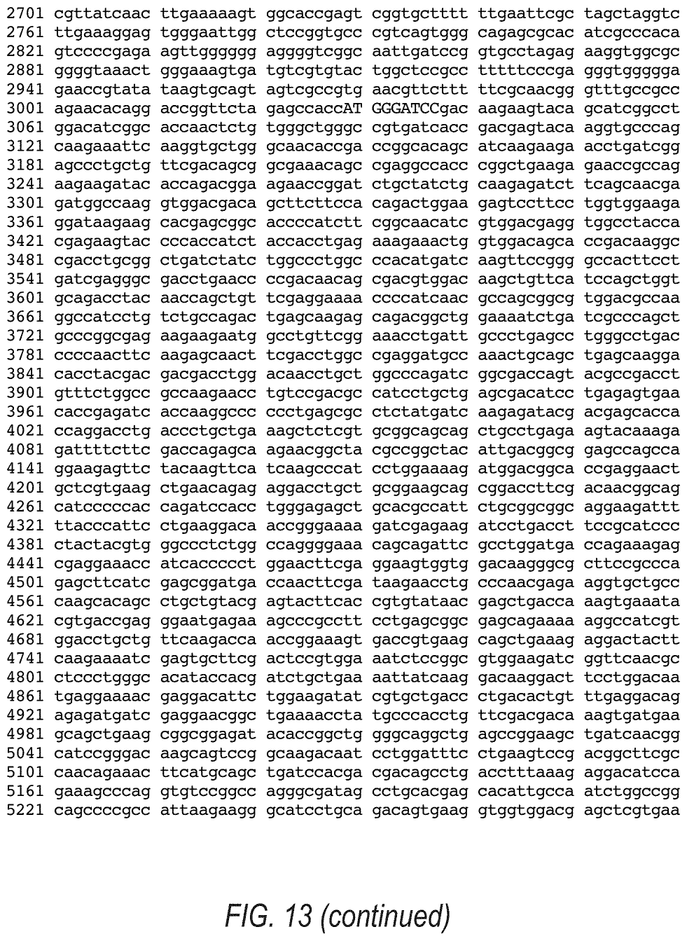

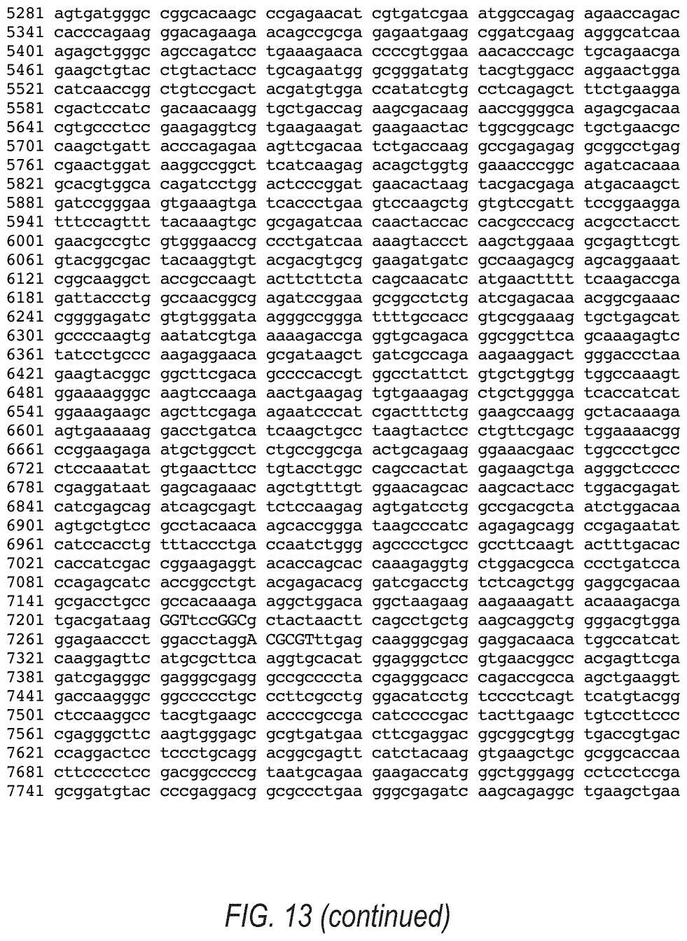

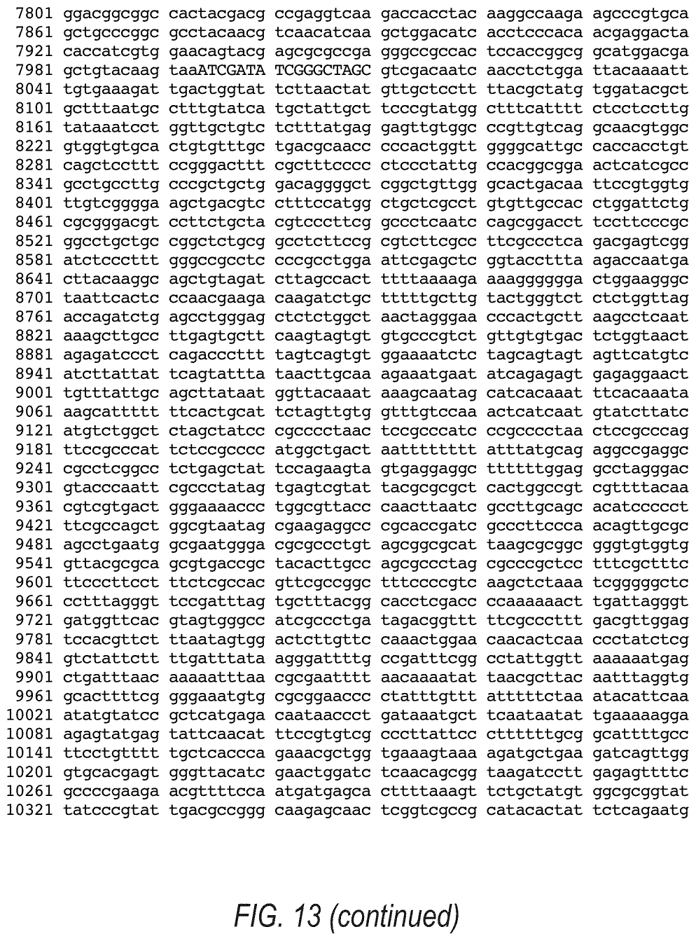

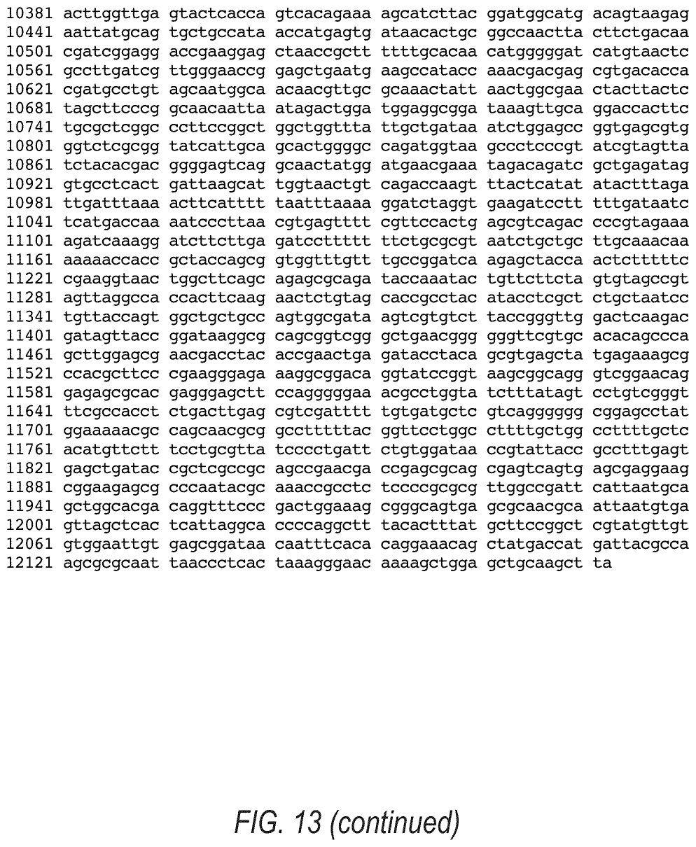

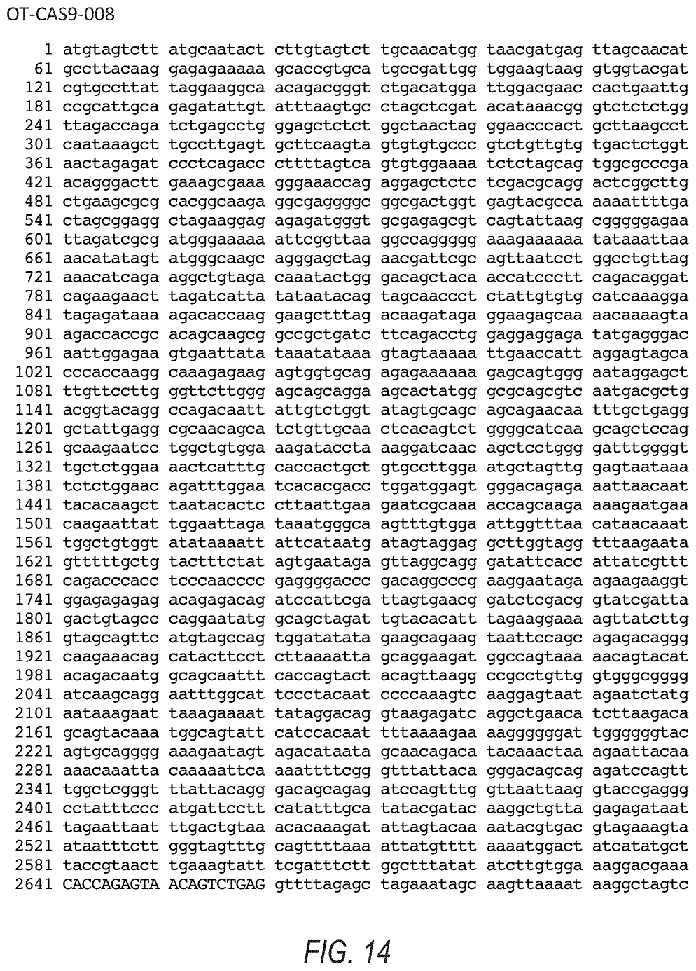

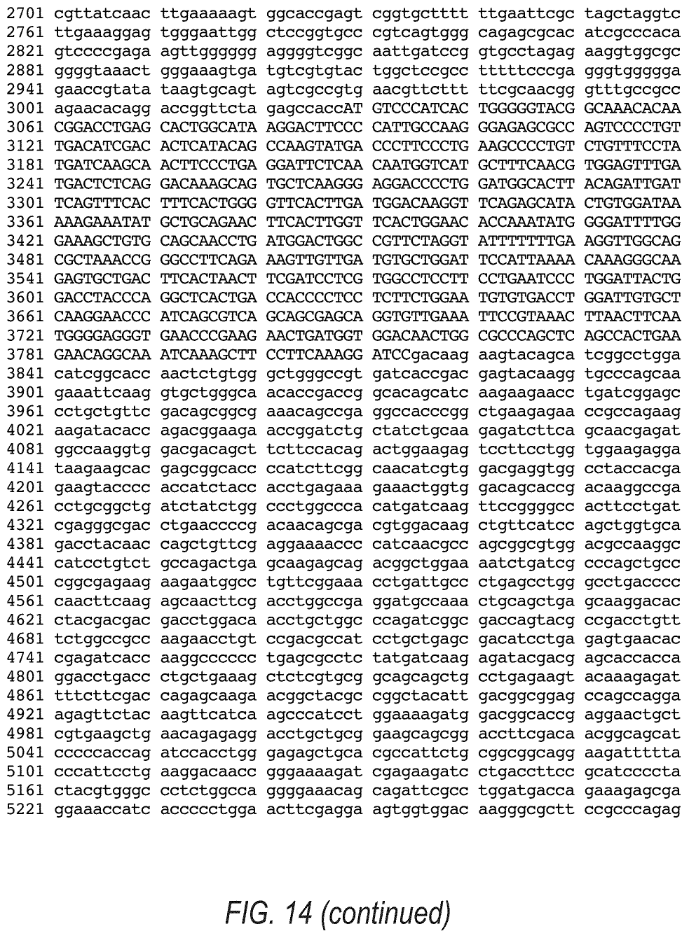

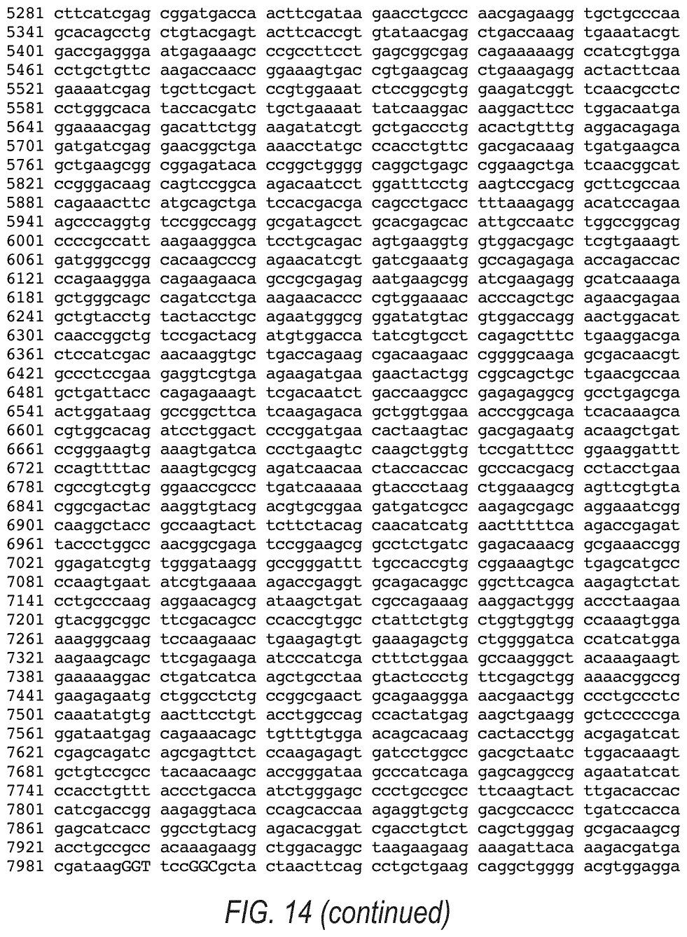

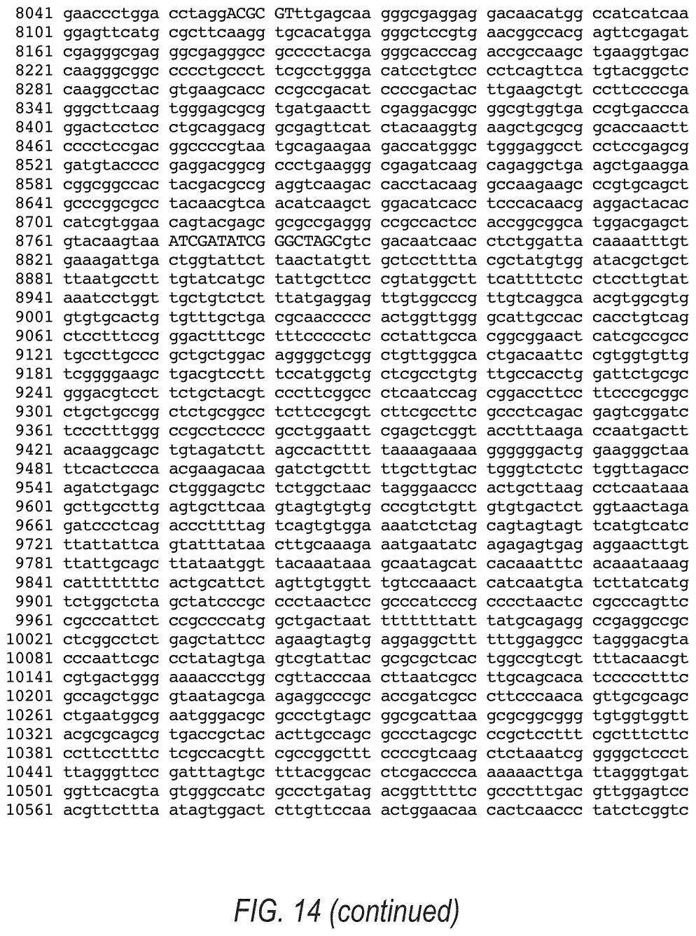

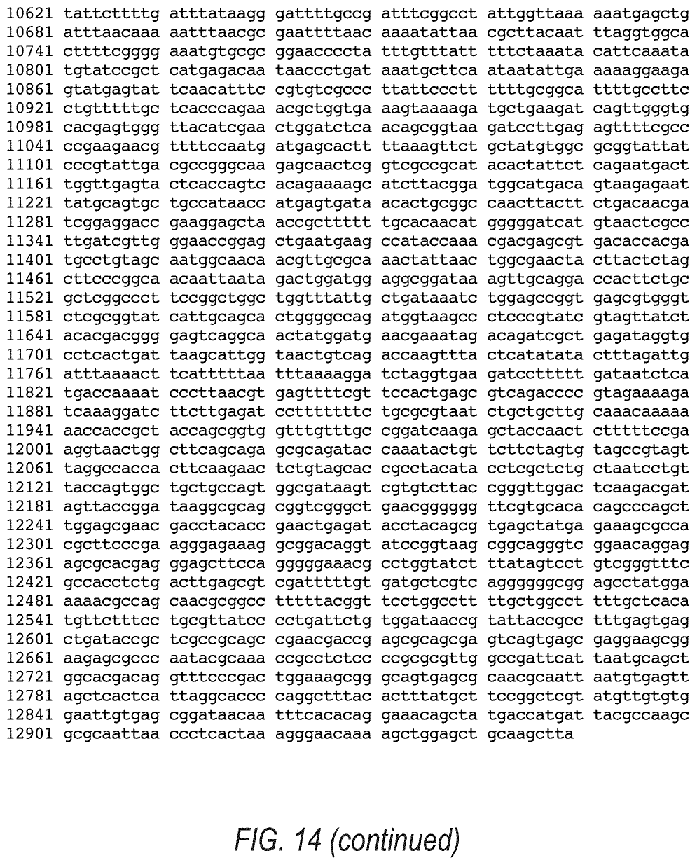

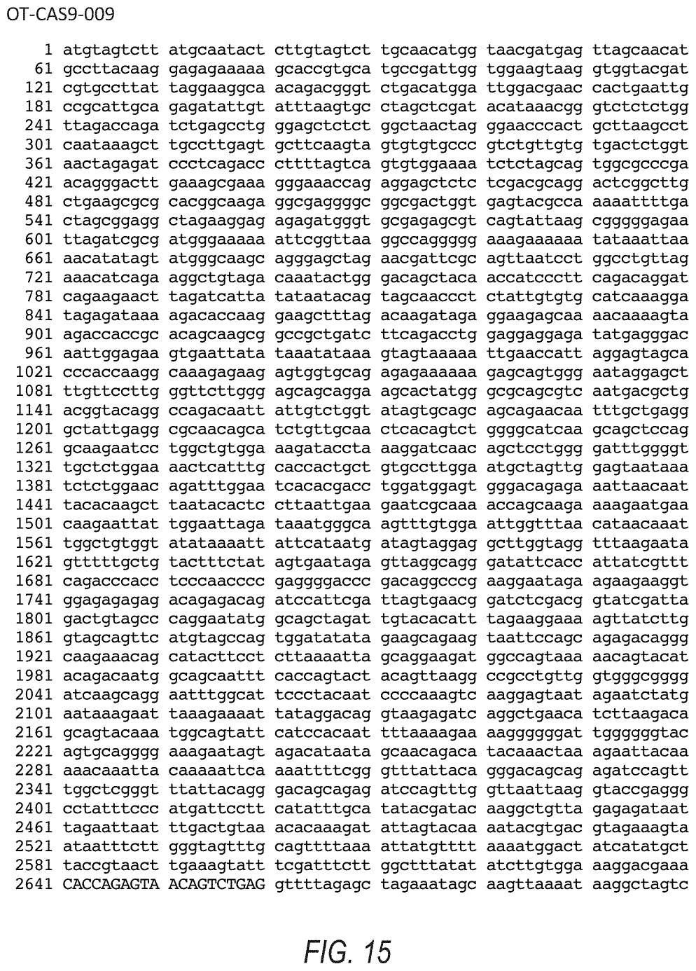

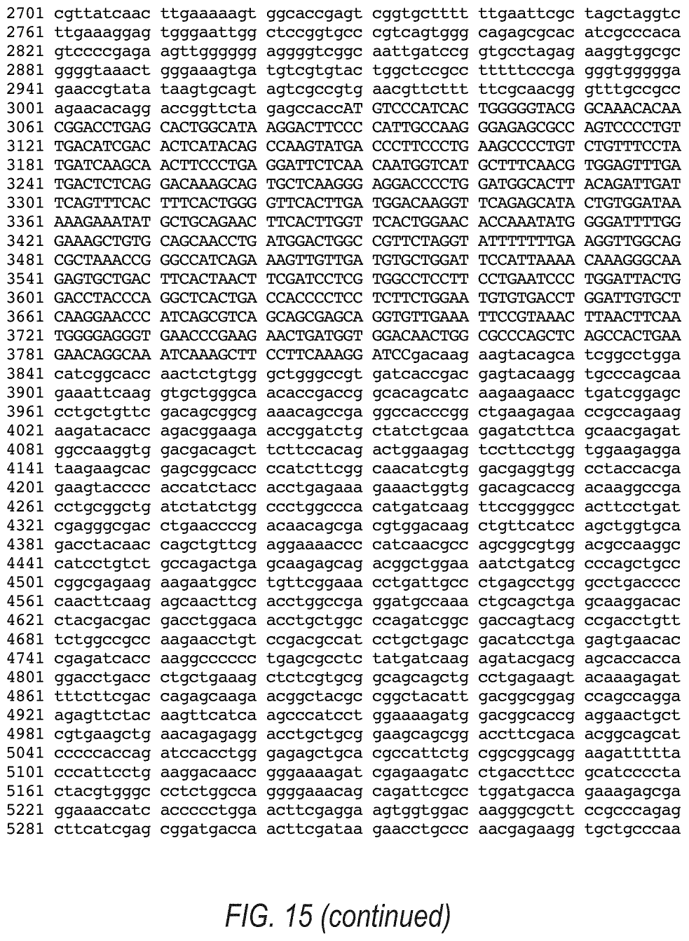

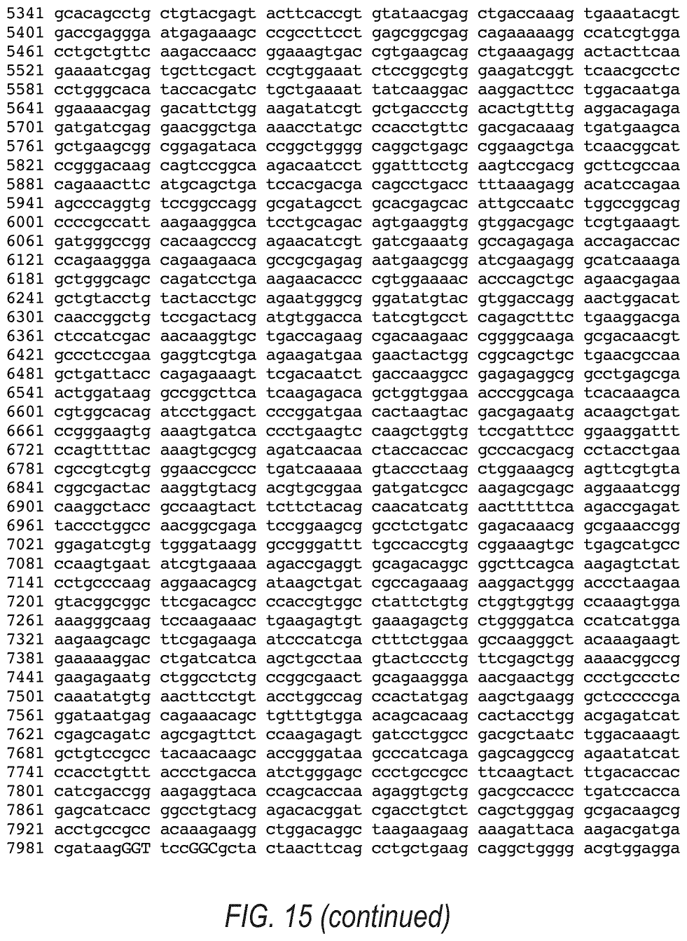

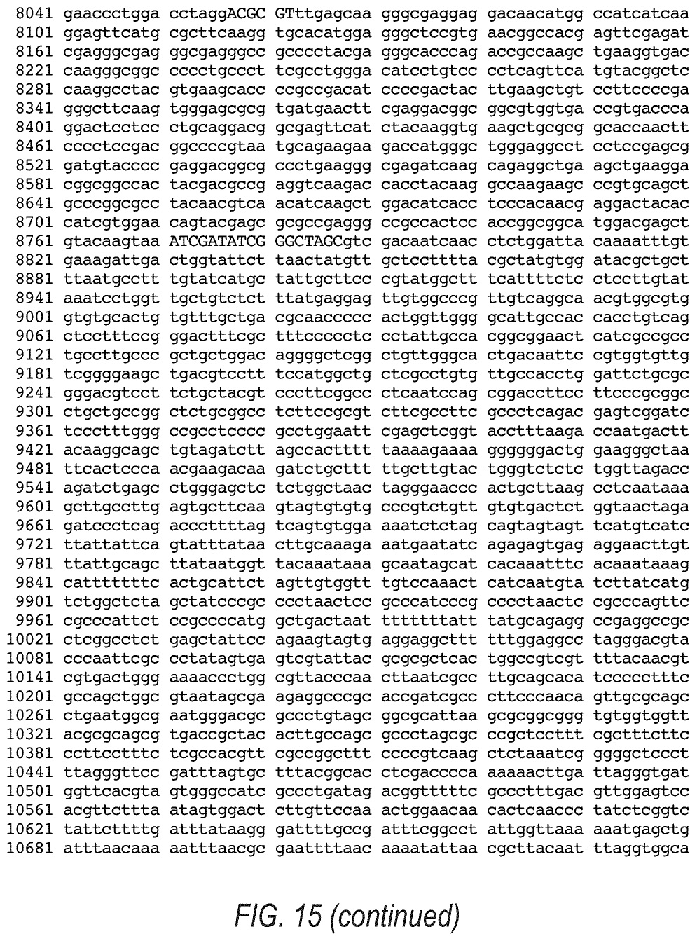

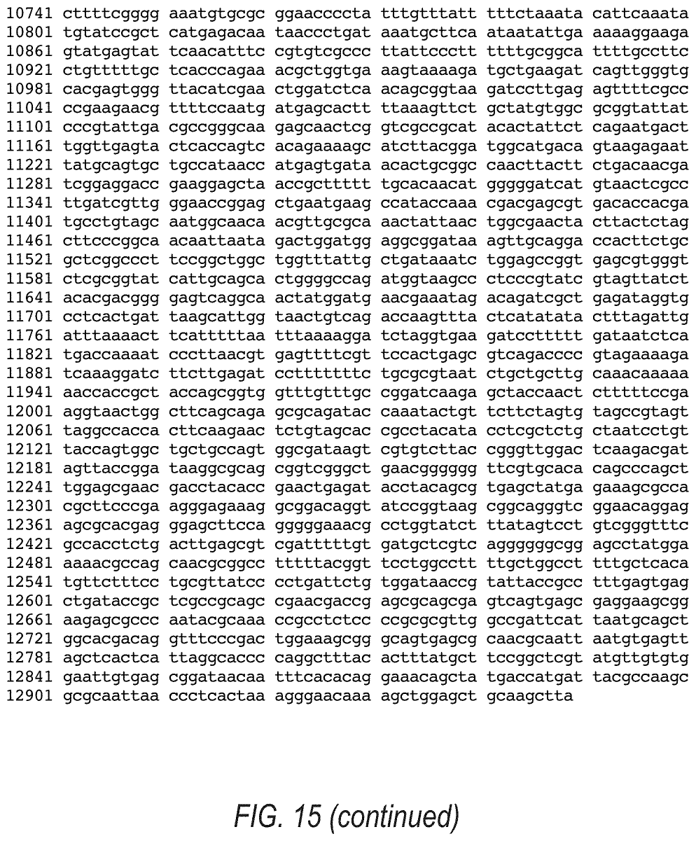

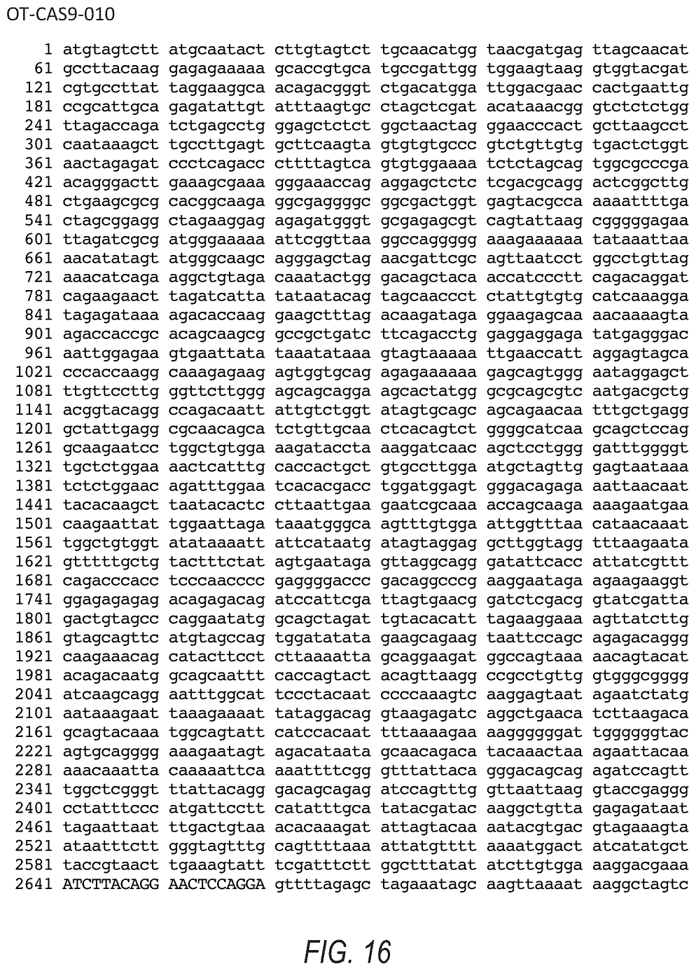

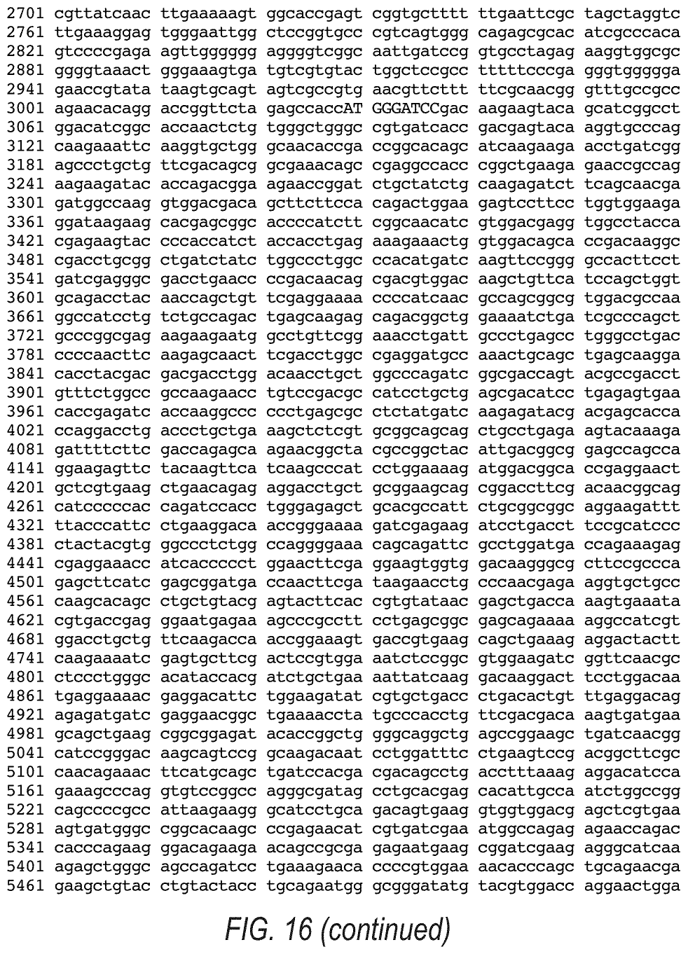

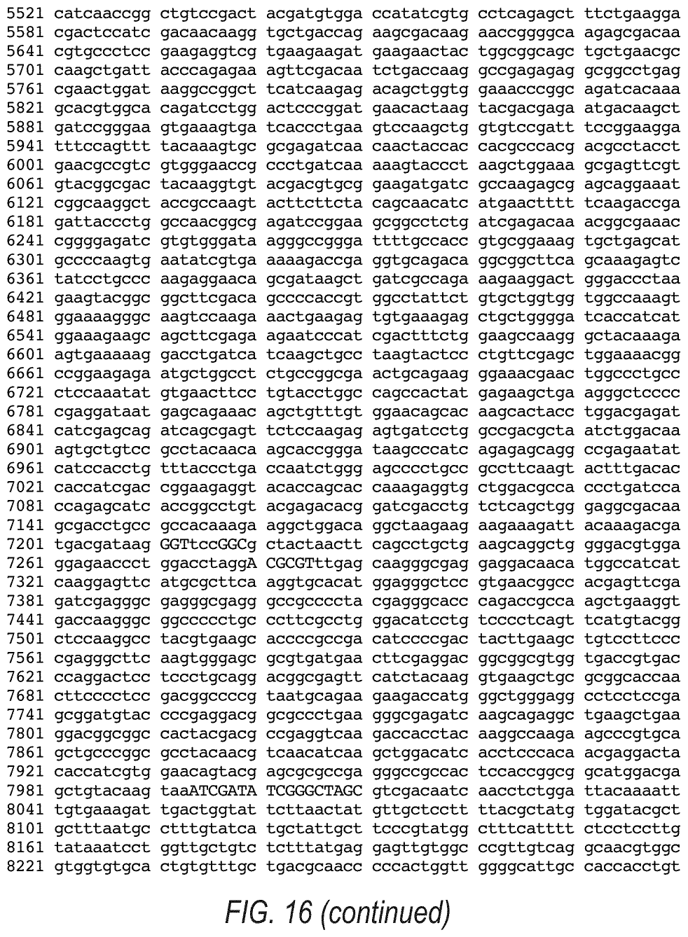

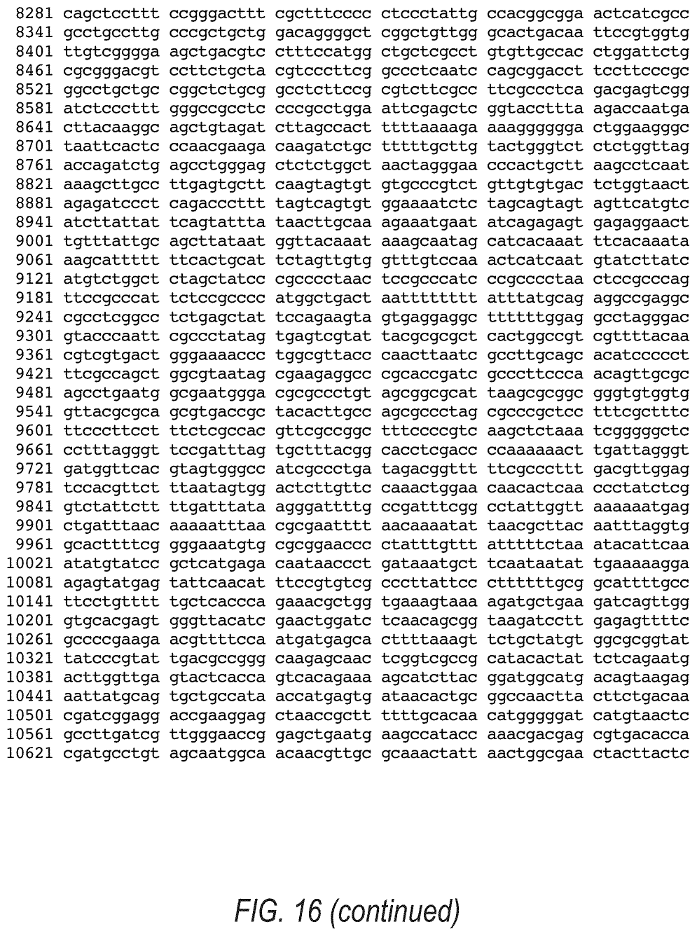

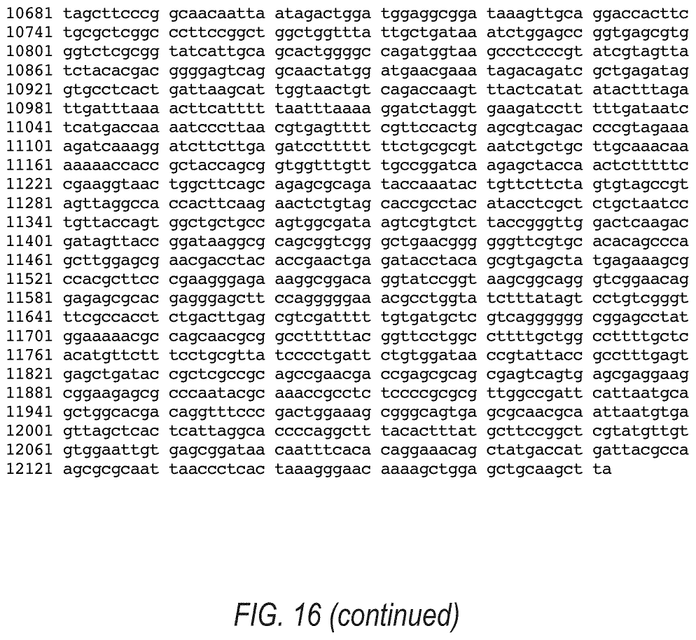

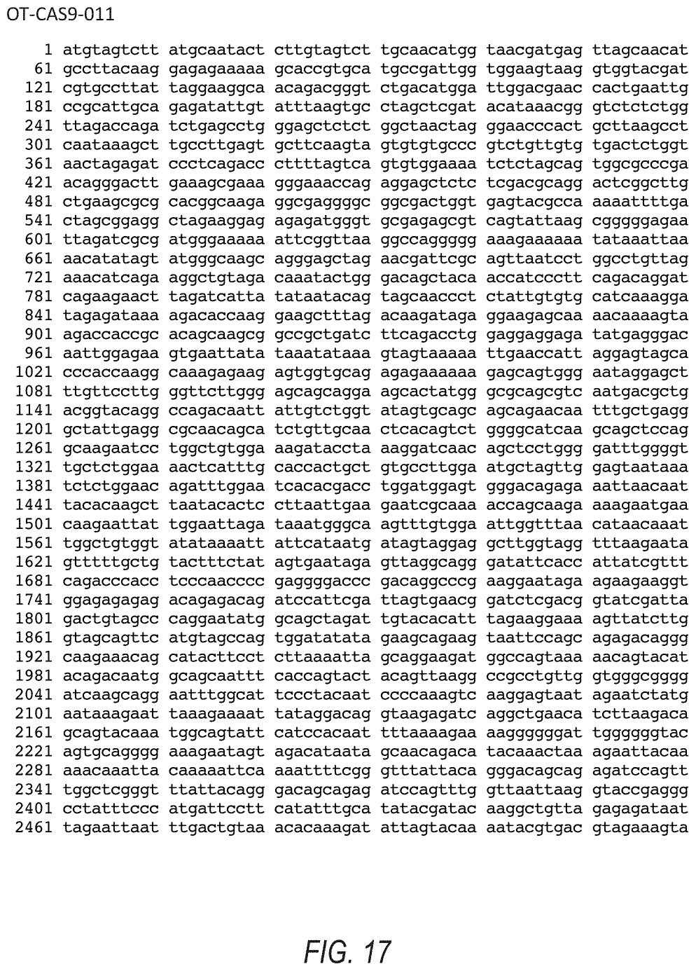

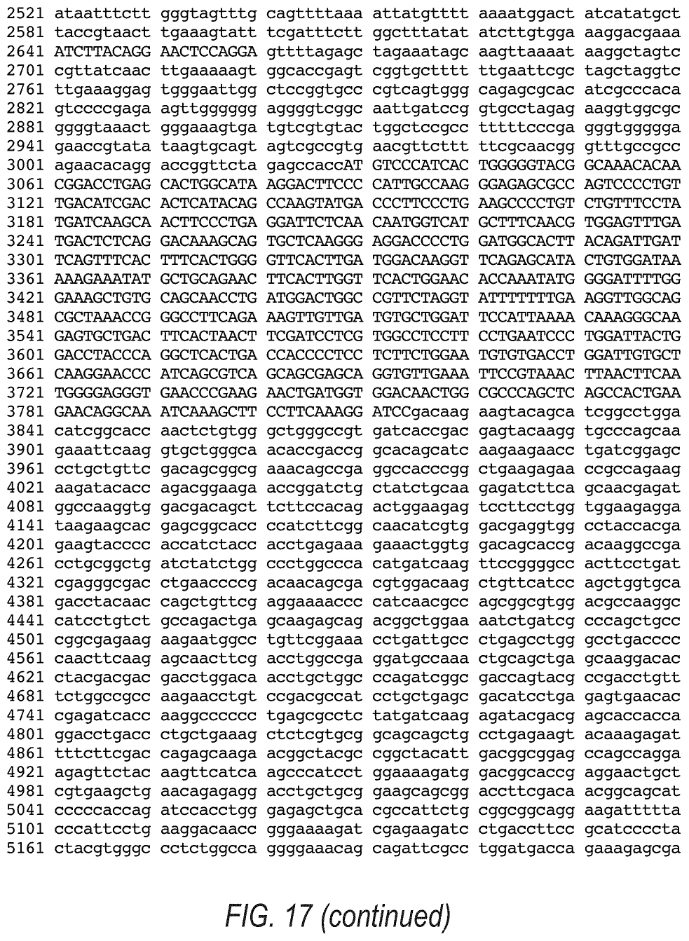

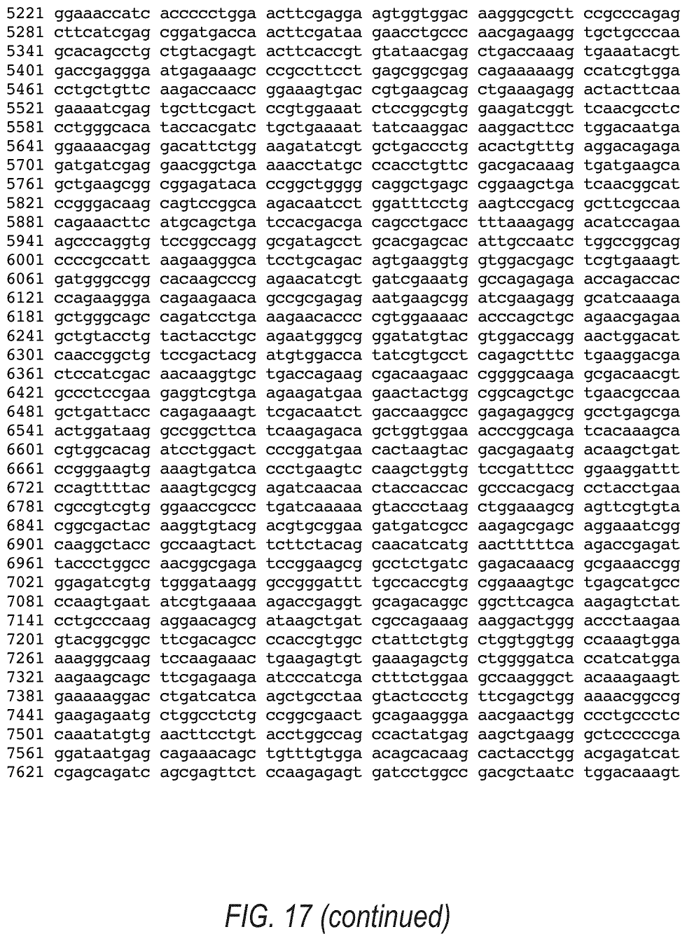

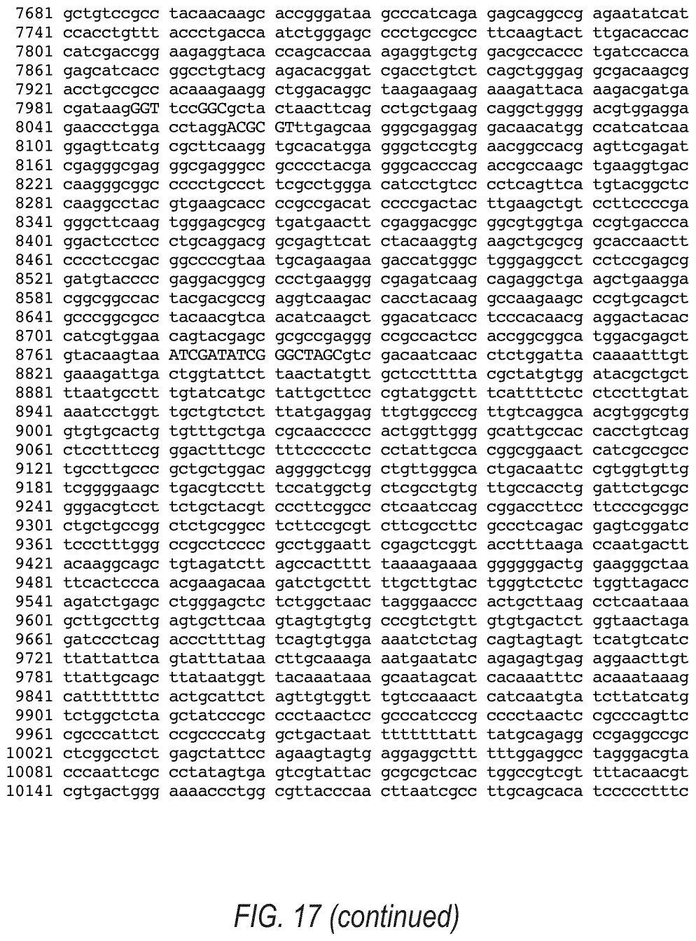

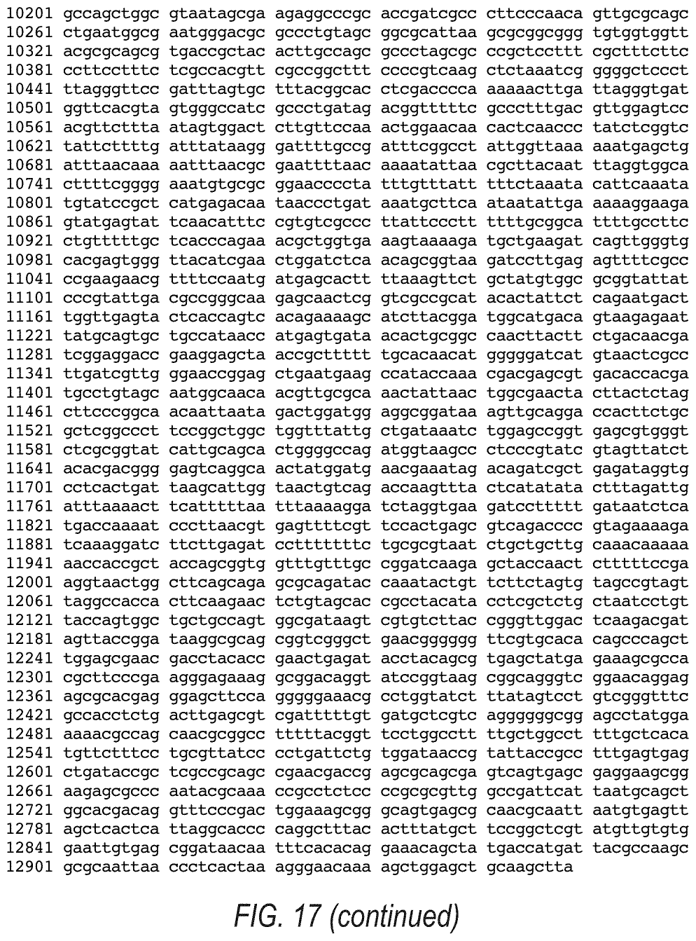

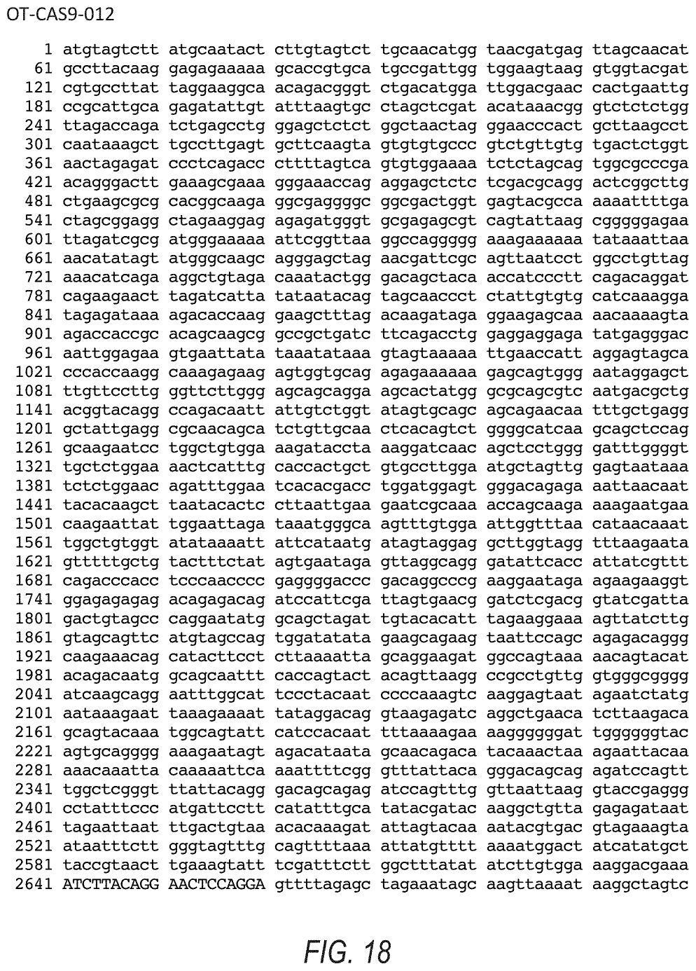

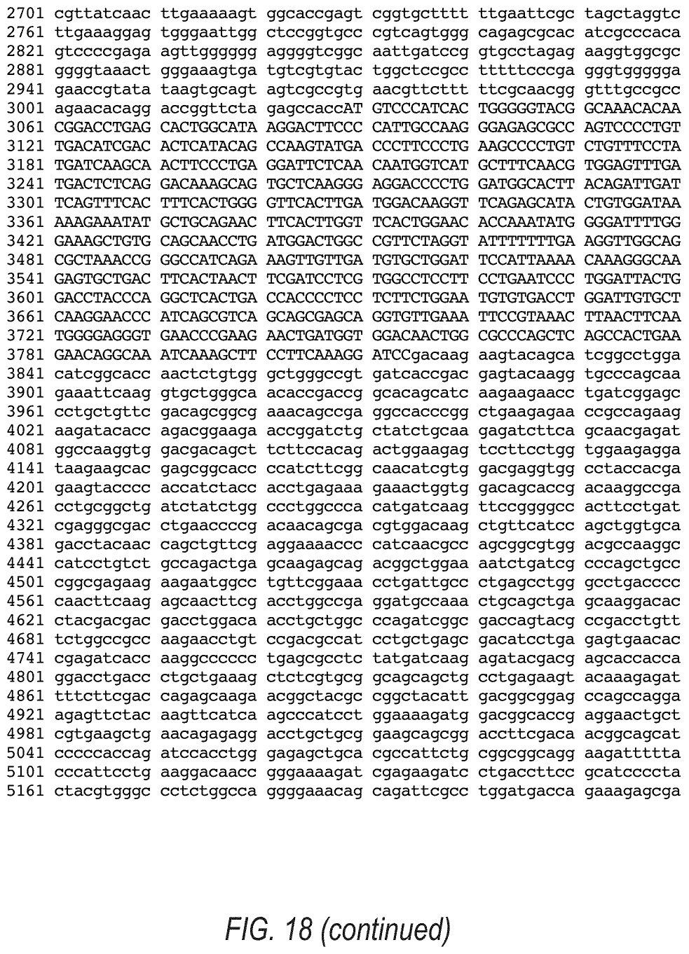

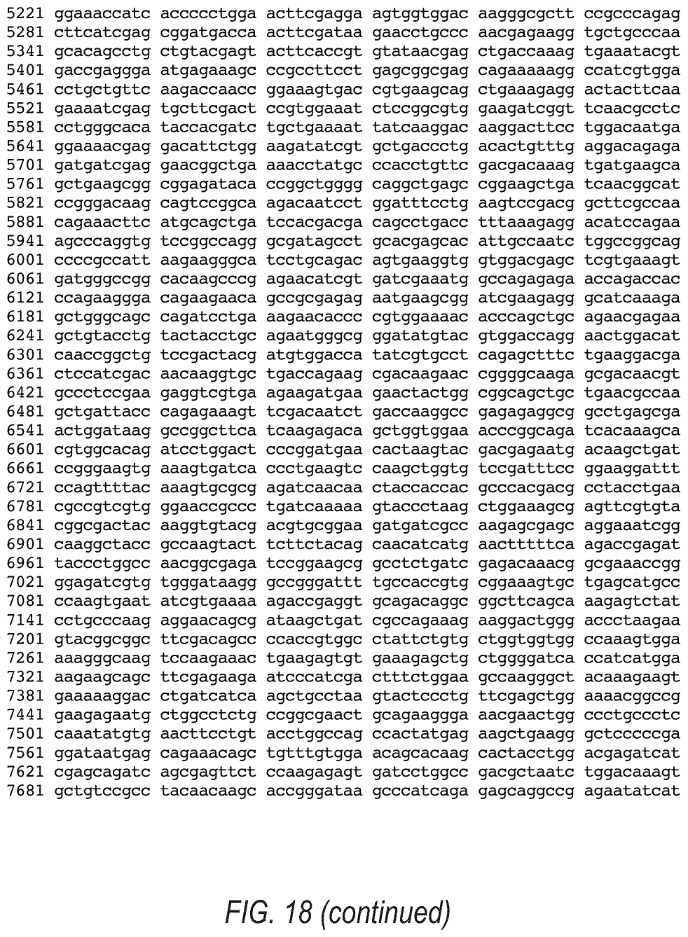

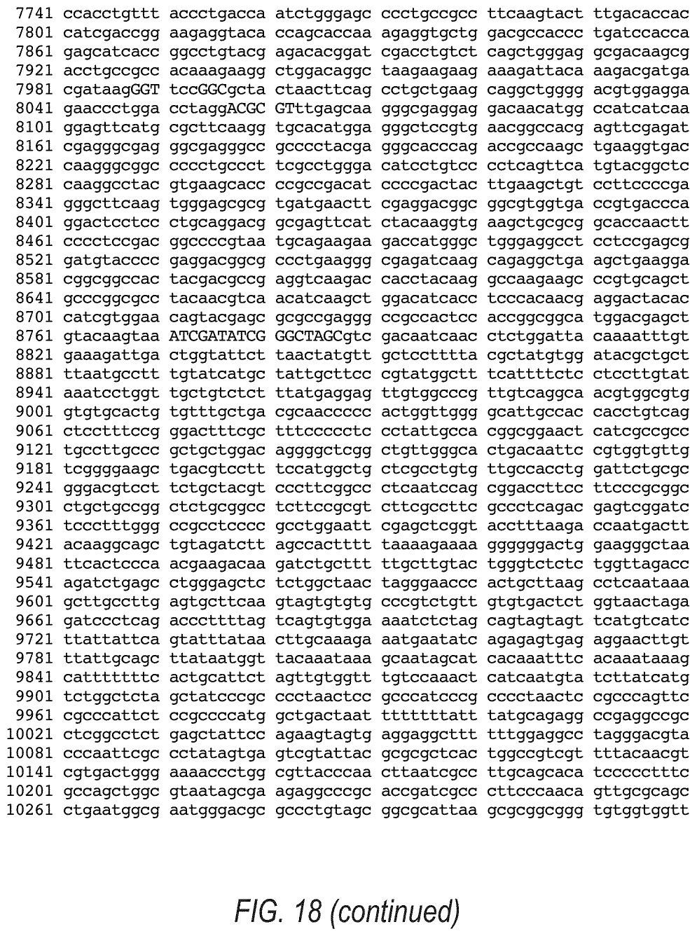

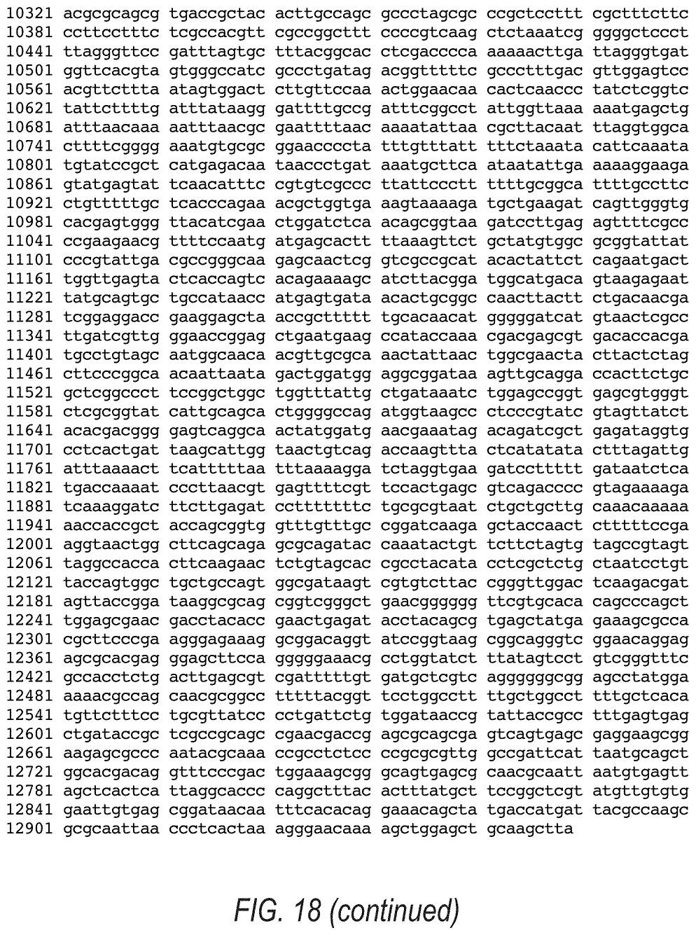

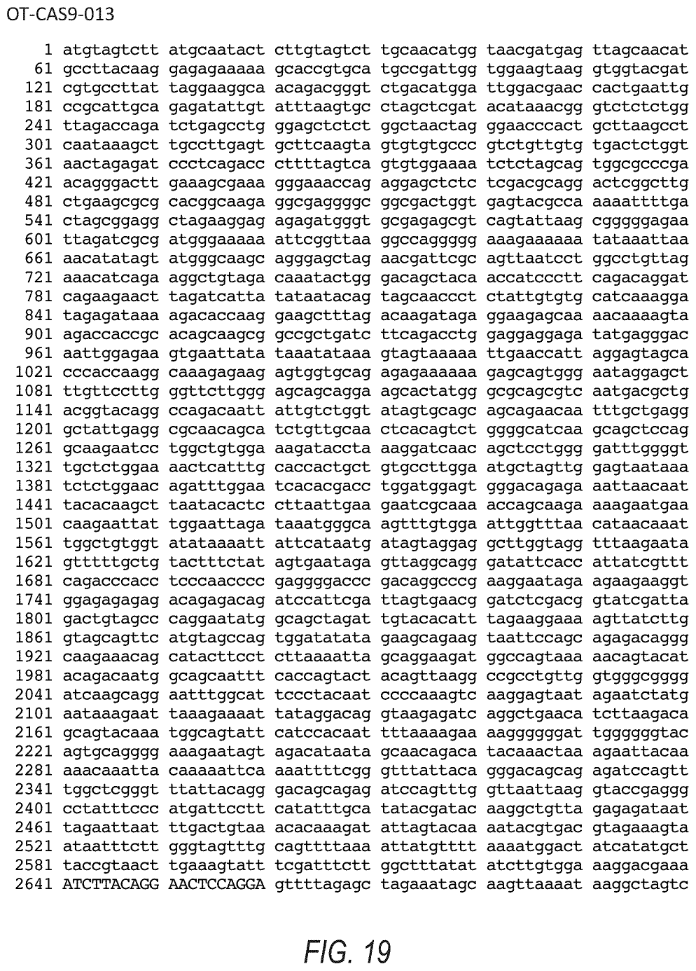

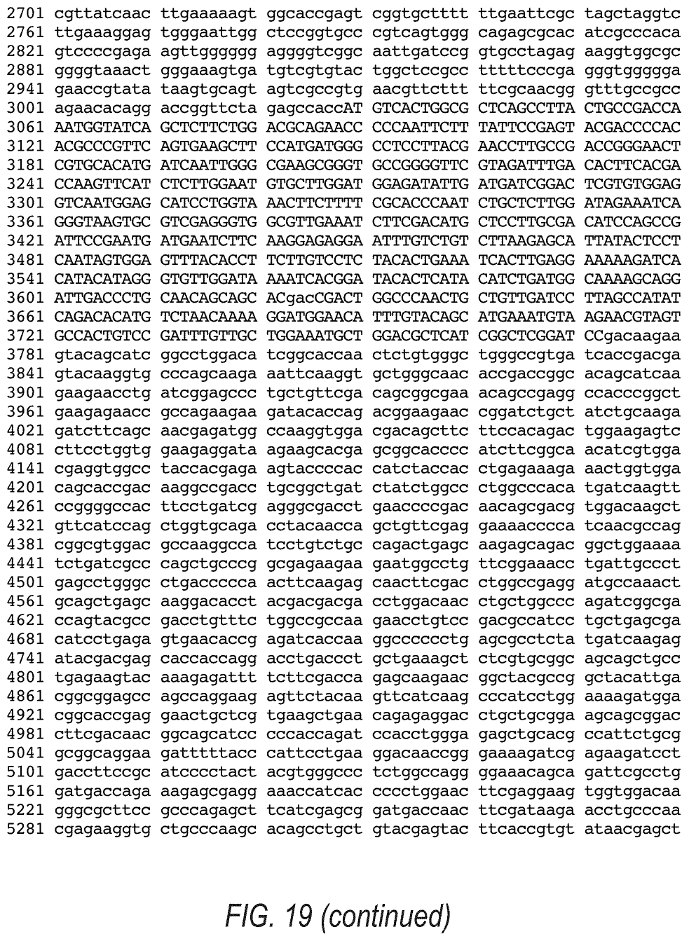

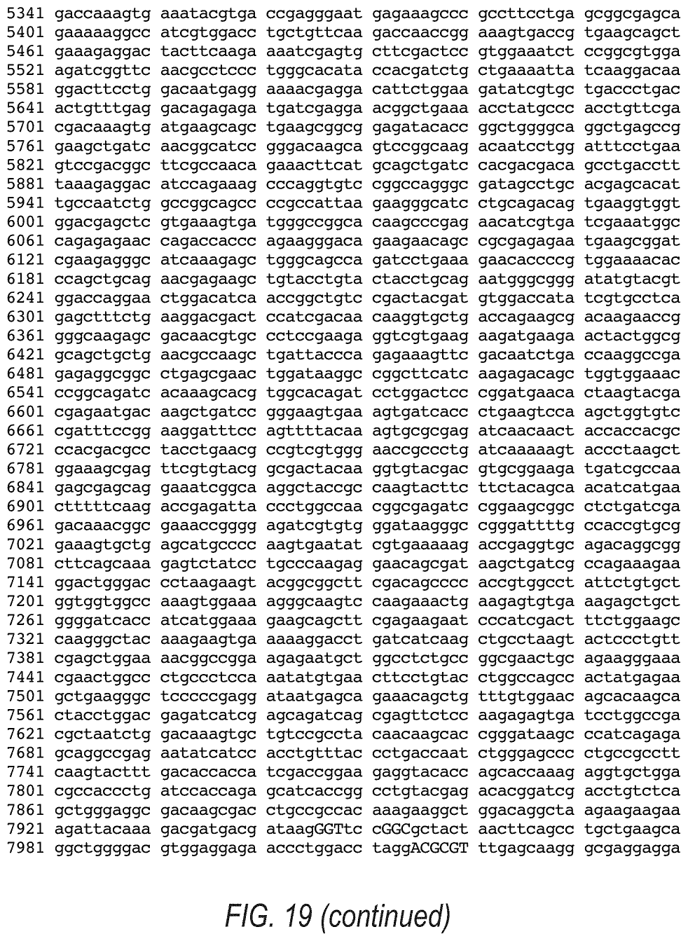

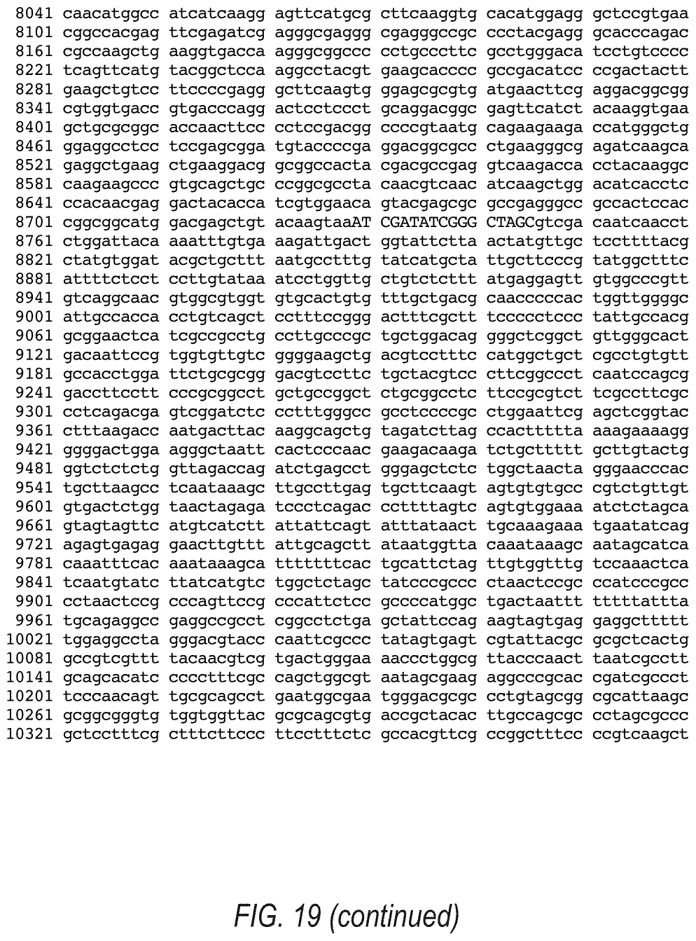

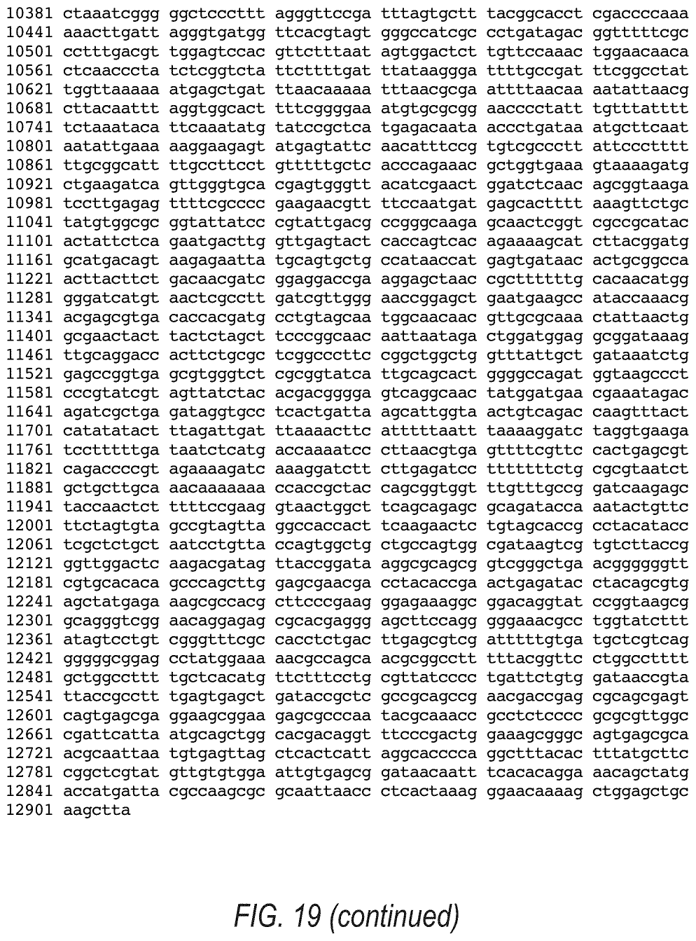

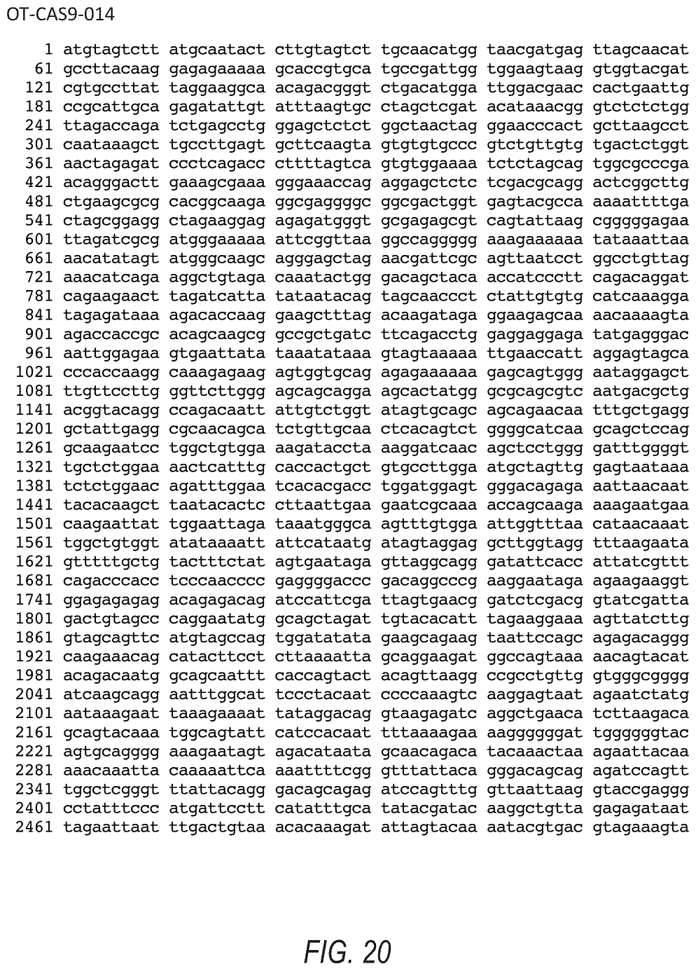

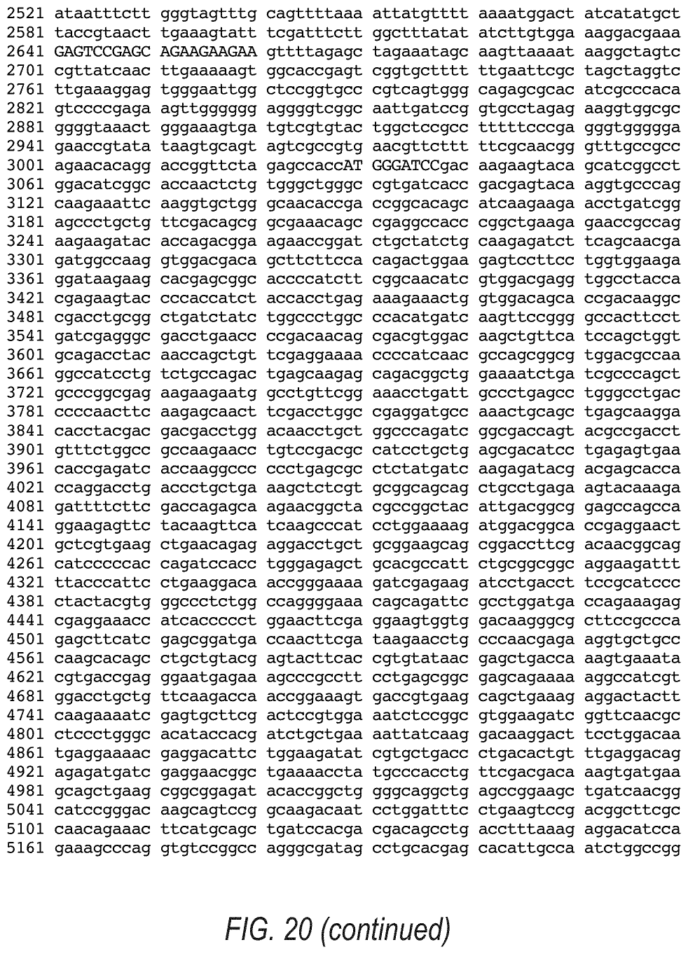

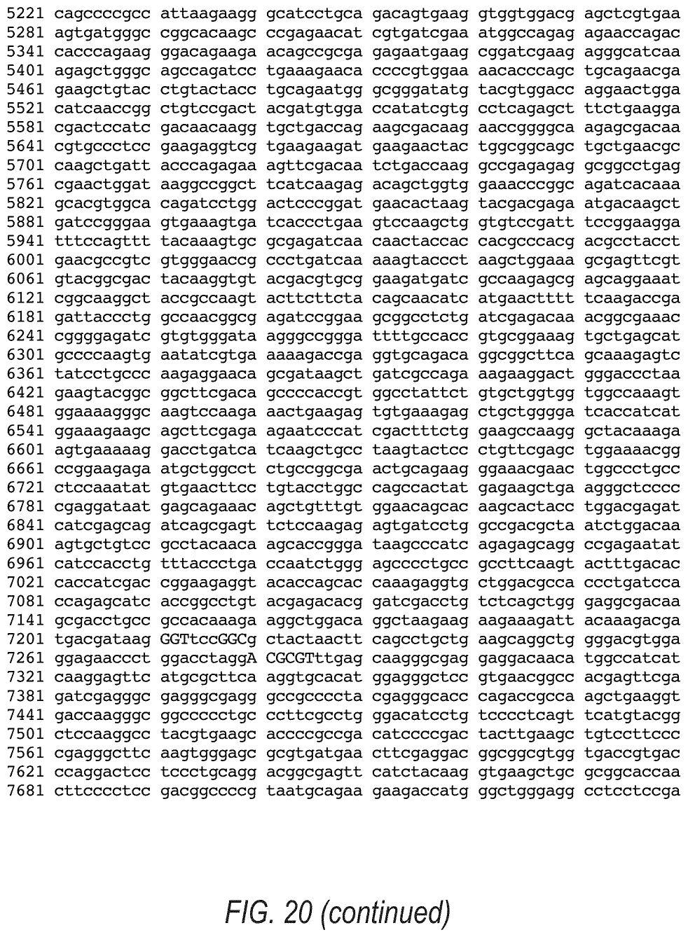

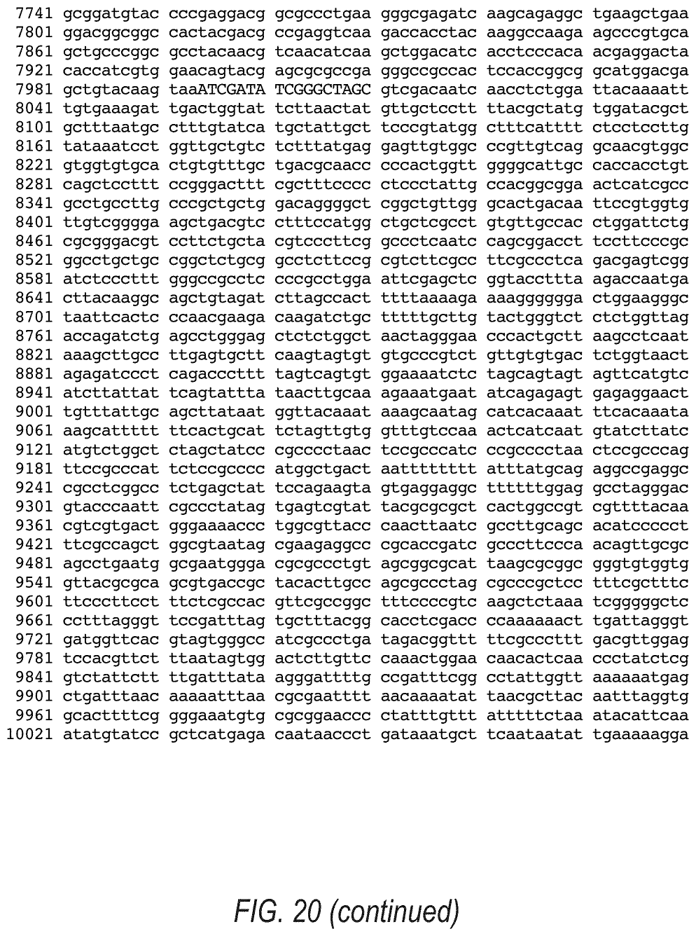

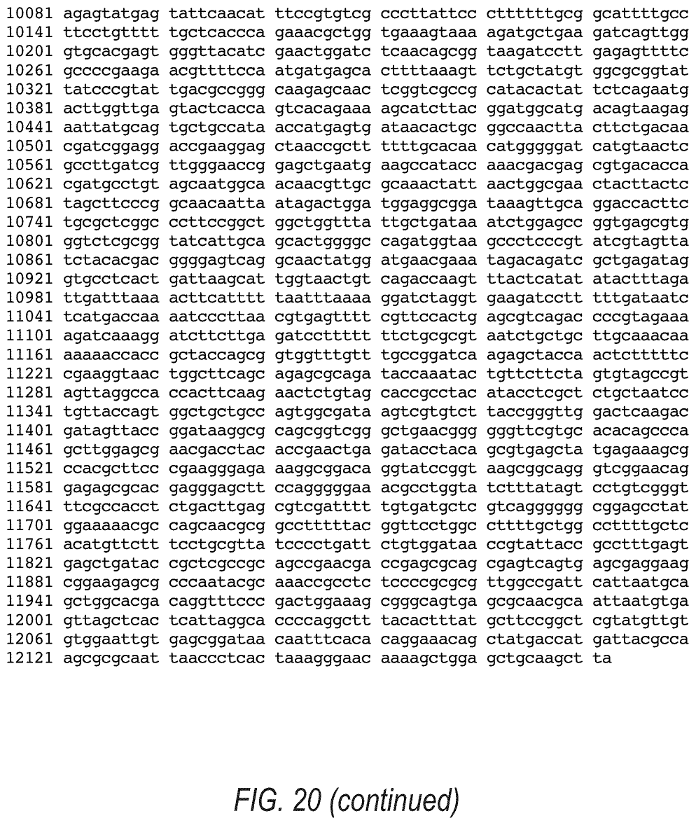

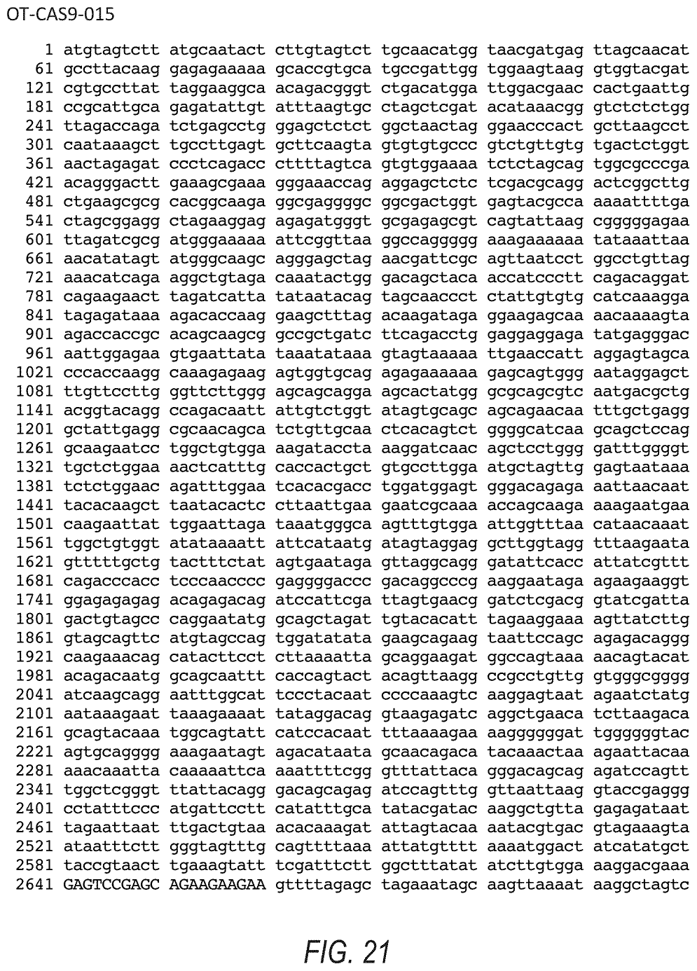

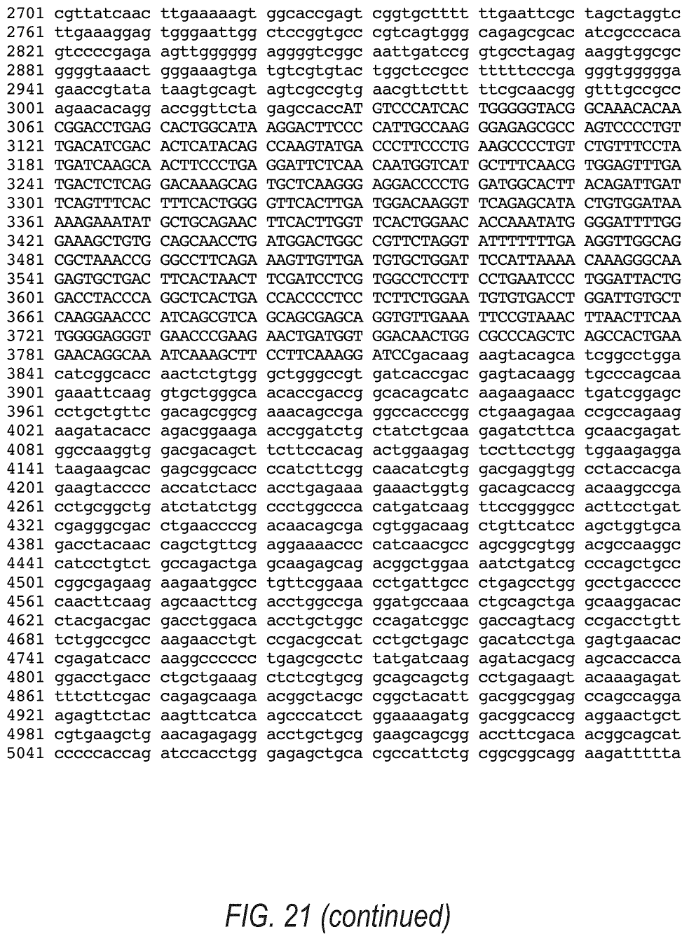

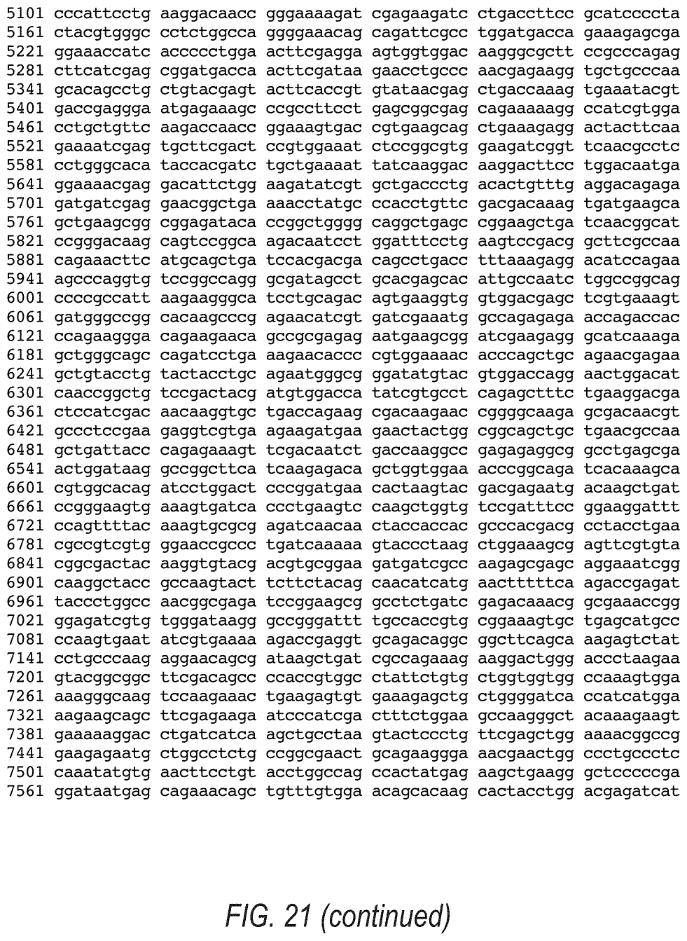

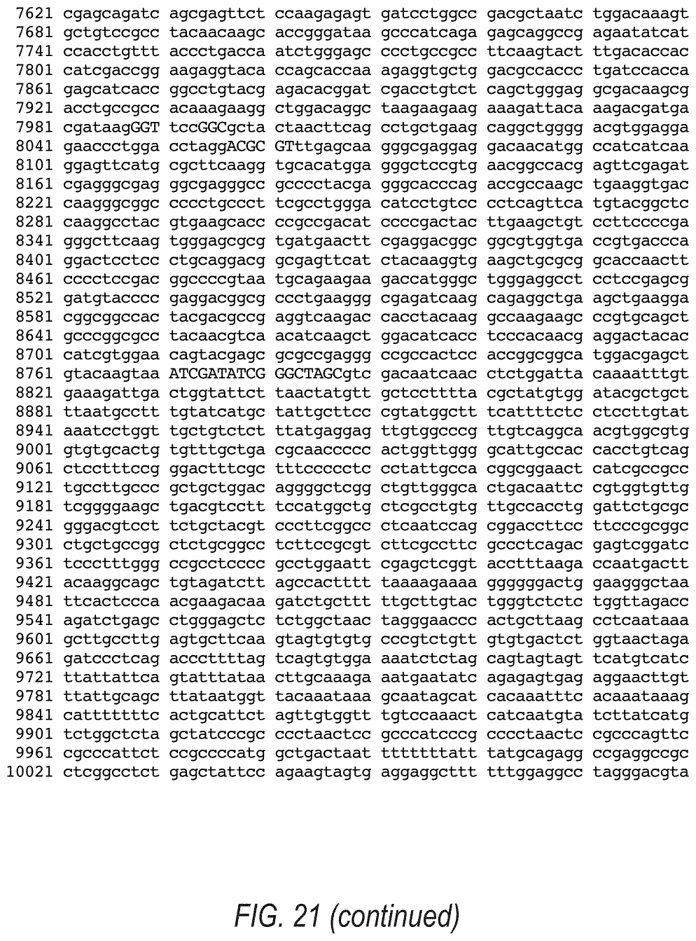

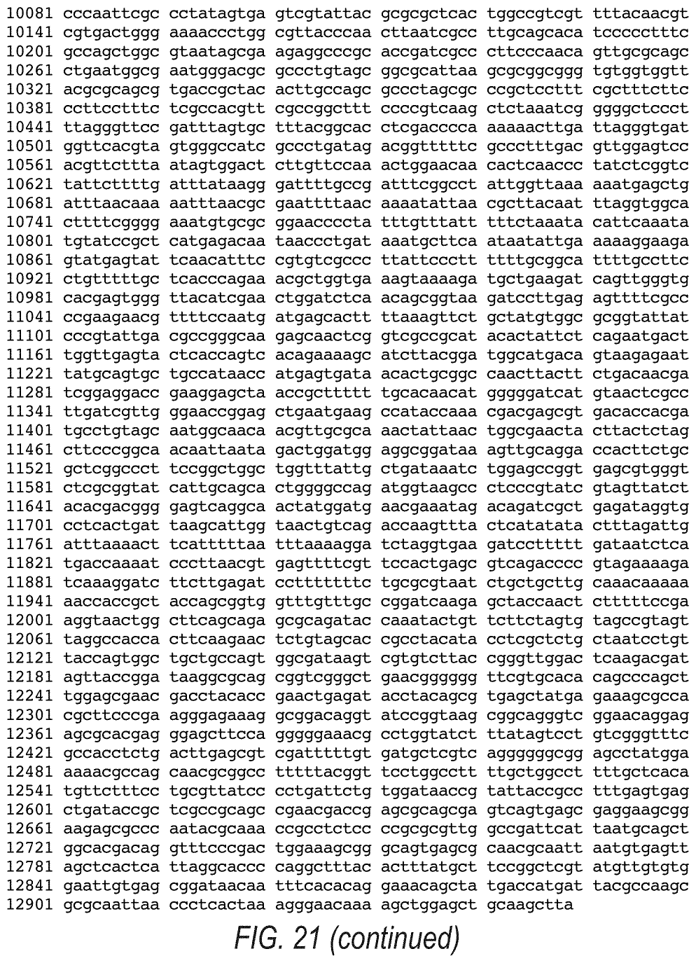

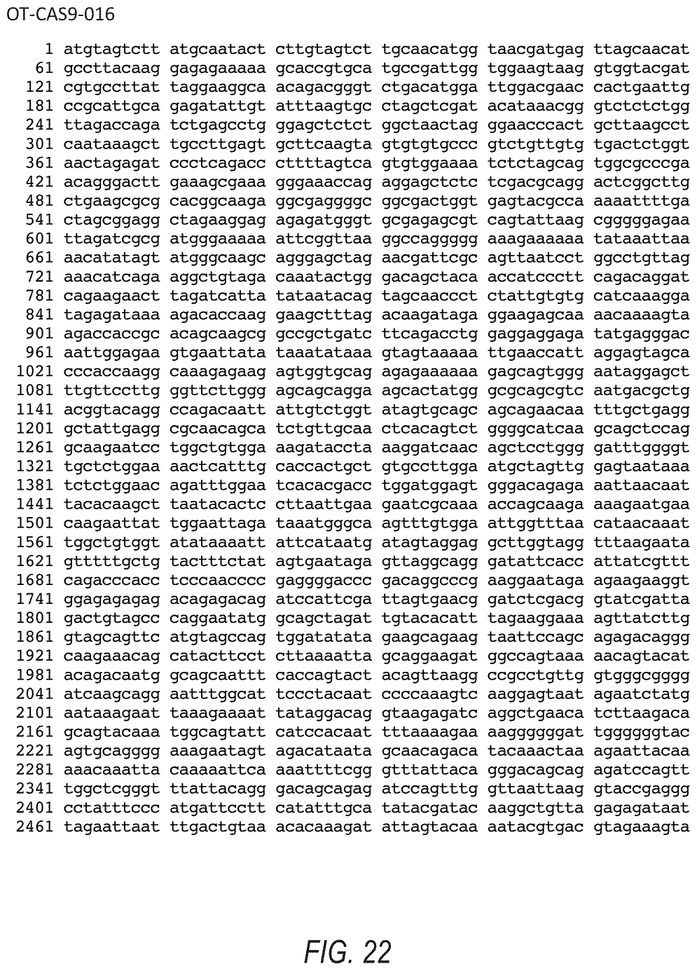

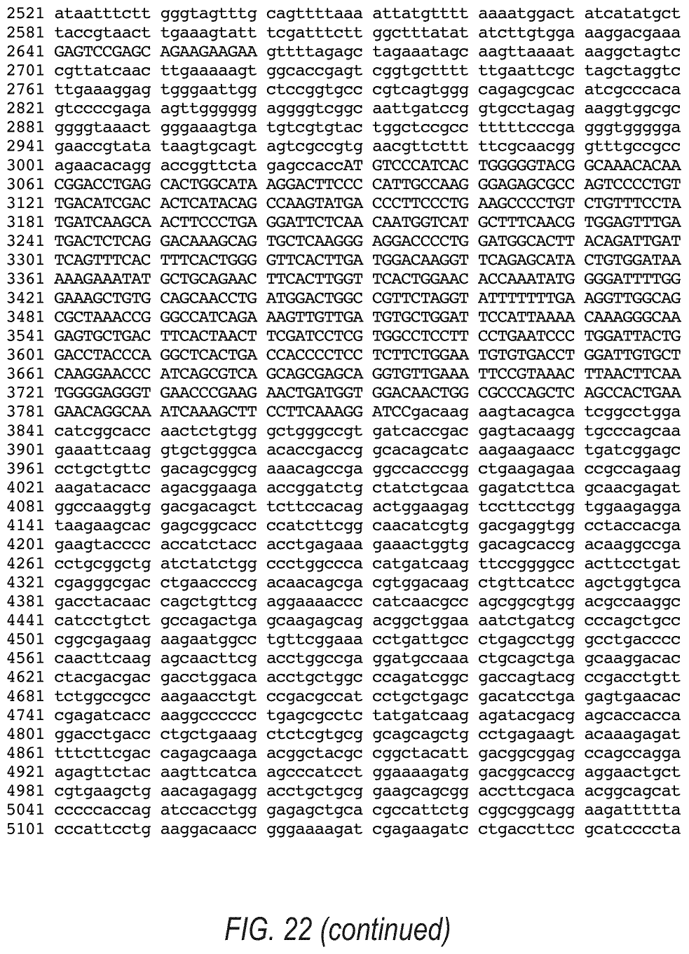

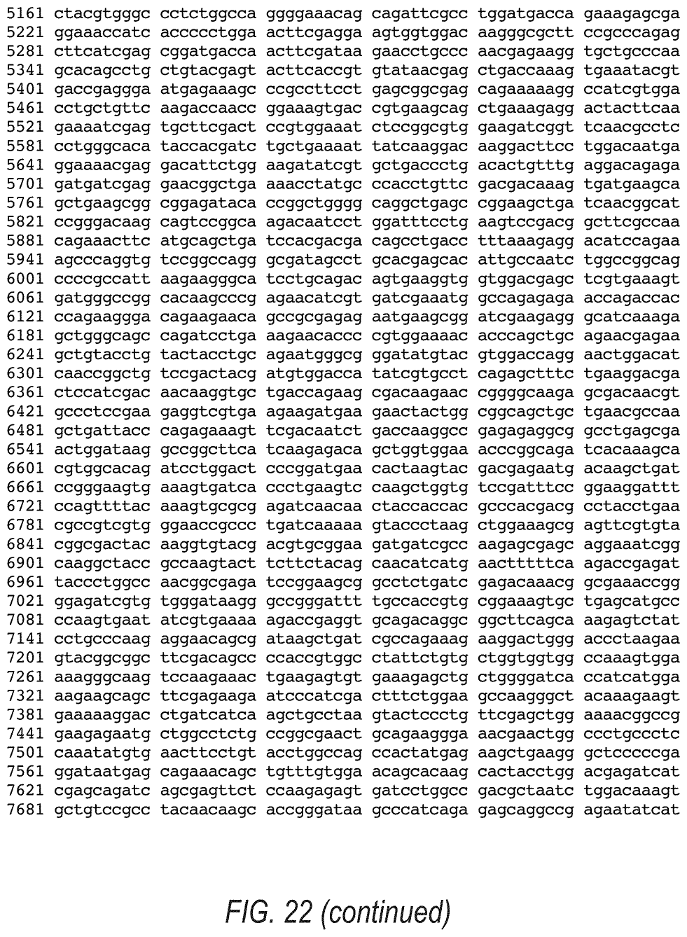

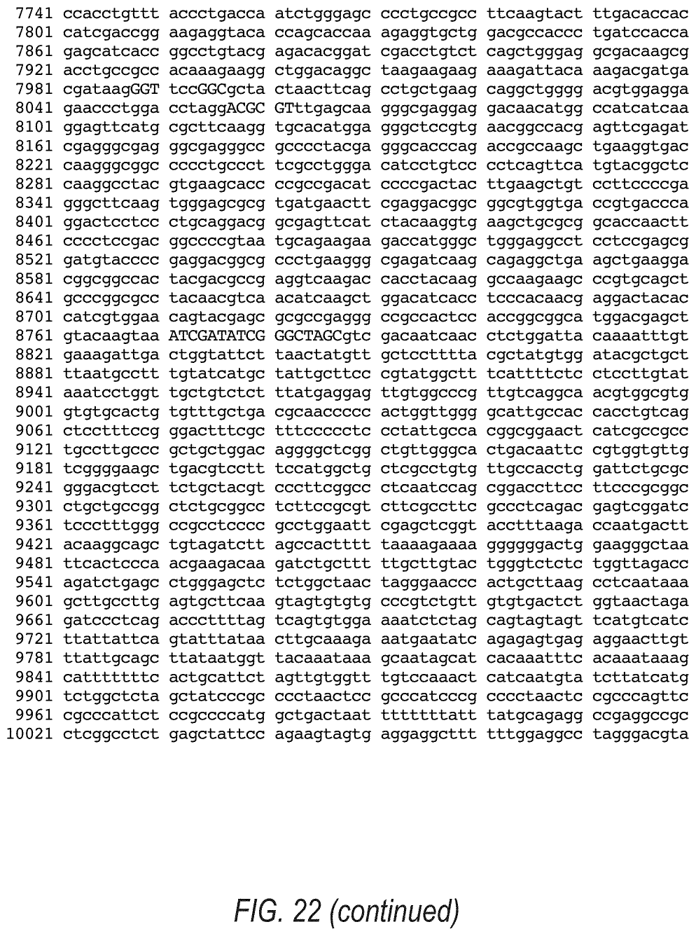

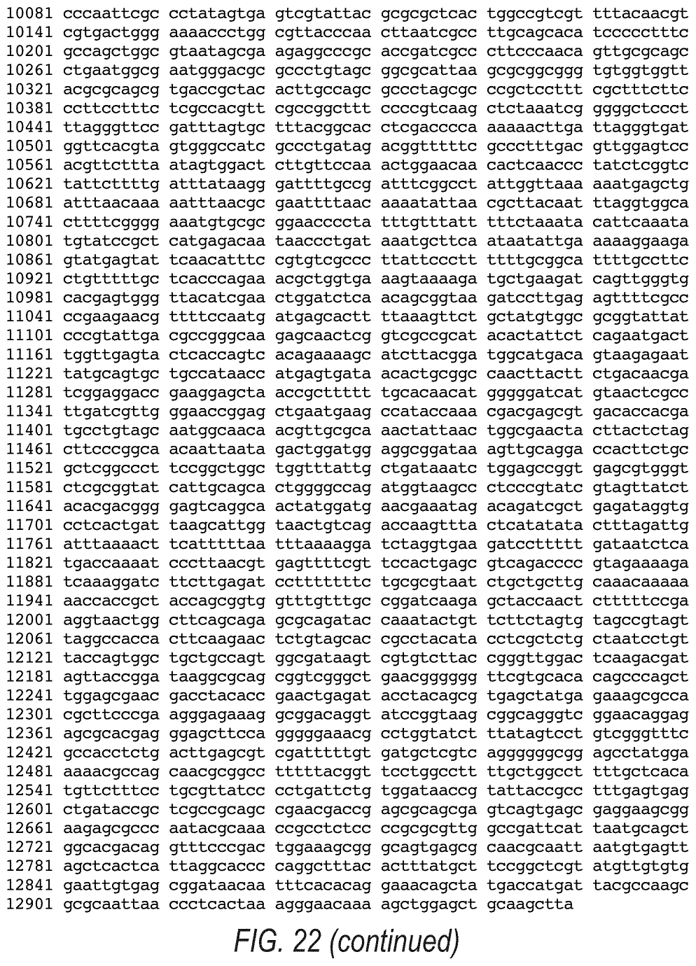

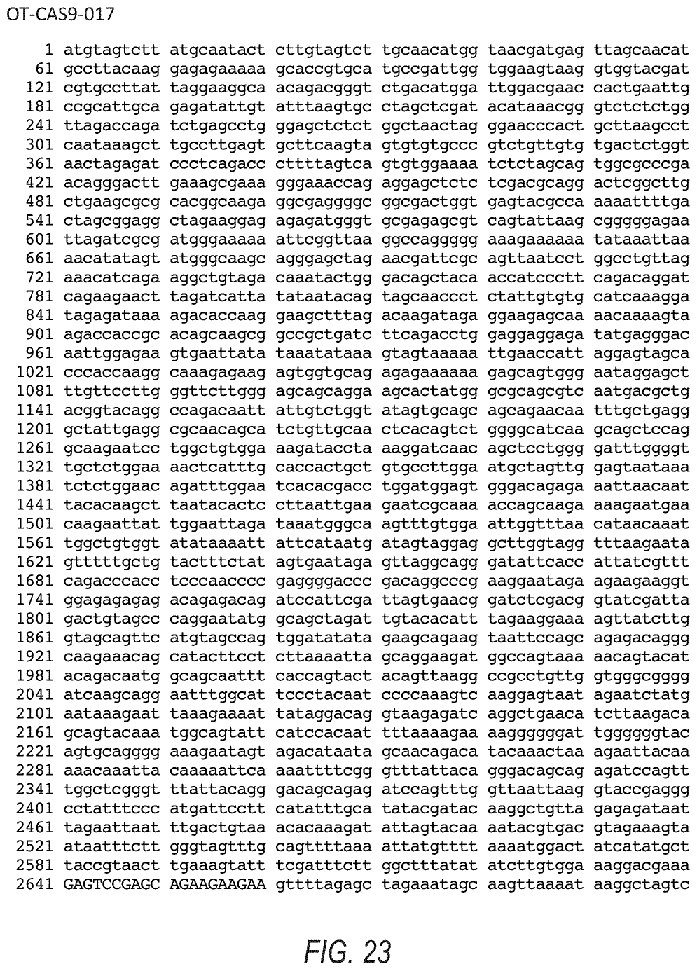

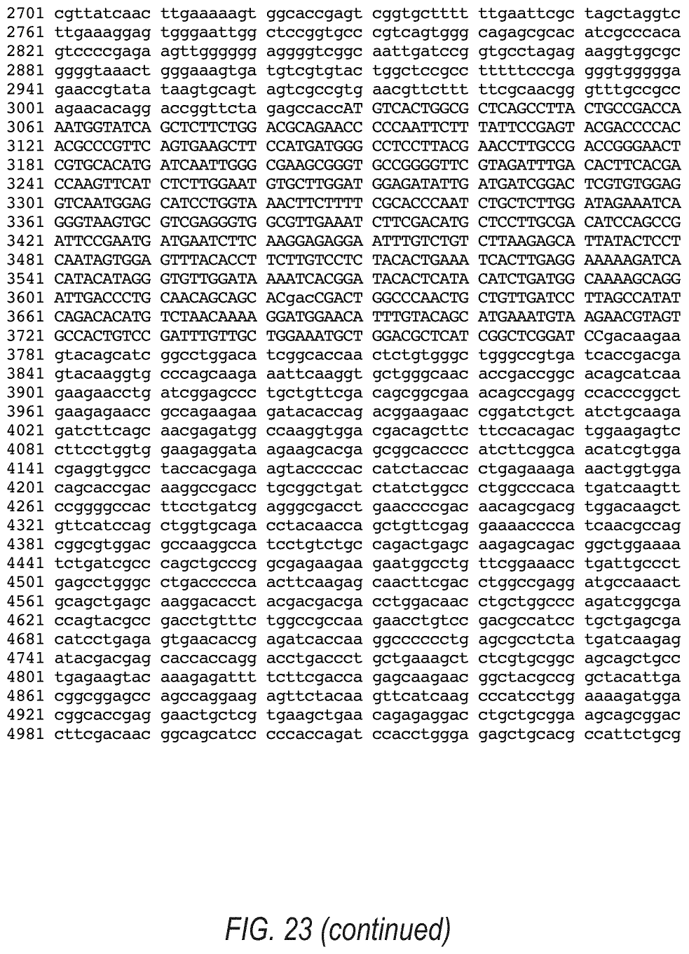

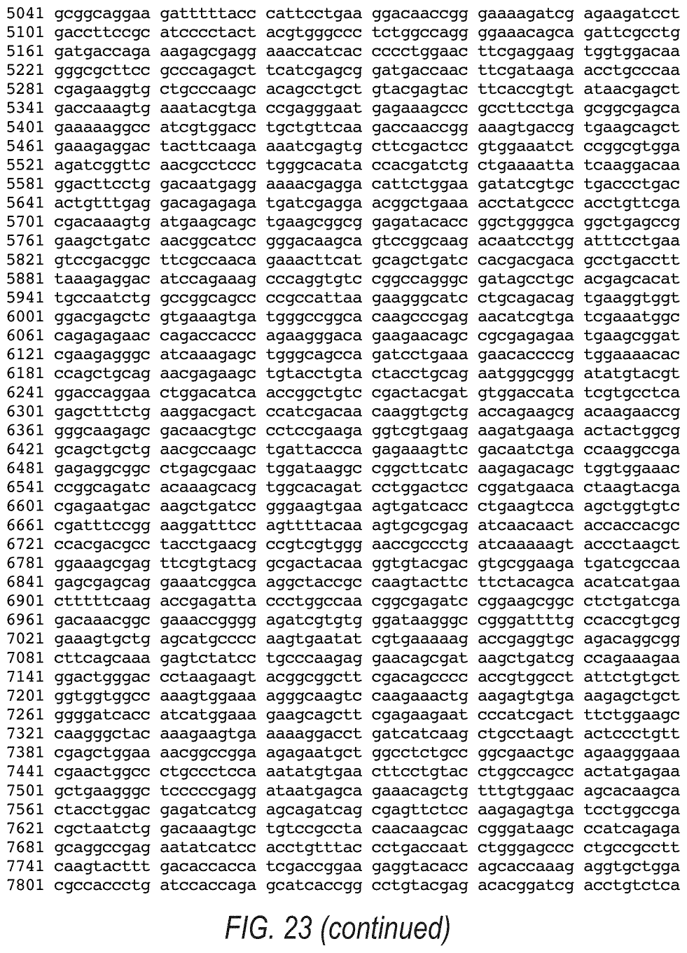

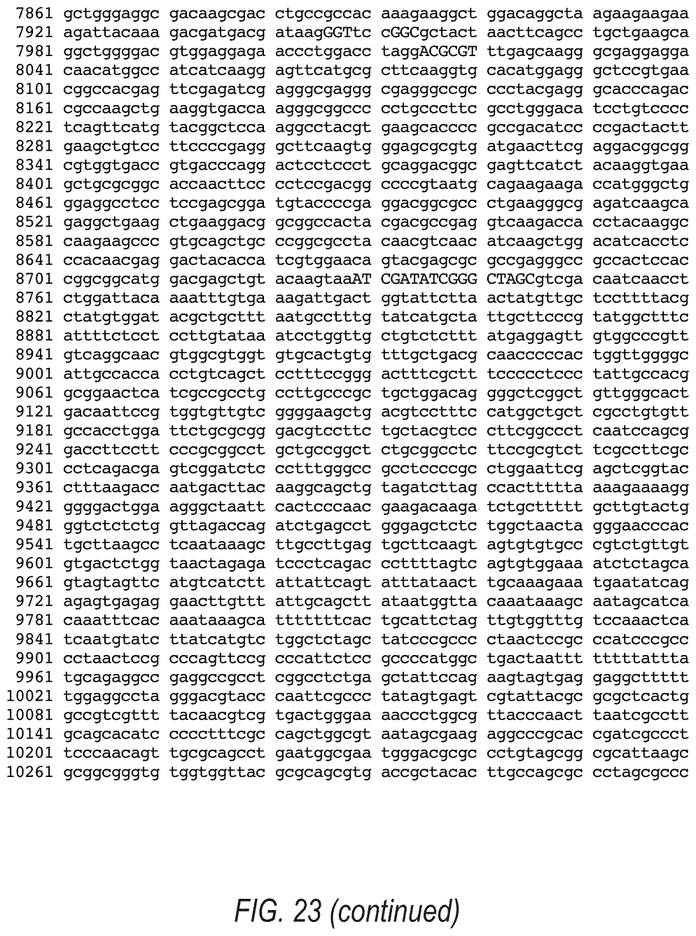

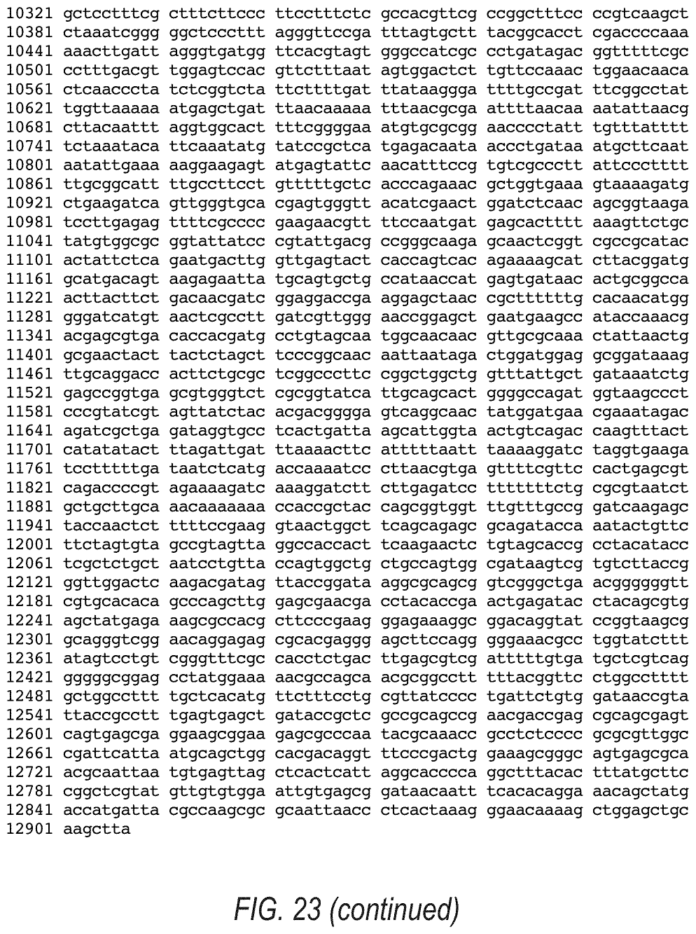

DRDs that can be used for constructs designed to transcriptionally regulate Cas may be selected from, for example, a ecDHFR DRD, FKBP DRD, CA2 DRD, an ER DRD, a hDHFR DRD, and a hPDE5 DRD. Transcription of the guide RNA is mediated by a Pol III promoter, such as a U6 promoter. Exemplary constructs of a Cas-transcription factor system are shown with specified elements of the present disclosure in FIG. 5A-FIG. 5B, FIG. 6 and Table 6.

TABLE 6
Example constructs for transcriptionally
regulating Cas

| Name | Description |
|---|---|
| OT-ZFHD-073 | pELDS-8xZFHD1 BS-min |
| | promoter-ZFHD1-p65-GGSGGGSGG- |
| | CA2(L156H)-WPRE-SV40-Thy1.2 |
| | (“GGSGGGSGG” disclosed as |
| | SEQ ID NO: 20) |
| OT-ZFHD-074 | pELDS-8xZFHD1 BS-min cmv- |
| | ZFHD1-p65-GGSGGGSGG- |
| | CA2(L156H)-WPRE-SV40- |
| | Thy1.2 |
| | (“GGSGGGSGG” disclosed as |
| | SEQ ID NO: 20) |
| OT-ZFHD-075 | pELDS-8xZFHD1 BS-min |
| | promoter-CA2(L156H)- |
| | GSGSG-EGFP- |
| | WPRE-SV40-Thy1.2 |
| | (“GSGSG” disclosed |
| | as SEQ ID NO: 21) |
| OT-ZFHD-076 | pELDS-EF1a-ZFHD1-p65- |
| | GGSGGGSGG-CA2(L156H)- |
| | P2A-TagBFP- |
| | WPRE |
| | (“GGSGGGSGG” disclosed |
| | as SEQ ID NO: 20) |
| OT-ZFHD-077 | pELDS-EF1a-ZFHD1-p65- |
| | GGSGGGSGG-ER(Q502D)-P2A- |
| | TagBFP- |
| | WPRE |
| | (“GGSGGGSGG” disclosed |
| | as SEQ ID NO: 20) |
| OT-ZFHD-079 | pELDS-U6prom-DMDe51 sgRNA- |
| | 8xZFHD1 BS-min promoter-Cas9- |
| | NLS-FLAG-WPRE-SV40-mCherry |

Illustrative components of constructs engineered according to the design for a Cas-transcription factor system, such as the constructs in Table 6, are provided in Table 3 above and Table 7.

TABLE 7
Components of illustrative constructs for
regulation of Cas protein expression and
activity

| | Descrip- | Nucleic Acid | Amino Acid |
|---|---|---|---|
| Component | tion | Sequence | Sequence |
| 8XzFHd1BS | eight (8) | taatgatgggcgcac | not |
| | nucleic | gagtaatgatgggcg | applicable |
| | acid | gacgactaatgatgg | |
| | sites | gcgcacgagtaatga | |
| | that are | tgggcgtctagctaa | |
| | recog- | tgatgggcgctagag | |
| | nized | taatgatgggcggta | |
| | by a | gactaatgatgggcg | |
| | ZFUD1 DNA | ctccagtaatgatgg | |
| | binding | gcgttctagc | |
| | domain | (SEQ ID NO: 55) | |
| ZFHD1 | synthetic | GCACCTAAGaaaAAG | APKKKRKVERP |
| | tran- | AGGAAGGTTgaacgc | YACTVESCDRR |
| | scrip- | ccatatgct | FSRSDELTRHI |
| | tion | tgccctgtcgagtcc | RIHTGQKPFQC |
| | factor | tgcgatcgccgcttt | RICMRNFSRSD |
| | | tctcgctcggatgag | HLITHIRTHTG |
| | | cttacccgccatatc | GGRRRKKRTSI |
| | | cgcatccacacaggc | ETNIRVALEKS |
| | | cagaagcccttccag | FLENQKPTSEE |
| | | tgtcgaatctgcatg | ITMIADQLNME |
| | | cgtaacttcagtcgt | KEVIRVWFCNR |
| | | agtgaccaccttacc | RQKEKRIN |
| | | acccacatccgcacc | (SEQ ID |
| | | cacacaggcggcggc | NO: 74) |
| | | cgcaggaggaagaaa | |
| | | cgcaccagcatagag | |
| | | accaacatccgtgtg | |
| | | gccttagagaagagt | |
| | | ttcttggagaatcaa | |
| | | aagcctacctcggaa | |
| | | gagatcactatgatt | |
| | | gctgatcagctcaat | |
| | | atggaaaaagaggtg | |
| | | attcgtgtttggttc | |
| | | tgtaaccgccgccag | |
| | | aaagaaaaaagaatc | |
| | | aac | |
| | | (SEQ ID NO: 56) | |
| min | Minimal | TCTAGAGGGTATATA | not |
| promoter | TATA | ATGGGGGCCA | applicable |
| | promoter | (SEQ ID NO: 57) | |
| | (YB_TATA) | | |
| min CMV | minimal | TAGGCGTGTACGGTG | not |
| | CMV | GGAGGCCTATATAAG | applicable |
| | promoter | CAGAGCTCGTTTAGT | |
| | | GAACCGTCAGATCGC | |
| | | CTGGA | |
| | | (SEQ ID NO: 58) | |
| EF1a | EF1 alpha | cgtgaggctccggtg | not |
| | promoter | cccgtcagtgggcag | applicable |
| | | agcgcacatcgccca | |
| | | cagtccccgagaagt | |
| | | tggggggaggggtcg | |
| | | gcaattgaaccggtg | |
| | | cctagagaaggtggc | |
| | | gcggggtaaactggg | |
| | | aaagtgatgtcgtgt | |
| | | actggctccgccttt | |
| | | ttcccgagggtgggg | |
| | | gagaaccgtatataa | |
| | | gtgcagtagtcgccg | |
| | | tgaacgttctttttc | |
| | | gcaacgggtttgccg | |
| | | ccagaacacaggtaa | |
| | | gtgccgtgtgtggtt | |
| | | cccgcgggcctggcc | |
| | | tctttacgggttatg | |
| | | gcccttgcgtgcctt | |
| | | gaattacttccacct | |
| | | ggctgcagtacgtga | |
| | | ttcttgatcccgagc | |
| | | ttcgggttggaagtg | |
| | | ggtgggagagttcga | |
| | | ggccttgcgcttaag | |
| | | gagccccttcgcctc | |
| | | gtgcttgagttgagg | |
| | | cctggcctgggcgct | |
| | | ggggccgccgcgtgc | |
| | | gaatctggtggcacc | |
| | | ttcgcgcctgtctcg | |
| | | ctgctttcgataagt | |
| | | ctctagccatttaaa | |
| | | atttttgatgacctgc | |
| | | tgcgacgctttttttc | |
| | | tggcaagatagtcttg | |
| | | taaatgcgggccaag | |
| | | atctgcacactggta | |
| | | tttcggtttttgggg | |
| | | ccgcgggcggcgacg | |
| | | gggcccgtgcgtccc | |
| | | agcgcacatgttcgg | |
| | | cgaggcggggcctgc | |
| | | gagcgcggccaccga | |
| | | gaatcggacgggggt | |
| | | agtctcaagctggcc | |
| | | ggcctgctctggtgc | |
| | | ctggcctcgcgccgc | |
| | | cgtgtatcgccccgc | |
| | | cctgggcggcaaggc | |
| | | tggcccggtcggcac | |
| | | cagttgcgtgagcgg | |
| | | aaagatggccgcttc | |
| | | ccggccctgctgcag | |
| | | ggagctcaaaatgga | |
| | | ggacgcggcgctcgg | |
| | | gagagcgggcgggtg | |
| | | agtcacccacacaaa | |
| | | ggaaaagggcctttc | |
| | | cgtcctcagccgtcg | |
| | | cttcatgtgactcca | |
| | | ctgagtaccgggcgc | |
| | | cgtccaggcacctcg | |
| | | attagttctcga | |
| | | gcttttggagtacgt | |
| | | cgtctttaggttggg | |
| | | gggaggggttttatg | |
| | | cgatggagtttcccc | |
| | | acactgagtgggtgg | |
| | | agactgaagttaggc | |
| | | cagcttggcacttga | |
| | | tgtaattctccttgg | |
| | | aatttgccctttttg | |
| | | agtttggatcttggt | |
| | | tcattctcaagcctc | |
| | | agacagtggttcaaa | |
| | | gtttttttcttccat | |
| | | ttcaggtgtcgtga | |
| | | (SEQ ID NO: 59) | |
| p65 | p65 | ctgggggccttgctt | LGALLGNSTD |
| | activa- | ggcaacagcacagac | PAVFTDLASV |
| | tion | ccagctgtgttcaca | DNSEFQQLLN |
| | domain | gacctggcatccGTG | QGIPVAPHTT |
| | | gacaactccgagttt | EPMLMEYPEA |
| | | cagcagctgctgaac | ITRLVTGAQR |
| | | cagggcatacctgtg | PPDPAPAPLG |
| | | gccccccacacaact | APGLPNGLLS |
| | | gagcccatgctgatg | GDEDFSSIAD |
| | | gagtaccctgaggct | MDFSALLSQI |
| | | ataactcgcctagtg | SS |
| | | acaggggcccagagg | (SEQ ID |
| | | ccccccgacccagct | NO: 75) |
| | | cctgctccactgggg | |
| | | gccccggggctcccc | |
| | | aatggcctcctttca | |
| | | ggagatgaagacttc | |
| | | tcctccattgcggac | |
| | | atggacttctcagcc | |
| | | ctgctgagtcagatc | |
| | | agctcc | |
| | | (SEQ ID | |
| | | NO: 60) | |
| TagBFP | TagBFP | TCTGAGCTGATTAAG | SELIKENMHM |
| | protein | GAGAATATGCACATG | KLYMEGTVDN |
| | | AAGCTGTACATGGAA | HHFKCTSEGE |
| | | GGAACTGTGGACAAT | GKPYEGTQTM |
| | | CATCACTTTAAGTGC | RIKVVEGGPL |
| | | ACATCGGAGGGAGAA | PFAFDILATS |
| | | GGCAAGCCCTACGAA | FLYGSKTFIN |
| | | GGCACCCAGACCATG | HTQGIPDFFK |
| | | AGGATCAAGGTGGTT | QSFPEGFTWE |
| | | GAGGGCGGACCGCTG | RVTTYEDGGV |
| | | CCCTTCGCCTTCGAT | LTATQDTSLQ |
| | | ATCCTGGCGACITCA | DGCLIYNVKI |
| | | TTCCTCTACGGAAGC | RGVNFTSNGP |
| | | AAAACCTTTATTAAC | VMQKKTLGWE |
| | | CACACTCAGGGTATA | AFTETLYPAD |
| | | CCAGACTTCTTTAAG | GGLEGRNDMA |
| | | CAATCCTTCCCTGAG | LKLVGGSHLI |
| | | GGTTTTACATGGGAG | ANIKTTYRSK |
| | | AGAGTCACTACATAT | KPAKNLKMPG |
| | | GAAGAIGGGGGCGTG | VYYVDYRLER |
| | | CIAACCGC1ACTCAG | IKEANNETYV |
| | | GACACCTCTTTACAA | EQHEVAVARY |
| | | GATGGATGTCTCATC | CDLPSKLGHK |
| | | TACAACGTAAAAATT | LN |
| | | AGGGGGGTGAACTTC | (SEQ ID |
| | | ACATCCAACGGCCCT | NO: 76) |
| | | GTGATGCAGAAGAAA | |
| | | ACATTGGGGTGGGAA | |
| | | GCCTTTACGGAGACG | |
| | | CTGTATCCAGCTGAT | |
| | | GGCGGACTGGAAGGC | |
| | | CGGAATGATATGGCC | |
| | | CTTAAGTTAGTTGGT | |
| | | GGGTCACATTTGATA | |
| | | GCAAACATCAAGACC | |
| | | ACATATCGTAGTAAG | |
| | | AAACCCGCTAAAAAC | |
| | | CTCAAGATGCCTGGT | |
| | | GTCTACTATGTTGAC | |
| | | TATAGACTGGAACGA | |
| | | ATCAAAGAGGCAAAT | |
| | | AATGAGACCTACGTC | |
| | | GAGCAGCATGAAGTA | |
| | | GCAGTGGCCCGCTAC | |
| | | TGCGACCTCCCAAGC | |
| | | AAACTGGGGCACAAA | |
| | | CTTAAT | |
| | | (SEQ ID | |
| | | NO: 61) | |
| WPRE | Woodchuck | atcaacctctggatt | not |
| | Hepatitis | acaaaatttgtgaaa | applicable |
| | Virus | gattgactggtattc | |
| | (WHV) | ttaactatgttgctc | |
| | Post- | cttttacgctatgtg | |
| | tran- | gatacgctgctttaa | |
| | scrip- | tgcctttgtatcatg | |
| | tional | ctattgcttcccgta | |
| | Regula- | tggctttcattttct | |
| | tory | cctccttgtataaat | |
| | Element | cctggttgctgtctc | |
| | (WPRE) | tttatgaggagttgt | |
| | | ggcccgttgtcaggc | |
| | | aacgtggcgtggtgt | |
| | | gcactgtgtttgctg | |
| | | acgcaacccccactg | |
| | | gttggggcattgcca | |
| | | ccacctgtcagctcc | |
| | | tttccgggactttcg | |
| | | ctttccccctcccta | |
| | | ttgccacggcggaac | |
| | | tcatcgccgcctgcc | |
| | | ttgcccgctgctgga | |
| | | caggggctcggctgt | |
| | | tgggcactgacaatt | |
| | | ccgtggtgttgtcgg | |
| | | ggaagctgacgtcct | |
| | | ttccatggctgctcg | |
| | | cctgtg | |
| | | ttgccacctggattc | |
| | | tgcgcgggacgtcct | |
| | | tctgctacgtccctt | |
| | | cggccctcaatccag | |
| | | cggaccttccttccc | |
| | | gcggcctgctgccgg | |
| | | ctctgcggcctcttc | |
| | | cgcgtcttcgccttc | |
| | | gccctcagacgagtc | |
| | | ggatctccctttggg | |
| | | ccgcctccccgcctg | |
| | | (SEQ ID NO: 62) | |
| SV40 | SV40 | ggtgtggaaagtccc | not |
| | promoter | caggctccccagcag | applicable |
| | | gcagaagtatgcaaa | |
| | | gcatgcatctcaatt | |
| | | agtcagcaaccaggt | |
| | | gtggaaagtccccag | |
| | | gctccccagcaggca | |
| | | gaagtatgcaaagca | |
| | | tgcatctcaattagt | |
| | | cagcaaccatagtcc | |
| | | cgcccctaactccgc | |
| | | ccatcccgcccctaa | |
| | | ctccgcccagttccg | |
| | | cccattctccgcccc | |
| | | atggctgactaattt | |
| | | tttttatttatgcag | |
| | | aggccgaggccgcct | |
| | | ctgcctctgagctat | |
| | | tccagaagtagtgag | |
| | | gaggcttttttggag | |
| | | gcctaggct | |
| | | (SEQ ID NO: 63) | |
| Thy 1.2 | Thy 1.2 | AACCCAGCCATCAGCG | NPAISVALLLS |
| | protein | TCGCTCTCCTGCTCT | VLQVSRGQKV |
| | | CAGTCTTGCAGGTGT | TSLTACLVNQ |
| | | CCCGAGGGCAGAAGG | NLRLDCRHEN |
| | | TGACCAGCCTGACAG | NTKDNSIQHE |
| | | CCTGCCTGGTGAACC | FSLTREKRKH |
| | | AAAACCTTCGCCTGG | VLSGTLGIPE |
| | | ACTGCCGCCATGAGA | HTYRSRVTLS |
| | | ATAACACCAAGGATA | NQPYIKVLTL |
| | | ACTCCATCCAGCATG | ANFTTKDEGD |
| | | AGTTCAGCCTGACCC | YFCELQVSGA |
| | | GAGAGAAGAGGAAGC | NPMSSNKSIS |
| | | ACGTGCTCTCAGGCA | VYRDKLVKCG |
| | | CCCTTGGGATACCCG | GISLLVQNTS |
| | | AGCACACGTACCGCT | WMLLLLLSLS |
| | | CCCGCGTCACCCTCT | LLQALDFISL |
| | | CCAACCAGCCCTATA | (SEQ ID |
| | | TCAAGGTCCTTACCC | NO: 77) |
| | | TAGCCAACTTCACCA | |
| | | CCAAGGATGAGGGCG | |
| | | ACTACTTTTGTGAGC | |
| | | TTCAAGTCTCGGGCG | |
| | | CGAATCCCATGAGCT | |
| | | CCAATAAAAGTATCA | |
| | | GTGTGTATAGAGACA | |
| | | AGCTGGTCAAGTGTG | |
| | | GCGGCATAAGCCTGC | |
| | | TGGTTCAGAACACAT | |
| | | CCTGGATGCTGCTGC | |
| | | TGCTGCTTTCCCTCT | |
| | | CCCTCCTCCAAGCCC | |
| | | TGGACTTCATTTCTC | |
| | | TG | |
| | | (SEQ ID NO: 64) | |

### Example 4: Testing Ligand-Dependent Cas Expression and Activity for Systems Designed to Transcriptionally Regulate Cas

The present example demonstrates methods of detecting and analyzing Cas protein levels and gene editing activity for constructs designed to transcriptionally regulate Cas. Methods described in the present example use a construct comprising nucleic acid sequences encoding a Cas9 protein and an mCherry protein tag and a construct comprising nucleic acid sequences encoding a transcription factor and a BFP tag (e.g., as shown in FIG. 5B). These methods are also applicable to similar constructs without protein tags as well as other constructs that are designed to transcriptionally regulate Cas in accordance with the present disclosure, such as constructs that are components of Cas-transcription factor systems. The present example also demonstrates application of these methods for combinations of constructs shown in Table 6.

Cas expression and activity is analyzed in cells transduced with lentivirus made from constructs encoding the transcription factor and Cas9. As a non-limiting example, the U2OS cell line or HEK293 cell line may be used for these methods. Untransduced (parental) U2OS cells or HEK293 cells may be used as control cell lines.

For transcriptionally regulated Cas systems comprising two constructs, such as shown in FIG. 5A-5B and in FIG. 6, each construct can be delivered to cells separately on two separate vectors. For example, U2OS cells or HEK293 cells are first transduced with a construct encoding a transcription factor and a first construct marker (e.g., BFP) and the cells sorted for marker positive cells. Then, the transcription factor-transduced U2OS cells (TF-U2OS) or HEK293 cells (TF-HEK293) are transduced with a construct encoding Cas9 and a second construct marker (e.g., mCherry) and the cells are sorted for mCherry and BFP positive cells.

Transduced cells are treated with vehicle control (e.g., DMSO) or drug (e.g., ACZ for constructs comprising a CA2 DRD or bazedoxifene for constructs comprising an ER DRD). For dose response studies, multiple doses are tested (e.g., a 10-point dose response assay including 100 μM ACZ or 1 μM bazedoxifene as top concentrations). Cells are treated for 24, 48, and/or 72 hours. Cas9 and transcription factor protein levels can be assessed by immunoassay. Cas9 and transcription factor mRNA levels can be measured by RT-PCR. To detect and analyze Cas9 activity, genomic DNA is isolated and genome editing is measured. Methods of measuring genome editing include the T7E1 assay (Alt-R Genome Editing Detection Kit from IDT), the TIDE assay and the ICE assay.

Cells comprising constructs having a DRD operably linked to a transcription factor are expected to show ligand-dependent transcription factor protein levels and ligand-dependent Cas9 protein levels. These constructs are also expected to show ligand-dependent genome editing.

The methods described by the present example can be employed for other constructs that are components of Cas-transcription factor systems of the present disclosure. Illustrative application of these methods for the constructs in Table 6 are described below.

### Transcriptional System for Cas Regulation:

Combinations of constructs OT-ZFHD-076 or OT-ZFHD-077 with OT-ZFHD-079 can be assessed according to the methods described above. Briefly, a stable cell line is generated with OT-ZHFD-076 or OT-ZHFD-077 by sorting for BFP-positive cells. The transcription factor transduced cells are then transduced with OT-ZFHD-079 and sorted for mCherry and BFP positive cells. The cells are analyzed in presence and absence of ligands as described above.

### Double-Off Transcription System for Cas Regulation: Self-Inducing Transcription Factor

As described by the present disclosure, a self-inducing transcription factor is encoded by a nucleic acid sequence that is operably linked to an inducible promoter comprising the specific polynucleotide binding site to which the transcription factor is able to bind and activate transcription. Also as described by the present disclosure, a system comprising a self-inducing transcription factor, wherein the transcription factor is operably linked to a DRD is a type of double-off transcription system. Combinations of constructs OT-ZHFD-073 or OT-ZHFD-074 with OT-ZFHD-079 are illustrative of a double-off transcription system for Cas regulation with a self-inducing transcription factor. Such constructs can be assessed according to methods described above. Briefly, a stable cell line is generated with OT-ZHFD-073 or OT-ZHFD-074 by sorting for BFP-positive cells. The transcription factor transduced cells are then transduced with OT-ZFHD-079 and sorted for mCherry and BFP positive cells. The cells are analyzed in the presence and absence of a CA2 ligand (e.g., acetazolamide) as described above.

### Double-Off Transcription System for Cas Regulation: DRD-Cas9

Combinations of constructs OT-ZHFD-076 or OT-ZHFD-077 with OT-ZFHD-075 are illustrative of a double-off transcription system comprising a DRD operably linked to a transcription factor and a DRD operably linked to a protein that is transcriptionally regulated by the transcription factor (in the case of OT-ZFHD-075, said protein is EGFP). A similar construct design to that of OT-ZFHD-075 comprising a nucleic acid sequence encoding a Cas operably linked to a DRD (instead of an EGFP operably linked to a DRD) is another example of a double-off transcription system that can be combined with transcription factor constructs such as OT-ZFHD-076 or OT-ZFHD-077 according to methods described herein for Cas regulation.

Combinations of constructs OT-ZHFD-076 or OT-ZHFD-077 with OT-ZFHD-075 can be assessed according to methods described above. Briefly, a stable cell line is generated with OT-ZHFD-076 or OT-ZHFD-077 by sorting for BFP-positive cells. The transcription factor transduced cells are then transduced with OT-ZFHD-075 or a similar construct comprising a nucleic acid sequence encoding Cas9 and sorted for mCherry and BFP positive cells. The cells are analyzed in presence and absence of ligands as described above. GFP or Cas9, and transcription factor protein levels can be assessed by immunoassay. GFP levels can also be measured by flow cytometry. GFP or Cas9, and transcription factor mRNA levels can be measured by RT-PCR.

### Example 5: In Vitro Ligand-Dependent Cas Expression and Activity Using a System Designed to Directly Regulate Cas

The present example demonstrates ligand-dependent regulation of Cas9 expression and activity using a direct Cas-DRD regulation system in accordance with the present disclosure. As a non-limiting illustration of a direct Cas-DRD regulation system, the DRD of the present example is a CA2 DRD, the Cas9 is a SpCas9 and the guide RNA target is EGFP. The present example also demonstrates ligand dose-dependent regulation of Cas expression using this direct Cas-DRD regulation system.

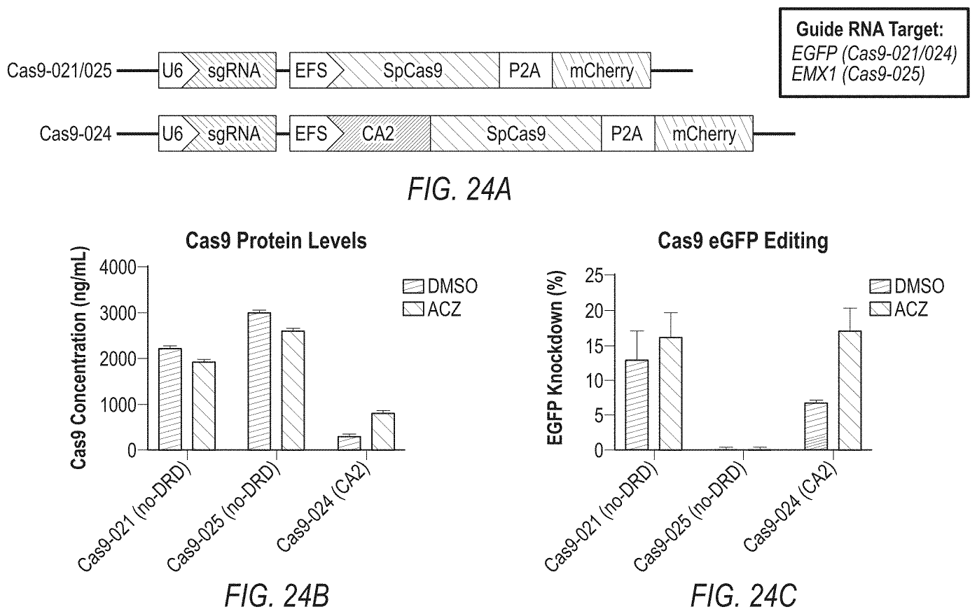

HEK293T cells expressing EGFP were transfected with Cas constructs (OT-Cas9-021, OT-Cas9-024, OT-Cas9-025)(FIG. 24A and Table 2). Transfected cells were treated after 24 hours with vehicle control (e.g., DMSO) or acetazolamide (ACZ). Cells were treated for 48 hours before collection for measurement of Cas9 expression by ELISA kit (Cell Signaling Technology) or 120 hours before analysis by flow cytometry for EGFP expression knockdown. Cas9 protein levels in cells transfected with OT-Cas9-024 is regulated by treatment with ACZ while constitutive controls are not (FIG. 24B). Cas9 activity levels are also regulated by ACZ in OT-Cas9-024 transfected cells as seen by an increase in EGFP negative cells as measured by flow cytometry (FIG. 24C).

HEK293T cells were transfected either with plasmid encoding constitutive (OT-Cas9-006) or CA2 DRD regulated (OT-Cas9-012) Cas9. One day post transfection, each transfected pool of cells were split into 30 wells and treated with 10 doses of acetazolamide (60 μM final concentration as maximum with 3-fold serial dilution for 9 wells and one well treated with vehicle (DMSO)) in triplicate to set up dose response assay. Two days post treatment, cells were collected and analyzed by flow cytometry staining for Cas9 (Abcam ab189380) and mCherry (Invitrogen M11241) following the protocol for intracellular staining from Cell Signaling Technology (www.cellsignal.com/learn-and-support/protocols/protocol-flow-methanol-permeabilization).

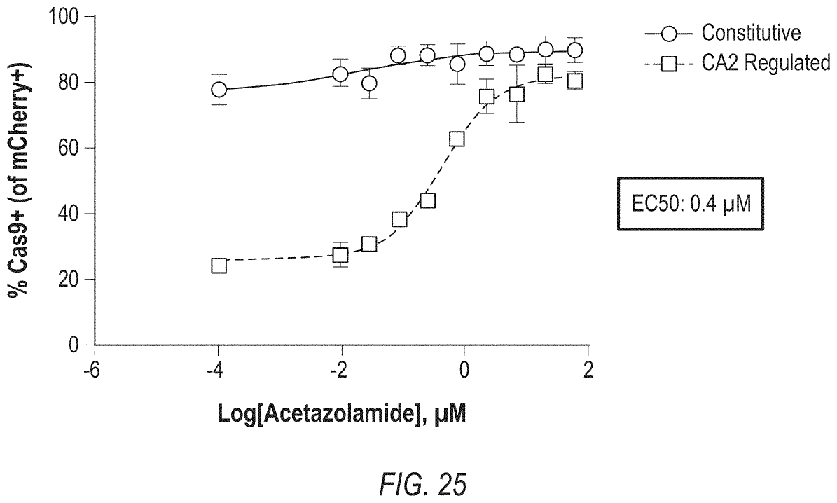

EC50 was calculated using GraphPad Prism. CA2-Cas9 was stabilized by ACZ with an EC50 of 0.41 μM (FIG. 25).

### Example 6: Illustrative Construct Sequences for Direct Regulation of Cas and for Transcriptionally Regulating Cas

The present example provides sequences of constructs that may be designed for use as components of direct Cas-DRD regulation systems or Cas-transcription factor systems. Table 8 provides nucleic acid sequences of vectors comprising constructs for direct regulation of Cas and for transcriptionally regulating Cas. The constructs listed in Table 8 correspond to constructs described in the preceding examples. The sequences provided by the present example are not intended to be limiting in scope, but rather are illustrative of approaches for designing Cas-DRD regulation system or Cas-transcription factor system constructs. Variations on these sequences as well as other constructs and other sequences are encompassed by the present disclosure in accordance with the descriptions of Cas-DRD regulation systems or Cas-transcription factor systems throughout the present disclosure.

TABLE 8
Vector sequences comprising constructs
for direct regulation of Cas or for
transcriptionally regulating Cas

| Construct | |
|---|---|
| Name | Sequence |
| OT-Cas9-021 | ttattcccttttttgcggcattttgccttc |
| | ctgtttttgctcacccagaaacgctggtga |
| | aagtaaaagatgctgaagatcagttgggtg |
| | cacgagtgggttacatcgaactggatctca |
| | acagcggtaagatccttgagagttttcgcc |
| | ccgaagaacgttttccaatgatgagcactt |
| | ttaaagttctgctatgtggcgcggtattat |
| | cccgtattgacgccgggcaagagcaactcg |
| | gtcgccgcatacactattctcagaatgact |
| | tggttgagtactcaccagtcacagaaaagc |
| | atcttacggatggcatgacagtaagagaat |
| | tatgcagtgctgccataaccatgagtgata |
| | acactgcggccaacttacttctgacaacga |
| | tcggaggaccgaaggagctaaccgcttttt |
| | tgcacaacatgggggatcatgtaactcgcc |
| | ttgatcgttgggaaccggagctgaatgaag |
| | ccataccaaacgacgagcgtgacaccacga |
| | tgcctgtagcaatggcaacaacgttgcgca |
| | aactattaactggcgaactacttactctag |
| | cttcccggcaacaattaatagactggatgg |
| | aggcggataaagttgcaggaccacttctgc |
| | gctcggcccttccggctggctggtttattg |
| | ctgataaatctggagccggtgagcgtgggt |
| | ctcgcggtatcattgcagcactggggccag |
| | atggtaagccctcccgtatcgtagttatct |
| | acacgacggggagtcaggcaactatggatg |
| | aacgaaatagacagatcgctgagataggtg |
| | cctcactgattaagcattggtaactgtcag |
| | accaagtttactcatatatactttagattg |
| | atttaaaacttcatttttaatttaaaagga |
| | tctaggtgaagatcctttttgataatctca |
| | tgaccaaaatcccttaacgtgagttttcgt |
| | tccactgagcgtcagaccccgtagaaaaga |
| | tcaaaggatcttcttgagatcctttttttc |
| | tgcgcgtaatctgctgcttgcaaacaaaaa |
| | aaccaccgctaccagcggtggtttgtttgc |
| | cggatcaagagctaccaactctttttccga |
| | aggtaactggcttcagcagagcgcagatac |
| | caaatactgttcttctagtgtagccgtagt |
| | taggccaccacttcaagaactctgtagcac |
| | cgcctacatacctcgctctgctaatcctgt |
| | taccagtggctgctgccagtggcgataagt |
| | cgtgtcttaccgggttggactcaagacgat |
| | agttaccggataaggcgcagcggtcgggct |
| | gaacggggggttcgtgcacacagcccagct |
| | tggagcgaacgacctacaccgaactgagat |
| | acctacagcgtgagctatgagaaagcgcca |
| | cgcttcccgaagggagaaaggcggacaggt |
| | atccggtaagcggcagggtcggaacaggag |
| | agcgcacgagggagcttccagggggaaacg |
| | cctggtatctttatagtcctgtcgggtttc |
| | gccacctctgacttgagcgtcgatttttgt |
| | gatgctcgtcaggggggcggagcctatgga |
| | aaaacgccagcaacgcggcctttttacggt |
| | tcctggccttttgctggccttttgctcaca |
| | tgttctttcctgcgttatcccctgattctg |
| | tggataaccgtattaccgcctttgagtgag |
| | ctgataccgctcgccgcagccgaacgaccg |
| | agcgcagcgagtcagtgagcgaggaagcgg |
| | aagagcgcccaatacgcaaaccgcctctcc |
| | ccgcgcgttggccgattcattaatgcagct |
| | ggcacgacaggtttcccgactggaaagcgg |
| | gcagtgagcgcaacgcaattaatgtgagtt |
| | agctcactcattaggcaccccaggctttac |
| | actttatgcttccggctcgtatgttgtgtg |
| | gaattgtgagcggataacaatttcacacag |
| | gaaacagctatgaccatgattacgccaagc |
| | gcgcaattaaccctcactaaagggaacaaa |
| | agctggagctgcaagcttagacattgatta |
| | ttgactagttattaatagtaatcaattacg |
| | gggtcattagttcatagcccatatatggag |
| | ttccgcgttacataacttacggtaaatggc |
| | ccgcctggctgaccgcccaacgacccccgc |
| | ccattgacgtcaataatgacgtatgttccc |
| | atagtaacgccaatagggactttccattga |
| | cgtcaatgggtggagtatttacggtaaact |
| | gcccacttggcagtacatcaagtgtatcat |
| | atgccaagtacgccccctattgacgtcaat |
| | gacggtaaatggcccgcctggcattatgcc |
| | cagtacatgaccttatgggactttcctact |
| | tggcagtacatctacgtattagtcatcgct |
| | attaccatggtgatgcggttttggcagtac |
| | atcaatgggcgtggatagcggtttgactca |
| | cggggatttccaagtctccaccccattgac |
| | gtcaatgggagtttgttttggcaccaaaat |
| | caacgggactttccaaaatgtcgtaacaac |
| | tccgccccattgacgcaaatgggcggtagg |
| | cgtgtacggtgggaggtctatataagcagc |
| | gcgttttgcctgtactgggtctctctggtt |
| | agaccagatctgagcctgggagctctctgg |
| | ctaactagggaacccactgcttaagcctca |
| | ataaagcttgccttgagtgcttcaagtagt |
| | gtgtgcccgtctgttgtgtgactctggtaa |
| | ctagagatccctcagacccttttagtcagt |
| | gtggaaaatctctagcagtggcgcccgaac |
| | agggacttgaaagcgaaagggaaaccagag |
| | gagctctctcgacgcaggactcggcttgct |
| | gaagcgcgcacggcaagaggcgaggggcgg |
| | cgactggtgagtacgccaaaaattttgact |
| | agcggaggctagaaggagagagatgggtgc |
| | gagagcgtcagtattaagcgggggagaatt |
| | agatcgcgatgggaaaaaattcggttaagg |
| | ccagggggaaagaaaaaatataaattaaaa |
| | catatagtatgggcaagcagggagctagaa |
| | cgattcgcagttaatcctggcctgttagaa |
| | acatcagaaggctgtagacaaatactggga |
| | cagctacaaccatcccttcagacaggatca |
| | gaagaacttagatcattatataatacagta |
| | gcaaccctctattgtgtgcatcaaaggata |
| | gagataaaagacaccaaggaagctttagac |
| | aagatagaggaagagcaaaacaaaagtaag |
| | accaccgcacagcaagcggccgctgatctt |
| | cagacctggaggaggagatatgagggacaa |
| | ttggagaagtgaattatataaatataaagt |
| | agtaaaaattgaaccattaggagtagcacc |
| | caccaaggcaaagagaagagtggtgcagag |
| | agaaaaaagagcagtgggaataggagcttt |
| | gttccttgggttcttgggagcagcaggaag |
| | cactatgggcgcagcgtcaatgacgctgac |
| | ggtacaggccagacaattattgtctggtat |
| | agtgcagcagcagaacaaMgctgagggcta |
| | ttgaggcgcaacagcatctgttgcaactca |
| | cagtctggggcatcaagcagctccaggcaa |
| | gaatcctggctgtggaaagatacctaaagg |
| | atcaacagctcctggggatttggggttgct |
| | ctggaaaactcatttgcaccactgctgtgc |
| | cttggaatgctagttggagtaataaatctc |
| | tggaacagatttggaatcacacgacctgga |
| | tggagtgggacagagaaattaacaattaca |
| | caagcttaatacactccttaattgaagaat |
| | cgcaaaaccagcaagaaaagaatgaacaag |
| | aattattggaattagataaatgggcaagtt |
| | tgtggaattggtttaacataacaaattggc |
| | tgtggtatataaaattattcataatgatag |
| | taggaggcttggtaggtttaagaatagttt |
| | ttgctgtactttctatagtgaatagagtta |
| | ggcagggatattcaccattatcgtttcaga |
| | cccacctcccaaccccgaggggacccgaca |
| | ggcccgaaggaatagaagaagaaggtggag |
| | agagagacagagacagatccattcgattag |
| | tgaacggatctcgacggtatcgattagact |
| | gtagcccaggaatatggcagctagattgta |
| | cacatttagaaggaaaagttatcttggtag |
| | cagttcatgtagccagtggatatatagaag |
| | cagaagtaattccagcagagacagggcaag |
| | aaacagcatacttcctcttaaaattagcag |
| | gaagatggccagtaaaaacagtacatacag |
| | acaatggcagcaatttcaccagtactacag |
| | ttaaggccgcctgttggtgggcggggatca |
| | agcaggaatttggcattccctacaatcccc |
| | aaagtcaaggagtaatagaatctatgaata |
| | aagaattaaagaaaattataggacaggtaa |
| | gagatcaggctgaacatcttaagacagcag |
| | tacaaatggcagtattcatccacaatttta |
| | aaagaaaaggggggattggggggtacagtg |
| | caggggaaagaatagtagacataatagcaa |
| | cagacatacaaactaaagaattacaaaaac |
| | aaattacaaaaattcaaaattttcgggttt |
| | attacagggacagcagagatccagtttggc |
| | tcgggtttattacagggacagcagagatcc |
| | agtttggttaattaaggtaccgagggccta |
| | tttcccatgattccttcatatttgcatata |
| | cgatacaaggctgttagagagataattaga |
| | attaatttgactgtaaacacaaagatatta |
| | gtacaaaatacgtgacgtagaaagtaataa |
| | tttcttgggtagtttgcagttttaaaatta |
| | tgttttaaaatggactatcatatgcttacc |
| | gtaacttgaaagtatttcgatttcttggct |
| | ttatatatcttgtggaaaggacgaaaGAAG |
| | TTCGAGGGCGACACCCgttttagagctaga |
| | aatagcaagttaaaataaggctagtccgtt |
| | atcaacttgaaaaagtggcaccgagtcggt |
| | gcttttttgaattcgctagctaggtcttga |
| | aaggagtgggaattggctccggtgcccgtc |
| | agtgggcagagcgcacatcgcccacagtcc |
| | ccgagaagttggggggaggggtcggcaatt |
| | gatccggtgcctagagaaggtggcgcgggg |
| | taaactgggaaagtgatgtcgtgtactggc |
| | tccgcctttttcccgagggtgggggagaac |
| | cgtatataagtgcagtagtcgccgtgaacg |
| | ttctttttcgcaacgggtttgccgccagaa |
| | cacaggaccggttctagagccaccATGGGA |
| | TCCgacaagaagtacagcatcggcctggac |
| | atcggcaccaactctgtgggctgggccgtg |
| | atcaccgacgagtacaaggtgcccagcaag |
| | aaattcaaggtgctgggcaacaccgaccgg |
| | cacagcatcaagaagaacctgatcggagcc |
| | ctgctgttcgacagcggcgaaacagccgag |
| | gccacccggctgaagagaaccgccagaaga |
| | agatacaccagacggaagaaccggatctgc |
| | tatctgcaagagatcttcagcaacgagatg |
| | gccaaggtggacgacagcttcttccacaga |
| | ctggaagagtccttcctggtggaagaggat |
| | aagaagcacgagcggcaccccatcttcggc |
| | aacatcgtggacgaggtggcctaccacgag |
| | aagtaccccaccatctaccacctgagaaag |
| | aaactggtggacagcaccgacaaggccgac |
| | ctgcggctgatctatctggccctggcccac |
| | atgatcaagttccggggccacttcctgatc |
| | gagggcgacctgaaccccgacaacagcgac |
| | gtggacaagctgttcatccagctggtgcag |
| | acctacaaccagctgttcgaggaaaacccc |
| | atcaacgccagcggcgtggacgccaaggcc |
| | atcctgtctgccagactgagcaagagcaga |
| | cggctggaaaatctgatcgcccagctgccc |
| | ggcgagaagaagaatggcctgttcggaaac |
| | ctgattgccctgagcctgggcctgaccccc |
| | aacttcaagagcaacttcgacctggccgag |
| | gatgccaaactgcagctgagcaaggacacc |
| | tacgacgacgacctggacaacctgctggcc |
| | cagatcggcgaccagtacgccgacctgttt |
| | ctggccgccaagaacctgtccgacgccatc |
| | ctgctgagcgacatcctgagagtgaacacc |
| | gagatcaccaaggcccccctgagcgcctct |
| | atgatcaagagatacgacgagcaccaccag |
| | gacctgaccctgctgaaagctctcgtgcgg |
| | cagcagctgcctgagaagtacaaagagatt |
| | ttcttcgaccagagcaagaacggctacgcc |
| | ggctacattgacggcggagccagccaggaa |
| | gagttctacaagttcatcaagcccatcctg |
| | gaaaagatggacggcaccgaggaactgctc |
| | gtgaagctgaacagagaggacctgctgcgg |
| | aagcagcggaccttcgacaacggcagcatc |
| | ccccaccagatccacctgggagagctgcac |
| | gccattctgcggcggcaggaagatttttac |
| | ccattcctgaaggacaaccgggaaaagatc |
| | gagaagatcctgaccttccgcatcccctac |
| | tacgtgggccctctggccaggggaaacagc |
| | agattcgcctggatgaccagaaagagcgag |
| | gaaaccatcaccccctggaacttcgaggaa |
| | gtggtggacaagggcgcttccgcccagagc |
| | ttcatcgagcggatgaccaacttcgataag |
| | aacctgcccaacgagaaggtgctgcccaag |
| | cacagcctgctgtacgagtacttcaccgtg |
| | tataacgagctgaccaaagtgaaatacgtg |
| | accgagggaatgagaaagcccgccttcctg |
| | agcggcgagcagaaaaaggccatcgtggac |
| | ctgctgttcaagaccaaccggaaagtgacc |
| | gtgaagcagctgaaagaggactacttcaag |
| | aaaatcgagtgcttcgactccgtggaaatc |
| | tccggcgtggaagatcggttcaacgcctcc |
| | ctgggcacataccacgatctgctgaaaatt |
| | atcaaggacaaggacttcctggacaatgag |
| | gaaaacgaggacattctggaagatatcgtg |
| | ctgaccctgacactgtttgaggacagagag |
| | atgatcgaggaacggctgaaaacctatgcc |
| | cacctgttcgacgacaaagtgatgaagcag |
| | ctgaagcggcggagatacaccggctggggc |
| | aggctgagccggaagctgatcaacggcatc |
| | cgggacaagcagtccggcaagacaatcctg |
| | gatttcctgaagtccgacggcttcgccaac |
| | agaaacttcatgcagctgatccacgacgac |
| | agcctgacctttaaagaggacatccagaaa |
| | gcccaggtgtccggccagggcgatagcctg |
| | cacgagcacattgccaatctggccggcagc |
| | cccgccattaagaagggcatcctgcagaca |
| | gtgaaggtggtggacgagctcgtgaaagtg |
| | atgggccggcacaagcccgagaacatcgtg |
| | atcgaaatggccagagagaaccagaccacc |
| | cagaagggacagaagaacagccgcgagaga |
| | atgaagcggatcgaagagggcatcaaagag |
| | ctgggcagccagatcctgaaagaacacccc |
| | gtggaaaacacccagctgcagaacgagaag |
| | ctgtacctgtactacctgcagaatgggcgg |
| | gatatgtacgtggaccaggaactggacatc |
| | aaccggctgtccgactacgatgtggaccat |
| | atcgtgcctcagagctttctgaaggacgac |
| | tccatcgacaacaaggtgctgaccagaagc |
| | gacaagaaccggggcaagagcgacaacgtg |
| | ccctccgaagaggtcgtgaagaagatgaag |
| | aactactggcggcagctgctgaacgccaag |
| | ctgattacccagagaaagttcgacaatctg |
| | accaaggccgagagaggcggcctgagcgaa |
| | ctggataaggccggcttcatcaagagacag |
| | ctggtggaaacccggcagatcacaaagcac |
| | gtggcacagatcctggactcccggatgaac |
| | actaagtacgacgagaatgacaagctgatc |
| | cgggaagtgaaagtgatcaccctgaagtcc |
| | aagctggtgtccgatttccggaaggatttc |
| | cagttttacaaagtgcgcgagatcaacaac |
| | taccaccacgcccacgacgcctacctgaac |
| | gccgtcgtgggaaccgccctgatcaaaaag |
| | taccctaagctggaaagcgagttcgtgtac |
| | ggcgactacaaggtgtacgacgtgcggaag |
| | atgatcgccaagagcgagcaggaaatcggc |
| | aaggctaccgccaagtacttcttctacagc |
| | aacatcatgaactttttcaagaccgagatt |
| | accctggccaacggcgagatccggaagcgg |
| | cctctgatcgagacaaacggcgaaaccggg |
| | gagatcgtgtgggataagggccgggatttt |
| | gccaccgtgcggaaagtgctgagcatgccc |
| | caagtgaatatcgtgaaaaagaccgaggtg |
| | cagacaggcggcttcagcaaagagtctatc |
| | ctgcccaagaggaacagcgataagctgatc |
| | gccagaaagaaggactgggaccctaagaag |
| | tacggcggcttcgacagccccaccgtggcc |
| | tattctgtgctggtggtggccaaagtggaa |
| | aagggcaagtccaagaaactgaagagtgtg |
| | aaagagctgctggggatcaccatcatggaa |
| | agaagcagcttcgagaagaatcccatcgac |
| | tttctggaagccaagggctacaaagaagtg |
| | aaaaaggacctgatcatcaagctgcctaag |
| | tactccctgttcgagctggaaaacggccgg |
| | aagagaatgctggcctctgccggcgaactg |
| | cagaagggaaacgaactggccctgccctcc |
| | aaatatgtgaacttcctgtacctggccagc |
| | cactatgagaagctgaagggctcccccgag |
| | gataatgagcagaaacagctgtttgtggaa |
| | cagcacaagcactacctggacgagatcatc |
| | gagcagatcagcgagttctccaagagagtg |
| | atcctggccgacgctaatctggacaaagtg |
| | ctgtccgcctacaacaagcaccgggataag |
| | cccatcagagagcaggccgagaatatcatc |
| | cacctgtttaccctgaccaatctgggagcc |
| | cctgccgccttcaagtactttgacaccacc |
| | atcgaccggaagaggtacaccagcaccaaa |
| | gaggtgctggacgccaccctgatccaccag |
| | agcatcaccggcctgtacgagacacggatc |
| | gacctgtctcagctgggaggcgacaagcga |
| | cctgccgccacaaagaaggctggacaggct |
| | aagaagaagaaagattacaaagacgatgac |
| | gataagGGTtccGGCgctactaacttcagc |
| | ctgctgaagcaggctggggacgtggaggag |
| | aaccctggacctaggACGCGTttgagcaag |
| | ggcgaggaggacaacatggccatcatcaag |
| | gagttcatgcgcttcaaggtgcacatggag |
| | ggctccgtgaacggccacgagttcgagatc |
| | gagggcgagggcgagggccgcccctacgag |
| | ggcacccagaccgccaagctgaaggtgacc |
| | aagggcggccccctgcccttcgcctgggac |
| | atcctgtcccctcagttcatgtacggctcc |
| | aaggcctacgtgaagcaccccgccgacatc |
| | cccgactacttgaagctgtccttccccgag |
| | ggcttcaagtgggagcgcgtgatgaacttc |
| | gaggacggcggcgtggtgaccgtgacccag |
| | gactcctccctgcaggacggcgagttcatc |
| | tacaaggtgaagctgcgcggcaccaacttc |
| | ccctccgacggccccgtaatgcagaagaag |
| | accatgggctgggaggcctcctccgagcgg |
| | atgtaccccgaggacggcgccctgaagggc |
| | gagatcaagcagaggctgaagctgaaggac |
| | ggcggccactacgacgccgaggtcaagacc |
| | acctacaaggccaagaagcccgtgcagctg |
| | cccggcgcctacaacgtcaacatcaagctg |
| | gacatcacctcccacaacgaggactacacc |
| | atcgtggaacagtacgagcgcgccgagggc |
| | cgccactccaccggcggcatggacgagctg |
| | tacaagtaaATCGATATCGGGCTAGCgtcg |
| | acaatcaacctctggattacaaaatttgtg |
| | aaagattgactggtattcttaactatgttg |
| | ctccttttacgctatgtggatacgctgctt |
| | taatgcctttgtatcatgctattgcttccc |
| | gtatggctttcattttctcctccttgtata |
| | aatcctggttgctgtctctttatgaggagt |
| | tgtggcccgttgtcaggcaacgtggcgtgg |
| | tgtgcactgtgtttgctgacgcaaccccca |
| | ctggttggggcattgccaccacctgtcagc |
| | tcctttccgggactttcgctttccccctcc |
| | ctattgccacggcggaactcatcgccgcct |
| | gccttgcccgctgctggacaggggctcggc |
| | tgttgggcactgacaattccgtggtgttgt |
| | cggggaagctgacgtcctttccatggctgc |
| | tcgcctgtgttgccacctggattctgcgcg |
| | ggacgtccttctgctacgtcccttcggccc |
| | tcaatccagcggaccttccttcccgcggcc |
| | tgctgccggctctgcggcctcttccgcgtc |
| | ttcgccttcgccctcagacgagtcggatct |
| | ccctttgggccgcctccccgcctggaattc |
| | gagctcggtacctttaagaccaatgactta |
| | caaggcagctgtagatcttagccacttttt |
| | aaaagaaaaggggggactggaagggctaat |
| | tcactcccaacgaagacaagatctgctttt |
| | tgcttgtactgggtctctctggttagacca |
| | gatctgagcctgggagctctctggctaact |
| | agggaacccactgcttaagcctcaataaag |
| | cttgccttgagtgcttcaagtagtgtgtgc |
| | ccgtctgttgtgtgactctggtaactagag |
| | atccctcagacccttttagtcagtgtggaa |
| | aatctctagcagtagtagttcatgtcatct |
| | tattattcagtatttataacttgcaaagaa |
| | atgaatatcagagagtgagaggaacttgtt |
| | tattgcagcttataatggttacaaataaag |
| | caatagcatcacaaatttcacaaataaagc |
| | atttttttcactgcattctagttgtggttt |
| | gtccaaactcatcaatgtatcttatcatgt |
| | ctggctctagctatcccgcccctaactccg |
| | cccatcccgcccctaactccgcccagttcc |
| | gcccattctccgccccatggctgactaatt |
| | ttttttatttatgcagaggccgaggccgcc |
| | tcggcctctgagctattccagaagtagtga |
| | ggaggcttttttggaggcctagggacgtac |
| | ccaattcgcCCTATAGTGAGTCGTATTAcg |
| | cgcgctcactggccgtcgttttacaacgtc |
| | gtgactgggaaaaccctggcgttacccaac |
| | ttaatcgccttgcagcacatccccctttcg |
| | ccagctggcgtaatagcgaagaggcccgca |
| | ccgatcgcccttcccaacagttgcgcagcc |
| | tgaatggcgaatgggacgcgccctgtagcg |
| | gcgcattaagcgcggcgggtgtggtggtta |
| | cgcgcagcgtgaccgctacacttgccagcg |
| | ccctagcgcccgctcctttcgctttcttcc |
| | cttcctttctcgccacgttcgccggctttc |
| | cccgtcaagctctaaatcgggggctccctt |
| | tagggttccgatttagtgctttacggcacc |
| | tcgaccccaaaaaacttgattagggtgatg |
| | gttcacgtagtgggccatcgccctgataga |
| | cggtttttcgccctttgacgttggagtcca |
| | cgttctttaatagtggactcttgttccaaa |
| | ctggaacaacactcaaccctatctcggtct |
| | attcttttgatttataagggattttgccga |
| | tttcggcctattggttaaaaaatgagctga |
| | tttaacaaaaatttaacgcgaattttaaca |
| | aaatattaacgcttacaatttaggtggcac |
| | ttttcggggaaatgtgcgcggaacccctat |
| | ttgtttatttttctaaatacattcaaatat |
| | gtatccgctcatgagacaataaccctgata |
| | aatgcttcaataatattgaaaaaggaagag |
| | tatgagtattcaacatttccgtgtcgccc( |
| | SEQ ID NO: 39) |
| OT-Cas9-024 | gacattgattattgactagttattaatagt |
| | aatcaattacggggtcattagttcatagcc |
| | catatatggagttccgcgttacataactta |
| | cggtaaatggcccgcctggctgaccgccca |
| | acgacccccgcccattgacgtcaataatga |
| | cgtatgttcccatagtaacgccaataggga |
| | ctttccattgacgtcaatgggtggagtatt |
| | tacggtaaactgcccacttggcagtacatc |
| | aagtgtatcatatgccaagtacgcccccta |
| | ttgacgtcaatgacggtaaatggcccgcct |
| | ggcattatgcccagtacatgaccttatggg |
| | actttcctacttggcagtacatctacgtat |
| | tagtcatcgctattaccatggtgatgcggt |
| | tttggcagtacatcaatgggcgtggatagc |
| | ggtttgactcacggggatttccaagtctcc |
| | accccattgacgtcaatgggagtttgtttt |
| | ggcaccaaaatcaacgggactttccaaaat |
| | gtcgtaacaactccgccccattgacgcaaa |
| | tgggcggtaggcgtgtacggtgggaggtct |
| | atataagcagcgcgttttgcctgtactggg |
| | tctctctggttagaccagatctgagcctgg |
| | gagctctctggctaactagggaacccactg |
| | cttaagcctcaataaagcttgccttgagtg |
| | cttcaagtagtgtgtgcccgtctgttgtgt |
| | gactctggtaactagagatccctcagaccc |
| | ttttagtcagtgtggaaaatctctagcagt |
| | ggcgcccgaacagggacttgaaagcgaaag |
| | ggaaaccagaggagctctctcgacgcagga |
| | ctcggcttgctgaagcgcgcacggcaagag |
| | gcgaggggcggcgactggtgagtacgccaa |
| | aaattttgactagcggaggctagaaggaga |
| | gagatgggtgcgagagcgtcagtattaagc |
| | gggggagaattagatcgcgatgggaaaaaa |
| | ttcggttaaggccagggggaaagaaaaaat |
| | ataaattaaaacatatagtatgggcaagca |
| | gggagctagaacgattcgcagttaatcctg |
| | gcctgttagaaacatcagaaggctgtagac |
| | aaatactgggacagctacaaccatcccttc |
| | agacaggatcagaagaacttagatcattat |
| | ataatacagtagcaaccctctattgtgtgc |
| | atcaaaggatagagataaaagacaccaagg |
| | aagctttagacaagatagaggaagagcaaa |
| | acaaaagtaagaccaccgcacagcaagcgg |
| | ccgctgatcttcagacctggaggaggagat |
| | atgagggacaattggagaagtgaattatat |
| | aaatataaagtagtaaaaattgaaccatta |
| | ggagtagcacccaccaaggcaaagagaaga |
| | gtggtgcagagagaaaaaagagcagtggga |
| | ataggagctttgttccttgggttcttggga |
| | gcagcaggaagcactatgggcgcagcgtca |
| | atgacgctgacggtacaggccagacaatta |
| | ttgtctggtatagtgcagcagcagaacaat |
| | ttgctgagggctattgaggcgcaacagcat |
| | ctgttgcaactcacagtctggggcatcaag |
| | cagctccaggcaagaatcctggctgtggaa |
| | agatacctaaaggatcaacagctcctgggg |
| | atttggggttgctctggaaaactcatttgc |
| | accactgctgtgccttggaatgctagttgg |
| | agtaataaatctctggaacagatttggaat |
| | cacacgacctggatggagtgggacagagaa |
| | attaacaattacacaagcttaatacactcc |
| | ttaattgaagaatcgcaaaaccagcaagaa |
| | aagaatgaacaagaattattggaattagat |
| | aaatgggcaagtttgtggaattggtttaac |
| | ataacaaattggctgtggtatataaaatta |
| | ttcataatgatagtaggaggcttggtaggt |
| | ttaagaatagttMgctgtactttctatagt |
| | gaatagagttaggcagggatattcaccatt |
| | atcgtttcagacccacctcccaaccccgag |
| | gggacccgacaggcccgaaggaatagaaga |
| | agaaggtggagagagagacagagacagatc |
| | cattcgattagtgaacggatctcgacggta |
| | tcgattagactgtagcccaggaatatggca |
| | gctagattgtacacatttagaaggaaaagt |
| | tatcttggtagcagttcatgtagccagtgg |
| | atatatagaagcagaagtaattccagcaga |
| | gacagggcaagaaacagcatacttcctctt |
| | aaaattagcaggaagatggccagtaaaaac |
| | agtacatacagacaatggcagcaatttcac |
| | cagtactacagttaaggccgcctgttggtg |
| | ggcggggatcaagcaggaatttggcattcc |
| | ctacaatccccaaagtcaaggagtaataga |
| | atctatgaataaagaattaaagaaaattat |
| | aggacaggtaagagatcaggctgaacatct |
| | taagacagcagtacaaatggcagtattcat |
| | ccacaattttaaaagaaaaggggggattgg |
| | ggggtacagtgcaggggaaagaatagtaga |
| | cataatagcaacagacatacaaactaaaga |
| | attacaaaaacaaattacaaaaattcaaaa |
| | ttttcgggtttattacagggacagcagaga |
| | tccagtttggctcgggtttattacagggac |
| | agcagagatccagtttggttaattaaggta |
| | ccgagggcctatttcccatgattccttcat |
| | atttgcatatacgatacaaggctgttagag |
| | agataattagaattaatttgactgtaaaca |
| | caaagatattagtacaaaatacgtgacgta |
| | gaaagtaataatttcttgggtagtttgcag |
| | ttttaaaattatgttttaaaatggactatc |
| | atatgcttaccgtaacttgaaagtatttcg |
| | atttcttggctttatatatcttgtggaaag |
| | gacgaaaGAAGTTCGAGGGCGACACCCgtt |
| | ttagagctagaaatagcaagttaaaataag |
| | gctagtccgttatcaacttgaaaaagtggc |
| | accgagtcggtgctatttgaattcgctagc |
| | taggtcttgaaaggagtgggaattggctcc |
| | ggtgcccgtcagtgggcagagcgcacatcg |
| | cccacagtccccgagaagttggggggaggg |
| | gtcggcaattgatccggtgcctagagaagg |
| | tggcgcggggtaaactgggaaagtgatgtc |
| | gtgtactggctccgcctttttcccgagggt |
| | gggggagaaccgtatataagtgcagtagtc |
| | gccgtgaacgttctttttcgcaacgggttt |
| | gccgccagaacacaggaccggttctagagc |
| | caccATGTCCCATCACTGGGGGTACGGCAA |
| | ACACAACGGACCTGAGCACTGGCATAAGGA |
| | CTTCCCCATTGCCAAGGGAGAGCGCCAGTC |
| | CCCTGTTGACATCGACACTCATACAGCCAA |
| | GTATGACCCTTCCCTGAAGCCCCTGTCTGT |
| | TTCCTATGATCAAGCAACTTCCCTGAGGAT |
| | TCTCAACAATGGTCATGCTTTCAACGTGGA |
| | GTTTGATGACTCTCAGGACAAAGCAGTGCT |
| | CAAGGGAGGACCCCTGGATGGCACTTACAG |
| | ATTGATTCAGTTTCACTTTCACTGGGGTTC |
| | ACTTGATGGACAAGGTTCAGAGCATACTGT |
| | GGATAAAAAGAAATATGCTGCAGAACTTCA |
| | CTTGGTTCACTGGAACACCAAATATGGGGA |
| | TTTTGGGAAAGCTGTGCAGCAACCTGATGG |
| | ACTGGCCGTTCTAGGTATTTTTTTGAAGGT |
| | TGGCAGCGCTAAACCGGGCCATCAGAAAGT |
| | TGTTGATGTGCTGGATTCCATTAAAACAAA |
| | GGGCAAGAGTGCTGACTTCACTAACTTCGA |
| | TCCTCGTGGCCTCCTTCCTGAATCCCTGGA |
| | TTACTGGACCTACCCAGGCTCACTGACCAC |
| | CCCTCCTCTTCTGGAATGTGTGACCTGGAT |
| | TGTGCTCAAGGAACCCATCAGCGTCAGCAG |
| | CGAGCAGGTGTTGAAATTCCGTAAACTTAA |
| | CTTCAATGGGGAGGGTGAACCCGAAGAACT |
| | GATGGTGGACAACTGGCGCCCAGCTCAGCC |
| | ACTGAAGAACAGGCAAATCAAAGCTTCCTT |
| | CAAAGGATCCgacaagaagtacagcatcgg |
| | cctggacatcggcaccaactctgtgggctg |
| | ggccgtgatcaccgacgagtacaaggtgcc |
| | cagcaagaaattcaaggtgctgggcaacac |
| | cgaccggcacagcatcaagaagaacctgat |
| | cggagccctgctgttcgacagcggcgaaac |
| | agccgaggccacccggctgaagagaaccgc |
| | cagaagaagatacaccagacggaagaaccg |
| | gatctgctatctgcaagagatcttcagcaa |
| | cgagatggccaaggtggacgacagcttctt |
| | ccacagactggaagagtccttcctggtgga |
| | agaggataagaagcacgagcggcaccccat |
| | cttcggcaacatcgtggacgaggtggccta |
| | ccacgagaagtaccccaccatctaccacct |
| | gagaaagaaactggtggacagcaccgacaa |
| | ggccgacctgcggctgatctatctggccct |
| | ggcccacatgatcaagttccggggccactt |
| | cctgatcgagggcgacctgaaccccgacaa |
| | cagcgacgtggacaagctgttcatccagct |
| | ggtgcagacctacaaccagctgttcgagga |
| | aaaccccatcaacgccagcggcgtggacgc |
| | caaggccatcctgtctgccagactgagcaa |
| | gagcagacggctggaaaatctgatcgccca |
| | gctgcccggcgagaagaagaatggcctgtt |
| | cggaaacctgattgccctgagcctgggcct |
| | gacccccaacttcaagagcaacttcgacct |
| | ggccgaggatgccaaactgcagctgagcaa |
| | ggacacctacgacgacgacctggacaacct |
| | gctggcccagatcggcgaccagtacgccga |
| | cctgtttctggccgccaagaacctgtccga |
| | cgccatcctgctgagcgacatcctgagagt |
| | gaacaccgagatcaccaaggcccccctgag |
| | cgcctctatgatcaagagatacgacgagca |
| | ccaccaggacctgaccctgctgaaagctct |
| | cgtgcggcagcagctgcctgagaagtacaa |
| | agagattttcttcgaccagagcaagaacgg |
| | ctacgccggctacattgacggcggagccag |
| | ccaggaagagttctacaagttcatcaagcc |
| | catcctggaaaagatggacggcaccgagga |
| | actgctcgtgaagctgaacagagaggacct |
| | gctgcggaagcagcggaccttcgacaacgg |
| | cagcatcccccaccagatccacctgggaga |
| | gctgcacgccattctgcggcggcaggaaga |
| | tttttacccattcctgaaggacaaccggga |
| | aaagatcgagaagatcctgaccttccgcat |
| | cccctactacgtgggccctctggccagggg |
| | aaacagcagattcgcctggatgaccagaaa |
| | gagcgaggaaaccatcaccccctggaactt |
| | cgaggaagtggtggacaagggcgcttccgc |
| | ccagagcttcatcgagcggatgaccaactt |
| | cgataagaacctgcccaacgagaaggtgct |
| | gcccaagcacagcctgctgtacgagtactt |
| | caccgtgtataacgagctgaccaaagtgaa |
| | atacgtgaccgagggaatgagaaagcccgc |
| | cttcctgagcggcgagcagaaaaaggccat |
| | cgtggacctgctgttcaagaccaaccggaa |
| | agtgaccgtgaagcagctgaaagaggacta |
| | cttcaagaaaatcgagtgcttcgactccgt |
| | ggaaatctccggcgtggaagatcggttcaa |
| | cgcctccctgggcacataccacgatctgct |
| | gaaaattatcaaggacaaggacttcctgga |
| | caatgaggaaaacgaggacattctggaaga |
| | tatcgtgctgaccctgacactgtttgagga |
| | cagagagatgatcgaggaacggctgaaaac |
| | ctatgcccacctgttcgacgacaaagtgat |
| | gaagcagctgaagcggcggagatacaccgg |
| | ctggggcaggctgagccggaagctgatcaa |
| | cggcatccgggacaagcagtccggcaagac |
| | aatcctggatttcctgaagtccgacggctt |
| | cgccaacagaaacttcatgcagctgatcca |
| | cgacgacagcctgacctttaaagaggacat |
| | ccagaaagcccaggtgtccggccagggcga |
| | tagcctgcacgagcacattgccaatctggc |
| | cggcagccccgccattaagaagggcatcct |
| | gcagacagtgaaggtggtggacgagctcgt |
| | gaaagtgatgggccggcacaagcccgagaa |
| | catcgtgatcgaaatggccagagagaacca |
| | gaccacccagaagggacagaagaacagccg |
| | cgagagaatgaagcggatcgaagagggcat |
| | caaagagctgggcagccagatcctgaaaga |
| | acaccccgtggaaaacacccagctgcagaa |
| | cgagaagctgtacctgtactacctgcagaa |
| | tgggcgggatatgtacgtggaccaggaact |
| | ggacatcaaccggctgtccgactacgatgt |
| | ggaccatatcgtgcctcagagctttctgaa |
| | ggacgactccatcgacaacaaggtgctgac |
| | cagaagcgacaagaaccggggcaagagcga |
| | caacgtgccctccgaagaggtcgtgaagaa |
| | gatgaagaactactggcggcagctgctgaa |
| | cgccaagctgattacccagagaaagttcga |
| | caatctgaccaaggccgagagaggcggcct |
| | gagcgaactggataaggccggcttcatcaa |
| | gagacagctggtggaaacccggcagatcac |
| | aaagcacgtggcacagatcctggactcccg |
| | gatgaacactaagtacgacgagaatgacaa |
| | gctgatccgggaagtgaaagtgatcaccct |
| | gaagtccaagctggtgtccgatttccggaa |
| | ggatttccagttttacaaagtgcgcgagat |
| | caacaactaccaccacgcccacgacgccta |
| | cctgaacgccgtcgtgggaaccgccctgat |
| | caaaaagtaccctaagctggaaagcgagtt |
| | cgtgtacggcgactacaaggtgtacgacgt |
| | gcggaagatgatcgccaagagcgagcagga |
| | aatcggcaaggctaccgccaagtacttctt |
| | ctacagcaacatcatgaactttttcaagac |
| | cgagattaccctggccaacggcgagatccg |
| | gaagcggcctctgatcgagacaaacggcga |
| | aaccggggagatcgtgtgggataagggccg |
| | ggattttgccaccgtgcggaaagtgctgag |
| | catgccccaagtgaatatcgtgaaaaagac |
| | cgaggtgcagacaggcggcttcagcaaaga |
| | gtctatcctgcccaagaggaacagcgataa |
| | gctgatcgccagaaagaaggactgggaccc |
| | taagaagtacggcggcttcgacagccccac |
| | cgtggcctattctgtgctggtggtggccaa |
| | agtggaaaagggcaagtccaagaaactgaa |
| | gagtgtgaaagagctgctggggatcaccat |
| | catggaaagaagcagcttcgagaagaatcc |
| | catcgactttctggaagccaagggctacaa |
| | agaagtgaaaaaggacctgatcatcaagct |
| | gcctaagtactccctgttcgagctggaaaa |
| | cggccggaagagaatgctggcctctgccgg |
| | cgaactgcagaagggaaacgaactggccct |
| | gccctccaaatatgtgaacttcctgtacct |
| | ggccagccactatgagaagctgaagggctc |
| | ccccgaggataatgagcagaaacagctgtt |
| | tgtggaacagcacaagcactacctggacga |
| | gatcatcgagcagatcagcgagttctccaa |
| | gagagtgatcctggccgacgctaatctgga |
| | caaagtgctgtccgcctacaacaagcaccg |
| | ggataagcccatcagagagcaggccgagaa |
| | tatcatccacctgtttaccctgaccaatct |
| | gggagcccctgccgccttcaagtactttga |
| | caccaccatcgaccggaagaggtacaccag |
| | caccaaagaggtgctggacgccaccctgat |
| | ccaccagagcatcaccggcctgtacgagac |
| | acggatcgacctgtctcagctgggaggcga |
| | caagcgacctgccgccacaaagaaggctgg |
| | acaggctaagaagaagaaagattacaaaga |
| | cgatgacgataagGGTtccGGCgctactaa |
| | cttcagcctgctgaagcaggctggggacgt |
| | ggaggagaaccctggacctaggACGCGTtt |
| | gagcaagggcgaggaggacaacatggccat |
| | catcaaggagttcatgcgcttcaaggtgca |
| | catggagggctccgtgaacggccacgagtt |
| | cgagatcgagggcgagggcgagggccgccc |
| | ctacgagggcacccagaccgccaagctgaa |
| | ggtgaccaagggcggccccctgcccttcgc |
| | ctgggacatcctgtcccctcagttcatgta |
| | cggctccaaggcctacgtgaagcaccccgc |
| | cgacatccccgactacttgaagctgtcctt |
| | ccccgagggcttcaagtgggagcgcgtgat |
| | gaacttcgaggacggcggcgtggtgaccgt |
| | gacccaggactcctccctgcaggacggcga |
| | gttcatctacaaggtgaagctgcgcggcac |
| | caacttcccctccgacggccccgtaatgca |
| | gaagaagaccatgggctgggaggcctcctc |
| | cgagcggatgtaccccgaggacggcgccct |
| | gaagggcgagatcaagcagaggctgaagct |
| | gaaggacggcggccactacgacgccgaggt |
| | caagaccacctacaaggccaagaagcccgt |
| | gcagctgcccggcgcctacaacgtcaacat |
| | caagctggacatcacctcccacaacgagga |
| | ctacaccatcgtggaacagtacgagcgcgc |
| | cgagggccgccactccaccggcggcatgga |
| | cgagctgtacaagtaaATCGATATCGGGCT |
| | AGCgtcgacaatcaacctctggattacaaa |
| | atttgtgaaagattgactggtattcttaac |
| | tatgttgctccttttacgctatgtggatac |
| | gctgctttaatgcctttgtatcatgctatt |
| | gcttcccgtatggctttcattttctcctcc |
| | ttgtataaatcctggttgctgtctctttat |
| | gaggagttgtggcccgttgtcaggcaacgt |
| | ggcgtggtgtgcactgtgtttgctgacgca |
| | acccccactggttggggcattgccaccacc |
| | tgtcagctcctttccgggactttcgctttc |
| | cccctccctattgccacggcggaactcatc |
| | gccgcctgccttgcccgctgctggacaggg |
| | gctcggctgttgggcactgacaattccgtg |
| | gtgttgtcggggaagctgacgtcctttcca |
| | tggctgctcgcctgtgttgccacctggatt |
| | ctgcgcgggacgtccttctgctacgtccct |
| | tcggccctcaatccagcggaccttccttcc |
| | cgcggcctgctgccggctctgcggcctctt |
| | ccgcgtcttcgccttcgccctcagacgagt |
| | cggatctccctttgggccgcctccccgcct |
| | ggaattcgagctcggtacctttaagaccaa |
| | tgacttacaaggcagctgtagatcttagcc |
| | actttttaaaagaaaaggggggactggaag |
| | ggctaattcactcccaacgaagacaagatc |
| | tgctttttgcttgtactgggtctctctggt |
| | tagaccagatctgagcctgggagctctctg |
| | gctaactagggaacccactgcttaagcctc |
| | aataaagcttgccttgagtgcttcaagtag |
| | tgtgtgcccgtctgttgtgtgactctggta |
| | actagagatccctcagacccttttagtcag |
| | tgtggaaaatctctagcagtagtagttcat |
| | gtcatcttattattcagtatttataacttg |
| | caaagaaatgaatatcagagagtgagagga |
| | acttgtttattgcagcttataatggttaca |
| | aataaagcaatagcatcacaaatttcacaa |
| | ataaagcatttttttcactgcattctagtt |
| | gtggtttgtccaaactcatcaatgtatctt |
| | atcatgtctggctctagctatcccgcccct |
| | aactccgcccatcccgcccctaactccgcc |
| | cagttccgcccattctccgccccatggctg |
| | actaattttttttatttatgcagaggccga |
| | ggccgcctcggcctctgagctattccagaa |
| | gtagtgaggaggcttttttggaggcctagg |
| | gacgtacccaattcgcCCTATAGTGAGTCG |
| | TATTAcgcgcgctcactggccgtcgtttta |
| | caacgtcgtgactgggaaaaccctggcgtt |
| | acccaacttaatcgccttgcagcacatccc |
| | cctttcgccagctggcgtaatagcgaagag |
| | gcccgcaccgatcgcccttcccaacagttg |
| | cgcagcctgaatggcgaatgggacgcgccc |
| | tgtagcggcgcattaagcgcggcgggtgtg |
| | gtggttacgcgcagcgtgaccgctacactt |
| | gccagcgccctagcgcccgctcctttcgct |
| | ttcttcccttcctttctcgccacgttcgcc |
| | ggctttccccgtcaagctctaaatcggggg |
| | ctccctttagggttccgatttagtgcttta |
| | cggcacctcgaccccaaaaaacttgattag |
| | ggtgatggttcacgtagtgggccatcgccc |
| | tgatagacggtttttcgccctttgacgttg |
| | gagtccacgttctttaatagtggactcttg |
| | ttccaaactggaacaacactcaaccctatc |
| | tcggtctattcttttgatttataagggatt |
| | ttgccgatttcggcctattggttaaaaaat |
| | gagctgatttaacaaaaatttaacgcgaat |
| | tttaacaaaatattaacgcttacaatttag |
| | gtggcacttttcggggaaatgtgcgcggaa |
| | cccctatttgtttatttttctaaatacatt |
| | caaatatgtatccgctcatgagacaataac |
| | cctgataaatgcttcaataatattgaaaaa |
| | ggaagagtatgagtattcaacatttccgtg |
| | tcgcccttattcccttttttgcggcatttt |
| | gccttcctgtttttgctcacccagaaacgc |
| | tggtgaaagtaaaagatgctgaagatcagt |
| | tgggtgcacgagtgggttacatcgaactgg |
| | atctcaacagcggtaagatccttgagagtt |
| | ttcgccccgaagaacgttttccaatgatga |
| | gcacttttaaagttctgctatgtggcgcgg |
| | tattatcccgtattgacgccgggcaagagc |
| | aactcggtcgccgcatacactattctcaga |
| | atgacttggttgagtactcaccagtcacag |
| | aaaagcatcttacggatggcatgacagtaa |
| | gagaattatgcagtgctgccataaccatga |
| | gtgataacactgcggccaacttacttctga |
| | caacgatcggaggaccgaaggagctaaccg |
| | cttttttgcacaacatgggggatcatgtaa |
| | ctcgccttgatcgttgggaaccggagctga |
| | atgaagccataccaaacgacgagcgtgaca |
| | ccacgatgcctgtagcaatggcaacaacgt |
| | tgcgcaaactattaactggcgaactactta |
| | ctctagcttcccggcaacaattaatagact |
| | ggatggaggcggataaagttgcaggaccac |
| | ttctgcgctcggcccttccggctggctggt |
| | ttattgctgataaatctggagccggtgagc |
| | gtgggtctcgcggtatcattgcagcactgg |
| | ggccagatggtaagccctcccgtatcgtag |
| | ttatctacacgacggggagtcaggcaacta |
| | tggatgaacgaaatagacagatcgctgaga |
| | taggtgcctcactgattaagcattggtaac |
| | tgtcagaccaagtttactcatatatacttt |
| | agattgatttaaaacttcatttttaattta |
| | aaaggatctaggtgaagatcctttttgata |
| | atctcatgaccaaaatcccttaacgtgagt |
| | tttcgttccactgagcgtcagaccccgtag |
| | aaaagatcaaaggatcttcttgagatcctt |
| | tttttctgcgcgtaatctgctgcttgcaaa |
| | caaaaaaaccaccgctaccagcggtggttt |
| | gtttgccggatcaagagctaccaactcttt |
| | ttccgaaggtaactggcttcagcagagcgc |
| | agataccaaatactgttcttctagtgtagc |
| | cgtagttaggccaccacttcaagaactctg |
| | tagcaccgcctacatacctcgctctgctaa |
| | tcctgttaccagtggctgctgccagtggcg |
| | ataagtcgtgtcttaccgggttggactcaa |
| | gacgatagttaccggataaggcgcagcggt |
| | cgggctgaacggggggttcgtgcacacagc |
| | ccagcttggagcgaacgacctacaccgaac |
| | tgagatacctacagcgtgagctatgagaaa |
| | gcgccacgcttcccgaagggagaaaggcgg |
| | acaggtatccggtaagcggcagggtcggaa |
| | caggagagcgcacgagggagcttccagggg |
| | gaaacgcctggtatctttatagtcctgtcg |
| | ggtttcgccacctctgacttgagcgtcgat |
| | ttttgtgatgctcgtcaggggggcggagcc |
| | tatggaaaaacgccagcaacgcggcctttt |
| | tacggttcctggccttttgctggccttttg |
| | ctcacatgttctttcctgcgttatcccctg |
| | attctgtggataaccgtattaccgcctttg |
| | agtgagctgataccgctcgccgcagccgaa |
| | cgaccgagcgcagcgagtcagtgagcgagg |
| | aagcggaagagcgcccaatacgcaaaccgc |
| | ctctccccgcgcgttggccgattcattaat |
| | gcagctggcacgacaggtttcccgactgga |
| | aagcgggcagtgagcgcaacgcaattaatg |
| | tgagttagctcactcattaggcaccccagg |
| | ctttacactttatgcttccggctcgtatgt |
| | tgtgtggaattgtgagcggataacaatttc |
| | acacaggaaacagctatgaccatgattacg |
| | ccaagcgcgcaattaaccctcactaaaggg |
| | aacaaaagctggagctgcaagctta |
| (SEQ ID NO: 40) | |
| OT-Cas9-025 | ctgtttttgctcacccagaaacgctggtga |
| | aagtaaaagatgctgaagatcagttgggtg |
| | cacgagtgggttacatcgaactggatctca |
| | acagcggtaagatccttgagagttttcgcc |
| | ccgaagaacgttttccaatgatgagcactt |
| | ttaaagttctgctatgtggcgcggtattat |
| | cccgtattgacgccgggcaagagcaactcg |
| | gtcgccgcatacactattctcagaatgact |
| | tggttgagtactcaccagtcacagaaaagc |
| | atcttacggatggcatgacagtaagagaat |
| | tatgcagtgctgccataaccatgagtgata |
| | acactgcggccaacttacttctgacaacga |
| | tcggaggaccgaaggagctaaccgcttttt |
| | tgcacaacatgggggatcatgtaactcgcc |
| | ttgatcgttgggaaccggagctgaatgaag |
| | ccataccaaacgacgagcgtgacaccacga |
| | tgcctgtagcaatggcaacaacgttgcgca |
| | aactattaactggcgaactacttactctag |
| | cttcccggcaacaattaatagactggatgg |
| | aggcggataaagttgcaggaccacttctgc |
| | gctcggcccttccggctggctggtttattg |
| | ctgataaatctggagccggtgagcgtgggt |
| | ctcgcggtatcattgcagcactggggccag |
| | atggtaagccctcccgtatcgtagttatct |
| | acacgacggggagtcaggcaactatggatg |
| | aacgaaatagacagatcgctgagataggtg |
| | cctcactgattaagcattggtaactgtcag |
| | accaagtttactcatatatactttagattg |
| | atttaaaacttcatttttaatttaaaagga |
| | tctaggtgaagatcctttttgataatctca |
| | tgaccaaaatcccttaacgtgagttttcgt |
| | tccactgagcgtcagaccccgtagaaaaga |
| | tcaaaggatcttcttgagatcctttttttc |
| | tgcgcgtaatctgctgcttgcaaacaaaaa |
| | aaccaccgctaccagcggtggtttgtttgc |
| | cggatcaagagctaccaactctttttccga |
| | aggtaactggcttcagcagagcgcagatac |
| | caaatactgttcttctagtgtagccgtagt |
| | taggccaccacttcaagaactctgtagcac |
| | cgcctacatacctcgctctgctaatcctgt |
| | taccagtggctgctgccagtggcgataagt |
| | cgtgtcttaccgggttggactcaagacgat |
| | agttaccggataaggcgcagcggtcgggct |
| | gaacggggggttcgtgcacacagcccagct |
| | tggagcgaacgacctacaccgaactgagat |
| | acctacagcgtgagctatgagaaagcgcca |
| | cgcttcccgaagggagaaaggcggacaggt |
| | atccggtaagcggcagggtcggaacaggag |
| | agcgcacgagggagcttccagggggaaacg |
| | cctggtatctttatagtcctgtcgggtttc |
| | gccacctctgacttgagcgtcgatttttgt |
| | gatgctcgtcaggggggcggagcctatgga |
| | aaaacgccagcaacgcggcctttttacggt |
| | tcctggccttttgctggccttttgctcaca |
| | tgttctttcctgcgttatcccctgattctg |
| | tggataaccgtattaccgcctttgagtgag |
| | ctgataccgctcgccgcagccgaacgaccg |
| | agcgcagcgagtcagtgagcgaggaagcgg |
| | aagagcgcccaatacgcaaaccgcctctcc |
| | ccgcgcgttggccgattcattaatgcagct |
| | ggcacgacaggtttcccgactggaaagcgg |
| | gcagtgagcgcaacgcaattaatgtgagtt |
| | agctcactcattaggcaccccaggctttac |
| | actttatgcttccggctcgtatgttgtgtg |
| | gaattgtgagcggataacaatttcacacag |
| | gaaacagctatgaccatgattacgccaagc |
| | gcgcaattaaccctcactaaagggaacaaa |
| | agctggagctgcaagcttagacattgatta |
| | ttgactagttattaatagtaatcaattacg |
| | gggtcattagttcatagcccatatatggag |
| | ttccgcgttacataacttacggtaaatggc |
| | ccgcctggctgaccgcccaacgacccccgc |
| | ccattgacgtcaataatgacgtatgttccc |
| | atagtaacgccaatagggactttccattga |
| | cgtcaatgggtggagtatttacggtaaact |
| | gcccacttggcagtacatcaagtgtatcat |
| | atgccaagtacgccccctattgacgtcaat |
| | gacggtaaatggcccgcctggcattatgcc |
| | cagtacatgaccttatgggactttcctact |
| | tggcagtacatctacgtattagtcatcgct |
| | attaccatggtgatgcggttttggcagtac |
| | atcaatgggcgtggatagcggtttgactca |
| | cggggatttccaagtctccaccccattgac |
| | gtcaatgggagtttgttttggcaccaaaat |
| | caacgggactttccaaaatgtcgtaacaac |
| | tccgccccattgacgcaaatgggcggtagg |
| | cgtgtacggtgggaggtctatataagcagc |
| | gcgttttgcctgtactgggtctctctggtt |
| | agaccagatctgagcctgggagctctctgg |
| | ctaactagggaacccactgcttaagcctca |
| | ataaagcttgccttgagtgcttcaagtagt |
| | gtgtgcccgtctgttgtgtgactctggtaa |
| | ctagagatccctcagacccttttagtcagt |
| | gtggaaaatctctagcagtggcgcccgaac |
| | agggacttgaaagcgaaagggaaaccagag |
| | gagctctctcgacgcaggactcggcttgct |
| | gaagcgcgcacggcaagaggcgaggggcgg |
| | cgactggtgagtacgccaaaaattttgact |
| | agcggaggctagaaggagagagatgggtgc |
| | gagagcgtcagtattaagcgggggagaatt |
| | agatcgcgatgggaaaaaattcggttaagg |
| | ccagggggaaagaaaaaatataaattaaaa |
| | catatagtatgggcaagcagggagctagaa |
| | cgattcgcagttaatcctggcctgttagaa |
| | acatcagaaggctgtagacaaatactggga |
| | cagctacaaccatcccttcagacaggatca |
| | gaagaacttagatcattatataatacagta |
| | gcaaccctctattgtgtgcatcaaaggata |
| | gagataaaagacaccaaggaagctttagac |
| | aagatagaggaagagcaaaacaaaagtaag |
| | accaccgcacagcaagcggccgctgatctt |
| | cagacctggaggaggagatatgagggacaa |
| | ttggagaagtgaattatataaatataaagt |
| | agtaaaaattgaaccattaggagtagcacc |
| | caccaaggcaaagagaagagtggtgcagag |
| | agaaaaaagagcagtgggaataggagcttt |
| | gttccttgggttcttgggagcagcaggaag |
| | cactatgggcgcagcgtcaatgacgctgac |
| | ggtacaggccagacaattattgtctggtat |
| | agtgcagcagcagaacaatttgctgagggc |
| | tattgaggcgcaacagcatctgttgcaact |
| | cacagtctggggcatcaagcagctccaggc |
| | aagaatcctggctgtggaaagatacctaaa |
| | ggatcaacagctcctggggatttggggttg |
| | ctctggaaaactcatttgcaccactgctgt |
| | gccttggaatgctagttggagtaataaatc |
| | tctggaacagatttggaatcacacgacctg |
| | gatggagtgggacagagaaattaacaatta |
| | cacaagcttaatacactccttaattgaaga |
| | atcgcaaaaccagcaagaaaagaatgaaca |
| | agaattattggaattagataaatgggcaag |
| | tttgtggaattggtttaacataacaaattg |
| | gctgtggtatataaaattattcataatgat |
| | agtaggaggcttggtaggtttaagaatagt |
| | ttttgctgtactttctatagtgaatagagt |
| | taggcagggatattcaccattatcgtttca |
| | gacccacctcccaaccccgaggggacccga |
| | caggcccgaaggaatagaagaagaaggtgg |
| | agagagagacagagacagatccattcgatt |
| | agtgaacggatctcgacggtatcgattaga |
| | ctgtagcccaggaatatggcagctagattg |
| | tacacatttagaaggaaaagttatcttggt |
| | agcagttcatgtagccagtggatatataga |
| | agcagaagtaattccagcagagacagggca |
| | agaaacagcatacttcctcttaaaattagc |
| | aggaagatggccagtaaaaacagtacatac |
| | agacaatggcagcaatttcaccagtactac |
| | agttaaggccgcctgttggtgggcggggat |
| | caagcaggaatttggcattccctacaatcc |
| | ccaaagtcaaggagtaatagaatctatgaa |
| | taaagaattaaagaaaattataggacaggt |
| | aagagatcaggctgaacatcttaagacagc |
| | agtacaaatggcagtattcatccacaattt |
| | taaaagaaaaggggggattggggggtacag |
| | tgcaggggaaagaatagtagacataatagc |
| | aacagacatacaaactaaagaattacaaaa |
| | acaaattacaaaaattcaaaattttcgggt |
| | ttattacagggacagcagagatccagtttg |
| | gctcgggtttattacagggacagcagagat |
| | ccagtttggttaattaaggtaccgagggcc |
| | tatttcccatgattccttcatatttgcata |
| | tacgatacaaggctgttagagagataatta |
| | gaattaatttgactgtaaacacaaagatat |
| | tagtacaaaatacgtgacgtagaaagtaat |
| | aatttcttgggtagtttgcagttttaaaat |
| | tatgttttaaaatggactatcatatgctta |
| | ccgtaacttgaaagtatttcgatttcttgg |
| | ctttatatatcttgtggaaaggacgaaaGA |
| | GTCCGAGCAGAAGAAGAAgttttagagcta |
| | gaaatagcaagttaaaataaggctagtccg |
| | ttatcaacttgaaaaagtggcaccgagtcg |
| | gtgcttttttgaattcgctagctaggtctt |
| | gaaaggagtgggaattggctccggtgcccg |
| | tcagtgggcagagcgcacatcgcccacagt |
| | ccccgagaagttggggggaggggtcggcaa |
| | ttgatccggtgcctagagaaggtggcgcgg |
| | ggtaaactgggaaagtgatgtcgtgtactg |
| | gctccgcctttttcccgagggtgggggaga |
| | accgtatataagtgcagtagtcgccgtgaa |
| | cgttctttttcgcaacgggtttgccgccag |
| | aacacaggaccggttctagagccaccATGG |
| | GATCCgacaagaagtacagcatcggcctgg |
| | acatcggcaccaactctgtgggctgggccg |
| | tgatcaccgacgagtacaaggtgcccagca |
| | agaaattcaaggtgctgggcaacaccgacc |
| | ggcacagcatcaagaagaacctgatcggag |
| | ccctgctgttcgacagcggcgaaacagccg |
| | aggccacccggctgaagagaaccgccagaa |
| | gaagatacaccagacggaagaaccggatct |
| | gctatctgcaagagatcttcagcaacgaga |
| | tggccaaggtggacgacagcttcttccaca |
| | gactggaagagtccttcctggtggaagagg |
| | ataagaagcacgagcggcaccccatcttcg |
| | gcaacatcgtggacgaggtggcctaccacg |
| | agaagtaccccaccatctaccacctgagaa |
| | agaaactggtggacagcaccgacaaggccg |
| | acctgcggctgatctatctggccctggccc |
| | acatgatcaagttccggggccacttcctga |
| | tcgagggcgacctgaaccccgacaacagcg |
| | acgtggacaagctgttcatccagctggtgc |
| | agacctacaaccagctgttcgaggaaaacc |
| | ccatcaacgccagcggcgtggacgccaagg |
| | ccatcctgtctgccagactgagcaagagca |
| | gacggctggaaaatctgatcgcccagctgc |
| | ccggcgagaagaagaatggcctgttcggaa |
| | acctgattgccctgagcctgggcctgaccc |
| | ccaacttcaagagcaacttcgacctggccg |
| | aggatgccaaactgcagctgagcaaggaca |
| | cctacgacgacgacctggacaacctgctgg |
| | cccagatcggcgaccagtacgccgacctgt |
| | ttctggccgccaagaacctgtccgacgcca |
| | tcctgctgagcgacatcctgagagtgaaca |
| | ccgagatcaccaaggcccccctgagcgcct |
| | ctatgatcaagagatacgacgagcaccacc |
| | aggacctgaccctgctgaaagctctcgtgc |
| | ggcagcagctgcctgagaagtacaaagaga |
| | ttttcttcgaccagagcaagaacggctacg |
| | ccggctacattgacggcggagccagccagg |
| | aagagttctacaagttcatcaagcccatcc |
| | tggaaaagatggacggcaccgaggaactgc |
| | tcgtgaagctgaacagagaggacctgctgc |
| | ggaagcagcggaccttcgacaacggcagca |
| | tcccccaccagatccacctgggagagctgc |
| | acgccattctgcggcggcaggaagattttt |
| | acccattcctgaaggacaaccgggaaaaga |
| | tcgagaagatcctgaccttccgcatcccct |
| | actacgtgggccctctggccaggggaaaca |
| | gcagattcgcctggatgaccagaaagagcg |
| | aggaaaccatcaccccctggaacttcgagg |
| | aagtggtggacaagggcgcttccgcccaga |
| | gcttcatcgagcggatgaccaacttcgata |
| | agaacctgcccaacgagaaggtgctgccca |
| | agcacagcctgctgtacgagtacttcaccg |
| | tgtataacgagctgaccaaagtgaaatacg |
| | tgaccgagggaatgagaaagcccgccttcc |
| | tgagcggcgagcagaaaaaggccatcgtgg |
| | acctgctgttcaagaccaaccggaaagtga |
| | ccgtgaagcagctgaaagaggactacttca |
| | agaaaatcgagtgcttcgactccgtggaaa |
| | tctccggcgtggaagatcggttcaacgcct |
| | ccctgggcacataccacgatctgctgaaaa |
| | ttatcaaggacaaggacttcctggacaatg |
| | aggaaaacgaggacattctggaagatatcg |
| | tgctgaccctgacactgtttgaggacagag |
| | agatgatcgaggaacggctgaaaacctatg |
| | cccacctgttcgacgacaaagtgatgaagc |
| | agctgaagcggcggagatacaccggctggg |
| | gcaggctgagccggaagctgatcaacggca |
| | tccgggacaagcagtccggcaagacaatcc |
| | tggatttcctgaagtccgacggcttcgcca |
| | acagaaacttcatgcagctgatccacgacg |
| | acagcctgacctttaaagaggacatccaga |
| | aagcccaggtgtccggccagggcgatagcc |
| | tgcacgagcacattgccaatctggccggca |
| | gccccgccattaagaagggcatcctgcaga |
| | cagtgaaggtggtggacgagctcgtgaaag |
| | tgatgggccggcacaagcccgagaacatcg |
| | tgatcgaaatggccagagagaaccagacca |
| | cccagaagggacagaagaacagccgcgaga |
| | gaatgaagcggatcgaagagggcatcaaag |
| | agctgggcagccagatcctgaaagaacacc |
| | ccgtggaaaacacccagctgcagaacgaga |
| | agctgtacctgtactacctgcagaatgggc |
| | gggatatgtacgtggaccaggaactggaca |
| | tcaaccggctgtccgactacgatgtggacc |
| | atatcgtgcctcagagctttctgaaggacg |
| | actccatcgacaacaaggtgctgaccagaa |
| | gcgacaagaaccggggcaagagcgacaacg |
| | tgccctccgaagaggtcgtgaagaagatga |
| | agaactactggcggcagctgctgaacgcca |
| | agctgattacccagagaaagttcgacaatc |
| | tgaccaaggccgagagaggcggcctgagcg |
| | aactggataaggccggcttcatcaagagac |
| | agctggtggaaacccggcagatcacaaagc |
| | acgtggcacagatcctggactcccggatga |
| | acactaagtacgacgagaatgacaagctga |
| | tccgggaagtgaaagtgatcaccctgaagt |
| | ccaagctggtgtccgatttccggaaggatt |
| | tccagttttacaaagtgcgcgagatcaaca |
| | actaccaccacgcccacgacgcctacctga |
| | acgccgtcgtgggaaccgccctgatcaaaa |
| | agtaccctaagctggaaagcgagttcgtgt |
| | acggcgactacaaggtgtacgacgtgcgga |
| | agatgatcgccaagagcgagcaggaaatcg |
| | gcaaggctaccgccaagtacttcttctaca |
| | gcaacatcatgaactttttcaagaccgaga |
| | ttaccctggccaacggcgagatccggaagc |
| | ggcctctgatcgagacaaacggcgaaaccg |
| | gggagatcgtgtgggataagggccgggatt |
| | ttgccaccgtgcggaaagtgctgagcatgc |
| | cccaagtgaatatcgtgaaaaagaccgagg |
| | tgcagacaggcggcttcagcaaagagtcta |
| | tcctgcccaagaggaacagcgataagctga |
| | tcgccagaaagaaggactgggaccctaaga |
| | agtacggcggcttcgacagccccaccgtgg |
| | cctattctgtgctggtggtggccaaagtgg |
| | aaaagggcaagtccaagaaactgaagagtg |
| | tgaaagagctgctggggatcaccatcatgg |
| | aaagaagcagcttcgagaagaatcccatcg |
| | actttctggaagccaagggctacaaagaag |
| | tgaaaaaggacctgatcatcaagctgccta |
| | agtactccctgttcgagctggaaaacggcc |
| | ggaagagaatgctggcctctgccggcgaac |
| | tgcagaagggaaacgaactggccctgccct |
| | ccaaatatgtgaacttcctgtacctggcca |
| | gccactatgagaagctgaagggctcccccg |
| | aggataatgagcagaaacagctgtttgtgg |
| | aacagcacaagcactacctggacgagatca |
| | tcgagcagatcagcgagttctccaagagag |
| | tgatcctggccgacgctaatctggacaaag |
| | tgctgtccgcctacaacaagcaccgggata |
| | agcccatcagagagcaggccgagaatatca |
| | tccacctgtttaccctgaccaatctgggag |
| | cccctgccgccttcaagtactttgacacca |
| | ccatcgaccggaagaggtacaccagcacca |
| | aagaggtgctggacgccaccctgatccacc |
| | agagcatcaccggcctgtacgagacacgga |
| | tcgacctgtctcagctgggaggcgacaagc |
| | gacctgccgccacaaagaaggctggacagg |
| | ctaagaagaagaaagattacaaagacgatg |
| | acgataagGGTtccGGCgctactaacttca |
| | gcctgctgaagcaggctggggacgtggagg |
| | agaaccctggacctaggACGCGTttgagca |
| | agggcgaggaggacaacatggccatcatca |
| | aggagttcatgcgcttcaaggtgcacatgg |
| | agggctccgtgaacggccacgagttcgaga |
| | tcgagggcgagggcgagggccgcccctacg |
| | agggcacccagaccgccaagctgaaggtga |
| | ccaagggcggccccctgcccttcgcctggg |
| | acatcctgtcccctcagttcatgtacggct |
| | ccaaggcctacgtgaagcaccccgccgaca |
| | tccccgactacttgaagctgtccttccccg |
| | agggcttcaagtgggagcgcgtgatgaact |
| | tcgaggacggcggcgtggtgaccgtgaccc |
| | aggactcctccctgcaggacggcgagttca |
| | tctacaaggtgaagctgcgcggcaccaact |
| | tcccctccgacggccccgtaatgcagaaga |
| | agaccatgggctgggaggcctcctccgagc |
| | ggatgtaccccgaggacggcgccctgaagg |
| | gcgagatcaagcagaggctgaagctgaagg |
| | acggcggccactacgacgccgaggtcaaga |
| | ccacctacaaggccaagaagcccgtgcagc |
| | tgcccggcgcctacaacgtcaacatcaagc |
| | tggacatcacctcccacaacgaggactaca |
| | ccatcgtggaacagtacgagcgcgccgagg |
| | gccgccactccaccggcggcatggacgagc |
| | tgtacaagtaaATCGATATCGGGCTAGCgt |
| | cgacaatcaacctctggattacaaaatttg |
| | tgaaagattgactggtattcttaactatgt |
| | tgctccttttacgctatgtggatacgctgc |
| | tttaatgcctttgtatcatgctattgcttc |
| | ccgtatggctttcattttctcctccttgta |
| | taaatcctggttgctgtctctttatgagga |
| | gttgtggcccgttgtcaggcaacgtggcgt |
| | ggtgtgcactgtgtttgctgacgcaacccc |
| | cactggttggggcattgccaccacctgtca |
| | gctcctttccgggactttcgctttccccct |
| | ccctattgccacggcggaactcatcgccgc |
| | ctgccttgcccgctgctggacaggggctcg |
| | gctgttgggcactgacaattccgtggtgtt |
| | gtcggggaagctgacgtcctttccatggct |
| | gctcgcctgtgttgccacctggattctgcg |
| | cgggacgtccttctgctacgtcccttcggc |
| | cctcaatccagcggaccttccttcccgcgg |
| | cctgctgccggctctgcggcctcttccgcg |
| | tcttcgccttcgccctcagacgagtcggat |
| | ctccctttgggccgcctccccgcctggaat |
| | tcgagctcggtacctttaagaccaatgact |
| | tacaaggcagctgtagatcttagccacttt |
| | ttaaaagaaaaggggggactggaagggcta |
| | attcactcccaacgaagacaagatctgctt |
| | tttgcttgtactgggtctctctggttagac |
| | cagatctgagcctgggagctctctggctaa |
| | ctagggaacccactgcttaagcctcaataa |
| | agcttgccttgagtgcttcaagtagtgtgt |
| | gcccgtctgttgtgtgactctggtaactag |
| | agatccctcagacccttttagtcagtgtgg |
| | aaaatctctagcagtagtagttcatgtcat |
| | cttattattcagtatttataacttgcaaag |
| | aaatgaatatcagagagtgagaggaacttg |
| | tttattgcagcttataatggttacaaataa |
| | agcaatagcatcacaaatttcacaaataaa |
| | gcatttttttcactgcattctagttgtggt |
| | ttgtccaaactcatcaatgtatcttatcat |
| | gtctggctctagctatcccgcccctaactc |
| | cgcccatcccgcccctaactccgcccagtt |
| | ccgcccattctccgccccatggctgactaa |
| | ttttttttatttatgcagaggccgaggccg |
| | cctcggcctctgagctattccagaagtagt |
| | gaggaggcttttttggaggcctagggacgt |
| | acccaattcgcCCTATAGTGAGTCGTATTA |
| | cgcgcgctcactggccgtcgttttacaacg |
| | tcgtgactgggaaaaccctggcgttaccca |
| | acttaatcgccttgcagcacatcccccttt |
| | cgccagctggcgtaatagcgaagaggcccg |
| | caccgatcgcccttcccaacagttgcgcag |
| | cctgaatggcgaatgggacgcgccctgtag |
| | cggcgcattaagcgcggcgggtgtggtggt |
| | tacgcgcagcgtgaccgctacacttgccag |
| | cgccctagcgcccgctcctttcgctttctt |
| | cccttcctttctcgccacgttcgccggctt |
| | tccccgtcaagctctaaatcgggggctccc |
| | tttagggttccgatttagtgctttacggca |
| | cctcgaccccaaaaaacttgattagggtga |
| | tggttcacgtagtgggccatcgccctgata |
| | gacggtttttcgccctttgacgttggagtc |
| | cacgttctttaatagtggactcttgttcca |
| | aactggaacaacactcaaccctatctcggt |
| | ctattcttttgatttataagggattttgcc |
| | gatttcggcctattggttaaaaaatgagct |
| | gatttaacaaaaatttaacgcgaattttaa |
| | caaaatattaacgcttacaatttaggtggc |
| | acttttcggggaaatgtgcgcggaacccct |
| | atttgtttatttttctaaatacattcaaat |
| | atgtatccgctcatgagacaataaccctga |
| | taaatgcttcaataatattgaaaaaggaag |
| | agtatgagtattcaacatttccgtgtcgcc |
| | cttattcccttttttgcggcattttgcctt |
| | c(SEQ ID NO: 41) |
| OT-ZFHD -073 | atgtagtcttatgcaatactcttgtagtct |
| | tgcaacatggtaacgatgagttagcaacat |
| | gccttacaaggagagaaaaagcaccgtgca |
| | tgccgattggtggaagtaaggtggtacgat |
| | cgtgccttattaggaaggcaacagacgggt |
| | ctgacatggattggacgaaccactgaattg |
| | ccgcattgcagagatattgtatttaagtgc |
| | ctagctcgatacataaacgggtctctctgg |
| | ttagaccagatctgagcctgggagctctct |
| | ggctaactagggaacccactgcttaagcct |
| | caataaagcttgccttgagtgcttcaagta |
| | gtgtgtgcccgtctgttgtgtgactctggt |
| | aactagagatccctcagacccttttagtca |
| | gtgtggaaaatctctagcagtggcgcccga |
| | acagggacttgaaagcgaaagggaaaccag |
| | aggagctctctcgacgcaggactcggcttg |
| | ctgaagcgcgcacggcaagaggcgaggggc |
| | ggcgactggtgagtacgccaaaaattttga |
| | ctagcggaggctagaaggagagagatgggt |
| | gcgagagcgtcagtattaagcgggggagaa |
| | ttagatcgcgatgggaaaaaattcggttaa |
| | ggccagggggaaagaaaaaatataaattaa |
| | aacatatagtatgggcaagcagggagctag |
| | aacgattcgcagttaatcctggcctgttag |
| | aaacatcagaaggctgtagacaaatactgg |
| | gacagctacaaccatcccttcagacaggat |
| | cagaagaacttagatcattatataatacag |
| | tagcaaccctctattgtgtgcatcaaagga |
| | tagagataaaagacaccaaggaagctttag |
| | acaagatagaggaagagcaaaacaaaagta |
| | agaccaccgcacagcaagcggccgctgatc |
| | ttcagacctggaggaggagatatgagggac |
| | aattggagaagtgaattatataaatataaa |
| | gtagtaaaaattgaaccattaggagtagca |
| | cccaccaaggcaaagagaagagtggtgcag |
| | agagaaaaaagagcagtgggaataggagct |
| | ttgttccttgggttcttgggagcagcagga |
| | agcactatgggcgcagcgtcaatgacgctg |
| | acggtacaggccagacaattattgtctggt |
| | atagtgcagcagcagaacaatttgctgagg |
| | gctattgaggcgcaacagcatctgttgcaa |
| | ctcacagtctggggcatcaagcagctccag |
| | gcaagaatcctggctgtggaaagataccta |
| | aaggatcaacagctcctggggatttggggt |
| | tgctctggaaaactcatttgcaccactgct |
| | gtgccttggaatgctagttggagtaataaa |
| | tctctggaacagatttggaatcacacgacc |
| | tggatggagtgggacagagaaattaacaat |
| | tacacaagcttaatacactccttaattgaa |
| | gaatcgcaaaaccagcaagaaaagaatgaa |
| | caagaattattggaattagataaatgggca |
| | agtttgtggaattggtttaacataacaaat |
| | tggctgtggtatataaaattattcataatg |
| | atagtaggaggcttggtaggtttaagaata |
| | gtttttgctgtactttctatagtgaataga |
| | gttaggcagggatattcaccattatcgttt |
| | cagacccacctcccaaccccgaggggaccc |
| | gacaggcccgaaggaatagaagaagaaggt |
| | ggagagagagacagagacagatccattcga |
| | ttagtgaacggatctcgacggtatcgatta |
| | gactgtagcccaggaatatggcagctagat |
| | tgtacacatttagaaggaaaagttatcttg |
| | gtagcagttcatgtagccagtggatatata |
| | gaagcagaagtaattccagcagagacaggg |
| | caagaaacagcatacttcctcttaaaatta |
| | gcaggaagatggccagtaaaaacagtacat |
| | acagacaatggcagcaatttcaccagtact |
| | acagttaaggccgcctgttggtgggcgggg |
| | atcaagcaggaatttggcattccctacaat |
| | ccccaaagtcaaggagtaatagaatctatg |
| | aataaagaattaaagaaaattataggacag |
| | gtaagagatcaggctgaacatcttaagaca |
| | gcagtacaaatggcagtattcatccacaat |
| | tttaaaagaaaaggggggattggggggtac |
| | agtgcaggggaaagaatagtagacataata |
| | gcaacagacatacaaactaaagaattacaa |
| | aaacaaattacaaaaattcaaaattttcgg |
| | gtttattacagggacagcagagatccagtt |
| | tggctgcattgatcaacgcgtagatctcta |
| | gctaatgatgggcgcacgagtaatgatggg |
| | cggacgactaatgatgggcgcacgagtaat |
| | gatgggcgtctagctaatgatgggcgctag |
| | agtaatgatgggcggtagactaatgatggg |
| | cgctccagtaatgatgggcgttctagcTCT |
| | AGAGGGTATATAATGGGGGCCACTAGCTAC |
| | TACCAGAtAGCTTGGTActagaggatcACT |
| | AGTgccaccatgGCACCTAAGaaaAAGAGG |
| | AAGGTTgaacgcccatatgcttgccctgtc |
| | gagtcctgcgatcgccgcttttctcgctcg |
| | gatgagcttacccgccatatccgcatccac |
| | acaggccagaagcccttccagtgtcgaatc |
| | tgcatgcgtaacttcagtcgtagtgaccac |
| | cttaccacccacatccgcacccacacaggc |
| | ggcggccgcaggaggaagaaacgcaccagc |
| | atagagaccaacatccgtgtggccttagag |
| | aagagtttcttggagaatcaaaagcctacc |
| | tcggaagagatcactatgattgctgatcag |
| | ctcaatatggaaaaagaggtgattcgtgtt |
| | tggttctgtaaccgccgccagaaagaaaaa |
| | agaatcaacactagactgggggccttgctt |
| | ggcaacagcacagacccagctgtgttcaca |
| | gacctggcatccGTGgacaactccgagttt |
| | cagcagctgctgaaccagggcatacctgtg |
| | gccccccacacaactgagcccatgctgatg |
| | gagtaccctgaggctataactcgcctagtg |
| | acaggggcccagaggccccccgacccagct |
| | cctgctccactgggggccccggggctcccc |
| | aatggcctcctttcaggagatgaagacttc |
| | tcctccattgcggacatggacttctcagcc |
| | ctgctgagtcagatcagctccggaggtagt |
| | ggtggaggcagtggtGGTTCCCATCACTGG |
| | GGGTACGGCAAACACAACGGACCTGAGCAC |
| | TGGCATAAGGACTTCCCCATTGCCAAGGGA |
| | GAGCGCCAGTCCCCTGTTGACATCGACACT |
| | CATACAGCCAAGTATGACCCTTCCCTGAAG |
| | CCCCTGTCTGTTTCCTATGATCAAGCAACT |
| | TCCCTGAGGATCCTCAACAATGGTCATGCT |
| | TTCAACGTGGAGTTTGATGACTCTCAGGAC |
| | AAAGCAGTGCTCAAGGGAGGACCCCTGGAT |
| | GGCACTTACAGATTGATTCAGTTTCACTTT |
| | CACTGGGGTTCACTTGATGGACAAGGTTCA |
| | GAGCATACTGTGGATAAAAAGAAATATGCT |
| | GCAGAACTTCACTTGGTTCACTGGAACACC |
| | AAATATGGGGATTTTGGGAAAGCTGTGCAG |
| | CAACCTGATGGACTGGCCGTTCTAGGTATT |
| | TTTTTGAAGGTTGGCAGCGCTAAACCGGGC |
| | CATCAGAAAGTTGTTGATGTGCTGGATTCC |
| | ATTAAAACAAAGGGCAAGAGTGCTGACTTC |
| | ACTAACTTCGATCCTCGTGGCCTCCTTCCT |
| | GAATCCCTGGATTACTGGACCTACCCAGGC |
| | TCACTGACCACCCCTCCTCTTCTGGAATGT |
| | GTGACCTGGATTGTGCTCAAGGAACCCATC |
| | AGCGTCAGCAGCGAGCAGGTGTTGAAATTC |
| | CGTAAACTTAACTTCAATGGGGAGGGTGAA |
| | CCCGAAGAACTGATGGTGGACAACTGGCGC |
| | CCAGCTCAGCCACTGAAGAACAGGCAAATC |
| | AAAGCTTCCTTCAAAggatcctgaATCGGG |
| | CTAGCgtcgacaatcaacctctggattaca |
| | aaatttgtgaaagattgactggtattctta |
| | actatgttgctccttttacgctatgtggat |
| | acgctgctttaatgcctttgtatcatgcta |
| | ttgcttcccgtatggctttcattttctcct |
| | ccttgtataaatcctggttgctgtctcttt |
| | atgaggagttgtggcccgttgtcaggcaac |
| | gtggcgtggtgtgcactgtgtttgctgacg |
| | caacccccactggttggggcattgccacca |
| | cctgtcagctcctttccgggactttcgctt |
| | tccccctccctattgccacggcggaactca |
| | tcgccgcctgccttgcccgctgctggacag |
| | gggctcggctgttgggcactgacaattccg |
| | tggtgttgtcggggaagctgacgtcctttc |
| | catggctgctcgcctgtgttgccacctgga |
| | ttctgcgcgggacgtccttctgctacgtcc |
| | cttcggccctcaatccagcggaccttcctt |
| | cccgcggcctgctgccggctctgcggcctc |
| | ttccgcgtcttcgccttcgccctcagacga |
| | gtcggatctccctttgggccgcctccccgc |
| | ctggaattcgagctcggtaccggtgtggaa |
| | agtccccaggctccccagcaggcagaagta |
| | tgcaaagcatgcatctcaattagtcagcaa |
| | ccaggtgtggaaagtccccaggctccccag |
| | caggcagaagtatgcaaagcatgcatctca |
| | attagtcagcaaccatagtcccgcccctaa |
| | ctccgcccatcccgcccctaactccgccca |
| | gttccgcccattctccgccccatggctgac |
| | taattttttttatttatgcagaggccgagg |
| | ccgcctctgcctctgagctattccagaagt |
| | agtgaggaggcttttttggaggcctaggct |
| | tttgcaaaaagctcccgggagcttgtatat |
| | ccattttcggatctgatcagcacTTCGAAG |
| | CCACCATGAACCCAGCCATCAGCGTCGCTC |
| | TCCTGCTCTCAGTCTTGCAGGTGTCCCGAG |
| | GGCAGAAGGTGACCAGCCTGACAGCCTGCC |
| | TGGTGAACCAAAACCTTCGCCTGGACTGCC |
| | GCCATGAGAATAACACCAAGGATAACTCCA |
| | TCCAGCATGAGTTCAGCCTGACCCGAGAGA |
| | AGAGGAAGCACGTGCTCTCAGGCACCCTTG |
| | GGATACCCGAGCACACGTACCGCTCCCGCG |
| | TCACCCTCTCCAACCAGCCCTATATCAAGG |
| | TCCTTACCCTAGCCAACTTCACCACCAAGG |
| | ATGAGGGCGACTACTTTTGTGAGCTTCAAG |
| | TCTCGGGCGCGAATCCCATGAGCTCCAATA |
| | AAAGTATCAGTGTGTATAGAGACAAGCTGG |
| | TCAAGTGTGGCGGCATAAGCCTGCTGGTTC |
| | AGAACACATCCTGGATGCTGCTGCTGCTGC |
| | TTTCCCTCTCCCTCCTCCAAGCCCTGGACT |
| | TCATTTCTCTGTAATTCGAAgcgaattcga |
| | gctcggtacctttaagaccaatgacttaca |
| | aggcagctgtagatcttagccactttttaa |
| | aagaaaaggggggactggaagggctaattc |
| | actcccaacgaagacaagatctgctttttg |
| | cttgtactgggtctctctggttagaccaga |
| | tctgagcctgggagctctctggctaactag |
| | ggaacccactgcttaagcctcaataaagct |
| | tgccttgagtgcttcaagtagtgtgtgccc |
| | gtctgttgtgtgactctggtaactagagat |
| | ccctcagacccttttagtcagtgtggaaaa |
| | tctctagcagtagtagttcatgtcatctta |
| | ttattcagtatttataacttgcaaagaaat |
| | gaatatcagagagtgagaggaacttgttta |
| | ttgcagcttataatggttacaaataaagca |
| | atagcatcacaaatttcacaaataaagcat |
| | ttttttcactgcattctagttgtggtttgt |
| | ccaaactcatcaatgtatcttatcatgtct |
| | ggctctagctatcccgcccctaactccgcc |
| | catcccgcccctaactccgcccagttccgc |
| | ccattctccgccccatggctgactaatttt |
| | ttttatttatgcagaggccgaggccgcctc |
| | ggcctctgagctattccagaagtagtgagg |
| | aggcttttttggaggcctagggacgtaccc |
| | aattcgccctatagtgagtcgtattacgcg |
| | cgctcactggccgtcgttttacaacgtcgt |
| | gactgggaaaaccctggcgttacccaactt |
| | aatcgccttgcagcacatccccctttcgcc |
| | agctggcgtaatagcgaagaggcccgcacc |
| | gatcgcccttcccaacagttgcgcagcctg |
| | aatggcgaatgggacgcgccctgtagcggc |
| | gcattaagcgcggcgggtgtggtggttacg |
| | cgcagcgtgaccgctacacttgccagcgcc |
| | ctagcgcccgctcctttcgctttcttccct |
| | tcctttctcgccacgttcgccggctttccc |
| | cgtcaagctctaaatcgggggctcccttta |
| | gggttccgatttagtgctttacggcacctc |
| | gaccccaaaaaacttgattagggtgatggt |
| | tcacgtagtgggccatcgccctgatagacg |
| | gtttttcgccctttgacgttggagtccacg |
| | ttctttaatagtggactcttgttccaaact |
| | ggaacaacactcaaccctatctcggtctat |
| | tcttttgatttataagggattttgccgatt |
| | tcggcctattggttaaaaaatgagctgatt |
| | taacaaaaatttaacgcgaattttaacaaa |
| | atattaacgcttacaatttaggtggcactt |
| | ttcggggaaatgtgcgcggaacccctattt |
| | gtttatttttctaaatacattcaaatatgt |
| | atccgctcatgagacaataaccctgataaa |
| | tgcttcaataatattgaaaaaggaagagta |
| | tgagtattcaacatttccgtgtcgccctta |
| | ttcccttttttgcggcattttgccttcctg |
| | tttttgctcacccagaaacgctggtgaaag |
| | taaaagatgctgaagatcagttgggtgcac |
| | gagtgggttacatcgaactggatctcaaca |
| | gcggtaagatccttgagagttttcgccccg |
| | aagaacgttttccaatgatgagcactttta |
| | aagttctgctatgtggcgcggtattatccc |
| | gtattgacgccgggcaagagcaactcggtc |
| | gccgcatacactattctcagaatgacttgg |
| | ttgagtactcaccagtcacagaaaagcatc |
| | ttacggatggcatgacagtaagagaattat |
| | gcagtgctgccataaccatgagtgataaca |
| | ctgcggccaacttacttctgacaacgatcg |
| | gaggaccgaaggagctaaccgcttttttgc |
| | acaacatgggggatcatgtaactcgccttg |
| | atcgttgggaaccggagctgaatgaagcca |
| | taccaaacgacgagcgtgacaccacgatgc |
| | ctgtagcaatggcaacaacgttgcgcaaac |
| | tattaactggcgaactacttactctagctt |
| | cccggcaacaattaatagactggatggagg |
| | cggataaagttgcaggaccacttctgcgct |
| | cggcccttccggctggctggtttattgctg |
| | ataaatctggagccggtgagcgtgggtctc |
| | gcggtatcattgcagcactggggccagatg |
| | gtaagccctcccgtatcgtagttatctaca |
| | cgacggggagtcaggcaactatggatgaac |
| | gaaatagacagatcgctgagataggtgcct |
| | cactgattaagcattggtaactgtcagacc |
| | aagtttactcatatatactttagattgatt |
| | taaaacttcatttttaatttaaaaggatct |
| | aggtgaagatcctttttgataatctcatga |
| | ccaaaatcccttaacgtgagttttcgttcc |
| | actgagcgtcagaccccgtagaaaagatca |
| | aaggatcttcttgagatcctttttttctgc |
| | gcgtaatctgctgcttgcaaacaaaaaaac |
| | caccgctaccagcggtggtttgtttgccgg |
| | atcaagagctaccaactctttttccgaagg |
| | taactggcttcagcagagcgcagataccaa |
| | atactgttcttctagtgtagccgtagttag |
| | gccaccacttcaagaactctgtagcaccgc |
| | ctacatacctcgctctgctaatcctgttac |
| | cagtggctgctgccagtggcgataagtcgt |
| | gtcttaccgggttggactcaagacgatagt |
| | taccggataaggcgcagcggtcgggctgaa |
| | cggggggttcgtgcacacagcccagcttgg |
| | agcgaacgacctacaccgaactgagatacc |
| | tacagcgtgagctatgagaaagcgccacgc |
| | ttcccgaagggagaaaggcggacaggtatc |
| | cggtaagcggcagggtcggaacaggagagc |
| | gcacgagggagcttccagggggaaacgcct |
| | ggtatctttatagtcctgtcgggtttcgcc |
| | acctctgacttgagcgtcgatttttgtgat |
| | gctcgtcaggggggcggagcctatggaaaa |
| | acgccagcaacgcggcctttttacggttcc |
| | tggccttttgctggccttttgctcacatgt |
| | tctttcctgcgttatcccctgattctgtgg |
| | ataaccgtattaccgcctttgagtgagctg |
| | ataccgctcgccgcagccgaacgaccgagc |
| | gcagcgagtcagtgagcgaggaagcggaag |
| | agcgcccaatacgcaaaccgcctctccccg |
| | cgcgttggccgattcattaatgcagctggc |
| | acgacaggtttcccgactggaaagcgggca |
| | gtgagcgcaacgcaattaatgtgagttagc |
| | tcactcattaggcaccccaggctttacact |
| | ttatgcttccggctcgtatgttgtgtggaa |
| | ttgtgagcggataacaatttcacacaggaa |
| | acagctatgaccatgattacgccaagcgcg |
| | caattaaccctcactaaagggaacaaaagc |
| | tggagctgcaagctta(SEQ ID NO: 42) |
| OT-ZFHD-074 | atgtagtcttatgcaatactcttgtagtct |
| | tgcaacatggtaacgatgagttagcaacat |
| | gccttacaaggagagaaaaagcaccgtgca |
| | tgccgattggtggaagtaaggtggtacgat |
| | cgtgccttattaggaaggcaacagacgggt |
| | ctgacatggattggacgaaccactgaattg |
| | ccgcattgcagagatattgtatttaagtgc |
| | ctagctcgatacataaacgggtctctctgg |
| | ttagaccagatctgagcctgggagctctct |
| | ggctaactagggaacccactgcttaagcct |
| | caataaagcttgccttgagtgcttcaagta |
| | gtgtgtgcccgtctgttgtgtgactctggt |
| | aactagagatccctcagacccttttagtca |
| | gtgtggaaaatctctagcagtggcgcccga |
| | acagggacttgaaagcgaaagggaaaccag |
| | aggagctctctcgacgcaggactcggcttg |
| | ctgaagcgcgcacggcaagaggcgaggggc |
| | ggcgactggtgagtacgccaaaaattttga |
| | ctagcggaggctagaaggagagagatgggt |
| | gcgagagcgtcagtattaagcgggggagaa |
| | ttagatcgcgatgggaaaaaattcggttaa |
| | ggccagggggaaagaaaaaatataaattaa |
| | aacatatagtatgggcaagcagggagctag |
| | aacgattcgcagttaatcctggcctgttag |
| | aaacatcagaaggctgtagacaaatactgg |
| | gacagctacaaccatcccttcagacaggat |
| | cagaagaacttagatcattatataatacag |
| | tagcaaccctctattgtgtgcatcaaagga |
| | tagagataaaagacaccaaggaagctttag |
| | acaagatagaggaagagcaaaacaaaagta |
| | agaccaccgcacagcaagcggccgctgatc |
| | ttcagacctggaggaggagatatgagggac |
| | aattggagaagtgaattatataaatataaa |
| | gtagtaaaaattgaaccattaggagtagca |
| | cccaccaaggcaaagagaagagtggtgcag |
| | agagaaaaaagagcagtgggaataggagct |
| | ttgttccttgggttcttgggagcagcagga |
| | agcactatgggcgcagcgtcaatgacgctg |
| | acggtacaggccagacaattattgtctggt |
| | atagtgcagcagcagaacaatttgctgagg |
| | gctattgaggcgcaacagcatctgttgcaa |
| | ctcacagtctggggcatcaagcagctccag |
| | gcaagaatcctggctgtggaaagataccta |
| | aaggatcaacagctcctggggatttggggt |
| | tgctctggaaaactcatttgcaccactgct |
| | gtgccttggaatgctagttggagtaataaa |
| | tctctggaacagatttggaatcacacgacc |
| | tggatggagtgggacagagaaattaacaat |
| | tacacaagcttaatacactccttaattgaa |
| | gaatcgcaaaaccagcaagaaaagaatgaa |
| | caagaattattggaattagataaatgggca |
| | agtttgtggaattggtttaacataacaaat |
| | tggctgtggtatataaaattattcataatg |
| | atagtaggaggcttggtaggtttaagaata |
| | gtttttgctgtactttctatagtgaataga |
| | gttaggcagggatattcaccattatcgttt |
| | cagacccacctcccaaccccgaggggaccc |
| | gacaggcccgaaggaatagaagaagaaggt |
| | ggagagagagacagagacagatccattcga |
| | ttagtgaacggatctcgacggtatcgatta |
| | gactgtagcccaggaatatggcagctagat |
| | tgtacacatttagaaggaaaagttatcttg |
| | gtagcagttcatgtagccagtggatatata |
| | gaagcagaagtaattccagcagagacaggg |
| | caagaaacagcatacttcctcttaaaatta |
| | gcaggaagatggccagtaaaaacagtacat |
| | acagacaatggcagcaatttcaccagtact |
| | acagttaaggccgcctgttggtgggcgggg |
| | atcaagcaggaatttggcattccctacaat |
| | ccccaaagtcaaggagtaatagaatctatg |
| | aataaagaattaaagaaaattataggacag |
| | gtaagagatcaggctgaacatcttaagaca |
| | gcagtacaaatggcagtattcatccacaat |
| | tttaaaagaaaaggggggattggggggtac |
| | agtgcaggggaaagaatagtagacataata |
| | gcaacagacatacaaactaaagaattacaa |
| | aaacaaattacaaaaattcaaaattttcgg |
| | gtttattacagggacagcagagatccagtt |
| | tggctgcattgatcaacgcgtagatctcta |
| | gctaatgatgggcgcacgagtaatgatggg |
| | cggacgactaatgatgggcgcacgagtaat |
| | gatgggcgtctagctaatgatgggcgctag |
| | agtaatgatgggcggtagactaatgatggg |
| | cgctccagtaatgatgggcgttctagcTCT |
| | AGATAGGCGTGTACGGTGGGAGGCCTATAT |
| | AAGCAGAGCTCGTTTAGTGAACCGTCAGAT |
| | CGCCTGGACTAGCTACTACCAGAtAGCTTG |
| | GTActagaggatcACTAGTgccaccatgGC |
| | ACCTAAGaaaAAGAGGAAGGTTgaacgccc |
| | atatgcttgccctgtcgagtcctgcgatcg |
| | ccgcttttctcgctcggatgagcttacccg |
| | ccatatccgcatccacacaggccagaagcc |
| | cttccagtgtcgaatctgcatgcgtaactt |
| | cagtcgtagtgaccaccttaccacccacat |
| | ccgcacccacacaggcggcggccgcaggag |
| | gaagaaacgcaccagcatagagaccaacat |
| | ccgtgtggccttagagaagagtttcttgga |
| | gaatcaaaagcctacctcggaagagatcac |
| | tatgattgctgatcagctcaatatggaaaa |
| | agaggtgattcgtgtttggttctgtaaccg |
| | ccgccagaaagaaaaaagaatcaacactag |
| | actgggggccttgcttggcaacagcacaga |
| | cccagctgtgttcacagacctggcatccGT |
| | Ggacaactccgagtttcagcagctgctgaa |
| | ccagggcatacctgtggccccccacacaac |
| | tgagcccatgctgatggagtaccctgaggc |
| | tataactcgcctagtgacaggggcccagag |
| | gccccccgacccagctcctgctccactggg |
| | ggccccggggctccccaatggcctcctttc |
| | aggagatgaagacttctcctccattgcgga |
| | catggacttctcagccctgctgagtcagat |
| | cagctccggaggtagtggtggaggcagtgg |
| | tGGTTCCCATCACTGGGGGTACGGCAAACA |
| | CAACGGACCTGAGCACTGGCATAAGGACTT |
| | CCCCATTGCCAAGGGAGAGCGCCAGTCCCC |
| | TGTTGACATCGACACTCATACAGCCAAGTA |
| | TGACCCTTCCCTGAAGCCCCTGTCTGTTTC |
| | CTATGATCAAGCAACTTCCCTGAGGATCCT |
| | CAACAATGGTCATGCTTTCAACGTGGAGTT |
| | TGATGACTCTCAGGACAAAGCAGTGCTCAA |
| | GGGAGGACCCCTGGATGGCACTTACAGATT |
| | GATTCAGTTTCACTTTCACTGGGGTTCACT |
| | TGATGGACAAGGTTCAGAGCATACTGTGGA |
| | TAAAAAGAAATATGCTGCAGAACTTCACTT |
| | GGTTCACTGGAACACCAAATATGGGGATTT |
| | TGGGAAAGCTGTGCAGCAACCTGATGGACT |
| | GGCCGTTCTAGGTATTTTTTTGAAGGTTGG |
| | CAGCGCTAAACCGGGCCATCAGAAAGTTGT |
| | TGATGTGCTGGATTCCATTAAAACAAAGGG |
| | CAAGAGTGCTGACTTCACTAACTTCGATCC |
| | TCGTGGCCTCCTTCCTGAATCCCTGGATTA |
| | CTGGACCTACCCAGGCTCACTGACCACCCC |
| | TCCTCTTCTGGAATGTGTGACCTGGATTGT |
| | GCTCAAGGAACCCATCAGCGTCAGCAGCGA |
| | GCAGGTGTTGAAATTCCGTAAACTTAACTT |
| | CAATGGGGAGGGTGAACCCGAAGAACTGAT |
| | GGTGGACAACTGGCGCCCAGCTCAGCCACT |
| | GAAGAACAGGCAAATCAAAGCTTCCTTCAA |
| | AggatcctgaATCGGGCTAGCgtcgacaat |
| | caacctctggattacaaaatttgtgaaaga |
| | ttgactggtattcttaactatgttgctcct |
| | tttacgctatgtggatacgctgctttaatg |
| | cctttgtatcatgctattgcttcccgtatg |
| | gctttcattttctcctccttgtataaatcc |
| | tggttgctgtctctttatgaggagttgtgg |
| | cccgttgtcaggcaacgtggcgtggtgtgc |
| | actgtgtttgctgacgcaacccccactggt |
| | tggggcattgccaccacctgtcagctcctt |
| | tccgggactttcgctttccccctccctatt |
| | gccacggcggaactcatcgccgcctgcctt |
| | gcccgctgctggacaggggctcggctgttg |
| | ggcactgacaattccgtggtgttgtcgggg |
| | aagctgacgtcctttccatggctgctcgcc |
| | tgtgttgccacctggattctgcgcgggacg |
| | tccttctgctacgtcccttcggccctcaat |
| | ccagcggaccttccttcccgcggcctgctg |
| | ccggctctgcggcctcttccgcgtcttcgc |
| | cttcgccctcagacgagtcggatctccctt |
| | tgggccgcctccccgcctggaattcgagct |
| | cggtaccggtgtggaaagtccccaggctcc |
| | ccagcaggcagaagtatgcaaagcatgcat |
| | ctcaattagtcagcaaccaggtgtggaaag |
| | tccccaggctccccagcaggcagaagtatg |
| | caaagcatgcatctcaattagtcagcaacc |
| | atagtcccgcccctaactccgcccatcccg |
| | cccctaactccgcccagttccgcccattct |
| | ccgccccatggctgactaattttttttatt |
| | tatgcagaggccgaggccgcctctgcctct |
| | gagctattccagaagtagtgaggaggcttt |
| | tttggaggcctaggcttttgcaaaaagctc |
| | ccgggagcttgtatatccattttcggatct |
| | gatcagcacTTCGAAGCCACCATGAACCCA |
| | GCCATCAGCGTCGCTCTCCTGCTCTCAGTC |
| | TTGCAGGTGTCCCGAGGGCAGAAGGTGACC |
| | AGCCTGACAGCCTGCCTGGTGAACCAAAAC |
| | CTTCGCCTGGACTGCCGCCATGAGAATAAC |
| | ACCAAGGATAACTCCATCCAGCATGAGTTC |
| | AGCCTGACCCGAGAGAAGAGGAAGCACGTG |
| | CTCTCAGGCACCCTTGGGATACCCGAGCAC |
| | ACGTACCGCTCCCGCGTCACCCTCTCCAAC |
| | CAGCCCTATATCAAGGTCCTTACCCTAGCC |
| | AACTTCACCACCAAGGATGAGGGCGACTAC |
| | TTTTGTGAGCTTCAAGTCTCGGGCGCGAAT |
| | CCCATGAGCTCCAATAAAAGTATCAGTGTG |
| | TATAGAGACAAGCTGGTCAAGTGTGGCGGC |
| | ATAAGCCTGCTGGTTCAGAACACATCCTGG |
| | ATGCTGCTGCTGCTGCTTTCCCTCTCCCTC |
| | CTCCAAGCCCTGGACTTCATTTCTCTGTAA |
| | TTCGAAgcgaattcgagctcggtaccttta |
| | agaccaatgacttacaaggcagctgtagat |
| | cttagccactttttaaaagaaaagggggga |
| | ctggaagggctaattcactcccaacgaaga |
| | caagatctgctttttgcttgtactgggtct |
| | ctctggttagaccagatctgagcctgggag |
| | ctctctggctaactagggaacccactgctt |
| | aagcctcaataaagcttgccttgagtgctt |
| | caagtagtgtgtgcccgtctgttgtgtgac |
| | tctggtaactagagatccctcagacccttt |
| | tagtcagtgtggaaaatctctagcagtagt |
| | agttcatgtcatcttattattcagtattta |
| | taacttgcaaagaaatgaatatcagagagt |
| | gagaggaacttgtttattgcagcttataat |
| | ggttacaaataaagcaatagcatcacaaat |
| | ttcacaaataaagcatttttttcactgcat |
| | tctagttgtggtttgtccaaactcatcaat |
| | gtatcttatcatgtctggctctagctatcc |
| | cgcccctaactccgcccatcccgcccctaa |
| | ctccgcccagttccgcccattctccgcccc |
| | atggctgactaatttatttatttatgcaga |
| | ggccgaggccgcctcggcctctgagctatt |
| | ccagaagtagtgaggaggcttttttggagg |
| | cctagggacgtacccaattcgccctatagt |
| | gagtcgtattacgcgcgctcactggccgtc |
| | gttttacaacgtcgtgactgggaaaaccct |
| | ggcgttacccaacttaatcgccttgcagca |
| | catccccctttcgccagctggcgtaatagc |
| | gaagaggcccgcaccgatcgcccttcccaa |
| | cagttgcgcagcctgaatggcgaatgggac |
| | gcgccctgtagcggcgcattaagcgcggcg |
| | ggtgtggtggttacgcgcagcgtgaccgct |
| | acacttgccagcgccctagcgcccgctcct |
| | ttcgctttcttcccttcctttctcgccacg |
| | ttcgccggctttccccgtcaagctctaaat |
| | cgggggctccctttagggttccgatttagt |
| | gctttacggcacctcgaccccaaaaaactt |
| | gattagggtgatggttcacgtagtgggcca |
| | tcgccctgatagacggtttttcgccctttg |
| | acgttggagtccacgttctttaatagtgga |
| | ctcttgttccaaactggaacaacactcaac |
| | cctatctcggtctattcttttgatttataa |
| | gggattttgccgatttcggcctattggtta |
| | aaaaatgagctgatttaacaaaaatttaac |
| | gcgaattttaacaaaatattaacgcttaca |
| | atttaggtggcacttttcggggaaatgtgc |
| | gcggaacccctatttgtttatttttctaaa |
| | tacattcaaatatgtatccgctcatgagac |
| | aataaccctgataaatgcttcaataatatt |
| | gaaaaaggaagagtatgagtattcaacatt |
| | tccgtgtcgcccttattcccttttttgcgg |
| | cattttgccttcctgtttttgctcacccag |
| | aaacgctggtgaaagtaaaagatgctgaag |
| | atcagttgggtgcacgagtgggttacatcg |
| | aactggatctcaacagcggtaagatccttg |
| | agagttttcgccccgaagaacgttttccaa |
| | tgatgagcacttttaaagttctgctatgtg |
| | gcgcggtattatcccgtattgacgccgggc |
| | aagagcaactcggtcgccgcatacactatt |
| | ctcagaatgacttggttgagtactcaccag |
| | tcacagaaaagcatcttacggatggcatga |
| | cagtaagagaattatgcagtgctgccataa |
| | ccatgagtgataacactgcggccaacttac |
| | ttctgacaacgatcggaggaccgaaggagc |
| | taaccgcttttttgcacaacatgggggatc |
| | atgtaactcgccttgatcgttgggaaccgg |
| | agctgaatgaagccataccaaacgacgagc |
| | gtgacaccacgatgcctgtagcaatggcaa |
| | caacgttgcgcaaactattaactggcgaac |
| | tacttactctagcttcccggcaacaattaa |
| | tagactggatggaggcggataaagttgcag |
| | gaccacttctgcgctcggcccttccggctg |
| | gctggtttattgctgataaatctggagccg |
| | gtgagcgtgggtctcgcggtatcattgcag |
| | cactggggccagatggtaagccctcccgta |
| | tcgtagttatctacacgacggggagtcagg |
| | caactatggatgaacgaaatagacagatcg |
| | ctgagataggtgcctcactgattaagcatt |
| | ggtaactgtcagaccaagtttactcatata |
| | tactttagattgatttaaaacttcattttt |
| | aatttaaaaggatctaggtgaagatccttt |
| | ttgataatctcatgaccaaaatcccttaac |
| | gtgagttttcgttccactgagcgtcagacc |
| | ccgtagaaaagatcaaaggatcttcttgag |
| | atcctttttttctgcgcgtaatctgctgct |
| | tgcaaacaaaaaaaccaccgctaccagcgg |
| | tggtttgtttgccggatcaagagctaccaa |
| | ctctttttccgaaggtaactggcttcagca |
| | gagcgcagataccaaatactgttcttctag |
| | tgtagccgtagttaggccaccacttcaaga |
| | actctgtagcaccgcctacatacctcgctc |
| | tgctaatcctgttaccagtggctgctgcca |
| | gtggcgataagtcgtgtcttaccgggttgg |
| | actcaagacgatagttaccggataaggcgc |
| | agcggtcgggctgaacggggggttcgtgca |
| | cacagcccagcttggagcgaacgacctaca |
| | ccgaactgagatacctacagcgtgagctat |
| | gagaaagcgccacgcttcccgaagggagaa |
| | aggcggacaggtatccggtaagcggcaggg |
| | tcggaacaggagagcgcacgagggagcttc |
| | cagggggaaacgcctggtatctttatagtc |
| | ctgtcgggtttcgccacctctgacttgagc |
| | gtcgatttttgtgatgctcgtcaggggggc |
| | ggagcctatggaaaaacgccagcaacgcgg |
| | cctttttacggttcctggccttttgctggc |
| | cttttgctcacatgttctttcctgcgttat |
| | cccctgattctgtggataaccgtattaccg |
| | cctttgagtgagctgataccgctcgccgca |
| | gccgaacgaccgagcgcagcgagtcagtga |
| | gcgaggaagcggaagagcgcccaatacgca |
| | aaccgcctctccccgcgcgttggccgattc |
| | attaatgcagctggcacgacaggtttcccg |
| | actggaaagcgggcagtgagcgcaacgcaa |
| | ttaatgtgagttagctcactcattaggcac |
| | cccaggctttacactttatgcttccggctc |
| | gtatgttgtgtggaattgtgagcggataac |
| | aatttcacacaggaaacagctatgaccatg |
| | attacgccaagcgcgcaattaaccctcact |
| | aaagggaacaaaagctggagctgcaagctt |
| | a(SEQ ID NO: 43) |
| OT-ZFHD-075 | atgtagtcttatgcaatactcttgtagtct |
| | tgcaacatggtaacgatgagttagcaacat |
| | gccttacaaggagagaaaaagcaccgtgca |
| | tgccgattggtggaagtaaggtggtacgat |
| | cgtgccttattaggaaggcaacagacgggt |
| | ctgacatggattggacgaaccactgaattg |
| | ccgcattgcagagatattgtatttaagtgc |
| | ctagctcgatacataaacgggtctctctgg |
| | ttagaccagatctgagcctgggagctctct |
| | ggctaactagggaacccactgcttaagcct |
| | caataaagcttgccttgagtgcttcaagta |
| | gtgtgtgcccgtctgttgtgtgactctggt |
| | aactagagatccctcagacccttttagtca |
| | gtgtggaaaatctctagcagtggcgcccga |
| | acagggacttgaaagcgaaagggaaaccag |
| | aggagctctctcgacgcaggactcggcttg |
| | ctgaagcgcgcacggcaagaggcgaggggc |
| | ggcgactggtgagtacgccaaaaattttga |
| | ctagcggaggctagaaggagagagatgggt |
| | gcgagagcgtcagtattaagcgggggagaa |
| | ttagatcgcgatgggaaaaaattcggttaa |
| | ggccagggggaaagaaaaaatataaattaa |
| | aacatatagtatgggcaagcagggagctag |
| | aacgattcgcagttaatcctggcctgttag |
| | aaacatcagaaggctgtagacaaatactgg |
| | gacagctacaaccatcccttcagacaggat |
| | cagaagaacttagatcattatataatacag |
| | tagcaaccctctattgtgtgcatcaaagga |
| | tagagataaaagacaccaaggaagctttag |
| | acaagatagaggaagagcaaaacaaaagta |
| | agaccaccgcacagcaagcggccgctgatc |
| | ttcagacctggaggaggagatatgagggac |
| | aattggagaagtgaattatataaatataaa |
| | gtagtaaaaattgaaccattaggagtagca |
| | cccaccaaggcaaagagaagagtggtgcag |
| | agagaaaaaagagcagtgggaataggagct |
| | ttgttccttgggttcttgggagcagcagga |
| | agcactatgggcgcagcgtcaatgacgctg |
| | acggtacaggccagacaattattgtctggt |
| | atagtgcagcagcagaacaatttgctgagg |
| | gctattgaggcgcaacagcatctgttgcaa |
| | ctcacagtctggggcatcaagcagctccag |
| | gcaagaatcctggctgtggaaagataccta |
| | aaggatcaacagctcctggggatttggggt |
| | tgctctggaaaactcatttgcaccactgct |
| | gtgccttggaatgctagttggagtaataaa |
| | tctctggaacagatttggaatcacacgacc |
| | tggatggagtgggacagagaaattaacaat |
| | tacacaagcttaatacactccttaattgaa |
| | gaatcgcaaaaccagcaagaaaagaatgaa |
| | caagaattattggaattagataaatgggca |
| | agtttgtggaattggtttaacataacaaat |
| | tggctgtggtatataaaattattcataatg |
| | atagtaggaggcttggtaggtttaagaata |
| | gtttttgctgtactttctatagtgaataga |
| | gttaggcagggatattcaccattatcgttt |
| | cagacccacctcccaaccccgaggggaccc |
| | gacaggcccgaaggaatagaagaagaaggt |
| | ggagagagagacagagacagatccattcga |
| | ttagtgaacggatctcgacggtatcgatta |
| | gactgtagcccaggaatatggcagctagat |
| | tgtacacatttagaaggaaaagttatcttg |
| | gtagcagttcatgtagccagtggatatata |
| | gaagcagaagtaattccagcagagacaggg |
| | caagaaacagcatacttcctcttaaaatta |
| | gcaggaagatggccagtaaaaacagtacat |
| | acagacaatggcagcaatttcaccagtact |
| | acagttaaggccgcctgttggtgggcgggg |
| | atcaagcaggaatttggcattccctacaat |
| | ccccaaagtcaaggagtaatagaatctatg |
| | aataaagaattaaagaaaattataggacag |
| | gtaagagatcaggctgaacatcttaagaca |
| | gcagtacaaatggcagtattcatccacaat |
| | tttaaaagaaaaggggggattggggggtac |
| | agtgcaggggaaagaatagtagacataata |
| | gcaacagacatacaaactaaagaattacaa |
| | aaacaaattacaaaaattcaaaattttcgg |
| | gtttattacagggacagcagagatccagtt |
| | tggctgcattgatcaacgcgtagatctcta |
| | gctaatgatgggcgcacgagtaatgatggg |
| | cggacgactaatgatgggcgcacgagtaat |
| | gatgggcgtctagctaatgatgggcgctag |
| | agtaatgatgggcggtagactaatgatggg |
| | cgctccagtaatgatgggcgttctagcTCT |
| | AGAGGGTATATAATGGGGGCCACTACTACC |
| | AGAtAGCTTGGTACCGAGCTCtGATCCACT |
| | AGTGCCACCatgTCCCATCACTGGGGGTAC |
| | GGCAAACACAACGGACCTGAGCACTGGCAT |
| | AAGGACTTCCCCATTGCCAAGGGAGAGCGC |
| | CAGTCCCCTGTTGACATCGACACTCATACA |
| | GCCAAGTATGACCCTTCCCTGAAGCCCCTG |
| | TCTGTTTCCTATGATCAAGCAACTTCCCTG |
| | AGAATCCTCAACAATGGTCATGCTTTCAAC |
| | GTGGAGTTTGATGACTCTCAGGACAAAGCA |
| | GTGCTCAAGGGAGGACCCCTGGATGGCACT |
| | TACAGATTGATTCAGTTTCACTTTCACTGG |
| | GGTTCACTTGATGGACAAGGTTCAGAGCAT |
| | ACTGTGGATAAAAAGAAATATGCTGCAGAA |
| | CTTCACTTGGTTCACTGGAACACCAAATAT |
| | GGGGATTTTGGGAAAGCTGTGCAGCAACCT |
| | GATGGACTGGCCGTTCTAGGTATTTTTTTG |
| | AAGGTTGGCAGCGCTAAACCGGGCCATCAG |
| | AAAGTTGTTGATGTGCTGGATTCCATTAAA |
| | ACAAAGGGCAAGAGTGCTGACTTCACTAAC |
| | TTCGATCCTCGTGGCCTCCTTCCTGAATCC |
| | CTGGATTACTGGACCTACCCAGGCTCACTG |
| | ACCACCCCTCCTCTTCTGGAATGTGTGACC |
| | TGGATTGTGCTCAAGGAACCCATCAGCGTC |
| | AGCAGCGAGCAGGTGTTGAAATTCCGTAAA |
| | CTTAACTTCAATGGGGAGGGTGAACCCGAA |
| | GAACTGATGGTGGACAACTGGCGCCCAGCT |
| | CAGCCACTGAAGAACAGGCAAATCAAAGCT |
| | TCCTTCAAAggatccggtTCAGGGgtgagc |
| | aagggcgaggagctgttcaccggggtggtg |
| | cccatcctggtcgagctggacggcgacgta |
| | aacggccacaagttcagcgtgtccggcgag |
| | ggcgagggcgatgccacctacggcaagctg |
| | accctgaagttcatctgcaccaccggcaag |
| | ctgcccgtgccctggcccaccctcgtgacc |
| | accctgacctacggcgtgcagtgcttcagc |
| | cgctaccccgaccacatgaagcagcacgac |
| | ttcttcaagtccgccatgcccgaaggctac |
| | gtccaggagcgcaccatcttcttcaaggac |
| | gacggcaactacaagacccgcgccgaggtg |
| | aagttcgagggcgacaccctggtgaaccgc |
| | atcgagctgaagggcatcgacttcaaggag |
| | gacggcaacatcctggggcacaagctggag |
| | tacaactacaacagccacaacgtctatatc |
| | atggccgacaagcagaagaacggcatcaag |
| | gtgaacttcaagatccgccacaacatcgag |
| | gacggcagcgtgcagctcgccgaccactac |
| | cagcagaacacccccatcggcgacggcccc |
| | gtgctgctgcccgacaaccactacctgagc |
| | acccagtccgccctgagcaaagaccccaac |
| | gagaagcgcgatcacatggtcctgctggag |
| | ttcgtgaccgccgccgggatcactctcggc |
| | atggacgagctgtacaagggatcctaaATC |
| | GGGCTAGCgtcgacaatcaacctctggatt |
| | acaaaatttgtgaaagattgactggtattc |
| | ttaactatgttgctccttttacgctatgtg |
| | gatacgctgctttaatgcctttgtatcatg |
| | ctattgcttcccgtatggctttcattttct |
| | cctccttgtataaatcctggttgctgtctc |
| | tttatgaggagttgtggcccgttgtcaggc |
| | aacgtggcgtggtgtgcactgtgtttgctg |
| | acgcaacccccactggttggggcattgcca |
| | ccacctgtcagctcctttccgggactttcg |
| | ctttccccctccctattgccacggcggaac |
| | tcatcgccgcctgccttgcccgctgctgga |
| | caggggctcggctgttgggcactgacaatt |
| | ccgtggtgttgtcggggaagctgacgtcct |
| | ttccatggctgctcgcctgtgttgccacct |
| | ggattctgcgcgggacgtccttctgctacg |
| | tcccttcggccctcaatccagcggaccttc |
| | cttcccgcggcctgctgccggctctgcggc |
| | ctcttccgcgtcttcgccttcgccctcaga |
| | cgagtcggatctccctttgggccgcctccc |
| | cgcctggaattcgagctcggtaccggtgtg |
| | gaaagtccccaggctccccagcaggcagaa |
| | gtatgcaaagcatgcatctcaattagtcag |
| | caaccaggtgtggaaagtccccaggctccc |
| | cagcaggcagaagtatgcaaagcatgcatc |
| | tcaattagtcagcaaccatagtcccgcccc |
| | taactccgcccatcccgcccctaactccgc |
| | ccagttccgcccattctccgccccatggct |
| | gactaattttttttatttatgcagaggccg |
| | aggccgcctctgcctctgagctattccaga |
| | agtagtgaggaggcttttttggaggcctag |
| | gcttttgcaaaaagctcccgggagcttgta |
| | tatccattttcggatctgatcagcacTTCG |
| | AAGCCACCATGAACCCAGCCATCAGCGTCG |
| | CTCTCCTGCTCTCAGTCTTGCAGGTGTCCC |
| | GAGGGCAGAAGGTGACCAGCCTGACAGCCT |
| | GCCTGGTGAACCAAAACCTTCGCCTGGACT |
| | GCCGCCATGAGAATAACACCAAGGATAACT |
| | CCATCCAGCATGAGTTCAGCCTGACCCGAG |
| | AGAAGAGGAAGCACGTGCTCTCAGGCACCC |
| | TTGGGATACCCGAGCACACGTACCGCTCCC |
| | GCGTCACCCTCTCCAACCAGCCCTATATCA |
| | AGGTCCTTACCCTAGCCAACTTCACCACCA |
| | AGGATGAGGGCGACTACTTTTGTGAGCTTC |
| | AAGTCTCGGGCGCGAATCCCATGAGCTCCA |
| | ATAAAAGTATCAGTGTGTATAGAGACAAGC |
| | TGGTCAAGTGTGGCGGCATAAGCCTGCTGG |
| | TTCAGAACACATCCTGGATGCTGCTGCTGC |
| | TGCTTTCCCTCTCCCTCCTCCAAGCCCTGG |
| | ACTTCATTTCTCTGTAATTCGAAgcgaatt |
| | cgagctcggtacctttaagaccaatgactt |
| | acaaggcagctgtagatcttagccactttt |
| | taaaagaaaaggggggactggaagggctaa |
| | ttcactcccaacgaagacaagatctgcttt |
| | ttgcttgtactgggtctctctggttagacc |
| | agatctgagcctgggagctctctggctaac |
| | tagggaacccactgcttaagcctcaataaa |
| | gcttgccttgagtgcttcaagtagtgtgtg |
| | cccgtctgttgtgtgactctggtaactaga |
| | gatccctcagacccttttagtcagtgtgga |
| | aaatctctagcagtagtagttcatgtcatc |
| | ttattattcagtatttataacttgcaaaga |
| | aatgaatatcagagagtgagaggaacttgt |
| | ttattgcagcttataatggttacaaataaa |
| | gcaatagcatcacaaatttcacaaataaag |
| | catttttttcactgcattctagttgtggtt |
| | tgtccaaactcatcaatgtatcttatcatg |
| | tctggctctagctatcccgcccctaactcc |
| | gcccatcccgcccctaactccgcccagttc |
| | cgcccattctccgccccatggctgactaat |
| | tttttttatttatgcagaggccgaggccgc |
| | ctcggcctctgagctattccagaagtagtg |
| | aggaggcttttttggaggcctagggacgta |
| | cccaattcgccctatagtgagtcgtattac |
| | gcgcgctcactggccgtcgttttacaacgt |
| | cgtgactgggaaaaccctggcgttacccaa |
| | cttaatcgccttgcagcacatccccattcg |
| | ccagctggcgtaatagcgaagaggcccgca |
| | ccgatcgcccttcccaacagttgcgcagcc |
| | tgaatggcgaatgggacgcgccctgtagcg |
| | gcgcattaagcgcggcgggtgtggtggtta |
| | cgcgcagcgtgaccgctacacttgccagcg |
| | ccctagcgcccgctcctttcgctttcttcc |
| | cttcctttctcgccacgttcgccggctttc |
| | cccgtcaagctctaaatcgggggctccctt |
| | tagggttccgatttagtgctttacggcacc |
| | tcgaccccaaaaaacttgattagggtgatg |
| | gttcacgtagtgggccatcgccctgataga |
| | cggtttttcgccctttgacgttggagtcca |
| | cgttctttaatagtggactcttgttccaaa |
| | ctggaacaacactcaaccctatctcggtct |
| | attcttttgatttataagggattttgccga |
| | tttcggcctattggttaaaaaatgagctga |
| | tttaacaaaaatttaacgcgaattttaaca |
| | aaatattaacgcttacaatttaggtggcac |
| | ttttcggggaaatgtgcgcggaacccctat |
| | ttgtttatttttctaaatacattcaaatat |
| | gtatccgctcatgagacaataaccctgata |
| | aatgcttcaataatattgaaaaaggaagag |
| | tatgagtattcaacatttccgtgtcgccct |
| | tattcccttttttgcggcattttgccttcc |
| | tgtttttgctcacccagaaacgctggtgaa |
| | agtaaaagatgctgaagatcagttgggtgc |
| | acgagtgggttacatcgaactggatctcaa |
| | cagcggtaagatccttgagagttttcgccc |
| | cgaagaacgttttccaatgatgagcacttt |
| | taaagttctgctatgtggcgcggtattatc |
| | ccgtattgacgccgggcaagagcaactcgg |
| | tcgccgcatacactattctcagaatgactt |
| | ggttgagtactcaccagtcacagaaaagca |
| | tcttacggatggcatgacagtaagagaatt |
| | atgcagtgctgccataaccatgagtgataa |
| | cactgcggccaacttacttctgacaacgat |
| | cggaggaccgaaggagctaaccgctttttt |
| | gcacaacatgggggatcatgtaactcgcct |
| | tgatcgttgggaaccggagctgaatgaagc |
| | cataccaaacgacgagcgtgacaccacgat |
| | gcctgtagcaatggcaacaacgttgcgcaa |
| | actattaactggcgaactacttactctagc |
| | ttcccggcaacaattaatagactggatgga |
| | ggcggataaagttgcaggaccacttctgcg |
| | ctcggcccttccggctggctggtttattgc |
| | tgataaatctggagccggtgagcgtgggtc |
| | tcgcggtatcattgcagcactggggccaga |
| | tggtaagccctcccgtatcgtagttatcta |
| | cacgacggggagtcaggcaactatggatga |
| | acgaaatagacagatcgctgagataggtgc |
| | ctcactgattaagcattggtaactgtcaga |
| | ccaagtttactcatatatactttagattga |
| | tttaaaacttcatttttaatttaaaaggat |
| | ctaggtgaagatcctttttgataatctcat |
| | gaccaaaatcccttaacgtgagttttcgtt |
| | ccactgagcgtcagaccccgtagaaaagat |
| | caaaggatcttcttgagatcctttttttct |
| | gcgcgtaatctgctgcttgcaaacaaaaaa |
| | accaccgctaccagcggtggtttgtttgcc |
| | ggatcaagagctaccaactctttttccgaa |
| | ggtaactggcttcagcagagcgcagatacc |
| | aaatactgttcttctagtgtagccgtagtt |
| | aggccaccacttcaagaactctgtagcacc |
| | gcctacatacctcgctctgctaatcctgtt |
| | accagtggctgctgccagtggcgataagtc |
| | gtgtcttaccgggttggactcaagacgata |
| | gttaccggataaggcgcagcggtcgggctg |
| | aacggggggttcgtgcacacagcccagctt |
| | ggagcgaacgacctacaccgaactgagata |
| | cctacagcgtgagctatgagaaagcgccac |
| | gcttcccgaagggagaaaggcggacaggta |
| | tccggtaagcggcagggtcggaacaggaga |
| | gcgcacgagggagcttccagggggaaacgc |
| | ctggtatctttatagtcctgtcgggtttcg |
| | ccacctctgacttgagcgtcgatttttgtg |
| | atgctcgtcaggggggcggagcctatggaa |
| | aaacgccagcaacgcggcctttttacggtt |
| | cctggccttttgctggccttttgctcacat |
| | gttctttcctgcgttatcccctgattctgt |
| | ggataaccgtattaccgcctttgagtgagc |
| | tgataccgctcgccgcagccgaacgaccga |
| | gcgcagcgagtcagtgagcgaggaagcgga |
| | agagcgcccaatacgcaaaccgcctctccc |
| | cgcgcgttggccgattcattaatgcagctg |
| | gcacgacaggtttcccgactggaaagcggg |
| | cagtgagcgcaacgcaattaatgtgagtta |
| | gctcactcattaggcaccccaggctttaca |
| | ctttatgcttccggctcgtatgttgtgtgg |
| | aattgtgagcggataacaatttcacacagg |
| | aaacagctatgaccatgattacgccaagcg |
| | cgcaattaaccctcactaaagggaacaaaa |
| | gctggagctgcaagctta |
| | (SEQ ID NO: 44) |
| OT-ZFHD-076 | tttgagtgagctgataccgctcgccgcagc |
| | cgaacgaccgagcgcagcgagtcagtgagc |
| | gaggaagcggaagagcgcccaatacgcaaa |
| | ccgcctctccccgcgcgttggccgattcat |
| | taatgcagctggcacgacaggtttcccgac |
| | tggaaagcgggcagtgagcgcaacgcaatt |
| | aatgtgagttagctcactcattaggcaccc |
| | caggctttacactttatgcttccggctcgt |
| | atgttgtgtggaattgtgagcggataacaa |
| | tttcacacaggaaacagctatgaccatgat |
| | tacgccaagcgcgcaattaaccctcactaa |
| | agggaacaaaagctggagctgcaagcttaa |
| | tgtagtcttatgcaatactcttgtagtctt |
| | gcaacatggtaacgatgagttagcaacatg |
| | ccttacaaggagagaaaaagcaccgtgcat |
| | gccgattggtggaagtaaggtggtacgatc |
| | gtgccttattaggaaggcaacagacgggtc |
| | tgacatggattggacgaaccactgaattgc |
| | cgcattgcagagatattgtatttaagtgcc |
| | tagctcgatacataaacgggtctctctggt |
| | tagaccagatctgagcctgggagctctctg |
| | gctaactagggaacccactgcttaagcctc |
| | aataaagcttgccttgagtgcttcaagtag |
| | tgtgtgcccgtctgttgtgtgactctggta |
| | actagagatccctcagacccttttagtcag |
| | tgtggaaaatctctagcagtggcgcccgaa |
| | cagggacttgaaagcgaaagggaaaccaga |
| | ggagctctctcgacgcaggactcggcttgc |
| | tgaagcgcgcacggcaagaggcgaggggcg |
| | gcgactggtgagtacgccaaaaattttgac |
| | tagcggaggctagaaggagagagatgggtg |
| | cgagagcgtcagtattaagcgggggagaat |
| | tagatcgcgatgggaaaaaattcggttaag |
| | gccagggggaaagaaaaaatataaattaaa |
| | acatatagtatgggcaagcagggagctaga |
| | acgattcgcagttaatcctggcctgttaga |
| | aacatcagaaggctgtagacaaatactggg |
| | acagctacaaccatcccttcagacaggatc |
| | agaagaacttagatcattatataatacagt |
| | agcaaccctctattgtgtgcatcaaaggat |
| | agagataaaagacaccaaggaagctttaga |
| | caagatagaggaagagcaaaacaaaagtaa |
| | gaccaccgcacagcaagcggccgctgatct |
| | tcagacctggaggaggagatatgagggaca |
| | attggagaagtgaattatataaatataaag |
| | tagtaaaaattgaaccattaggagtagcac |
| | ccaccaaggcaaagagaagagtggtgcaga |
| | gagaaaaaagagcagtgggaataggagctt |
| | tgttccttgggttcttgggagcagcaggaa |
| | gcactatgggcgcagcgtcaatgacgctga |
| | cggtacaggccagacaattattgtctggta |
| | tagtgcagcagcagaacaatttgctgaggg |
| | ctattgaggcgcaacagcatctgttgcaac |
| | tcacagtctggggcatcaagcagctccagg |
| | caagaatcctggctgtggaaagatacctaa |
| | aggatcaacagctcctggggatttggggtt |
| | gctctggaaaactcatttgcaccactgctg |
| | tgccttggaatgctagttggagtaataaat |
| | ctctggaacagatttggaatcacacgacct |
| | ggatggagtgggacagagaaattaacaatt |
| | acacaagcttaatacactccttaattgaag |
| | aatcgcaaaaccagcaagaaaagaatgaac |
| | aagaattattggaattagataaatgggcaa |
| | gtttgtggaattggtttaacataacaaatt |
| | ggctgtggtatataaaattattcataatga |
| | tagtaggaggcttggtaggtttaagaatag |
| | tttttgctgtactttctatagtgaatagag |
| | ttaggcagggatattcaccattatcgtttc |
| | agacccacctcccaaccccgaggggacccg |
| | acaggcccgaaggaatagaagaagaaggtg |
| | gagagagagacagagacagatccattcgat |
| | tagtgaacggatctcgacggtatcgattag |
| | actgtagcccaggaatatggcagctagatt |
| | gtacacatttagaaggaaaagttatcttgg |
| | tagcagttcatgtagccagtggatatatag |
| | aagcagaagtaattccagcagagacagggc |
| | aagaaacagcatacttcctcttaaaattag |
| | caggaagatggccagtaaaaacagtacata |
| | cagacaatggcagcaatttcaccagtacta |
| | cagttaaggccgcctgttggtgggcgggga |
| | tcaagcaggaatttggcattccctacaatc |
| | cccaaagtcaaggagtaatagaatctatga |
| | ataaagaattaaagaaaattataggacagg |
| | taagagatcaggctgaacatcttaagacag |
| | cagtacaaatggcagtattcatccacaatt |
| | ttaaaagaaaaggggggattggggggtaca |
| | gtgcaggggaaagaatagtagacataatag |
| | caacagacatacaaactaaagaattacaaa |
| | aacaaattacaaaaattcaaaattttcggg |
| | tttattacagggacagcagagatccagttt |
| | ggctgcattgatcacgtgaggctccggtgc |
| | ccgtcagtgggcagagcgcacatcgcccac |
| | agtccccgagaagttggggggaggggtcgg |
| | caattgaaccggtgcctagagaaggtggcg |
| | cggggtaaactgggaaagtgatgtcgtgta |
| | ctggctccgcctttttcccgagggtggggg |
| | agaaccgtatataagtgcagtagtcgccgt |
| | gaacgttctttttcgcaacgggtttgccgc |
| | cagaacacaggtaagtgccgtgtgtggttc |
| | ccgcgggcctggcctctttacgggttatgg |
| | cccttgcgtgccttgaattacttccacctg |
| | gctgcagtacgtgattcttgatcccgagct |
| | tcgggttggaagtgggtgggagagttcgag |
| | gccttgcgcttaaggagccccttcgcctcg |
| | tgcttgagttgaggcctggcctgggcgctg |
| | gggccgccgcgtgcgaatctggtggcacct |
| | tcgcgcctgtctcgctgctttcgataagtc |
| | tctagccatttaaaatttttgatgacctgc |
| | tgcgacgctttttttctggcaagatagtct |
| | tgtaaatgcgggccaagatctgcacactgg |
| | tatttcggtttttggggccgcgggcggcga |
| | cggggcccgtgcgtcccagcgcacatgttc |
| | ggcgaggcggggcctgcgagcgcggccacc |
| | gagaatcggacgggggtagtctcaagctgg |
| | ccggcctgctctggtgcctggcctcgcgcc |
| | gccgtgtatcgccccgccctgggcggcaag |
| | gctggcccggtcggcaccagttgcgtgagc |
| | ggaaagatggccgcttcccggccctgctgc |
| | agggagctcaaaatggaggacgcggcgctc |
| | gggagagcgggcgggtgagtcacccacaca |
| | aaggaaaagggcctttccgtcctcagccgt |
| | cgcttcatgtgactccactgagtaccgggc |
| | gccgtccaggcacctcgattagttctcgag |
| | cttttggagtacgtcgtctttaggttgggg |
| | ggaggggttttatgcgatggagtttcccca |
| | cactgagtgggtggagactgaagttaggcc |
| | agcttggcacttgatgtaattctccttgga |
| | atttgccctttttgagtttggatcttggtt |
| | cattctcaagcctcagacagtggttcaaag |
| | tttttttcttccatttcaggtgtcgtgatc |
| | tagaggatcACTAGTgccaccatgGCACCT |
| | AAGaaaAAGAGGAAGGTTgaacgcccatat |
| | gcttgccctgtcgagtcctgcgatcgccgc |
| | ttttctcgctcggatgagcttacccgccat |
| | atccgcatccacacaggccagaagcccttc |
| | cagtgtcgaatctgcatgcgtaacttcagt |
| | cgtagtgaccaccttaccacccacatccgc |
| | acccacacaggcggcggccgcaggaggaag |
| | aaacgcaccagcatagagaccaacatccgt |
| | gtggccttagagaagagtttcttggagaat |
| | caaaagcctacctcggaagagatcactatg |
| | attgctgatcagctcaatatggaaaaagag |
| | gtgattcgtgtttggttctgtaaccgccgc |
| | cagaaagaaaaaagaatcaacactagactg |
| | ggggccttgcttggcaacagcacagaccca |
| | gctgtgttcacagacctggcatccGTGgac |
| | aactccgagtttcagcagctgctgaaccag |
| | ggcatacctgtggccccccacacaactgag |
| | cccatgctgatggagtaccctgaggctata |
| | actcgcctagtgacaggggcccagaggccc |
| | cccgacccagctcctgctccactgggggcc |
| | ccggggctccccaatggcctcctttcagga |
| | gatgaagacttctcctccattgcggacatg |
| | gacttctcagccctgctgagtcagatcagc |
| | tccggaggtagtggtggaggcagtggtGGT |
| | TCCCATCACTGGGGGTACGGCAAACACAAC |
| | GGACCTGAGCACTGGCATAAGGACTTCCCC |
| | ATTGCCAAGGGAGAGCGCCAGTCCCCTGTT |
| | GACATCGACACTCATACAGCCAAGTATGAC |
| | CCTTCCCTGAAGCCCCTGTCTGTTTCCTAT |
| | GATCAAGCAACTTCCCTGAGGATCCTCAAC |
| | AATGGTCATGCTTTCAACGTGGAGTTTGAT |
| | GACTCTCAGGACAAAGCAGTGCTCAAGGGA |
| | GGACCCCTGGATGGCACTTACAGATTGATT |
| | CAGTTTCACTTTCACTGGGGTTCACTTGAT |
| | GGACAAGGTTCAGAGCATACTGTGGATAAA |
| | AAGAAATATGCTGCAGAACTTCACTTGGTT |
| | CACTGGAACACCAAATATGGGGATTTTGGG |
| | AAAGCTGTGCAGCAACCTGATGGACTGGCC |
| | GTTCTAGGTATTTTTTTGAAGGTTGGCAGC |
| | GCTAAACCGGGCCATCAGAAAGTTGTTGAT |
| | GTGCTGGATTCCATTAAAACAAAGGGCAAG |
| | AGTGCTGACTTCACTAACTTCGATCCTCGT |
| | GGCCTCCTTCCTGAATCCCTGGATTACTGG |
| | ACCTACCCAGGCTCACTGACCACCCCTCCT |
| | CTTCTGGAATGTGTGACCTGGATTGTGCTC |
| | AAGGAACCCATCAGCGTCAGCAGCGAGCAG |
| | GTGTTGAAATTCCGTAAACTTAACTTCAAT |
| | GGGGAGGGTGAACCCGAAGAACTGATGGTG |
| | GACAACTGGCGCCCAGCTCAGCCACTGAAG |
| | AACAGGCAAATCAAAGCTTCCTTCAAAgga |
| | tccggagctactaacttcagcctgctgaag |
| | caggctggagacgtggaggagaaccctgga |
| | cctTCTGAGCTGATTAAGGAGAATATGCAC |
| | ATGAAGCTGTACATGGAAGGAACTGTGGAC |
| | AATCATCACTTTAAGTGCACATCGGAGGGA |
| | GAAGGCAAGCCCTACGAAGGCACCCAGACC |
| | ATGAGGATCAAGGTGGTTGAGGGCGGACCG |
| | CTGCCCTTCGCCTTCGATATCCTGGCGACT |
| | TCATTCCTCTACGGAAGCAAAACCTTTATT |
| | AACCACACTCAGGGTATACCAGACTTCTTT |
| | AAGCAATCCTTCCCTGAGGGTTTTACATGG |
| | GAGAGAGTCACTACATATGAAGATGGGGGC |
| | GTGCTAACCGCTACTCAGGACACCTCTTTA |
| | CAAGATGGATGTCTCATCTACAACGTAAAA |
| | ATTAGGGGGGTGAACTTCACATCCAACGGC |
| | CCTGTGATGCAGAAGAAAACATTGGGGTGG |
| | GAAGCCTTTACGGAGACGCTGTATCCAGCT |
| | GATGGCGGACTGGAAGGCCGGAATGATATG |
| | GCCCTTAAGTTAGTTGGTGGGTCACATTTG |
| | ATAGCAAACATCAAGACCACATATCGTAGT |
| | AAGAAACCCGCTAAAAACCTCAAGATGCCT |
| | GGTGTCTACTATGTTGACTATAGACTGGAA |
| | CGAATCAAAGAGGCAAATAATGAGACCTAC |
| | GTCGAGCAGCATGAAGTAGCAGTGGCCCGC |
| | TACTGCGACCTCCCAAGCAAACTGGGGCAC |
| | AAACTTAATtgaATCGGGCTAGCgtcgaca |
| | atcaacctctggattacaaaatttgtgaaa |
| | gattgactggtattcttaactatgttgctc |
| | cttttacgctatgtggatacgctgctttaa |
| | tgcctttgtatcatgctattgcttcccgta |
| | tggctttcattttctcctccttgtataaat |
| | cctggttgctgtctctttatgaggagttgt |
| | ggcccgttgtcaggcaacgtggcgtggtgt |
| | gcactgtgtttgctgacgcaacccccactg |
| | gttggggcattgccaccacctgtcagctcc |
| | tttccgggactttcgctttccccctcccta |
| | ttgccacggcggaactcatcgccgcctgcc |
| | ttgcccgctgctggacaggggctcggctgt |
| | tgggcactgacaattccgtggtgttgtcgg |
| | ggaagctgacgtcctttccatggctgctcg |
| | cctgtgttgccacctggattctgcgcggga |
| | cgtccttctgctacgtcccttcggccctca |
| | atccagcggaccttccttcccgcggcctgc |
| | tgccggctctgcggcctcttccgcgtcttc |
| | gccttcgccctcagacgagtcggatctccc |
| | tttgggccgcctccccgcctggaattcgag |
| | ctcggtacctttaagaccaatgacttacaa |
| | ggcagctgtagatcttagccactttttaaa |
| | agaaaaggggggactggaagggctaattca |
| | ctcccaacgaagacaagatctgctttttgc |
| | ttgtactgggtctctctggttagaccagat |
| | ctgagcctgggagctctctggctaactagg |
| | gaacccactgcttaagcctcaataaagctt |
| | gccttgagtgcttcaagtagtgtgtgcccg |
| | tctgttgtgtgactctggtaactagagatc |
| | cctcagacccttttagtcagtgtggaaaat |
| | ctctagcagtagtagttcatgtcatcttat |
| | tattcagtatttataacttgcaaagaaatg |
| | aatatcagagagtgagaggaacttgtttat |
| | tgcagcttataatggttacaaataaagcaa |
| | tagcatcacaaatttcacaaataaagcatt |
| | tttttcactgcattctagttgtggtttgtc |
| | caaactcatcaatgtatcttatcatgtctg |
| | gctctagctatcccgcccctaactccgccc |
| | atcccgcccctaactccgcccagttccgcc |
| | cattctccgccccatggctgactaattttt |
| | tttatttatgcagaggccgaggccgcctcg |
| | gcctctgagctattccagaagtagtgagga |
| | ggcttttttggaggcctagggacgtaccca |
| | attcgccctatagtgagtcgtattacgcgc |
| | gctcactggccgtcgttttacaacgtcgtg |
| | actgggaaaaccctggcgttacccaactta |
| | atcgccttgcagcacatccccctttcgcca |
| | gctggcgtaatagcgaagaggcccgcaccg |
| | atcgcccttcccaacagttgcgcagcctga |
| | atggcgaatgggacgcgccctgtagcggcg |
| | cattaagcgcggcgggtgtggtggttacgc |
| | gcagcgtgaccgctacacttgccagcgccc |
| | tagcgcccgctcctttcgctttcttccctt |
| | cctttctcgccacgttcgccggctttcccc |
| | gtcaagctctaaatcgggggctccctttag |
| | ggttccgatttagtgctttacggcacctcg |
| | accccaaaaaacttgattagggtgatggtt |
| | cacgtagtgggccatcgccctgatagacgg |
| | tttttcgccctttgacgttggagtccacgt |
| | tctttaatagtggactcttgttccaaactg |
| | gaacaacactcaaccctatctcggtctatt |
| | cttttgatttataagggattttgccgattt |
| | cggcctattggttaaaaaatgagctgattt |
| | aacaaaaatttaacgcgaattttaacaaaa |
| | tattaacgcttacaatttaggtggcacttt |
| | tcggggaaatgtgcgcggaacccctatttg |
| | tttatttttctaaatacattcaaatatgta |
| | tccgctcatgagacaataaccctgataaat |
| | gcttcaataatattgaaaaaggaagagtat |
| | gagtattcaacatttccgtgtcgcccttat |
| | tcccttttttgcggcattttgccttcctgt |
| | ttttgctcacccagaaacgctggtgaaagt |
| | aaaagatgctgaagatcagttgggtgcacg |
| | agtgggttacatcgaactggatctcaacag |
| | cggtaagatccttgagagttttcgccccga |
| | agaacgttttccaatgatgagcacttttaa |
| | agttctgctatgtggcgcggtattatcccg |
| | tattgacgccgggcaagagcaactcggtcg |
| | ccgcatacactattctcagaatgacttggt |
| | tgagtactcaccagtcacagaaaagcatct |
| | tacggatggcatgacagtaagagaattatg |
| | cagtgctgccataaccatgagtgataacac |
| | tgcggccaacttacttctgacaacgatcgg |
| | aggaccgaaggagctaaccgcttttttgca |
| | caacatgggggatcatgtaactcgccttga |
| | tcgttgggaaccggagctgaatgaagccat |
| | accaaacgacgagcgtgacaccacgatgcc |
| | tgtagcaatggcaacaacgttgcgcaaact |
| | attaactggcgaactacttactctagcttc |
| | ccggcaacaattaatagactggatggaggc |
| | ggataaagttgcaggaccacttctgcgctc |
| | ggcccttccggctggctggtttattgctga |
| | taaatctggagccggtgagcgtgggtctcg |
| | cggtatcattgcagcactggggccagatgg |
| | taagccctcccgtatcgtagttatctacac |
| | gacggggagtcaggcaactatggatgaacg |
| | aaatagacagatcgctgagataggtgcctc |
| | actgattaagcattggtaactgtcagacca |
| | agtttactcatatatactttagattgattt |
| | aaaacttcatttttaatttaaaaggatcta |
| | ggtgaagatcctttttgataatctcatgac |
| | caaaatcccttaacgtgagttttcgttcca |
| | ctgagcgtcagaccccgtagaaaagatcaa |
| | aggatcttcttgagatcattttttctgcgc |
| | gtaatctgctgcttgcaaacaaaaaaacca |
| | ccgctaccagcggtggtttgtttgccggat |
| | caagagctaccaactctttttccgaaggta |
| | actggcttcagcagagcgcagataccaaat |
| | actgttcttctagtgtagccgtagttaggc |
| | caccacttcaagaactctgtagcaccgcct |
| | acatacctcgctctgctaatcctgttacca |
| | gtggctgctgccagtggcgataagtcgtgt |
| | cttaccgggttggactcaagacgatagtta |
| | ccggataaggcgcagcggtcgggctgaacg |
| | gggggttcgtgcacacagcccagcttggag |
| | cgaacgacctacaccgaactgagataccta |
| | cagcgtgagctatgagaaagcgccacgctt |
| | cccgaagggagaaaggcggacaggtatccg |
| | gtaagcggcagggtcggaacaggagagcgc |
| | acgagggagcttccagggggaaacgcctgg |
| | tatctttatagtcctgtcgggtttcgccac |
| | ctctgacttgagcgtcgatttttgtgatgc |
| | tcgtcaggggggcggagcctatggaaaaac |
| | gccagcaacgcggcctttttacggttcctg |
| | gccttttgctggccttttgctcacatgttc |
| | tttcctgcgttatcccctgattctgtggat |
| | aaccgtattaccgcc |
| | (SEQ ID NO: 45) |
| OT-ZFHD-077 | tttgagtgagctgataccgctcgccgcagc |
| | cgaacgaccgagcgcagcgagtcagtgagc |
| | gaggaagcggaagagcgcccaatacgcaaa |
| | ccgcctctccccgcgcgttggccgattcat |
| | taatgcagctggcacgacaggtttcccgac |
| | tggaaagcgggcagtgagcgcaacgcaatt |
| | aatgtgagttagctcactcattaggcaccc |
| | caggctttacactttatgcttccggctcgt |
| | atgttgtgtggaattgtgagcggataacaa |
| | tttcacacaggaaacagctatgaccatgat |
| | tacgccaagcgcgcaattaaccctcactaa |
| | agggaacaaaagctggagctgcaagcttaa |
| | tgtagtcttatgcaatactcttgtagtctt |
| | gcaacatggtaacgatgagttagcaacatg |
| | ccttacaaggagagaaaaagcaccgtgcat |
| | gccgattggtggaagtaaggtggtacgatc |
| | gtgccttattaggaaggcaacagacgggtc |
| | tgacatggattggacgaaccactgaattgc |
| | cgcattgcagagatattgtatttaagtgcc |
| | tagctcgatacataaacgggtctctctggt |
| | tagaccagatctgagcctgggagctctctg |
| | gctaactagggaacccactgcttaagcctc |
| | aataaagcttgccttgagtgcttcaagtag |
| | tgtgtgcccgtctgttgtgtgactctggta |
| | actagagatccctcagacccttttagtcag |
| | tgtggaaaatctctagcagtggcgcccgaa |
| | cagggacttgaaagcgaaagggaaaccaga |
| | ggagctctctcgacgcaggactcggcttgc |
| | tgaagcgcgcacggcaagaggcgaggggcg |
| | gcgactggtgagtacgccaaaaattttgac |
| | tagcggaggctagaaggagagagatgggtg |
| | cgagagcgtcagtattaagcgggggagaat |
| | tagatcgcgatgggaaaaaattcggttaag |
| | gccagggggaaagaaaaaatataaattaaa |
| | acatatagtatgggcaagcagggagctaga |
| | acgattcgcagttaatcctggcctgttaga |
| | aacatcagaaggctgtagacaaatactggg |
| | acagctacaaccatcccttcagacaggatc |
| | agaagaacttagatcattatataatacagt |
| | agcaaccctctattgtgtgcatcaaaggat |
| | agagataaaagacaccaaggaagctttaga |
| | caagatagaggaagagcaaaacaaaagtaa |
| | gaccaccgcacagcaagcggccgctgatct |
| | tcagacctggaggaggagatatgagggaca |
| | attggagaagtgaattatataaatataaag |
| | tagtaaaaattgaaccattaggagtagcac |
| | ccaccaaggcaaagagaagagtggtgcaga |
| | gagaaaaaagagcagtgggaataggagctt |
| | tgttccttgggttcttgggagcagcaggaa |
| | gcactatgggcgcagcgtcaatgacgctga |
| | cggtacaggccagacaattattgtctggta |
| | tagtgcagcagcagaacaatttgctgaggg |
| | ctattgaggcgcaacagcatctgttgcaac |
| | tcacagtctggggcatcaagcagctccagg |
| | caagaatcctggctgtggaaagatacctaa |
| | aggatcaacagctcctggggatttggggtt |
| | gctctggaaaactcatttgcaccactgctg |
| | tgccttggaatgctagttggagtaataaat |
| | ctctggaacagatttggaatcacacgacct |
| | ggatggagtgggacagagaaattaacaatt |
| | acacaagcttaatacactccttaattgaag |
| | aatcgcaaaaccagcaagaaaagaatgaac |
| | aagaattattggaattagataaatgggcaa |
| | gtttgtggaattggtttaacataacaaatt |
| | ggctgtggtatataaaattattcataatga |
| | tagtaggaggcttggtaggtttaagaatag |
| | tttttgctgtactttctatagtgaatagag |
| | ttaggcagggatattcaccattatcgtttc |
| | agacccacctcccaaccccgaggggacccg |
| | acaggcccgaaggaatagaagaagaaggtg |
| | gagagagagacagagacagatccattcgat |
| | tagtgaacggatctcgacggtatcgattag |
| | actgtagcccaggaatatggcagctagatt |
| | gtacacatttagaaggaaaagttatcttgg |
| | tagcagttcatgtagccagtggatatatag |
| | aagcagaagtaattccagcagagacagggc |
| | aagaaacagcatacttcctcttaaaattag |
| | caggaagatggccagtaaaaacagtacata |
| | cagacaatggcagcaatttcaccagtacta |
| | cagttaaggccgcctgttggtgggcgggga |
| | tcaagcaggaatttggcattccctacaatc |
| | cccaaagtcaaggagtaatagaatctatga |
| | ataaagaattaaagaaaattataggacagg |
| | taagagatcaggctgaacatcttaagacag |
| | cagtacaaatggcagtattcatccacaatt |
| | ttaaaagaaaaggggggattggggggtaca |
| | gtgcaggggaaagaatagtagacataatag |
| | caacagacatacaaactaaagaattacaaa |
| | aacaaattacaaaaattcaaaattttcggg |
| | tttattacagggacagcagagatccagttt |
| | ggctgcattgatcacgtgaggctccggtgc |
| | ccgtcagtgggcagagcgcacatcgcccac |
| | agtccccgagaagttggggggaggggtcgg |
| | caattgaaccggtgcctagagaaggtggcg |
| | cggggtaaactgggaaagtgatgtcgtgta |
| | ctggctccgcctttttcccgagggtggggg |
| | agaaccgtatataagtgcagtagtcgccgt |
| | gaacgttctttttcgcaacgggtttgccgc |
| | cagaacacaggtaagtgccgtgtgtggttc |
| | ccgcgggcctggcctctttacgggttatgg |
| | cccttgcgtgccttgaattacttccacctg |
| | gctgcagtacgtgattcttgatcccgagct |
| | tcgggttggaagtgggtgggagagttcgag |
| | gccttgcgcttaaggagccccttcgcctcg |
| | tgcttgagttgaggcctggcctgggcgctg |
| | gggccgccgcgtgcgaatctggtggcacct |
| | tcgcgcctgtctcgctgctttcgataagtc |
| | tctagccatttaaaatttttgatgacctgc |
| | tgcgacgctattttctggcaagatagtctt |
| | gtaaatgcgggccaagatctgcacactggt |
| | atttcggtttttggggccgcgggcggcgac |
| | ggggcccgtgcgtcccagcgcacatgttcg |
| | gcgaggcggggcctgcgagcgcggccaccg |
| | agaatcggacgggggtagtctcaagctggc |
| | cggcctgctctggtgcctggcctcgcgccg |
| | ccgtgtatcgccccgccctgggcggcaagg |
| | ctggcccggtcggcaccagttgcgtgagcg |
| | gaaagatggccgcttcccggccctgctgca |
| | gggagctcaaaatggaggacgcggcgctcg |
| | ggagagcgggcgggtgagtcacccacacaa |
| | aggaaaagggcctttccgtcctcagccgtc |
| | gcttcatgtgactccactgagtaccgggcg |
| | ccgtccaggcacctcgattagttctcgagc |
| | ttttggagtacgtcgtctttaggttggggg |
| | gaggggttttatgcgatggagtttccccac |
| | actgagtgggtggagactgaagttaggcca |
| | gcttggcacttgatgtaattctccttggaa |
| | tttgccctttttgagtttggatcttggttc |
| | attctcaagcctcagacagtggttcaaagt |
| | ttttttcttccatttcaggtgtcgtgatct |
| | agaggatcACTAGTgccaccatgGCACCTA |
| | AGaaaAAGAGGAAGGTTgaacgcccatatg |
| | cttgccctgtcgagtcctgcgatcgccgct |
| | tttctcgctcggatgagcttacccgccata |
| | tccgcatccacacaggccagaagcccttcc |
| | agtgtcgaatctgcatgcgtaacttcagtc |
| | gtagtgaccaccttaccacccacatccgca |
| | cccacacaggcggcggccgcaggaggaaga |
| | aacgcaccagcatagagaccaacatccgtg |
| | tggccttagagaagagtttcttggagaatc |
| | aaaagcctacctcggaagagatcactatga |
| | ttgctgatcagctcaatatggaaaaagagg |
| | tgattcgtgtttggttctgtaaccgccgcc |
| | agaaagaaaaaagaatcaacactagactgg |
| | gggccttgcttggcaacagcacagacccag |
| | ctgtgttcacagacctggcatccGTGgaca |
| | actccgagtttcagcagctgctgaaccagg |
| | gcatacctgtggccccccacacaactgagc |
| | ccatgctgatggagtaccctgaggctataa |
| | ctcgcctagtgacaggggcccagaggcccc |
| | ccgacccagctcctgctccactgggggccc |
| | cggggctccccaatggcctcctttcaggag |
| | atgaagacttctcctccattgcggacatgg |
| | acttctcagccctgctgagtcagatcagct |
| | ccggaggtagtggtggaggcagtggtGGTT |
| | CACTGGCGCTCAGCCTTACTGCCGACCAAA |
| | TGGTATCAGCTCTTCTGGACGCAGAACCCC |
| | CAATTCTTTATTCCGAGTACGACCCCACAC |
| | GCCCGTTCAGTGAAGCTTCCATGATGGGCC |
| | TCCTTACGAACCTTGCCGACCGGGAACTCG |
| | TGCACATGATCAATTGGGCGAAGCGGGTGC |
| | CGGGGTTCGTAGATTTGACACTTCACGACC |
| | AAGTTCATCTCTTGGAATGTGCTTGGATGG |
| | AGATATTGATGATCGGACTCGTGTGGAGGT |
| | CAATGGAGCATCCTGGTAAACTTCTTTTCG |
| | CACCCAATCTGCTCTTGGATAGAAATCAGG |
| | GTAAGTGCGTCGAGGGTGGCGTTGAAATCT |
| | TCGACATGCTCCTTGCGACATCCAGCCGAT |
| | TCCGAATGATGAATCTTCAAGGAGAGGAAT |
| | TTGTCTGTCTTAAGAGCATTATACTCCTCA |
| | ATAGTGGAGTTTACACCTTCTTGTCCTCTA |
| | CACTGAAATCACTTGAGGAAAAAGATCACA |
| | TACATAGGGTGTTGGATAAAATCACGGATA |
| | CACTCATACATCTGATGGCAAAAGCAGGAT |
| | TGACCCTGCAACAGCAGCACgacCGACTGG |
| | CCCAACTGCTGTTGATCCTTAGCCATATCA |
| | GACACATGTCTAACAAAAGGATGGAACATT |
| | TGTACAGCATGAAATGTAAGAACGTAGTGC |
| | CACTGTCCGATTTGTTGCTGGAAATGCTGG |
| | ACGCTCATCGGCTCggatccggagctacta |
| | acttcagcctgctgaagcaggctggagacg |
| | tggaggagaaccctggacctTCTGAGCTGA |
| | TTAAGGAGAATATGCACATGAAGCTGTACA |
| | TGGAAGGAACTGTGGACAATCATCACTTTA |
| | AGTGCACATCGGAGGGAGAAGGCAAGCCCT |
| | ACGAAGGCACCCAGACCATGAGGATCAAGG |
| | TGGTTGAGGGCGGACCGCTGCCCTTCGCCT |
| | TCGATATCCTGGCGACTTCATTCCTCTACG |
| | GAAGCAAAACCTTTATTAACCACACTCAGG |
| | GTATACCAGACTTCTTTAAGCAATCCTTCC |
| | CTGAGGGTTTTACATGGGAGAGAGTCACTA |
| | CATATGAAGATGGGGGCGTGCTAACCGCTA |
| | CTCAGGACACCTCTTTACAAGATGGATGTC |
| | TCATCTACAACGTAAAAATTAGGGGGGTGA |
| | ACTTCACATCCAACGGCCCTGTGATGCAGA |
| | AGAAAACATTGGGGTGGGAAGCCTTTACGG |
| | AGACGCTGTATCCAGCTGATGGCGGACTGG |
| | AAGGCCGGAATGATATGGCCCTTAAGTTAG |
| | TTGGTGGGTCACATTTGATAGCAAACATCA |
| | AGACCACATATCGTAGTAAGAAACCCGCTA |
| | AAAACCTCAAGATGCCTGGTGTCTACTATG |
| | TTGACTATAGACTGGAACGAATCAAAGAGG |
| | CAAATAATGAGACCTACGTCGAGCAGCATG |
| | AAGTAGCAGTGGCCCGCTACTGCGACCTCC |
| | CAAGCAAACTGGGGCACAAACTTAATtgaA |
| | TCGGGCTAGCgtcgacaatcaacctctgga |
| | ttacaaaatttgtgaaagattgactggtat |
| | tcttaactatgttgctccttttacgctatg |
| | tggatacgctgctttaatgcctttgtatca |
| | tgctattgcttcccgtatggctttcatttt |
| | ctcctccttgtataaatcctggttgctgtc |
| | tctttatgaggagttgtggcccgttgtcag |
| | gcaacgtggcgtggtgtgcactgtgtttgc |
| | tgacgcaacccccactggttggggcattgc |
| | caccacctgtcagctcctttccgggacttt |
| | cgctttccccctccctattgccacggcgga |
| | actcatcgccgcctgccttgcccgctgctg |
| | gacaggggctcggctgttgggcactgacaa |
| | ttccgtggtgttgtcggggaagctgacgtc |
| | ctttccatggctgctcgcctgtgttgccac |
| | ctggattctgcgcgggacgtccttctgcta |
| | cgtcccttcggccctcaatccagcggacct |
| | tccttcccgcggcctgctgccggctctgcg |
| | gcctcttccgcgtcttcgccttcgccctca |
| | gacgagtcggatctccctttgggccgcctc |
| | cccgcctggaattcgagctcggtaccttta |
| | agaccaatgacttacaaggcagctgtagat |
| | cttagccactttttaaaagaaaagggggga |
| | ctggaagggctaattcactcccaacgaaga |
| | caagatctgctttttgcttgtactgggtct |
| | ctctggttagaccagatctgagcctgggag |
| | ctctctggctaactagggaacccactgctt |
| | aagcctcaataaagcttgccttgagtgctt |
| | caagtagtgtgtgcccgtctgttgtgtgac |
| | tctggtaactagagatccctcagacccttt |
| | tagtcagtgtggaaaatctctagcagtagt |
| | agttcatgtcatcttattattcagtattta |
| | taacttgcaaagaaatgaatatcagagagt |
| | gagaggaacttgtttattgcagcttataat |
| | ggttacaaataaagcaatagcatcacaaat |
| | ttcacaaataaagcatttttttcactgcat |
| | tctagttgtggtttgtccaaactcatcaat |
| | gtatcttatcatgtctggctctagctatcc |
| | cgcccctaactccgcccatcccgcccctaa |
| | ctccgcccagttccgcccattctccgcccc |
| | atggctgactaattttttttatttatgcag |
| | aggccgaggccgcctcggcctctgagctat |
| | tccagaagtagtgaggaggcttttttggag |
| | gcctagggacgtacccaattcgccctatag |
| | tgagtcgtattacgcgcgctcactggccgt |
| | cgttttacaacgtcgtgactgggaaaaccc |
| | tggcgttacccaacttaatcgccttgcagc |
| | acatccccctttcgccagctggcgtaatag |
| | cgaagaggcccgcaccgatcgcccttccca |
| | acagttgcgcagcctgaatggcgaatggga |
| | cgcgccctgtagcggcgcattaagcgcggc |
| | gggtgtggtggttacgcgcagcgtgaccgc |
| | tacacttgccagcgccctagcgcccgctcc |
| | tttcgctttcttcccttcctttctcgccac |
| | gttcgccggctttccccgtcaagctctaaa |
| | tcgggggctccctttagggttccgatttag |
| | tgctttacggcacctcgaccccaaaaaact |
| | tgattagggtgatggttcacgtagtgggcc |
| | atcgccctgatagacggtttttcgcccttt |
| | gacgttggagtccacgttctttaatagtgg |
| | actcttgttccaaactggaacaacactcaa |
| | ccctatctcggtctattcttttgatttata |
| | agggattttgccgatttcggcctattggtt |
| | aaaaaatgagctgatttaacaaaaatttaa |
| | cgcgaattttaacaaaatattaacgcttac |
| | aatttaggtggcacttttcggggaaatgtg |
| | cgcggaacccctatttgtttatttttctaa |
| | atacattcaaatatgtatccgctcatgaga |
| | caataaccctgataaatgcttcaataatat |
| | tgaaaaaggaagagtatgagtattcaacat |
| | ttccgtgtcgcccttattcccttttttgcg |
| | gcattttgccttcctgtttttgctcaccca |
| | gaaacgctggtgaaagtaaaagatgctgaa |
| | gatcagttgggtgcacgagtgggttacatc |
| | gaactggatctcaacagcggtaagatcctt |
| | gagagttttcgccccgaagaacgttttcca |
| | atgatgagcacttttaaagttctgctatgt |
| | ggcgcggtattatcccgtattgacgccggg |
| | caagagcaactcggtcgccgcatacactat |
| | tctcagaatgacttggttgagtactcacca |
| | gtcacagaaaagcatcttacggatggcatg |
| | acagtaagagaattatgcagtgctgccata |
| | accatgagtgataacactgcggccaactta |
| | cttctgacaacgatcggaggaccgaaggag |
| | ctaaccgcttttttgcacaacatgggggat |
| | catgtaactcgccttgatcgttgggaaccg |
| | gagctgaatgaagccataccaaacgacgag |
| | cgtgacaccacgatgcctgtagcaatggca |
| | acaacgttgcgcaaactattaactggcgaa |
| | ctacttactctagcttcccggcaacaatta |
| | atagactggatggaggcggataaagttgca |
| | ggaccacttctgcgctcggcccttccggct |
| | ggctggtttattgctgataaatctggagcc |
| | ggtgagcgtgggtctcgcggtatcattgca |
| | gcactggggccagatggtaagccctcccgt |
| | atcgtagttatctacacgacggggagtcag |
| | gcaactatggatgaacgaaatagacagatc |
| | gctgagataggtgcctcactgattaagcat |
| | tggtaactgtcagaccaagtttactcatat |
| | atactttagattgatttaaaacttcatttt |
| | taatttaaaaggatctaggtgaagatcctt |
| | tttgataatctcatgaccaaaatcccttaa |
| | cgtgagttttcgttccactgagcgtcagac |
| | cccgtagaaaagatcaaaggatcttcttga |
| | gatcctttttttctgcgcgtaatctgctgc |
| | ttgcaaacaaaaaaaccaccgctaccagcg |
| | gtggtttgtttgccggatcaagagctacca |
| | actctttttccgaaggtaactggcttcagc |
| | agagcgcagataccaaatactgttcttcta |
| | gtgtagccgtagttaggccaccacttcaag |
| | aactctgtagcaccgcctacatacctcgct |
| | ctgctaatcctgttaccagtggctgctgcc |
| | agtggcgataagtcgtgtcttaccgggttg |
| | gactcaagacgatagttaccggataaggcg |
| | cagcggtcgggctgaacggggggttcgtgc |
| | acacagcccagcttggagcgaacgacctac |
| | accgaactgagatacctacagcgtgagcta |
| | tgagaaagcgccacgcttcccgaagggaga |
| | aaggcggacaggtatccggtaagcggcagg |
| | gtcggaacaggagagcgcacgagggagctt |
| | ccagggggaaacgcctggtatctttatagt |
| | cctgtcgggtttcgccacctctgacttgag |
| | cgtcgatttttgtgatgctcgtcagggggg |
| | cggagcctatggaaaaacgccagcaacgcg |
| | gcctttttacggttcctggccttttgctgg |
| | ccttttgctcacatgttctttcctgcgtta |
| | tcccctgattctgtggataaccgtattacc |
| | gcc (SEQ ID NO: 46) |
| OT-ZFHD-079 | atgtagtcttatgcaatactcttgtagtct |
| | tgcaacatggtaacgatgagttagcaacat |
| | gccttacaaggagagaaaaagcaccgtgca |
| | tgccgattggtggaagtaaggtggtacgat |
| | cgtgccttattaggaaggcaacagacgggt |
| | ctgacatggattggacgaaccactgaattg |
| | ccgcattgcagagatattgtatttaagtgc |
| | ctagctcgatacataaacgggtctctctgg |
| | ttagaccagatctgagcctgggagctctct |
| | ggctaactagggaacccactgcttaagcct |
| | caataaagcttgccttgagtgcttcaagta |
| | gtgtgtgcccgtctgttgtgtgactctggt |
| | aactagagatccctcagacccttttagtca |
| | gtgtggaaaatctctagcagtggcgcccga |
| | acagggacttgaaagcgaaagggaaaccag |
| | aggagctctctcgacgcaggactcggcttg |
| | ctgaagcgcgcacggcaagaggcgaggggc |
| | ggcgactggtgagtacgccaaaaattttga |
| | ctagcggaggctagaaggagagagatgggt |
| | gcgagagcgtcagtattaagcgggggagaa |
| | ttagatcgcgatgggaaaaaattcggttaa |
| | ggccagggggaaagaaaaaatataaattaa |
| | aacatatagtatgggcaagcagggagctag |
| | aacgattcgcagttaatcctggcctgttag |
| | aaacatcagaaggctgtagacaaatactgg |
| | gacagctacaaccatcccttcagacaggat |
| | cagaagaacttagatcattatataatacag |
| | tagcaaccctctattgtgtgcatcaaagga |
| | tagagataaaagacaccaaggaagctttag |
| | acaagatagaggaagagcaaaacaaaagta |
| | agaccaccgcacagcaagcggccgctgatc |
| | ttcagacctggaggaggagatatgagggac |
| | aattggagaagtgaattatataaatataaa |
| | gtagtaaaaattgaaccattaggagtagca |
| | cccaccaaggcaaagagaagagtggtgcag |
| | agagaaaaaagagcagtgggaataggagct |
| | ttgttccttgggttcttgggagcagcagga |
| | agcactatgggcgcagcgtcaatgacgctg |
| | acggtacaggccagacaattattgtctggt |
| | atagtgcagcagcagaacaatttgctgagg |
| | gctattgaggcgcaacagcatctgttgcaa |
| | ctcacagtctggggcatcaagcagctccag |
| | gcaagaatcctggctgtggaaagataccta |
| | aaggatcaacagctcctggggatttggggt |
| | tgctctggaaaactcatttgcaccactgct |
| | gtgccttggaatgctagttggagtaataaa |
| | tctctggaacagatttggaatcacacgacc |
| | tggatggagtgggacagagaaattaacaat |
| | tacacaagcttaatacactccttaattgaa |
| | gaatcgcaaaaccagcaagaaaagaatgaa |
| | caagaattattggaattagataaatgggca |
| | agtttgtggaattggtttaacataacaaat |
| | tggctgtggtatataaaattattcataatg |
| | atagtaggaggcttggtaggtttaagaata |
| | gtttttgctgtactttctatagtgaataga |
| | gttaggcagggatattcaccattatcgttt |
| | cagacccacctcccaaccccgaggggaccc |
| | gacaggcccgaaggaatagaagaagaaggt |
| | ggagagagagacagagacagatccattcga |
| | ttagtgaacggatctcgacggtatcgatta |
| | gactgtagcccaggaatatggcagctagat |
| | tgtacacatttagaaggaaaagttatcttg |
| | gtagcagttcatgtagccagtggatatata |
| | gaagcagaagtaattccagcagagacaggg |
| | caagaaacagcatacttcctcttaaaatta |
| | gcaggaagatggccagtaaaaacagtacat |
| | acagacaatggcagcaatttcaccagtact |
| | acagttaaggccgcctgttggtgggcgggg |
| | atcaagcaggaatttggcattccctacaat |
| | ccccaaagtcaaggagtaatagaatctatg |
| | aataaagaattaaagaaaattataggacag |
| | gtaagagatcaggctgaacatcttaagaca |
| | gcagtacaaatggcagtattcatccacaat |
| | tttaaaagaaaaggggggattggggggtac |
| | agtgcaggggaaagaatagtagacataata |
| | gcaacagacatacaaactaaagaattacaa |
| | aaacaaattacaaaaattcaaaattttcgg |
| | gtttattacagggacagcagagatccagtt |
| | tggctgcattgatcaattaattaaggtacc |
| | gagggcctatttcccatgattccttcatat |
| | ttgcatatacgatacaaggctgttagagag |
| | ataattagaattaatttgactgtaaacaca |
| | aagatattagtacaaaatacgtgacgtaga |
| | aagtaataatttcttgggtagtttgcagtt |
| | ttaaaattatgttttaaaatggactatcat |
| | atgcttaccgtaacttgaaagtatttcgat |
| | ttcttggctttatatatcttgtggaaagga |
| | cgaaaCACCAGAGTAACAGTCTGAGgtttt |
| | agagctagaaatagcaagttaaaataaggc |
| | tagtccgttatcaacttgaaaaagtggcac |
| | cgagtcggtgcttttttgaattcgctagct |
| | aggtcttgaaaggagtgggaattggctccg |
| | gtgcccgtcagtcgcgtagatctctagcta |
| | atgatgggcgcacgagtaatgatgggcgga |
| | cgactaatgatgggcgcacgagtaatgatg |
| | ggcgtctagctaatgatgggcgctagagta |
| | atgatgggcggtagactaatgatgggcgct |
| | ccagtaatgatgggcgttctagcTCTAGAG |
| | GGTATATAATGGGGGCCACTAGTCTACTAC |
| | CAGAtAGCTTGGTACCGAGCTCtGATCCAG |
| | CCACCATGGGATCCgacaagaagtacagca |
| | tcggcctggacatcggcaccaactctgtgg |
| | gctgggccgtgatcaccgacgagtacaagg |
| | tgcccagcaagaaattcaaggtgctgggca |
| | acaccgaccggcacagcatcaagaagaacc |
| | tgatcggagccctgctgttcgacagcggcg |
| | aaacagccgaggccacccggctgaagagaa |
| | ccgccagaagaagatacaccagacggaaga |
| | accggatctgctatctgcaagagatcttca |
| | gcaacgagatggccaaggtggacgacagct |
| | tcttccacagactggaagagtccttcctgg |
| | tggaagaggataagaagcacgagcggcacc |
| | ccatcttcggcaacatcgtggacgaggtgg |
| | cctaccacgagaagtaccccaccatctacc |
| | acctgagaaagaaactggtggacagcaccg |
| | acaaggccgacctgcggctgatctatctgg |
| | ccctggcccacatgatcaagttccggggcc |
| | acttcctgatcgagggcgacctgaaccccg |
| | acaacagcgacgtggacaagctgttcatcc |
| | agctggtgcagacctacaaccagctgttcg |
| | aggaaaaccccatcaacgccagcggcgtgg |
| | acgccaaggccatcctgtctgccagactga |
| | gcaagagcagacggctggaaaatctgatcg |
| | cccagctgcccggcgagaagaagaatggcc |
| | tgttcggaaacctgattgccctgagcctgg |
| | gcctgacccccaacttcaagagcaacttcg |
| | acctggccgaggatgccaaactgcagctga |
| | gcaaggacacctacgacgacgacctggaca |
| | acctgctggcccagatcggcgaccagtacg |
| | ccgacctgtttctggccgccaagaacctgt |
| | ccgacgccatcctgctgagcgacatcctga |
| | gagtgaacaccgagatcaccaaggcccccc |
| | tgagcgcctctatgatcaagagatacgacg |
| | agcaccaccaggacctgaccctgctgaaag |
| | ctctcgtgcggcagcagctgcctgagaagt |
| | acaaagagattttcttcgaccagagcaaga |
| | acggctacgccggctacattgacggcggag |
| | ccagccaggaagagttctacaagttcatca |
| | agcccatcctggaaaagatggacggcaccg |
| | aggaactgctcgtgaagctgaacagagagg |
| | acctgctgcggaagcagcggaccttcgaca |
| | acggcagcatcccccaccagatccacctgg |
| | gagagctgcacgccattctgcggcggcagg |
| | aagatttttacccattcctgaaggacaacc |
| | gggaaaagatcgagaagatcctgaccttcc |
| | gcatcccctactacgtgggccctctggcca |
| | ggggaaacagcagattcgcctggatgacca |
| | gaaagagcgaggaaaccatcaccccctgga |
| | acttcgaggaagtggtggacaagggcgctt |
| | ccgcccagagcttcatcgagcggatgacca |
| | acttcgataagaacctgcccaacgagaagg |
| | tgctgcccaagcacagcctgctgtacgagt |
| | acttcaccgtgtataacgagctgaccaaag |
| | tgaaatacgtgaccgagggaatgagaaagc |
| | ccgccttcctgagcggcgagcagaaaaagg |
| | ccatcgtggacctgctgttcaagaccaacc |
| | ggaaagtgaccgtgaagcagctgaaagagg |
| | actacttcaagaaaatcgagtgcttcgact |
| | ccgtggaaatctccggcgtggaagatcggt |
| | tcaacgcctccctgggcacataccacgatc |
| | tgctgaaaattatcaaggacaaggacttcc |
| | tggacaatgaggaaaacgaggacattctgg |
| | aagatatcgtgctgaccctgacactgtttg |
| | aggacagagagatgatcgaggaacggctga |
| | aaacctatgcccacctgttcgacgacaaag |
| | tgatgaagcagctgaagcggcggagataca |
| | ccggctggggcaggctgagccggaagctga |
| | tcaacggcatccgggacaagcagtccggca |
| | agacaatcctggatttcctgaagtccgacg |
| | gcttcgccaacagaaacttcatgcagctga |
| | tccacgacgacagcctgacctttaaagagg |
| | acatccagaaagcccaggtgtccggccagg |
| | gcgatagcctgcacgagcacattgccaatc |
| | tggccggcagccccgccattaagaagggca |
| | tcctgcagacagtgaaggtggtggacgagc |
| | tcgtgaaagtgatgggccggcacaagcccg |
| | agaacatcgtgatcgaaatggccagagaga |
| | accagaccacccagaagggacagaagaaca |
| | gccgcgagagaatgaagcggatcgaagagg |
| | gcatcaaagagctgggcagccagatcctga |
| | aagaacaccccgtggaaaacacccagctgc |
| | agaacgagaagctgtacctgtactacctgc |
| | agaatgggcgggatatgtacgtggaccagg |
| | aactggacatcaaccggctgtccgactacg |
| | atgtggaccatatcgtgcctcagagctttc |
| | tgaaggacgactccatcgacaacaaggtgc |
| | tgaccagaagcgacaagaaccggggcaaga |
| | gcgacaacgtgccctccgaagaggtcgtga |
| | agaagatgaagaactactggcggcagctgc |
| | tgaacgccaagctgattacccagagaaagt |
| | tcgacaatctgaccaaggccgagagaggcg |
| | gcctgagcgaactggataaggccggcttca |
| | tcaagagacagctggtggaaacccggcaga |
| | tcacaaagcacgtggcacagatcctggact |
| | cccggatgaacactaagtacgacgagaatg |
| | acaagctgatccgggaagtgaaagtgatca |
| | ccctgaagtccaagctggtgtccgatttcc |
| | ggaaggatttccagttttacaaagtgcgcg |
| | agatcaacaactaccaccacgcccacgacg |
| | cctacctgaacgccgtcgtgggaaccgccc |
| | tgatcaaaaagtaccctaagctggaaagcg |
| | agttcgtgtacggcgactacaaggtgtacg |
| | acgtgcggaagatgatcgccaagagcgagc |
| | aggaaatcggcaaggctaccgccaagtact |
| | tcttctacagcaacatcatgaactttttca |
| | agaccgagattaccctggccaacggcgaga |
| | tccggaagcggcctctgatcgagacaaacg |
| | gcgaaaccggggagatcgtgtgggataagg |
| | gccgggattttgccaccgtgcggaaagtgc |
| | tgagcatgccccaagtgaatatcgtgaaaa |
| | agaccgaggtgcagacaggcggcttcagca |
| | aagagtctatcctgcccaagaggaacagcg |
| | ataagctgatcgccagaaagaaggactggg |
| | accctaagaagtacggcggcttcgacagcc |
| | ccaccgtggcctattctgtgctggtggtgg |
| | ccaaagtggaaaagggcaagtccaagaaac |
| | tgaagagtgtgaaagagctgctggggatca |
| | ccatcatggaaagaagcagcttcgagaaga |
| | atcccatcgactttctggaagccaagggct |
| | acaaagaagtgaaaaaggacctgatcatca |
| | agctgcctaagtactccctgttcgagctgg |
| | aaaacggccggaagagaatgctggcctctg |
| | ccggcgaactgcagaagggaaacgaactgg |
| | ccctgccctccaaatatgtgaacttcctgt |
| | acctggccagccactatgagaagctgaagg |
| | gctcccccgaggataatgagcagaaacagc |
| | tgtttgtggaacagcacaagcactacctgg |
| | acgagatcatcgagcagatcagcgagttct |
| | ccaagagagtgatcctggccgacgctaatc |
| | tggacaaagtgctgtccgcctacaacaagc |
| | accgggataagcccatcagagagcaggccg |
| | agaatatcatccacctgtttaccctgacca |
| | atctgggagcccctgccgccttcaagtact |
| | ttgacaccaccatcgaccggaagaggtaca |
| | ccagcaccaaagaggtgctggacgccaccc |
| | tgatccaccagagcatcaccggcctgtacg |
| | agacacggatcgacctgtctcagctgggag |
| | gcgacaagcgacctgccgccacaaagaagg |
| | ctggacaggctaagaagaagaaagattaca |
| | aagacgatgacgataagtaaATCGGGTAGC |
| | gtcgacaatcaacctctggattacaaaatt |
| | tgtgaaagattgactggtattcttaactat |
| | gttgctccttttacgctatgtggatacgct |
| | gctttaatgcctttgtatcatgctattgct |
| | tcccgtatggctttcattttctcctccttg |
| | tataaatcctggttgctgtctctttatgag |
| | gagttgtggcccgttgtcaggcaacgtggc |
| | gtggtgtgcactgtgtttgctgacgcaacc |
| | cccactggttggggcattgccaccacctgt |
| | cagctcctttccgggactttcgctttcccc |
| | ctccctattgccacggcggaactcatcgcc |
| | gcctgccttgcccgctgctggacaggggct |
| | cggctgttgggcactgacaattccgtggtg |
| | ttgtcggggaagctgacgtcctttccatgg |
| | ctgctcgcctgtgttgccacctggattctg |
| | cgcgggacgtccttctgctacgtcccttcg |
| | gccctcaatccagcggaccttccttcccgc |
| | ggcctgctgccggctctgcggcctcttccg |
| | cgtcttcgccttcgccctcagacgagtcgg |
| | atctccctttgggccgcctccccgcctgga |
| | attcgagctcggtaccggtgtggaaagtcc |
| | ccaggctccccagcaggcagaagtatgcaa |
| | agcatgcatctcaattagtcagcaaccagg |
| | tgtggaaagtccccaggctccccagcaggc |
| | agaagtatgcaaagcatgcatctcaattag |
| | tcagcaaccatagtcccgcccctaactccg |
| | cccatcccgcccctaactccgcccagttcc |
| | gcccattctccgccccatggctgactaatt |
| | ttttttatttatgcagaggccgaggccgcc |
| | tctgcctctgagctattccagaagtagtga |
| | ggaggcttttttggaggcctaggcttttgc |
| | aaaaagctcccgggagcttgtatatccatt |
| | ttcggatctgatcagcacTTCGAAGCCACC |
| | ATGttgagcaagggcgaggaggacaacatg |
| | gccatcatcaaggagttcatgcgcttcaag |
| | gtgcacatggagggctccgtgaacggccac |
| | gagttcgagatcgagggcgagggcgagggc |
| | cgcccctacgagggcacccagaccgccaag |
| | ctgaaggtgaccaagggcggccccctgccc |
| | ttcgcctgggacatcctgtcccctcagttc |
| | atgtacggctccaaggcctacgtgaagcac |
| | cccgccgacatccccgactacttgaagctg |
| | tccttccccgagggcttcaagtgggagcgc |
| | gtgatgaacttcgaggacggcggcgtggtg |
| | accgtgacccaggactcctccctgcaggac |
| | ggcgagttcatctacaaggtgaagctgcgc |
| | ggcaccaacttcccctccgacggccccgta |
| | atgcagaagaagaccatgggctgggaggcc |
| | tcctccgagcggatgtaccccgaggacggc |
| | gccctgaagggcgagatcaagcagaggctg |
| | aagctgaaggacggcggccactacgacgcc |
| | gaggtcaagaccacctacaaggccaagaag |
| | cccgtgcagctgcccggcgcctacaacgtc |
| | aacatcaagctggacatcacctcccacaac |
| | gaggactacaccatcgtggaacagtacgag |
| | cgcgccgagggccgccactccaccggcggc |
| | atggacgagctgtacaagtaaTTCGAAgcg |
| | aattcgagctcggtacctttaagaccaatg |
| | acttacaaggcagctgtagatcttagccac |
| | tttttaaaagaaaaggggggactggaaggg |
| | ctaattcactcccaacgaagacaagatctg |
| | ctttttgcttgtactgggtctctctggtta |
| | gaccagatctgagcctgggagctctctggc |
| | taactagggaacccactgcttaagcctcaa |
| | taaagcttgccttgagtgcttcaagtagtg |
| | tgtgcccgtctgttgtgtgactctggtaac |
| | tagagatccctcagacccttttagtcagtg |
| | tggaaaatctctagcagtagtagttcatgt |
| | catcttattattcagtatttataacttgca |
| | aagaaatgaatatcagagagtgagaggaac |
| | ttgtttattgcagcttataatggttacaaa |
| | taaagcaatagcatcacaaatttcacaaat |
| | aaagcatttttttcactgcattctagttgt |
| | ggtttgtccaaactcatcaatgtatcttat |
| | catgtctggctctagctatcccgcccctaa |
| | ctccgcccatcccgcccctaactccgccca |
| | gttccgcccattctccgccccatggctgac |
| | taattttttttatttatgcagaggccgagg |
| | ccgcctcggcctctgagctattccagaagt |
| | agtgaggaggcttttttggaggcctaggga |
| | cgtacccaattcgcCCTATAGTGAGTCGTA |
| | TTAcgcgcgctcactggccgtcgttttaca |
| | acgtcgtgactgggaaaaccctggcgttac |
| | ccaacttaatcgccttgcagcacatccccc |
| | tttcgccagctggcgtaatagcgaagaggc |
| | ccgcaccgatcgcccttcccaacagttgcg |
| | cagcctgaatggcgaatgggacgcgccctg |
| | tagcggcgcattaagcgcggcgggtgtggt |
| | ggttacgcgcagcgtgaccgctacacttgc |
| | cagcgccctagcgcccgctcctttcgcttt |
| | cttcccttcctttctcgccacgttcgccgg |
| | ctttccccgtcaagctctaaatcgggggct |
| | ccctttagggttccgatttagtgctttacg |
| | gcacctcgaccccaaaaaacttgattaggg |
| | tgatggttcacgtagtgggccatcgccctg |
| | atagacggatttcgccctttgacgttggag |
| | tccacgttctttaatagtggactcttgttc |
| | caaactggaacaacactcaaccctatctcg |
| | gtctattcttttgatttataagggattttg |
| | ccgatttcggcctattggttaaaaaatgag |
| | ctgatttaacaaaaatttaacgcgaatttt |
| | aacaaaatattaacgcttacaatttaggtg |
| | gcacttttcggggaaatgtgcgcggaaccc |
| | ctatttgtttatttttctaaatacattcaa |
| | atatgtatccgctcatgagacaataaccct |
| | gataaatgcttcaataatattgaaaaagga |
| | agagtatgagtattcaacatttccgtgtcg |
| | cccttattcccttttttgcggcattttgcc |
| | ttcctgtttttgctcacccagaaacgctgg |
| | tgaaagtaaaagatgctgaagatcagttgg |
| | gtgcacgagtgggttacatcgaactggatc |
| | tcaacagcggtaagatccttgagagttttc |
| | gccccgaagaacgttttccaatgatgagca |
| | cttttaaagttctgctatgtggcgcggtat |
| | tatcccgtattgacgccgggcaagagcaac |
| | tcggtcgccgcatacactattctcagaatg |
| | acttggttgagtactcaccagtcacagaaa |
| | agcatcttacggatggcatgacagtaagag |
| | aattatgcagtgctgccataaccatgagtg |
| | ataacactgcggccaacttacttctgacaa |
| | cgatcggaggaccgaaggagctaaccgctt |
| | ttttgcacaacatgggggatcatgtaactc |
| | gccttgatcgttgggaaccggagctgaatg |
| | aagccataccaaacgacgagcgtgacacca |
| | cgatgcctgtagcaatggcaacaacgttgc |
| | gcaaactattaactggcgaactacttactc |
| | tagcttcccggcaacaattaatagactgga |
| | tggaggcggataaagttgcaggaccacttc |
| | tgcgctcggcccttccggctggctggttta |
| | ttgctgataaatctggagccggtgagcgtg |
| | ggtctcgcggtatcattgcagcactggggc |
| | cagatggtaagccctcccgtatcgtagtta |
| | tctacacgacggggagtcaggcaactatgg |
| | atgaacgaaatagacagatcgctgagatag |
| | gtgcctcactgattaagcattggtaactgt |
| | cagaccaagtttactcatatatactttaga |
| | ttgatttaaaacttcatttttaatttaaaa |
| | ggatctaggtgaagatcctttttgataatc |
| | tcatgaccaaaatcccttaacgtgagtttt |
| | cgttccactgagcgtcagaccccgtagaaa |
| | agatcaaaggatcttcttgagatccttttt |
| | ttctgcgcgtaatctgctgcttgcaaacaa |
| | aaaaaccaccgctaccagcggtggtttgtt |
| | tgccggatcaagagctaccaactctttttc |
| | cgaaggtaactggcttcagcagagcgcaga |
| | taccaaatactgttcttctagtgtagccgt |
| | agttaggccaccacttcaagaactctgtag |
| | caccgcctacatacctcgctctgctaatcc |
| | tgttaccagtggctgctgccagtggcgata |
| | agtcgtgtcttaccgggttggactcaagac |
| | gatagttaccggataaggcgcagcggtcgg |
| | gctgaacggggggttcgtgcacacagccca |
| | gcttggagcgaacgacctacaccgaactga |
| | gatacctacagcgtgagctatgagaaagcg |
| | ccacgcttcccgaagggagaaaggcggaca |
| | ggtatccggtaagcggcagggtcggaacag |
| | gagagcgcacgagggagcttccagggggaa |
| | acgcctggtatctttatagtcctgtcgggt |
| | ttcgccacctctgacttgagcgtcgatttt |
| | tgtgatgctcgtcaggggggcggagcctat |
| | ggaaaaacgccagcaacgcggcctttttac |
| | ggttcctggccttttgctggccttttgctc |
| | acatgttctttcctgcgttatcccctgatt |
| | ctgtggataaccgtattaccgcctttgagt |
| | gagctgataccgctcgccgcagccgaacga |
| | ccgagcgcagcgagtcagtgagcgaggaag |
| | cggaagagcgcccaatacgcaaaccgcctc |
| | tccccgcgcgttggccgattcattaatgca |
| | gctggcacgacaggtttcccgactggaaag |
| | cgggcagtgagcgcaacgcaattaatgtga |
| | gttagctcactcattaggcaccccaggctt |
| | tacactttatgcttccggctcgtatgttgt |
| | gtggaattgtgagcggataacaatttcaca |
| | caggaaacagctatgaccatgattacgcca |
| | agcgcgcaattaaccctcactaaagggaac |
| | aaaagctggagctgcaagctta |
| | (SEQ ID NO: 47) |

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

Section headings, the materials, methods, and examples are illustrative only and not intended to be limiting.

1. A modified cell comprising one or more polynucleotides, said one or more polynucleotides comprising:
i) a first nucleic acid sequence that encodes a Cas protein;
ii) a first promoter operably linked to the first nucleic acid sequence;
iii) a second nucleic acid sequence that encodes a drug responsive domain (DRD);
iv) a third nucleic acid sequence that encodes a first guide RNA; and
v) a second promoter operably linked to the third nucleic acid sequence;
wherein the Cas protein is operably linked to the DRD; and
wherein the DRD is derived from a parent protein selected from human carbonic anhydrase 2 (CA2), human DHFR (hDHFR), human estrogen receptor (ER), and human PDE5 (hPDE5).

2. The modified cell of claim 1, wherein the one or more polynucleotides further comprise a second guide RNA and a third promoter that mediates transcription of the second guide RNA, wherein the second guide RNA is different from the first guide RNA.

3. The modified cell of claim 1, wherein the first, second and third nucleic acid sequences and the first and second promoters are components of the same polynucleotide construct.

4. A modified cell comprising:
a. a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and
b. a second polynucleotide comprising a fourth nucleic acid sequence that encodes a Cas protein, said fourth nucleic acid sequence being operably linked to an exogenous inducible first promoter comprising the specific polynucleotide binding site; a fifth nucleic acid sequence that encodes a first guide RNA, said fifth nucleic acid sequence being operably linked to an exogenous second promoter that mediates transcription of the first guide RNA;
wherein the transcription factor activation domain and the transcription factor DNA binding domain interact to form a transcription factor that is able to activate transcription upon binding to the specific polynucleotide binding site.

5. A modified cell comprising:
a. a first polynucleotide comprising a first nucleic acid sequence encoding a transcription factor that is able to bind to a specific polynucleotide binding site and activate transcription, and a second nucleic acid sequence encoding a drug responsive domain (DRD); wherein the transcription factor is operably linked to the DRD; and
b. a second polynucleotide comprising a third nucleic acid sequence encoding a Cas protein, said third nucleic acid sequence being operably linked to an exogenous inducible first promoter comprising the specific polynucleotide binding site; a fourth nucleic acid sequence that encodes a first guide RNA, said fourth nucleic acid sequence being operably linked to an exogenous second promoter that mediates transcription of the first guide RNA.

6. A modified cell comprising one or more polynucleotides, said one or more polynucleotides comprising:
i) a first nucleic acid sequence that encodes a transcription factor activation domain;
ii) a second nucleic acid sequence that encodes a transcription factor DNA binding domain that binds to a specific polynucleotide binding site;
iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD;
iv) a fourth nucleic acid sequence that encodes a Cas protein, said fourth nucleic acid sequence being operably linked to an exogenous inducible first promoter comprising the specific polynucleotide binding site;
v) a fifth nucleic acid sequence that encodes a first guide RNA, said fifth nucleic acid sequence being operably linked to an exogenous second promoter that mediates transcription of the first guide RNA.

7. The modified cell of claim 4, further comprising a second guide RNA and a third promoter that mediates transcription of the second guide RNA, wherein the second guide RNA is different from the first guide RNA.

8. The modified cell of claim 4 wherein:
i) the transcription factor DNA binding domain is derived from a parent protein selected from the group ZFHD1 and TAL;
ii) the transcription factor activation domain is derived from a parent protein, wherein said parent protein is p65; and/or
iii) the DRD is derived from a parent protein selected from human carbonic anhydrase 2 (CA2), human DHFR, ecDHFR, human estrogen receptor (ER), FKBP, human protein FKBP, and human PDE5.

9. The modified cell of claim 1, wherein the DRD is derived from a parent protein selected from human carbonic anhydrase 2 (CA2; SEQ ID NO: 5), human DHFR (SEQ ID NO: 2), ecDHFR (SEQ ID NO: 1), human estrogen receptor (ER; SEQ ID NO: 6), FKBP, human protein FKBP (SEQ ID NO: 6), and human PDE5 (SEQ ID NO: 7); and further comprises one or more mutations relative to the parent protein.

10. The modified cell of claim 1, wherein the first promoter is a Pol II promoter, wherein, optionally, the Poll II promoter is selected form CK8e, EFS, and PGK.

11. The modified cell of claim 1, wherein the second promoter is a Pol III promoter, wherein, optionally, the Poll III promoter is selected form H1, U6 and 7SK.

12. The modified cell of claim 1, wherein the nucleic acid sequence encoding the Cas protein is derived from a parent Cas9 or a parent Cas12a sequence.

13. The modified cell of claim 12, wherein the parent Cas9 protein is selected from Streptococcus pyogenes Cas 9 (SpCas9), Staphylococcus aureus (SaCas9), and Neisseria meningitidis Cas9 (NmeCas9).

14. The modified cell of claim 1, wherein the DRD is responsive to or interacts with a ligand selected from Acetazolamide (ACZ), Bazedoxifene (BZD), Celecoxib, Methotrexate (MTX), Raloxifene, Shield-1, Sildenafil, Tadalafil, Trimethoprim (TMP), and Vardenafil.

15. The modified cell of claim 1, wherein the cell is an immune cell, a stem cell, a liver cell, a blood cell, a pancreatic cell, a neuronal cell, an ocular cell, a muscle cell, or a bone cell.

16. A nucleic acid molecule comprising:
i) a first nucleic acid sequence that encodes a Cas protein;
ii) a first promoter that mediates transcription of the nucleic acid sequence encoding the Cas protein;
iii) a second nucleic acid sequence that encodes a drug responsive domain (DRD);
iv) a third nucleic acid sequence that encodes a guide RNA; and
v) a second promoter that mediates transcription of the guide RNA;
wherein the Cas protein is operably linked to the DRD; and
wherein the DRD is derived from a parent protein selected from human carbonic anhydrase 2 (CA2), human DHFR (hDHFR), human estrogen receptor (ER), and human PDE5 (hPDE5).

17. A nucleic acid molecule comprising:
i) a first nucleic acid sequence that encodes a Cas protein, said first nucleic acid sequence being operably linked to an exogenous inducible first promoter comprising a specific polynucleotide binding site for a transcription factor;
ii) a second nucleic acid sequence that encodes a first guide RNA, said second nucleic acid sequence being operably linked to an exogenous second promoter that mediates transcription of the first guide RNA.

18. The nucleic acid molecule of claim 17, further comprising a nucleic acid sequence that encodes a second guide RNA and a third promoter that mediates transcription of the second guide RNA, wherein the second guide RNA is different from the first guide RNA.

19. A nucleic acid molecule comprising:
i) a first nucleic acid sequence that encodes a transcription factor activation domain;
ii) a second nucleic acid sequence that encodes a transcription factor DNA binding domain that binds to a specific polynucleotide binding site;
iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD;
iv) a fourth nucleic acid sequence that encodes a Cas protein, said fourth nucleic acid sequence being operably linked to an exogenous inducible first promoter comprising the specific polynucleotide binding site; and
v) a fifth nucleic acid sequence that encodes a first guide RNA, said fifth nucleic acid sequence being operably linked to an exogenous second promoter that mediates transcription of the first guide RNA.

20. The nucleic acid molecule of claim 19, further comprising a nucleic acid sequence that encodes a second guide RNA and a third promoter that mediates transcription of the second guide RNA, wherein the second guide RNA is different from the first guide RNA.

21. A vector comprising the nucleic acid molecule according to claim 16.

22. The vector according to claim 21, wherein the vector is a plasmid or a viral vector.

23. The vector according to claim 22, wherein the viral vector is derived from an adenovirus, adeno-associated virus (AAV), alphavirus, flavivirus, herpes virus, measles virus, rhabdovirus, retrovirus, lentivirus, Newcastle disease virus (NDV), poxvirus, and picornavirus.

24. The vector according to claim 22, wherein the viral vector is selected from the group consisting of a lentivirus vector, a gamma retrovirus vector, adeno-associated virus (AAV) vector, adenovirus vector, and a herpes virus vector.

25. A method of producing a modified cell, said method comprising introducing into a cell a nucleic acid molecule comprising:
i) a first nucleic acid sequence that encodes a Cas protein;
ii) a first promoter that mediates transcription of the nucleic acid sequence encoding the Cas protein;
iii) a second nucleic acid sequence that encodes a drug responsive domain (DRD);
iv) a third nucleic acid sequence that encodes a guide RNA; and
v) a second promoter that mediates transcription of the guide RNA;
wherein the Cas protein is operably linked to the DRD; and
wherein the DRD is derived from a parent protein selected from human carbonic anhydrase 2 (CA2), human DHFR (hDHFR), human estrogen receptor (ER), and human PDE5 (hPDE5).

26. A method of producing a modified cell, said method comprising introducing into a cell a first nucleic acid molecule and a second nucleic acid molecule, wherein the first nucleic acid molecule comprises:
i) a first nucleic acid sequence that encodes a transcription factor activation domain;
ii) a second nucleic acid sequence that encodes a transcription factor DNA binding domain that binds to a specific polynucleotide binding site;
iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and
wherein the second nucleic acid molecule comprises:
i) a fourth nucleic acid sequence that encodes a Cas protein, said fourth nucleic acid sequence being operably linked to an exogenous inducible first promoter comprising the specific polynucleotide binding site; and
ii) a fifth nucleic acid sequence that encodes a guide RNA, said fifth nucleic acid sequence being operably linked to an exogenous second promoter that mediates transcription of the guide RNA.

27. A method of producing a modified cell, said method comprising introducing into a cell a nucleic acid molecule comprising:
i) a first nucleic acid sequence that encodes a transcription factor activation domain;
ii) a second nucleic acid sequence that encodes a transcription factor DNA binding domain that binds to a specific polynucleotide binding site;
iii) a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD;
iv) a fourth nucleic acid sequence that encodes a Cas protein, said fourth nucleic acid sequence being operably linked to an exogenous inducible first promoter comprising the specific polynucleotide binding site;
v) a fifth nucleic acid sequence that encodes a guide RNA, said fifth nucleic acid sequence being operably linked to an exogenous second promoter that mediates transcription of the guide RNA.

28. The method according to claim 25, wherein the nucleic acid molecule or nucleic acid molecules are introduced into the cell by one or more of a plasmid or one or more of a viral vector.

29. A method of producing a modified cell, said method comprising introducing into a cell a first nucleic acid molecule and a second nucleic acid molecule, wherein the first nucleic acid molecule comprises:
i) a first nucleic acid sequence that encodes a Cas protein;
ii) a first promoter that mediates transcription of the nucleic acid sequence encoding the Cas protein; and
iii) a second nucleic acid sequence that encodes a drug responsive domain (DRD); and
wherein the second nucleic acid molecule comprises:
i) a first nucleic acid sequence that encodes a first guide RNA operably linked to a first promoter that mediates transcription of the first guide RNA; and
ii) a second nucleic acid sequence that encodes a second guide RNA operably linked to a second promoter that mediates transcription of the second guide RNA;
wherein the Cas protein is operably linked to the DRD; and
wherein the DRD is derived from a parent protein selected from human carbonic anhydrase 2 (CA2), human DHFR (hDHFR), human estrogen receptor (ER), and human PDE5 (hPDE5); and
wherein the first nucleic acid molecule is introduced into the cell on a first plasmid or viral vector and the second nucleic acid molecule is introduced into the cell on a second plasmid or viral vector.

30. The method according to claim 28, wherein the viral vector is derived from an adenovirus, adeno-associated virus (AAV), alphavirus, flavivirus, herpes virus, measles virus, rhabdovirus, retrovirus, lentivirus, Newcastle disease virus (NDV), poxvirus, and picornavirus.

31. The method according to claim 28, wherein the viral vector is selected from the group consisting of a lentivirus vector, a gamma retrovirus vector, adeno-associated virus (AAV) vector, adenovirus vector, and a herpes virus vector.

32. The method according to claim 25, wherein the nucleic acid molecule or nucleic acid molecules are introduced into the cell by a non-viral delivery method.

33. The method of claim 25, wherein the cell is an immune cell, a stem cell, a liver cell, a blood cell, a pancreatic cell, a neuronal cell, an ocular cell, a muscle cell, or a bone cell.

34. A method for introducing a modified cell into a subject in need of disease treatment or prevention, the method comprising:
a. providing a population of cells;
b. introducing at least one nucleic acid molecule of claim 16 into at least one cell in the population of cells; and
c. delivering the cell into the subject.

35. A method for introducing a modified cell into a subject in need of disease treatment or prevention, the method comprising:
a. providing a population of cells;
b. introducing at least one nucleic acid molecule of claim 17 into at least one cell in the population of cells;
c. introducing at least one of a different nucleic acid molecule into the at least one cell, wherein the at least one different nucleic acid molecule comprises a first nucleic acid sequence that encodes a transcription factor activation domain; a second nucleic acid sequence that encodes a transcription factor DNA binding domain that binds to the specific polynucleotide binding site of the nucleic acid molecule of claim 17; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and
d. delivering the cell into the subject.

36. A method for treating or preventing a disease in a subject in need thereof, the method comprising:
a. providing a population of cells comprising at least one gene that requires gene editing;
b. introducing at least one nucleic acid molecule of claim 16 into at least one cell in the population of cells;
c. delivering the cell into the subject; and
d. administering a ligand to the subject that stabilizes the DRD sufficiently to enable expression of the Cas protein in an amount sufficient to cleave a target DNA site;
wherein expression of the Cas protein is regulated by the presence of ligand in the subject, and the amount and/or duration of ligand administration is sufficient to produce a therapeutically effective amount of the Cas protein, and
wherein the first guide RNA comprises a nucleic acid sequence that directs the Cas9 protein to edit the gene.

37. A method for treating or preventing a disease in a subject in need thereof, the method comprising:
a. providing a population of cells comprising at least one gene that requires gene editing;
b. introducing at least one nucleic acid molecule of claim 17 into at least one cell in the population of cells;
c. introducing at least one of a different nucleic acid molecule into the at least one cell, wherein the at least one different nucleic acid molecule comprises a first nucleic acid sequence that encodes a transcription factor activation domain; a second nucleic acid sequence that encodes a transcription factor DNA binding domain that binds to the specific polynucleotide binding site of the nucleic acid molecule of claim 17; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD;
d. delivering the cell into the subject; and
e. administering a ligand to the subject that stabilizes the DRD sufficiently to enable expression of the transcription factor activation domain and the transcription factor DNA binding domain in an amount sufficient to form a transcription factor that binds to the specific polynucleotide binding site and enables expression of the Cas protein in the cell;
wherein expression of the Cas protein is regulated by the presence of ligand in the subject, and the amount and/or duration of ligand administration is sufficient to produce a therapeutically effective amount of the Cas protein, and wherein the first guide RNA comprises a nucleic acid sequence that directs the Cas9 protein to edit the gene.

38. A method for genetically modifying one or more cells in a subject in need of disease treatment or prevention, the method comprising introducing at least one nucleic acid molecule of claim 16 into at least one cell of the subject.

39. The method of claim 38, further comprising administering a ligand to the subject that stabilizes the DRD sufficiently to enable expression of the Cas protein in an amount sufficient to cleave a target DNA site;
wherein expression of the Cas protein is regulated by the presence of ligand in the subject, and the amount and/or duration of ligand administration is sufficient to produce a therapeutically effective amount of the Cas protein.

40. A method for genetically modifying one or more cells in a subject in need of disease treatment or prevention, the method comprising:
a. introducing at least one nucleic acid molecule of claim 17 into at least one cell of the subject; and
b. introducing at least one of a different nucleic acid molecule into the at least one cell, wherein the at least one different nucleic acid molecule comprises a first nucleic acid sequence that encodes a transcription factor activation domain; a second nucleic acid sequence that encodes a transcription factor DNA binding domain that binds to the specific polynucleotide binding site of the nucleic acid molecule of claim 17; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD.

41. The method of claim 40, further comprising administering a ligand to the subject that stabilizes the DRD sufficiently to enable expression of the transcription factor activation domain and/or the transcription factor DNA binding domain in an amount sufficient to form a transcription factor that binds to the specific polynucleotide binding site and enables expression of the Cas protein in the cell;
wherein expression of the Cas protein is regulated by the presence of ligand in the subject, and the amount and/or duration of ligand administration is sufficient to produce a therapeutically effective amount of the Cas protein.

42. The method according to claim 34, wherein the nucleic acid molecule or nucleic acid molecules are introduced into the cell by one or more of a plasmid or one or more of a viral vector.

43. The method according to claim 42, wherein the viral vector is derived from an adenovirus, adeno-associated virus (AAV), alphavirus, flavivirus, herpes virus, measles virus, rhabdovirus, retrovirus, lentivirus, Newcastle disease virus (NDV), poxvirus, and picornavirus.

44. The method according to claim 43, wherein the viral vector is selected from the group consisting of a lentivirus vector, a gamma retrovirus vector, adeno-associated virus (AAV) vector, adenovirus vector, and a herpes virus vector.

45. The method according to claim 34, wherein the nucleic acid molecule or nucleic acid molecules are introduced into the cell by a non-viral delivery method.
